SEQUENCE LISTING (388 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 3 - protein, 36 aa, Homo sapiens
MAAEPVEDNCINFVAMKFIDNTLYFIAEDDENLESD

SEQ ID NO: 4 - protein, 157 aa, synthetic construct
YFGKLESKLSVIRNLNDQVLFIDQGNRPLFEDMTDSDSRDNAPRTIFIISKYSDSLARGLAVTISVKSEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 5 - protein, 157 aa, synthetic construct
YFGKLESKLSVIRNLNDQVLFIDQGNRPLFEDMTDSDCRDNAPRTIFIISKYSDSLARGLAVTISVKSEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 6 - protein, 157 aa, synthetic construct
YFGKLESKLSVIRNLNDQVLFIDQGNRPLFEDMTDSDSRDNAPRTIFIISKYSDSLARGLAVTISVKCEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 7 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 8 - protein, 157 aa, synthetic construct
YFGKLESKLSVIRNLNDQVLFIDQGNRPLFEDMTDSDSRDNAPRTIFIISKYSDSLARGLAVTISVKSEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 9 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 10 - protein, 157 aa, synthetic construct
YFGKLESKLSVIRNLNDQVLFIDQGNRPLFEDMTDSDSRDNAPRTIFIISKYSDSLARGLAVTISVKCEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 11 - protein, 157 aa, synthetic construct
YFGKLESKLSVIRNLNDQVLFIDQGNRPLFEDMTDSDSRDNAPRTIFIISKYSDSLARGLAVTISVKCEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 12 - protein, 157 aa, synthetic construct
YFGKLESKLSVIRNLNDQVLFIDQGNRPLFEDMTDSDCRDNAPRTIFIISKYSDSLARGLAVTISVKSEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 13 - protein, 157 aa, synthetic construct
YFGKLESKLSVIRNLNDQVLFIDQGNRPLFEDMTDSDCRDNAPRTIFIISKYSDSLARGLAVTISVKSEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 14 - protein, 157 aa, synthetic construct
YFGKLESKLSVIRNLNDQVLFIDQGNRPLFEDMTDSDCRDNAPRTIFIISKYSDSLARGLAVTISVKCEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 15 - protein, 157 aa, synthetic construct
YFGKLESKLSVIRNLNDQVLFIDQGNRPLFEDMTDSDSRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 16 - protein, 157 aa, synthetic construct
YFGKLESKLSVIRNLNDQVLFIDQGNRPLFEDMTDSDCRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 17 - protein, 157 aa, synthetic construct
YFGKLESKLSVIRNLNDQVLFIDQGNRPLFEDMTDSDCRDNAPRTIFIISKYSDSLARGLAVTISVKCEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 18 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 19 - protein, 157 aa, synthetic construct
YFGKLESKLSVIRNLNDQVLFIDQGNRPLFEDMTDSDSRDNAPRTIFIISKYSDSLARGLAVTISVKGEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 20 - protein, 157 aa, synthetic construct
YFGKLESKLSVIRNLNDQVLFIDQGNRPLFEDMTDSDSRDNAPRTIFIISKYSDSLARGLAVTISVKAEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 21 - protein, 157 aa, synthetic construct
YFGKLESKLSVIRNLNDQVLFIDQGNRPLFEDMTDSDSRDNAPRTIFIISKYSDSLARGLAVTISVKVEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 22 - protein, 157 aa, synthetic construct
YFGKLESKLSVIRNLNDQVLFIDQGNRPLFEDMTDSDSRDNAPRTIFIISKYSDSLARGLAVTISVKDEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 23 - protein, 157 aa, synthetic construct
YFGKLESKLSVIRNLNDQVLFIDQGNRPLFEDMTDSDSRDNAPRTIFIISKYSDSLARGLAVTISVKEEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 24 - protein, 157 aa, synthetic construct
YFGKLESKLSVIRNLNDQVLFIDQGNRPLFEDMTDSDSRDNAPRTIFIISKYSDSLARGLAVTISVKNEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 25 - protein, 157 aa, synthetic construct
YFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 26 - protein, 157 aa, synthetic construct
YFGKLESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 27 - protein, 157 aa, synthetic construct
RFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 28 - protein, 157 aa, synthetic construct
RFGKLESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRNVPGHKYKMQFESSSYEGYFLACEKERDLFKLILKKEDELGDRSIMFTVQNED

SEQ ID NO: 29 - protein, 157 aa, synthetic construct
YFGKLESQLSVIRNLNDQVLFIDQGNRPLFEDMTDSDCRDNAPRTIFIISTYKDKQPRTKAVTISVKCEKISTLSCENKIISFKEMNPPDNIKDTKSDIIFFQRRVPGHHNKMQFESSSYEGYFLACEKERDLFKLILKKEDELGDRSIMFTVQKED

SEQ ID NO: 30 - protein, 157 aa, synthetic construct
YFGKLESRLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 31 - protein, 157 aa, synthetic construct
YFGKLESRLSVIRNLNDQVLFIDQGNRPLFEDMTDSDCRDNAPRTIFIISDYKDKQPRAXAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 32 - protein, 157 aa, synthetic construct
YFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHNNKMQFESSSYEGYFLACEKERDLFKLILKKEDELGDRSIMFTTQNED

SEQ ID NO: 33 - protein, 157 aa, synthetic construct
YFGKIESKLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRKVPGHQHKMQFESSSYEGYFLACEKERDLFKLILKKEDELGDRSIMFTVQKED

SEQ ID NO: 34 - protein, 157 aa, synthetic construct
YFGKIESKLSVIRNLNDQVLFIDQGNRPLFEDMTDSDCRDNAPRTIFIISTYKDRQPRGKAVTISVKCEKISTLSCENKIISFKEMNPPDNIKDTKSDIIFFERDVPGHHHKMQFESSSYEGYFLACEKERDLFKLILKKEDELGDRSIMFTIQNED

SEQ ID NO: 35 - protein, 157 aa, synthetic construct
YFGKIESKLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHKHKMQFESSSYEGYFLACEKERDLFKLILKKEDELGDRSIMFTTQHED

SEQ ID NO: 36 - protein, 157 aa, synthetic construct
YFGKIESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRRVPGHHHKMQFESSSYEGYFLACEKERDLFKLILKKEDELGDRSIMFTIQKED

SEQ ID NO: 37 - protein, 157 aa, synthetic construct
YFGKIESRLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHDYKMQFESSSYEGYFLACEKERDLFKLILKKEDELGDRSIMFTIQKED

SEQ ID NO: 38 - protein, 157 aa, synthetic construct
YFGKYESRLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHEHKMQFESSSYEGYFLACEKERDLFKLILKKEDELGDRSIMFTVQKED

SEQ ID NO: 39 - protein, 157 aa, synthetic construct
HFGKYESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHHNKMQFESSSYEGYFLACEKERDLFKLILKKEDELGDRSIMFTVQKED

SEQ ID NO: 40 - protein, 157 aa, synthetic construct
RFGKLESKLSVIRNLNDQVLFIDQGNRPLFEDMTDSDCRDNAPRTIFIISTYRDSQPRAKAVTISVKCEKISTLSCENKIISFKEMNPPDNIKDTKSDIIFFQRDVPGHQHKMQFESSSYEGYFLACEKERDLFKLILKKEDELGDRSIMFTAQKED

SEQ ID NO: 41 - protein, 157 aa, synthetic construct
RFGKLESRLSVIRNLNDQVLFIDQGNRPLFEDMTDSDCRDNAPRTIFIISDYRDSQPRGRAVTISVKCEKISTLSCENKIISFKEMNPPDNIKDTKSDIIFFKRNVPGHKYKMQFESSSYEGYFLACEKERDLFKLILKKEDELGDRSIMFTVQHED

SEQ ID NO: 42 - protein, 157 aa, synthetic construct
RFGKLESRLSVIRNLNDQVLFIDQGNRPLFEDMTDSDCRDNAPRTIFIISNYRDSQPRGQAVTISVKCEKISTLSCENKIISFKEMNPPDNIKDTKSDIIFFKRRVPGHNHKMQFESSSYEGYFLACEKERDLFKLILKKEDELGDRSIMFTVQKED

SEQ ID NO: 43 - protein, 157 aa, synthetic construct
RFGKLESRLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 44 - protein, 157 aa, synthetic construct
RFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFERNVPGHKYKMQFESSSYEGYFLACEKERDLFKLILKKEDELGDRSIMFTVQNED

SEQ ID NO: 45 - protein, 157 aa, synthetic construct
RFGKLESRLSVIRNLNDQVLFIDQGNRPLFEDMTDSDCRDNAPRTIFIISTYRDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQXED

SEQ ID NO: 46 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRNVPGHHDKMQFESSSYEGYFLACEKERDLFKLILKKEDELGDRSIMFTVQHED

SEQ ID NO: 47 - protein, 157 aa, synthetic construct
RFGKLESRLSVIRNLNDQVLFIDQGNRPLFEDMTDSDCRDNAPRTIFIISTYKDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQKED

SEQ ID NO: 48 - protein, 157 aa, synthetic construct
RFGKYESKLSVIRNLNDQVLFIDQGNRPLFEDMTDSDCRDNAPRTIFIISTYKDSQPRTKAVTISVKCEKISTLSCDNKIISFKEMNPPDNIKDTKSDIIFFQRDVPGHKHKMQFESSSYEGYFLACEKERDLFKLILKKEDELGDRSIMFTVQNED

SEQ ID NO: 49 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRKVPGHNHKMQFESSSYEGYFLACEKERDLFKLILKKEDELGDRSIMFTVQKED

SEQ ID NO: 50 - protein, 157 aa, synthetic construct
YFGKLESKLSVIRNLNDQVLFIDQGNRPLFEDMTDSDCRDNAPRTIFIISEYKDSELRGRAVTISVKCEKISTLSCENKIISFKEMNPPDNIKDTKSDIIFFPRAVPGHNRKVQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISKYKDSAGRGLAVTISVKCEKISTLSCENKIISFKEMNPPDNIKDTKSDIIFFERDVPGHSNKVQFESSSYEGYFLACEKERDLFKLILKKEDELGDRSIMFTVQNED

SEQ ID NO: 52 - protein, 157 aa, synthetic construct
YFGKLESKLSVIRNLNDQVLFIDQGNRPLFEDMTDSDCRDNAPRTIFIISKYGDSAARGLAVTISVKCEKISTLSCENKIISFKEMNPPDNIKDTKSDIIFFQRSVPGHKRKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISKYGDSRGRGLAVTISVKCEKISTLSCENKIISFKEMNPPDNIKDTKSDIIFFERDVPGHNSKRQFESSSYEGYFLACEKERDLFKLILKKEDELGDRSIMFTVQNED

SEQ ID NO: 54 - protein, 157 aa, synthetic construct
YFGKLESKLSVIRNLNDQVLFIDQGNRPLFEDMTDSDCRDNAPRTIFIISKYGDSVPRGLAVTISVKCEKISTLSCENKIISFKEMNPPDNIKDTKSDIIFFARAVPGHSRKTQFESSSYEGYFLACEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDCRDNAPRTIFIISKYSDSGARGLAVTISVKCEKISTLSCENKIISFKEMNPPDNIKDTKSDIIFFARAVPGHGRKTQFESSSYEGYFLACEKERDLFKLILKKEDELGDRSIMFTVQNED

SEQ ID NO: 56 - protein, 157 aa, synthetic construct
YFGKLESKLSVIRNLNDQVLFIDQGNRPLFEDMTDSDCRDNAPRTIFIISKYSDSKARGMAVTISVKCEKISTLSCENKIISFKEMNPPDNIKDTKSDIIFFARDVPGHSSKRQFESSSYEGYFLACEKERDLFKLILKKEDELGDRSIMFTVQNED

SEQ ID NO: 57 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 58 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFQRNVPGHGRKMQFESSSYEGYFLACEKERDLFKLILKKEDELGDRSIMFTVQNED

SEQ ID NO: 59 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARSVPGHGRKTQFESSSYEGYFLACEKERDLFKLILKKEDELGDRSIMFTVQNED

SEQ ID NO: 60 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARDVPGHSGKRQFESSSYEGYFLACEKERDLFKLILKKEDELGDRSIMFTVQNED

SEQ ID NO: 61 - protein, 157 aa, synthetic construct
YFGKLESKLSVIRNLNDQVLFIDQGNRPLFEDMTDSDCRDNAPRTIFIISKYTDSRPRGLAVTISVKCEKISTLSCENKIISFKEMNPPDNIKDTKSDIIFFERDVPGHSSKKQFESSSYEGYFLACEKERDLFKLILKKEDELGDRSIMFTVQNED

SEQ ID NO: 62 - protein, 157 aa, synthetic construct
YFGKLESKLSVIRNLNDQVLFIDQGNRPLFEDMTDSDCRDNAPRTIFIISKYTDSRARGMAVTISVKCEKISTLSCENKIISFKEMNPPDNIKDTKSDIIFFERDVPGHNDKRQFESSSYEGYFLACEKERDLFKLILKKEDELGDRSIMFTVQNED

SEQ ID NO: 63 - protein, 157 aa, synthetic construct
YFGKLESKLSVIRNLNDQVLFIDQGNRPLFEDMTDSDCRDNAPRTIFIISRYKDSGKRGLAVTISVKCEKISTLSCENKIISFKEMNPPDNIKDTKSDIIFFRRSVPGHSRKVQFESSSYEGYFLACEKERDLFKLILKKEDELGDRSIMFTVQNED

SEQ ID NO: 64 - protein, 157 aa, synthetic construct
YFGKLESKLSVIRNLNDQVLFIDQGNRPLFEDMTDSDCRDNAPRTIFIISKYGDSGARGLAVTISVKCEKISTLSCENKIISFKEMNPPDNIKDTKSDIIFFERDVPGHSGKVQFESSSYEGYFLACEKERDLFKLILKKEDELGDRSIMFTVQNED

SEQ ID NO: 65 - protein, 157 aa, synthetic construct
YFGKLESKLSVIRNLNDQVLFIDQGNRPLFEDMTDSDCRDNAPRTIFIISKYGDSRPRGMAVTISVKCEKISTLSCENKIISFKEMNPPDNIKDTKSDIIFFQRAVPGHNRKMQFESSSYEGYFLACEKERDLFKLILKKEDELGDRSIMFTVQNED

SEQ ID NO: 66 - protein, 157 aa, synthetic construct
  VARIANT 1: note = X at pos. 1 is Y, R or H
  VARIANT 5: note = X at pos. 5 is L, H, I or Y
  VARIANT 8: note = X at pos. 8 is K, Q or R
  VARIANT 38: note = X at pos. 38 is C or S
  VARIANT 51: note = X at pos. 51 is M, T, K, D, N, E or R
  VARIANT 53: note = X at pos. 53 is K, R, G, S or T
  VARIANT 55: note = X at pos. 55 is S, K or R
  VARIANT 56: note = X at pos. 56 is Q, E, A, R, V, G, K, L or R
  VARIANT 57: note = X at pos. 57 is P, L, G, A or K
  VARIANT 59: note = X at pos. 59 is G, A or T
  VARIANT 60: note = X at pos. 60 is M, K, Q, R or L
  VARIANT 68: note = X at pos. 68 is C, S, G, A, V, D, E or N
  VARIANT 76: note = X at pos. 76 is C or S
  VARIANT 77: note = X at pos. 77 is E or D
  VARIANT 103: note = X at pos. 103 is Q, E, K, P, A or R
  VARIANT 105: note = X at pos. 105 is S, D, N, R, K or A
  VARIANT 110: note = X at pos. 110 is D, K, H, N, Q, E, S or G
  VARIANT 111: note = X at pos. 111 is N, H, Y, D, R, S or G
  VARIANT 113: note = X at pos. 113 is M, V, R, T or K
  VARIANT 127: note = X at pos. 127 is C or S
  VARIANT 153: note = X at pos. 153 is V, I, T or A
  VARIANT 155: note = X at pos. 155 is N, K or H
XFGKXESXLSVIRNLNDQVLFIDQGNRPLFEDMTDSDXRDNAPRTIFIISXYXDXXXRXXAVTISVKXEKISTLSXXNKIISFKEMNPPDNIKDTKSDIIFFXRXVPGHXXKXQFESSSYEGYFLAXEKERDLFKLILKKEDELGDRSIMFTXQXED

SEQ ID NO: 67 - DNA, 471 bp, synthetic construct
  source 1..471: mol_type = other DNA
tatttcggcaaactggaaagcaagctcagcgtgatccggaacctgaacgaccaagtgctgttcatcgaccagggcaaccggcctctgtttgaggacatgacagacagcgactgcagagataatgcccctcggaccatcttcatcatctctacctacaaggattctcagcctagaggcaaggccgtgaccatctctgtcaagtgcgagaagatcagcacactgagctgcgagaacaagatcattagcttcaaagaaatgaacccccccgacaatatcaaggacaccaagtccgacatcatctttttccagagagatgtgccaggccacaagcacaaaatgcagttcgagagcagctcctacgagggctacttcctggcttgtgaaaaggaaagagacctgttcaagctgatcctgaaaaaggaagatgagctgggagatagaagcatcatgttcaccgtgcagaacgaggac

SEQ ID NO: 68 - DNA, 474 bp, synthetic construct
  source 1..474: mol_type = other DNA
tacttcggcaaactggagtccaagctgagcgtgatcaggaatctgaacgatcaggtgctcttcatcgatcagggcaatagacctctgtttgaggacatgactgactccgactgtcgggacaacgccccccgaacaatctttattatcagcacctacaaggacagccagcccagggggaaggccgtgactatctccgtgaagtgcgaaaagatcagcaccctgtcatgtgagaacaagattatctcctttaaggagatgaacccccccgataacattaaggacaccaagagcgacatcatcttcttccagcgcgacgtgccaggacacaagcacaaaatgcagtttgagagcagcagctacgaagggtatttcctggcctgtgagaaggaaagagatctgttcaagctgatcctgaagaaggaggacgagctgggcgaccggagcatcatgtttaccgtgcagaacgaggactga

SEQ ID NO: 69 - DNA, 471 bp, synthetic construct
  source 1..471: mol_type = other DNA
tacttcggcaagctggaaagcaagctgagcgtgatccggaacctgaacgaccaagtgctgttcatcgaccagggaaatagacctctgttcgaggacatgaccgacagcgactgcagagataatgcccctagaacaatcttcatcatcagcacatacaaagataagcagcctcgggctaaggccgtgaccatcagcgtcaagtgcgagaaaatctctaccctgtcttgtgaaaacaagatcatctccttcaaggaaatgaacccccccgacaacatcaaggacaccaagtccgacatcattttcttccagcgggacgtgccaggccacaagcacaaaatgcagttcgagagcagctcttatgagggctactttctggcctgcgagaaggaaagagatctgtttaagctcatcctgaagaaagaagatgagctgggcgacagaagcatcatgttcaccatccagaacgaggat

SEQ ID NO: 70 - DNA, 474 bp, synthetic construct
  source 1..474: mol_type = other DNA
tactttggcaagctggagtccaaactgtccgtgatccgcaacctgaatgatcaggtgctgttcatcgaccagggcaaccgccccctgttcgaggacatgaccgatagcgactgcagggataacgcccctcggactatctttatcatcagcacatacaaggacaaacagcccagagccaaggccgtgaccatcagcgtcaagtgtgagaagattagcactctgtcctgtgagaacaagatcattagctttaaggaaatgaacccacctgataatatcaaggacaccaagagcgacattatcttctttcagagagatgtgcccggccacaagcacaagatgcagttcgagagctctagctacgagggctacttcctggcttgcgaaaaggagagagacctgtttaaactcattctgaagaaggaggacgaactgggcgatagatccatcatgttcaccatccagaacgaggattga

SEQ ID NO: 71 - DNA, 471 bp, synthetic construct
  source 1..471: mol_type = other DNA
cggttcggcaaactggaatctaagctgtctgtgatccggaacctgaacgaccaagtgctgttcatcgaccagggcaatcgccccctgtttgaggatatgaccgacagcgactgcagagataacgcccctagaacaatcttcatcatcagcacctacaaggatagccagcctcggggcaaggccgtgacaatcagcgtcaagtgcgaaaaaatctccaccctgagctgtgaaaacaagatcatctcttttaaggaaatgaacccacctgataatatcaaggacaccaagtccgacatcattttcttccagagagatgtgcccggacacaagcacaaaatgcagttcgagagcagcagctacgagggctacttcctggcttgcgagaaggaaagagacctgttcaagctcatcctgaagaaagaggacgagctgggcgacagaagcatcatgttcaccgtgcagaacgaggac

SEQ ID NO: 72 - DNA, 474 bp, synthetic construct
  source 1..474: mol_type = other DNA
cgcttcggcaaactggagtccaagctgagcgtgatcaggaatctgaacgatcaggtgctcttcatcgatcagggcaatagacctctgtttgaggacatgactgactccgactgtcgggacaacgccccccgaacaatctttattatcagcacctacaaggacagccagcccagggggaaggccgtgactatctccgtgaagtgcgaaaagatcagcaccctgtcatgtgagaacaagattatctcctttaaggagatgaacccccccgataacattaaggacaccaagagcgacatcatcttcttccagcgcgacgtgccaggacacaagcacaaaatgcagtttgagagcagcagctacgaagggtatttcctggcctgtgagaaggaaagagatctgttcaagctgatcctgaagaaggaggacgagctgggcgaccggagcatcatgtttaccgtgcagaacgaggactga

SEQ ID NO: 73 - DNA, 471 bp, synthetic construct
  source 1..471: mol_type = other DNA
aggttcggcaaactggaaagcaagctctccgtgatccggaacctgaacgaccaggtgctgtttatcgaccagggaaatagacctctgttcgaggacatgacagatagcgactgcagagataatgcccctcggaccatcttcatcatctctacctaccgggacagccagcctagaggcaaggccgtgaccatctctgtcaagtgcgagaagatcagcaccctgagctgcgaaaacaagatcatcagcttcaaggaaatgaaccccccagacaacatcaaggacacaaagtccgacatcattttcttccagagaaacgtgcccggccacaagtacaagatgcaatttgagtctagcagctacgagggctatttcctggcttgtgaaaaggaaagagatctgttcaaactgatcctgaagaaagaggacgagctgggcgatagaagcatcatgttcaccgtgcagaacgaggat

SEQ ID NO: 74 - DNA, 474 bp, synthetic construct
  source 1..474: mol_type = other DNA
cgcttcggcaaactggagtccaagctgagcgtgatcaggaatctgaacgatcaggtgctcttcatcgatcagggcaatagacctctgtttgaggacatgactgactccgactgtcgggacaacgccccccgaacaatctttattatcagcacctaccgggacagccagcccagggggaaggccgtgactatctccgtgaagtgcgaaaagatcagcaccctgtcatgtgagaacaagattatctcctttaaggagatgaacccccccgataacattaaggacaccaagagcgacatcatcttcttccagcgcaacgtgccaggacacaagtacaaaatgcagtttgagagcagcagctacgaagggtatttcctggcctgtgagaaggaaagagatctgttcaagctgatcctgaagaaggaggacgagctgggcgaccggagcatcatgtttaccgtgcagaacgaggactga

SEQ ID NO: 75 - DNA, 471 bp, synthetic construct
  source 1..471: mol_type = other DNA
tatttcggcaagctggaaagcaagctctctgtgatccggaacctgaacgaccaagtgctgttcatcgaccagggcaaccggcctctgttcgaggatatgaccgacagcgactgcagagataatgcccctcgcacaatcttcatcatttctaaatacagcgatagcctggccagaggcctggctgtgaccatcagcgtcaagtgcgagaaaatctccaccctgagctgcgagaacaagatcatctctttcaaggaaatgaacccccctgacaatatcaaggacacaaagtccgacatcatctttttccagagagatgtgccaggacacagcagaaagatgcagttcgagagcagctcctacgagggctacttcctggcctgtgaaaaggaacgggacctgtttaagctgatcctgaagaaagaagatgagctgggcgacagaagcatcatgttcaccgtgcagaacgaggac

SEQ ID NO: 76 - DNA, 474 bp, synthetic construct
  source 1..474: mol_type = other DNA
tatttcggcaaactggagagcaagctgagcgtgattcggaacctgaatgaccaggtgctgttcattgaccagggcaatcgccctctgttcgaagacatgaccgactccgattgtagagacaacgcccctcggaccatcttcattatcagcaagtacagcgacagcctggcccgcggcctggctgtgacaatcagcgtgaagtgcgagaagatcagcacactgtcctgcgagaacaaaatcatcagctttaaagagatgaatccccctgacaatatcaaagatactaaatctgacatcatcttcttccagcgggacgtgcctggccactctagaaaaatgcagttcgagtcctccagttacgaggggtacttcctggcctgcgagaaggagcgggacctgttcaaactgatcctgaaaaaagaagacgagctgggcgatcggagcatcatgttcactgtgcagaacgaggattga

SEQ ID NO: 77 - DNA, 471 bp, synthetic construct
  source 1..471: mol_type = other DNA
tacttcggcaagctggaaagcaagctcagcgtcatccggaacctgaacgaccaggtgctgttcatcgaccaaggcaaccggcctctgtttgaggacatgaccgactctgattctagagataatgcccctagaaccatcttcatcatctccaagtacagcgatagcctggccagaggcctggctgtgacaatctctgtgaagagcgagaaaatcagcaccctgtcttgtgaaaacaagatcatcagcttcaaggaaatgaaccctccagacaatatcaaggacacaaagtccgacatcattttcttccagagagatgtgcccggccacagcaggaagatgcagttcgagtccagcagctacgaaggatattttctggcctgcgagaaggaacgggacctgttcaaactgatcctgaagaaagaggacgagctgggcgacagaagcatcatgttcaccgtgcagaacgaggat

SEQ ID NO: 78 - DNA, 474 bp, synthetic construct
  source 1..474: mol_type = other DNA
tattttgggaagctggagagcaaactgagcgtgatccggaatctgaatgaccaggtgctgttcattgaccagggcaatcgaccactgttcgaagatatgaccgatagcgacagcagggacaatgcccccaggaccatctttatcatcagcaagtacagcgatagcctggccaggggactggccgtgacaatcagcgtgaagtccgagaagatcagcaccctgtcctgcgagaacaagatcatttctttcaaggagatgaacccccctgacaacatcaaggacacaaagagcgacatcatcttctttcagagagacgtgcccggacacagccgcaagatgcagtttgaatcttccagctacgaagggtacttcctggcctgtgaaaaagagcgcgatctgttcaaactgatcctcaagaaggaggacgagctgggcgatcgctctatcatgttcacagtgcagaacgaggactga

SEQ ID NO: 79 - DNA, 471 bp, synthetic construct
  source 1..471: mol_type = other DNA
tatttcggcaagctggaaagcaagctctctgtgatccggaacctgaacgaccaagtgctgtttatcgaccagggaaatagacctctgttcgaggacatgaccgacagcgacagccgggataacgcccctcgcaccatcttcatcatctccaagtacagcgatagcctggctagaggcctggccgtgaccatcagcgtcaagggcgagaagatctctacactgagctgtgaaaacaagatcatcagcttcaaggaaatgaacccccccgacaatatcaaggacacaaagtccgacatcattttcttccagagagatgtgcctggccacagcagaaagatgcagttcgagtccagcagctacgagggctactttctggcctgcgagaaagaacgggacctgttcaaactgatcctgaaaaaggaagacgagctgggcgatagatctatcatgttcaccgtgcagaacgaggat

SEQ ID NO: 80 - DNA, 474 bp, synthetic construct
  source 1..474: mol_type = other DNA
tattttgggaagctggagagcaaactgagcgtgatccggaatctgaatgaccaggtgctgttcattgaccagggcaatcgaccactgttcgaagatatgaccgatagcgacagcagggacaatgcccccaggaccatctttatcatcagcaagtacagcgatagcctggccaggggactggccgtgacaatcagcgtgaagggcgagaagatcagcaccctgtcctgcgagaacaagatcatttctttcaaggagatgaacccccctgacaacatcaaggacacaaagagcgacatcatcttctttcagagagacgtgcccggacacagccgcaagatgcagtttgaatcttccagctacgaagggtacttcctggcctgtgaaaaagagcgcgatctgttcaaactgatcctcaagaaggaggacgagctgggcgatcgctctatcatgttcacagtgcagaacgaggactga

SEQ ID NO: 81 - DNA, 471 bp, synthetic construct
  source 1..471: mol_type = other DNA
tatttcggcaagctggaaagcaagctgagcgtcatccggaacctcaacgaccaagtgctgtttatcgaccagggcaaccggcctctgttcgaggacatgacagacagcgacagcagagataatgcccctagaaccatcttcatcatttccaagtacagcgattctctggccagaggcctggccgtgaccatctctgtgaaggccgagaaaatctccaccctgagctgcgaaaacaagatcatcagcttcaaggagatgaaccctccagacaatatcaaggacaccaagagcgacatcatctttttccagcgggacgtgcccggccactctcgcaagatgcagttcgagtccagcagctacgagggctacttcctggcttgtgaaaaggaaagagatctgttcaaactgatcctgaaaaaggaagatgagctgggagatagaagcatcatgttcacagtgcagaacgaggac

SEQ ID NO: 82 - DNA, 474 bp, synthetic construct
  source 1..474: mol_type = other DNA
tattttgggaagctggagagcaaactgagcgtgatccggaatctgaatgaccaggtgctgttcattgaccagggcaatcgaccactgttcgaagatatgaccgatagcgacagcagggacaatgcccccaggaccatctttatcatcagcaagtacagcgatagcctggccaggggactggccgtgacaatcagcgtgaaggccgagaagatcagcaccctgtcctgcgagaacaagatcatttctttcaaggagatgaacccccctgacaacatcaaggacacaaagagcgacatcatcttctttcagagagacgtgcccggacacagccgcaagatgcagtttgaatcttccagctacgaagggtacttcctggcctgtgaaaaagagcgcgatctgttcaaactgatcctcaagaaggaggacgagctgggcgatcgctctatcatgttcacagtgcagaacgaggactga

SEQ ID NO: 83 - DNA, 471 bp, synthetic construct
  source 1..471: mol_type = other DNA
tatttcggcaagctggaatctaaactctccgtgatccggaacctgaacgaccaagtgctgttcatcgaccagggaaatagacctctgttcgaggatatgacagacagcgatagccgcgataatgcccctagaaccatctttatcattagcaagtacagcgacagcctggccagaggcctggccgtgacaatcagcgtcaaggacgagaaaatctccaccctgagctgcgagaacaagatcatcagcttcaaggaaatgaaccctccagacaacatcaaggacaccaagtccgatatcatcttcttccagcgggacgtgcccggccactctagaaagatgcagttcgagagctctagctacgagggctacttcctggcttgtgaaaaagaacgggacctgtttaagctgatcctgaagaaggaagatgagctgggcgacagaagcatcatgttcaccgtgcagaacgaggac

SEQ ID NO: 84 - DNA, 474 bp, synthetic construct
  source 1..474: mol_type = other DNA
tattttgggaagctggagagcaaactgagcgtgatccggaatctgaatgaccaggtgctgttcattgaccagggcaatcgaccactgttcgaagatatgaccgatagcgacagcagggacaatgcccccaggaccatctttatcatcagcaagtacagcgatagcctggccaggggactggccgtgacaatcagcgtgaaggacgagaagatcagcaccctgtcctgcgagaacaagatcatttctttcaaggagatgaacccccctgacaacatcaaggacacaaagagcgacatcatcttctttcagagagacgtgcccggacacagccgcaagatgcagtttgaatcttccagctacgaagggtacttcctggcctgtgaaaaagagcgcgatctgttcaaactgatcctcaagaaggaggacgagctgggcgatcgctctatcatgttcacagtgcagaacgaggactga

SEQ ID NO: 85 - DNA, 471 bp, synthetic construct
  source 1..471: mol_type = other DNA
tatttcggcaagctggaaagcaagctctccgtgatcagaaacctgaacgaccaagtgctgttcatcgaccagggcaacagacctctgtttgaggatatgaccgactctgattgcagagataatgcccctagaaccatctttatcatcagcatgtacaaggacagccagcctcggggaatggccgtgaccatcagcgtcaagtgcgagaagatctctaccctgagctgcgaaaacaaaatcatctccttcaaggaaatgaaccccccagacaatatcaaggatacaaagagcgacatcattttcttccagcggagcgtgcccggccacgacaacaagatgcagttcgagagctctagctacgagggctacttcctggcttgtgaaaaggaacgggacctgttcaaactgatcctgaagaaagaggacgagctgggcgatagaagcatcatgttcacagtgcagaacgaggac

SEQ ID NO: 86 - DNA, 474 bp, synthetic construct
  source 1..474: mol_type = other DNA
tacttcggcaaactggagtccaagctgagcgtgatcaggaatctgaacgatcaggtgctcttcatcgatcagggcaatagacctctgtttgaggacatgactgactccgactgtcgggacaacgccccccgaacaatctttattatcagcatgtacaaggacagccagcccagggggatggccgtgactatctccgtgaagtgcgaaaagatcagcaccctgtcatgtgagaacaagattatctcctttaaggagatgaacccccccgataacattaaggacaccaagagcgacatcatcttcttccagcgctccgtgccaggacacgataacaaaatgcagtttgagagcagcagctacgaagggtatttcctggcctgtgagaaggaaagagatctgttcaagctgatcctgaagaaggaggacgagctgggcgaccggagcatcatgtttaccgtgcagaacgaggactga

SEQ ID NO: 87 - DNA, 1184 bp, Homo sapiens
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 88 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 89 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 90 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 91 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 92 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 93 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 94 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 95 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 96 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 97 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 98 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 99 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 100 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 101 - protein, 328 aa, synthetic construct
MCHQQLVISWFSLVFLASPLVAIWELKKDVYVVELDWYAAAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 102 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 103 - protein, 328 aa, synthetic construct; identical to SEQ ID NO: 93

SEQ ID NO: 104 - protein, 328 aa, synthetic construct; identical to SEQ ID NO: 94

SEQ ID NO: 105 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 106 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 107 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 108 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKADGIWSTAILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 109 - protein, 481 aa, synthetic construct
MALPVTALLLPLALLLHAARPDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSTTTPAPRPPTPAPTIASQPLSLRPEACRPAAGGAVHTRGLDFACDIYIWAPLAGTCGVLLLSLVITRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 112 aa, synthetic construct
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 111 - protein, 114 aa, synthetic construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 112 - protein, 16 aa, synthetic construct
KSSQSLLDSDGKTYLN

SEQ ID NO: 113 - protein, 7 aa, synthetic construct
LVSKLDS

SEQ ID NO: 114 - protein, 9 aa, synthetic construct
WQGTHFPGT

SEQ ID NO: 115 - protein, 5 aa, synthetic construct
DYYIH

SEQ ID NO: 116 - protein, 17 aa, synthetic construct
RIDPENDETKYGPIFQG

SEQ ID NO: 117 - protein, 5 aa, synthetic construct
RGGVY

SEQ ID NO: 118 - protein, 25 aa, synthetic construct
MGVLLTQRTLLSLVLALLFPSMASM

SEQ ID NO: 119 - protein, 16 aa, synthetic construct
MKCLLYLAFLFIGVNC

SEQ ID NO: 120 - protein, 21 aa, synthetic construct
METDTLLLWVLLLWVPGSTGD

SEQ ID NO: 121 - protein, 19 aa, synthetic construct
MGWSCIILFLVATATGVHS

SEQ ID NO: 122 - protein, 17 aa, synthetic construct
MRAWIFFLLCLAGRALA

SEQ ID NO: 123 - protein, 21 aa, synthetic construct
MWWRLWWLLLLLLLLWPMVWA

SEQ ID NO: 124 - protein, 22 aa, synthetic construct
MDMRVPAQLLGLLLLWLRGARC

SEQ ID NO: 125 - protein, 16 aa, synthetic construct
MPLLLLLPLLWAGALA

SEQ ID NO: 126 - protein, 23 aa, synthetic construct
MDAMKRGLCCVLLLCGAVFVSPS

SEQ ID NO: 127 - protein, 18 aa, synthetic construct
MAFLWLLSCWALLGTTFG

SEQ ID NO: 128 - protein, 15 aa, synthetic construct
MNLLLILTFVAAAVA

SEQ ID NO: 129 - protein, 20 aa, synthetic construct
MYRMQLLSCIALSLALVTNS

SEQ ID NO: 130 - protein, 17 aa, synthetic construct
MGVKVLFALICIAVAEA

SEQ ID NO: 131 - protein, 16 aa, synthetic construct
MKWVTFISLLFSSAYS

SEQ ID NO: 132 - protein, 16 aa, synthetic construct
MKTIIALSYIFCLVLG

SEQ ID NO: 133 - protein, 24 aa, synthetic construct
MALWMRLLPLLALLALWGPDPAAA

SEQ ID NO: 134 - protein, 16 aa, synthetic construct
MKPIFLVLLVVTSAYA

SEQ ID NO: 135 - protein, 28 aa, synthetic construct
MRHNWTPDLSPLWVLLLCAHVVTLLVRA

SEQ ID NO: 136 - protein, 22 aa, synthetic construct
MCHQQLVISWFSLVFLASPLVA

SEQ ID NO: 137 - protein, 22 aa, synthetic construct
MCPARSLLLVATLVLLDHLSLA

SEQ ID NO: 138 - protein, 36 aa, synthetic construct; identical to SEQ ID NO: 3

SEQ ID NO: 139 - protein, 21 aa, synthetic construct
MALPVTALLLPLALLLHAARP

SEQ ID NO: 140 - protein, 19 aa, synthetic construct
MEFGLSWVFLVALFRGVQS

SEQ ID NO: 141 - protein, 22 aa, synthetic construct
MGYRMQLLSCIALSLALVTNSG

SEQ ID NO: 142 - DNA, 553 bp, synthetic construct
  source 1..553: mol_type = other DNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 143 - protein, 21 aa, synthetic construct
GSGEGRGSLLTCGDVEENPGP

SEQ ID NO: 144 - protein, 22 aa, synthetic construct
GSGATNFSLLKQAGDVEENPGP

SEQ ID NO: 145 - protein, 23 aa, synthetic construct
GSGQCTNYALLKLAGDVESNPGP

SEQ ID NO: 146 - protein, 25 aa, synthetic construct
GSGVKQTLNFDLLKLAGDVESNPGP

SEQ ID NO: 147 - protein, 121 aa, sythetic construct
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 148 - protein, 107 aa, sythetic construct
EIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIK

SEQ ID NO: 149 - protein, 120 aa, sythetic construct [sequence omitted]

SEQ ID NO: 150 - protein, 113 aa, sythetic construct [sequence omitted]

SEQ ID NO: 151 - protein, 120 aa, sythetic construct
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSS

SEQ ID NO: 152 - protein, 113 aa, sythetic construct
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 153 - protein, 120 aa, sythetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 154 - protein, 104 aa, sythetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 155 - protein, 120 aa, sythetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 156 - protein, 104 aa, sythetic construct
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIK

SEQ ID NO: 157 - protein, 120 aa, sythetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 158 - protein, 106 aa, sythetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIK

SEQ ID NO: 159 - protein, 121 aa, sythetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 160 - protein, 111 aa, sythetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 161 - protein, 122 aa, sythetic construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 162 - protein, 112 aa, sythetic construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 163 - protein, 120 aa, sythetic construct; identical to SEQ ID NO: 155

SEQ ID NO: 164 - protein, 104 aa, sythetic construct
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 165 - protein, 119 aa, sythetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 166 - protein, 112 aa, sythetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 167 - protein, 121 aa, sythetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 168 - protein, 112 aa, sythetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 169 - protein, 117 aa, sythetic construct
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSS

SEQ ID NO: 170 - protein, 112 aa, sythetic construct
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIK

SEQ ID NO: 171 - protein, 121 aa, sythetic construct; identical to SEQ ID NO: 167

SEQ ID NO: 172 - protein, 112 aa, sythetic construct; identical to SEQ ID NO: 168

SEQ ID NO: 173 - protein, 121 aa, sythetic construct
EVQLVQPGAEVVKPGASVKVSCKASGYTFTSYNMHWVRQAPGRGLEWMGAIYPGNGDTSYNQKFKGRVTMTRDKSTSTVYMELSSLRSEDTAVYYCARSTYYGGDWYFNVWGQGTLVTVSS

SEQ ID NO: 174 - protein, 106 aa, sythetic construct
QIVLSQSPAILSASPGERVTLTCRASSSVSYIHWFQQKPGKAPKPLIYATSNLASGVPSRFSGSGSGTDFSLTISRVEPEDFAVYYCQQWTSNPPTFGGGTKVEIK

SEQ ID NO: 175 - protein, 122 aa, sythetic construct [sequence omitted]

SEQ ID NO: 176 - protein, 106 aa, sythetic construct [sequence omitted]

SEQ ID NO: 177 - protein, 121 aa, sythetic construct
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSS

SEQ ID NO: 178 - protein, 106 aa, sythetic construct
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIK

SEQ ID NO: 179 - protein, 122 aa, sythetic construct [sequence omitted]

SEQ ID NO: 180 - protein, 106 aa, sythetic construct [sequence omitted]

SEQ ID NO: 181 - protein, 122 aa, sythetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 182 - protein, 107 aa, sythetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 183 - protein, 121 aa, sythetic construct [sequence omitted]

SEQ ID NO: 184 - protein, 106 aa, sythetic construct [sequence omitted]

SEQ ID NO: 185 - protein, 121 aa, sythetic construct [sequence omitted]

SEQ ID NO: 186 - protein, 106 aa, sythetic construct [sequence omitted]

SEQ ID NO: 187 - protein, 118 aa, sythetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 188 - protein, 106 aa, sythetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 189 - protein, 121 aa, sythetic construct
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSS

SEQ ID NO: 190 - protein, 106 aa, sythetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIK

SEQ ID NO: 191 - protein, 122 aa, sythetic construct [sequence omitted]

SEQ ID NO: 192 - protein, 106 aa, sythetic construct [sequence omitted]

SEQ ID NO: 193 - protein, 116 aa, sythetic construct [sequence omitted]

SEQ ID NO: 194 - protein, 112 aa, sythetic construct [sequence omitted]

SEQ ID NO: 195 - protein, 121 aa, sythetic construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 196 - protein, 112 aa, sythetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 197 - protein, 120 aa, sythetic construct
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSS

SEQ ID NO: 198 - protein, 112 aa, sythetic construct
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIK

SEQ ID NO: 199 - protein, 123 aa, sythetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSS

SEQ ID NO: 200 - protein, 107 aa, sythetic construct
DIQLTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 201 - protein, 117 aa, sythetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 202 - protein, 111 aa, sythetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 203 - protein, 112 aa, sythetic construct
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 204 - protein, 107 aa, sythetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIK

SEQ ID NO: 205 - protein, 116 aa, sythetic construct [sequence omitted]

SEQ ID NO: 206 - protein, 111 aa, sythetic construct [sequence omitted]

SEQ ID NO: 207 - protein, 116 aa, sythetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 208 - protein, 111 aa, sythetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 209 - protein, 117 aa, sythetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 210 - protein, 107 aa, sythetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 211 - protein, 122 aa, sythetic construct [sequence omitted]

SEQ ID NO: 212 - protein, 107 aa, sythetic construct [sequence omitted]

SEQ ID NO: 213 - protein, 121 aa, sythetic construct
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 214 - protein, 107 aa, sythetic construct
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 215 - protein, 120 aa, sythetic construct
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 216 - protein, 107 aa, sythetic construct
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVL

SEQ ID NO: 217 - protein, 120 aa, sythetic construct [sequence omitted]

SEQ ID NO: 218 - protein, 107 aa, sythetic construct [sequence omitted]

SEQ ID NO: 219 - protein, 119 aa, sythetic construct
QVQLQESGPGLVKPSETLSLTCTVSGFSLTSYGIHWLRQPPGKGLEWIGVIWRGGSTDYNPSLKSRVTISKDTSKSQVSLKLSSVTAADTAVYYCAKGKVTTGFYFDFWGQGTLVTVSS

SEQ ID NO: 220 - protein, 107 aa, sythetic construct
EIVMTQSPPTLSLSPGERVTLSCRASEDIYNRLVWYQQKPGQAPRLLISGVTSLETSIPARFSGSGSGTDYTLTISSLQPEDFAVYYCQQYWSTPYTFGQGTKVEIK

SEQ ID NO: 221 - protein, 123 aa, sythetic construct
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSS

SEQ ID NO: 222 - protein, 110 aa, sythetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVL

SEQ ID NO: 223 - protein, 120 aa, sythetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSS

SEQ ID NO: 224 - protein, 107 aa, sythetic construct
DIQMTQSPSSLSASVGDRVTITCKASQNVDSDVDWYQQKPGKAPKLLIYKASNDYTGVPSRFSGSGSGTDFTFTISSLQPEDIATYYCMQSNTHPRTFGGGTKVEIK

SEQ ID NO: 225 - protein, 122 aa, sythetic construct
EVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 226 - protein, 110 aa, sythetic construct
QAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVL

SEQ ID NO: 227 - protein, 118 aa, sythetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 228 - protein, 111 aa, sythetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 229 - protein, 118 aa, sythetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 230 - protein, 113 aa, sythetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 231 - protein, 118 aa, sythetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 232 - protein, 113 aa, sythetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 233 - protein, 118 aa, sythetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAKGTTGDWFDYWGQGTLVTVSS

SEQ ID NO: 234 - protein, 107 aa, sythetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 235 - protein, 118 aa, sythetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 236 - protein, 107 aa, sythetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 237 - protein, 118 aa, sythetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 238 - protein, 108 aa, sythetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 239 - protein, 119 aa, sythetic construct
QVQLQESGPGLVKPSETLSLTCTVSGFSLSNYDVHWVRQAPGKGLEWLGVIWSGGNTDYNTPFTSRLTISVDTSKNQFSLKLSSVTAADTAVYYCARALDYYDYEFAYWGQGTLVTVSS

SEQ ID NO: 240 - protein, 107 aa, sythetic construct
EIVLTQSPDFQSVTPKEKVTITCRASQSIGTNIHWYQQKPDQSPKLLIKYASESISGIPSRFSGSGSGTDFTLTINSLEAEDAATYYCQQNNEWPTSFGQGTKLEIK

SEQ ID NO: 241 - protein, 119 aa, sythetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 242 - protein, 107 aa, sythetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 243 - protein, 119 aa, sythetic construct [sequence omitted]

SEQ ID NO: 244 - protein, 107 aa, sythetic construct
DIQMTQSPSTLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 245 - protein, 123 aa, sythetic construct
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSS

SEQ ID NO: 246 - protein, 107 aa, sythetic construct
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIK

SEQ ID NO: 247 - protein, 120 aa, sythetic construct [sequence omitted]

SEQ ID NO: 248 - protein, 106 aa, sythetic construct [sequence omitted]

SEQ ID NO: 249 - protein, 121 aa, sythetic construct [sequence omitted]

SEQ ID NO: 250 - protein, 106 aa, sythetic construct [sequence omitted]

SEQ ID NO: 251 - protein, 119 aa, sythetic construct
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSS

SEQ ID NO: 252 - protein, 112 aa, sythetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 253 - protein, 121 aa, sythetic construct [sequence omitted]

SEQ ID NO: 254 - protein, 107 aa, sythetic construct [sequence omitted]

SEQ ID NO: 255 - protein, 123 aa, sythetic construct [sequence omitted]

SEQ ID NO: 256 - protein, 112 aa, sythetic construct [sequence omitted]

SEQ ID NO: 257 - protein, 119 aa, sythetic construct [sequence omitted]

SEQ ID NO: 258 - protein, 107 aa, sythetic construct [sequence omitted]

SEQ ID NO: 259 - protein, 119 aa, sythetic construct
EVQLVESGGGLVQPGGSLRLSCAASGFSLTNYGVHWVRQAPGKGLEWLGVIWSGGNTDYGNEFTSRFTISRDNAKNSLYLQMNSLRAEDTAVYYCARALDYYDYEFAYWGQGTMVTVSS

SEQ ID NO: 260 - protein, 107 aa, sythetic construct
EIVLTQSPATLSLSPGERATLSCRASQSIGTNIHWYQQKPGQAPRLLIKYASESISGIPARFSGSGSGTDFTLTISSLEPEDFAVYYCQQNNNWPTSFGGGTKVEIK

SEQ ID NO: 261 - protein, 119 aa, sythetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVST

SEQ ID NO: 262 - protein, 107 aa, sythetic construct; identical to SEQ ID NO: 242

SEQ ID NO: 263 - protein, 125 aa, sythetic construct [sequence omitted]

SEQ ID NO: 264 - protein, 107 aa, sythetic construct [sequence omitted]

SEQ ID NO: 265 - protein, 120 aa, sythetic construct
EVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSS

SEQ ID NO: 266 - protein, 112 aa, sythetic construct
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 267 - protein, 124 aa, sythetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 268 - protein, 107 aa, sythetic construct
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 269 - protein, 113 aa, sythetic construct [sequence omitted]

SEQ ID NO: 270 - protein, 113 aa, sythetic construct [sequence omitted]

SEQ ID NO: 271 - protein, 113 aa, sythetic construct
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSS

SEQ ID NO: 272 - protein, 113 aa, sythetic construct
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 273 - protein, 119 aa, sythetic construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 274 - protein, 104 aa, sythetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIK

SEQ ID NO: 275 - protein, 115 aa, sythetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 276 - protein, 112 aa, sythetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 277 - protein, 120 aa, sythetic construct [sequence omitted]

SEQ ID NO: 278 - protein, 107 aa, sythetic construct [sequence omitted]

SEQ ID NO: 279 - protein, 120 aa, sythetic construct [sequence omitted]

SEQ ID NO: 280 - protein, 107 aa, sythetic construct [sequence omitted]

SEQ ID NO: 281 - protein, 115 aa, sythetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 282 - protein, 105 aa, sythetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 283 - protein, 119 aa, sythetic construct
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSS

SEQ ID NO: 284 - protein, 111 aa, sythetic construct
QSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVL

SEQ ID NO: 285 - protein, 120 aa, sythetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 286 - protein, 107 aa, sythetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 287 - protein, 120 aa, sythetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSS

SEQ ID NO: 288 - protein, 112 aa, sythetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVFGGGTKLTVL

SEQ ID NO: 289 - protein, 119 aa, sythetic construct [sequence omitted]

SEQ ID NO: 290 - protein, 107 aa, sythetic construct [sequence omitted]

SEQ ID NO: 291 - protein, 120 aa, sythetic construct [sequence omitted]

SEQ ID NO: 292 - protein, 107 aa, sythetic construct [sequence omitted]

SEQ ID NO: 293 - protein, 120 aa, sythetic construct [sequence omitted]

SEQ ID NO: 294 - protein, 107 aa, sythetic construct [sequence omitted]

SEQ ID NO: 295 - protein, 119 aa, sythetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 296 - protein, 106 aa, sythetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 297 - protein, 120 aa, sythetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 298 - protein, 111 aa, sythetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 299 - protein, 117 aa, sythetic construct
QVQLVESGGGLVKPGGSLRLSCAASGFTFSDYYMSWIRQAPGKCLEWISYISSSESIIYYVDAVKGRFTISRDNAKNSLYLQMNSLRAEDTAVYYCARDVGSHFDYWGQGTLVTVSS

SEQ ID NO: 300 - protein, 107 aa, sythetic construct
DIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIK

SEQ ID NO: 301 - protein, 119 aa, sythetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 302 - protein, 112 aa, sythetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 303 - protein, 119 aa, sythetic construct
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSS

SEQ ID NO: 304 - protein, 108 aa, sythetic construct
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIK

SEQ ID NO: 305 - protein, 117 aa, sythetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 306 - protein, 112 aa, sythetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 307 - protein, 119 aa, sythetic construct
QVKLQESGAELARPGASVKLSCKASGYTFTNYWMQWVKQRPGQGLDWIGAIYPGDGNTRYTHKFKGKATLTADKSSSTAYMQLSSLASEDSGVYYCARGEGNYAWFAYWGQGTTVTVSS

SEQ ID NO: 308 - protein, 107 aa, sythetic construct
DIELTQSPASLSASVGETVTITCQASENIYSYLAWHQQKQGKSPQLLVYNAKTLAGGVSSRFSGSGSGTHFSLKIKSLQPEDFGIYYCQHHYGILPTFGGGTKLEIK

SEQ ID NO: 309 - protein, 116 aa, sythetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 310 - protein, 107 aa, sythetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 311 - protein, 117 aa, sythetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 312 - protein, 108 aa, sythetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 313 - protein, 115 aa, sythetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 314 - protein, 108 aa, sythetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 315 - protein, 114 aa, sythetic construct
QVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSS

SEQ ID NO: 316 - protein, 113 aa, sythetic construct
DIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIK

SEQ ID NO: 317 - protein, 113 aa, sythetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 318 - protein, 107 aa, sythetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 319 - protein, 118 aa, sythetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 320 - protein, 112 aa, sythetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 321 - protein, 116 aa, sythetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 322 - protein, 107 aa, sythetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 323 - protein, 117 aa, sythetic construct [sequence omitted]

SEQ ID NO: 324 - protein, 107 aa, sythetic construct [sequence omitted]

SEQ ID NO: 325 - protein, 123 aa, sythetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 326 - protein, 107 aa, sythetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 327 - protein, 116 aa, sythetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 328 - protein, 107 aa, sythetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 329 - protein, 113 aa, sythetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 330 - protein, 107 aa, sythetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 331 - protein, 120 aa, sythetic construct
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 332 - protein, 111 aa, sythetic construct
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIK

SEQ ID NO: 333 - protein, 118 aa, sythetic construct
EVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSS

SEQ ID NO: 334 - protein, 111 aa, sythetic construct
EIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIK

SEQ ID NO: 335 - protein, 114 aa, sythetic construct
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSS

SEQ ID NO: 336 - protein, 110 aa, sythetic construct
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIK

SEQ ID NO: 337 - protein, 119 aa, sythetic construct
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 338 - protein, 113 aa, sythetic construct
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 339 - protein, 120 aa, sythetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 340 - protein, 111 aa, sythetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 341 - protein, 113 aa, sythetic construct [sequence omitted]

SEQ ID NO: 342 - protein, 107 aa, sythetic construct [sequence omitted]

SEQ ID NO: 343 - protein, 115 aa, sythetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 344 - protein, 112 aa, sythetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 345 - protein, 118 aa, sythetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 346 - protein, 107 aa, sythetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 347 - protein, 119 aa, sythetic construct
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSS

SEQ ID NO: 348 - protein, 110 aa, sythetic construct
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIK

SEQ ID NO: 349 - protein, 117 aa, sythetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 350 - protein, 106 aa, sythetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 351 - protein, 118 aa, sythetic construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 352 - protein, 107 aa, sythetic construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 353 - protein, 121 aa, sythetic construct
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSS

SEQ ID NO: 354 - protein, 106 aa, sythetic construct
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWTTFGGGTKVEIK

SEQ ID NO: 355 - protein, 129 aa, sythetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSS

SEQ ID NO: 356 - protein, 106 aa, sythetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVL

SEQ ID NO: 357 - protein, 118 aa, sythetic construct
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSS

SEQ ID NO: 358 - protein, 111 aa, sythetic construct
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIK

SEQ ID NO: 359 - protein, 119 aa, sythetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 360 - protein, 111 aa, sythetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 361 - protein, 121 aa, sythetic construct
QVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSS

SEQ ID NO: 362 - protein, 108 aa, sythetic construct
EIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIK

SEQ ID NO: 363 - protein, 118 aa, sythetic construct
EVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSS

SEQ ID NO: 364 - protein, 111 aa, sythetic construct
EIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIK

SEQ ID NO: 365 - protein, 117 aa, sythetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 366 - protein, 113 aa, sythetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 367 - protein, 116 aa, sythetic construct
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 368 - protein, 107 aa, sythetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 369 - protein, 120 aa, sythetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 370 - protein, 107 aa, sythetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 371 - protein, 117 aa, sythetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 372 - protein, 113 aa, sythetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 373 - protein, 118 aa, sythetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 374 - protein, 107 aa, sythetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 375 - protein, 125 aa, sythetic construct [sequence omitted]

SEQ ID NO: 376 - protein, 112 aa, sythetic construct [sequence omitted]

SEQ ID NO: 377 - protein, 120 aa, sythetic construct
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 378 - protein, 107 aa, sythetic construct
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 379 - protein, 123 aa, sythetic construct
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 380 - protein, 110 aa, sythetic construct
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 381 - protein, 122 aa, sythetic construct
EVQLLESGGGLVQPGGSLRLSCAASGFTISRYHMTWVRQAPGKGLEWIGHIYVNNDDTDYASSAKGRFTISRDNSKNTLYLQMNSLRAEDTATYFCARLDVGGGGAYIGDIWGQGTLVTVSS

SEQ ID NO: 382 - protein, 113 aa, sythetic construct
DIQMTQSPSSLSASVGDRVTITCQSSQSVYNNNDLAWYQQKPGKVPKLLIYYASTLASGVPSRFSGSGSGTDFTLTISSLQPEDVATYYCAGGYDTDGLDTFAFGGGTKVEIK

SEQ ID NO: 383 - protein, 116 aa, sythetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 384 - protein, 107 aa, sythetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 385 - protein, 117 aa, sythetic construct
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 386 - protein, 113 aa, sythetic construct
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 387 - protein, 119 aa, sythetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 388 - protein, 107 aa, sythetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

(19) United States
(12) Patent Application Publication
(10) Pub. No.: US 2025/0282842 A1
(43) Pub. Date: Sep. 11, 2025

(54) DECOY-RESISTANT INTERLEUKIN 18 ARMORED CELLS AND RELATED METHODS

(71) Applicant: Simcha IL-18, Inc., New Haven, CT (US)

(72) Inventors: Hirdesh UPPAL, New Haven, CT (US); Jeffrey N. LINDQUIST, New Haven, CT (US)

(21) Appl. No.: 19/072,126

(22) Filed: Mar. 6, 2025

Related U.S. Application Data
(60) Provisional application No. 63/562,482, filed on Mar. 7, 2024.

(51) Int. Cl. C07K 14/54; A61K 40/11; A61K 40/31; A61K 40/35; A61K 40/42; A61P 35/00; C07K 16/28; C12N 5/0783

(52) U.S. Cl. CPC C07K 14/54; A61K 40/11; A61K 40/31; A61K 40/35; A61K 40/4204; A61K 40/4234; A61P 35/00; C07K 16/2863; C12N 5/0636; C07K 2319/02; C12N 2510/00

(57) ABSTRACT

Provided are nucleic acids that encode decoy-resistant interleukin 18 (DR-18) polypeptides, as well as vectors and cells comprising such nucleic acids and cells that comprise such vectors. Cells encoding DR-18 polypeptides may be referred to as DR-18 armored cells. The nucleic acids may further encode a chimeric antigen receptor (CAR) or multiple CARs, including CARs that bind to antigens described herein. Methods are also provided, including methods of making the nucleic acids, vectors, and/or cells, as well as methods of use, such as employing the nucleic acids, vectors, and/or cells, in the treatment of a subject having cancer.

### CROSS-REFERENCE

This application claims benefit of U.S. Provisional Patent Application No. 63/562,482 filed Mar. 7, 2024, which application is incorporated herein by reference in its entirety.

### REFERENCE TO SEQUENCE LISTING SUBMITTED ELECTRONICALLY

The sequence listing of the present application is submitted electronically with a file name “ST-016-US1 Seqlist.xml”, creation date of Mar. 5, 2025, and a size of 391 kb. This sequence listing submitted is part of the specification and is herein incorporated by reference in its entirety.

### Introduction

According to the National Cancer Institute (NCI) Surveillance, Epidemiology, and End Results Program (SEER), approximately 606,520 people will have died of cancer in the United States (US) in 2020. Despite advances in cancer prevention, diagnosis, and treatment, an estimated 1,806,590 people in the US will have been diagnosed with a new cancer in 2020. Thus, cancer remains a continuing public health concern of major proportions.

Genetically modified T cells expressing chimeric antigen receptors (CARs) so far have mostly failed in the treatment of solid tumors owing to a number of limitations, including an immunosuppressive tumor microenvironment and insufficient CAR T cell activation and persistence. See Zimmerman et al. Cancers (Basel). 2020 February; 12(2): 375.

### SUMMARY

Provided are nucleic acids that encode decoy-resistant interleukin 18 (DR-18) polypeptides, as well as vectors and cells comprising such nucleic acids and cells that comprise such vectors. Cells encoding DR-18 polypeptides may be referred to as DR-18 armored cells. The nucleic acids may further encode a chimeric antigen receptor (CAR) or multiple CARs, including CARs that bind to antigens described herein. Methods are also provided, including methods of making the nucleic acids, vectors, and/or cells, as well as methods of use, such as employing the nucleic acids, vectors, and/or cells, in the treatment of a subject having cancer.

Aspects of the present disclosure include a nucleic acid comprising a sequence encoding a decoy-resistant interleukin 18 (DR-18) polypeptide and a chimeric antigen receptor (CAR) sequence encoding a CAR, wherein the CAR is selected from: i) an anti-5T4 CAR comprising an antigen binding domain (ABD) that specifically binds 5T4; ii) an anti-AFP CAR comprising an ABD that specifically binds AFP; iii) an anti-ALPP CAR comprising an ABD that specifically binds ALPP; iv) an anti-ALPPL2 CAR comprising an ABD that specifically binds ALPPL2; v) an anti-APN (CD13) CAR comprising an ABD that specifically binds APN (CD13); vi) an anti-APRIL CAR comprising an ABD that specifically binds APRIL; vii) an anti-AXL CAR comprising an ABD that specifically binds AXL; viii) an anti-B7-H3 CAR comprising an ABD that specifically binds B7-H3; ix) an anti-B7-H4 CAR comprising an ABD that specifically binds B7-H4; x) an anti-BAFF-R CAR comprising an ABD that specifically binds BAFF-R; xi) an anti-BAFF-R CAR comprising an ABD that specifically binds BAFF-R; xii) an anti-BCMA CAR comprising an ABD that specifically binds BCMA; xiii) an anti-BSG CAR comprising an ABD that specifically binds BSG; xiv) an anti-CAIX CAR comprising an ABD that specifically binds CAIX; xv) an anti-CD123 CAR comprising an ABD that specifically binds CD123; xvi) an anti-CD133 CAR comprising an ABD that specifically binds CD133; xvii) an anti-CD138 CAR comprising an ABD that specifically binds CD138; xviii) an anti-CD19 CAR comprising an ABD that specifically binds CD19; xix) an anti-CD1A CAR comprising an ABD that specifically binds CD1A; xx) an anti-CD2 CAR comprising an ABD that specifically binds CD2; xxi) an anti-CD20 CAR comprising an ABD that specifically binds CD20; xxii) an anti-CD22 CAR comprising an ABD that specifically binds CD22; xxiii) an anti-CD24 CAR comprising an ABD that specifically binds CD24; xxiv) an anti-CD276 CAR comprising an ABD that specifically binds CD276; xxv) an anti-CD3 CAR comprising an ABD that specifically binds CD3; xxvi) an anti-CD30 CAR comprising an ABD that specifically binds CD30; xxvii) an anti-CD33 CAR comprising an ABD that specifically binds CD33; xxviii) an anti-CD38 CAR comprising an ABD that specifically binds CD38; xxix) an anti-CD4 CAR comprising an ABD that specifically binds CD4; xxx) an anti-CD43 CAR comprising an ABD that specifically binds CD43; xxxi) an anti-CD44 CAR comprising an ABD that specifically binds CD44; xxxii) an anti-CD44v6 CAR comprising an ABD that specifically binds CD44v6; xxxiii) an anti-CD45 CAR comprising an ABD that specifically binds CD45; xxxiv) an anti-CD5 CAR comprising an ABD that specifically binds CD5; xxxv) an anti-CD56 CAR comprising an ABD that specifically binds CD56; xxxvi) an anti-CD7 CAR comprising an ABD that specifically binds CD7; xxxvii) an anti-CD70 CAR comprising an ABD that specifically binds CD70; xxxviii) an anti-CD72 CAR comprising an ABD that specifically binds CD72; xxxix) an anti-CD83 CAR comprising an ABD that specifically binds CD83; xl) an anti-CD84 CAR comprising an ABD that specifically binds CD84; xli) an anti-CD99 CAR comprising an ABD that specifically binds CD99; xlii) an anti-CDCP1 CAR comprising an ABD that specifically binds CDCP1; xliii) an anti-CDH17 CAR comprising an ABD that specifically binds CDH17; xliv) an anti-CEA CAR comprising an ABD that specifically binds CEA; xlv) an anti-CEACAM5 CAR comprising an ABD that specifically binds CEACAM5; xlvi) an anti-CLDN18.2 CAR comprising an ABD that specifically binds CLDN18.2; xlvii) an anti-CLDN6 CAR comprising an ABD that specifically binds CLDN6; xlviii) an anti-CLEC4K (CD207) CAR comprising an ABD that specifically binds CLEC4K (CD207); xlix) an anti-CLL-1 CAR comprising an ABD that specifically binds CLL-1; l) an anti-CSPG4 CAR comprising an ABD that specifically binds CSPG4; li) an anti-DLL3 CAR comprising an ABD that specifically binds DLL3; lii) an anti-DR5 CAR comprising an ABD that specifically binds DR5; liii) an anti-EBV Protein CAR comprising an ABD that specifically binds EBV Protein; liv) an anti-EGFR CAR comprising an ABD that specifically binds EGFR; lv) an anti-EGFRvIII CAR comprising an ABD that specifically binds EGFRvIII; lvi) an anti-EMR1 CAR comprising an ABD that specifically binds EMR1; lvii) an anti-enkephalinase (CD10) CAR comprising an ABD that specifically binds enkephalinase (CD10); lviii) an anti-EpCAM CAR comprising an ABD that specifically binds EpCAM; lix) an anti-EphA2 CAR comprising an ABD that specifically binds EphA2; lx) an anti-EphA3 CAR comprising an ABD that specifically binds EphA3; lxi) an anti-FAP CAR comprising an ABD that specifically binds FAP; lxii) an anti-FGFR4 CAR comprising an ABD that specifically binds FGFR4; lxiii) an anti-FLT3 CAR comprising an ABD that specifically binds FLT3; lxiv) an anti-FOLR1 CAR comprising an ABD that specifically binds FOLR1; lxv) an anti-FSHR CAR comprising an ABD that specifically binds FSHR; lxvi) an anti-GC-C CAR comprising an ABD that specifically binds GC-C; lxvii) an anti-GD2 CAR comprising an ABD that specifically binds GD2; lxviii) an anti-GFRA4 CAR comprising an ABD that specifically binds GFRA4; lxix) an anti-Globo H CAR comprising an ABD that specifically binds Globo H; lxx) an anti-GM2 (ganglioside M2) CAR comprising an ABD that specifically binds GM2 (ganglioside M2); lxxi) an anti-gp100 CAR comprising an ABD that specifically binds gp100; lxxii) an anti-GPC1 CAR comprising an ABD that specifically binds GPC1; lxxiii) an anti-GPC2 CAR comprising an ABD that specifically binds GPC2; lxxiv) an anti-GPC3 CAR comprising an ABD that specifically binds GPC3; lxxv) an anti-HAAH (ASPH) CAR comprising an ABD that specifically binds HAAH (ASPH); lxxvi) an anti-HER2 CAR comprising an ABD that specifically binds HER2; lxxvii) an anti-HGF CAR comprising an ABD that specifically binds HGF; lxxviii) an anti-HIV envelope protein gp120 CAR comprising an ABD that specifically binds HIV envelope protein gp120; lxxix) an anti-HIV-1 pol CAR comprising an ABD that specifically binds HIV-1 pol; lxxx) an anti-HLA-A2 CAR comprising an ABD that specifically binds HLA-A2; lxxxi) an anti-HLA-G CAR comprising an ABD that specifically binds HLA-G; lxxxii) an anti-HSP70 heat-shock protein CAR comprising an ABD that specifically binds HSP70 heat-shock protein; lxxxiii) an anti-ICAM-1 CAR comprising an ABD that specifically binds ICAM-1; lxxxiv) an anti-IL-10R CAR comprising an ABD that specifically binds IL-10R; lxxxv) an anti-IL13Rα2 CAR comprising an ABD that specifically binds IL13Rα2; lxxxvi) an anti-IL-13Rα2 CAR comprising an ABD that specifically binds IL-13Rα2; lxxxvii) an anti-integrin αvβ6 CAR comprising an ABD that specifically binds integrin αvβ6; lxxxviii) an anti-ITGB7 CAR comprising an ABD that specifically binds ITGB7; lxxxix) an anti-KKLC1 CAR comprising an ABD that specifically binds KKLC1; xc) an anti-KMA CAR comprising an ABD that specifically binds KMA; xci) an anti-L1CAM CAR comprising an ABD that specifically binds L1CAM; xcii) an anti-Lewis-Y antigen CAR comprising an ABD that specifically binds Lewis-Y antigen; xciii) an anti-LGR5 CAR comprising an ABD that specifically binds LGR5; xciv) an anti-LILRB4 CAR comprising an ABD that specifically binds LILRB4; xcv) an anti-LMP1 CAR comprising an ABD that specifically binds LMP1; xcvi) an anti-MAGEA3 CAR comprising an ABD that specifically binds MAGEA3; xcvii) an anti-M-CSF CAR comprising an ABD that specifically binds M-CSF; xcviii) an anti-MG7 CAR comprising an ABD that specifically binds MG7; xcix) an anti-MICA CAR comprising an ABD that specifically binds MICA; c) an anti-MMP2 CAR comprising an ABD that specifically binds MMP2; ci) an anti-MSLN CAR comprising an ABD that specifically binds MSLN; cii) an anti-MUC1/MUC16 CAR comprising an ABD that specifically binds MUC1/MUC16; ciii) an anti-NCR3LG1 CAR comprising an ABD that specifically binds NCR3LG1; civ) an anti-NECTIN2 CAR comprising an ABD that specifically binds NECTIN2; cv) an anti-nectin-4 CAR comprising an ABD that specifically binds nectin-4; cvi) an anti-NKG2D CAR comprising an ABD that specifically binds NKG2D; cvii) an anti-NKG2DL CAR comprising an ABD that specifically binds NKG2DL; cviii) an anti-NR2F6 CAR comprising an ABD that specifically binds NR2F6; cix) an anti-NY-ESO-1 CAR comprising an ABD that specifically binds NY-ESO-1; cx) an anti-OPCML CAR comprising an ABD that specifically binds OPCML; cxi) an anti-PD-1 CAR comprising an ABD that specifically binds PD-1; cxii) an anti-PDGFRα CAR comprising an ABD that specifically binds PDGFRα; cxiii) an anti-PDL1 CAR comprising an ABD that specifically binds PDL1; cxiv) an anti-PLA2R CAR comprising an ABD that specifically binds PLA2R; cxv) an anti-PR1 CAR comprising an ABD that specifically binds PR1; cxvi) an anti-PSCA CAR comprising an ABD that specifically binds PSCA; cxvii) an anti-PTK7 CAR comprising an ABD that specifically binds PTK7; cxviii) an anti-PVR CAR comprising an ABD that specifically binds PVR; cxix) an anti-ROBO1 CAR comprising an ABD that specifically binds ROBO1; cxx) an anti-ROR1 CAR comprising an ABD that specifically binds ROR1; cxxi) an anti-ROR2 CAR comprising an ABD that specifically binds ROR2; cxxii) an anti-SEMA4A CAR comprising an ABD that specifically binds SEMA4A; cxxiii) an anti-SLAMF7 CAR comprising an ABD that specifically binds SLAMF7; cxxiv) an anti-TAG-72 CAR comprising an ABD that specifically binds TAG-72; cxxv) an anti-TGF-β CAR comprising an ABD that specifically binds TGF-β; cxxvi) an anti-TM4SF1 CAR comprising an ABD that specifically binds TM4SF1; cxxvii) an anti-TRAIL CAR comprising an ABD that specifically binds TRAIL; cxxviii) an anti-TRBC1 CAR comprising an ABD that specifically binds TRBC1; cxxix) an anti-TRBC2 CAR comprising an ABD that specifically binds TRBC2; cxxx) an anti-Trop-2 CAR comprising an ABD that specifically binds Trop-2; cxxxi) an anti-TSHR CAR comprising an ABD that specifically binds TSHR; cxxxii) an anti-TSLPR CAR comprising an ABD that specifically binds TSLPR; cxxxiii) an anti-ULBP1 CAR comprising an ABD that specifically binds ULBP1; cxxxiv) an anti-αvβ3 integrin CAR comprising an ABD that specifically binds αvβ3 integrin; and cxxxv) an anti-K light chain of human immunoglobulin CAR comprising an ABD that specifically binds K light chain of human immunoglobulin.

Aspects of the present disclosure include a nucleic acid comprising a sequence encoding a decoy-resistant interleukin 18 (DR-18) polypeptide and a chimeric antigen receptor (CAR) sequence encoding a CAR, wherein the CAR is selected from: i) an anti-5T4 CAR comprising a means for specifically binding 5T4; ii) an anti-AFP CAR comprising a means for specifically binding AFP; iii) an anti-ALPP CAR comprising a means for specifically binding ALPP; iv) an anti-ALPPL2 CAR comprising a means for specifically binding ALPPL2; v) an anti-APN (CD13) CAR comprising a means for specifically binding APN (CD13); vi) an anti-APRIL CAR comprising a means for specifically binding APRIL; vii) an anti-AXL CAR comprising a means for specifically binding AXL; viii) an anti-B7-H3 CAR comprising a means for specifically binding B7-H3; ix) an anti-B7-H4 CAR comprising a means for specifically binding B7-H4; x) an anti-BAFF-R CAR comprising a means for specifically binding BAFF-R; xi) an anti-BAFF-R CAR comprising a means for specifically binding BAFF-R; xii) an anti-BCMA CAR comprising a means for specifically binding BCMA; xiii) an anti-BSG CAR comprising a means for specifically binding BSG; xiv) an anti-CAIX CAR comprising a means for specifically binding CAIX; xv) an anti-CD123 CAR comprising a means for specifically binding CD123; xvi) an anti-CD133 CAR comprising a means for specifically binding CD133; xvii) an anti-CD138 CAR comprising a means for specifically binding CD138; xviii) an anti-CD19 CAR comprising a means for specifically binding CD19; xix) an anti-CD1A CAR comprising a means for specifically binding CD1A; xx) an anti-CD2 CAR comprising a means for specifically binding CD2; xxi) an anti-CD20 CAR comprising a means for specifically binding CD20; xxii) an anti-CD22 CAR comprising a means for specifically binding CD22; xxiii) an anti-CD24 CAR comprising a means for specifically binding CD24; xxiv) an anti-CD276 CAR comprising a means for specifically binding CD276; xxv) an anti-CD3 CAR comprising a means for specifically binding CD3; xxvi) an anti-CD30 CAR comprising a means for specifically binding CD30; xxvii) an anti-CD33 CAR comprising a means for specifically binding CD33; xxviii) an anti-CD38 CAR comprising a means for specifically binding CD38; xxix) an anti-CD4 CAR comprising a means for specifically binding CD4; xxx) an anti-CD43 CAR comprising a means for specifically binding CD43; xxxi) an anti-CD44 CAR comprising a means for specifically binding CD44; xxxii) an anti-CD44v6 CAR comprising a means for specifically binding CD44v6; xxxiii) an anti-CD45 CAR comprising a means for specifically binding CD45; xxxiv) an anti-CD5 CAR comprising a means for specifically binding CD5; xxxv) an anti-CD56 CAR comprising a means for specifically binding CD56; xxxvi) an anti-CD7 CAR comprising a means for specifically binding CD7; xxxvii) an anti-CD70 CAR comprising a means for specifically binding CD70; xxxviii) an anti-CD72 CAR comprising a means for specifically binding CD72; xxxix) an anti-CD83 CAR comprising a means for specifically binding CD83; xl) an anti-CD84 CAR comprising a means for specifically binding CD84; xli) an anti-CD99 CAR comprising a means for specifically binding CD99; xlii) an anti-CDCP1 CAR comprising a means for specifically binding CDCP1; xliii) an anti-CDH17 CAR comprising a means for specifically binding CDH17; xliv) an anti-CEA CAR comprising a means for specifically binding CEA; xlv) an anti-CEACAM5 CAR comprising a means for specifically binding CEACAM5; xlvi) an anti-CLDN18.2 CAR comprising a means for specifically binding CLDN18.2; xlvii) an anti-CLDN6 CAR comprising a means for specifically binding CLDN6; xlviii) an anti-CLEC4K (CD207) CAR comprising a means for specifically binding CLEC4K (CD207); xlix) an anti-CLL-1 CAR comprising a means for specifically binding CLL-1; l) an anti-CSPG4 CAR comprising a means for specifically binding CSPG4; li) an anti-DLL3 CAR comprising a means for specifically binding DLL3; lii) an anti-DR5 CAR comprising a means for specifically binding DR5; liii) an anti-EBV Protein CAR comprising a means for specifically binding EBV Protein; liv) an anti-EGFR CAR comprising a means for specifically binding EGFR; lv) an anti-EGFRvIII CAR comprising a means for specifically binding EGFRvIII; lvi) an anti-EMR1 CAR comprising a means for specifically binding EMR1; lvii) an anti-enkephalinase (CD10) CAR comprising a means for specifically binding enkephalinase (CD10); lviii) an anti-EpCAM CAR comprising a means for specifically binding EpCAM; lix) an anti-EphA2 CAR comprising a means for specifically binding EphA2; lx) an anti-EphA3 CAR comprising a means for specifically binding EphA3; lxi) an anti-FAP CAR comprising a means for specifically binding FAP; lxii) an anti-FGFR4 CAR comprising a means for specifically binding FGFR4; lxiii) an anti-FLT3 CAR comprising a means for specifically binding FLT3; lxiv) an anti-FOLR1 CAR comprising a means for specifically binding FOLR1; lxv) an anti-FSHR CAR comprising a means for specifically binding FSHR; lxvi) an anti-GC-C CAR comprising a means for specifically binding GC-C; lxvii) an anti-GD2 CAR comprising a means for specifically binding GD2; lxviii) an anti-GFRA4 CAR comprising a means for specifically binding GFRA4; lxix) an anti-Globo H CAR comprising a means for specifically binding Globo H; lxx) an anti-GM2 (ganglioside M2) CAR comprising a means for specifically binding GM2 (ganglioside M2); lxxi) an anti-gp100 CAR comprising a means for specifically binding gp100; lxxii) an anti-GPC1 CAR comprising a means for specifically binding GPC1; lxxiii) an anti-GPC2 CAR comprising a means for specifically binding GPC2; lxxiv) an anti-GPC3 CAR comprising a means for specifically binding GPC3; lxxv) an anti-HAAH (ASPH) CAR comprising a means for specifically binding HAAH (ASPH); lxxvi) an anti-HER2 CAR comprising a means for specifically binding HER2; lxxvii) an anti-HGF CAR comprising a means for specifically binding HGF; lxxviii) an anti-HIV envelope protein gp120 CAR comprising a means for specifically binding HIV envelope protein gp120; lxxix) an anti-HIV-1 pol CAR comprising a means for specifically binding HIV-1 pol; lxxx) an anti-HLA-A2 CAR comprising a means for specifically binding HLA-A2; lxxxi) an anti-HLA-G CAR comprising a means for specifically binding HLA-G; lxxxii) an anti-HSP70 heat-shock protein CAR comprising a means for specifically binding HSP70 heat-shock protein; lxxxiii) an anti-ICAM-1 CAR comprising a means for specifically binding ICAM-1; lxxxiv) an anti-IL-10R CAR comprising a means for specifically binding IL-10R; lxxxv) an anti-IL13Rα2 CAR comprising a means for specifically binding IL13Rα2; lxxxvi) an anti-IL-13Rα2 CAR comprising a means for specifically binding IL-13Rα2; lxxxvii) an anti-integrin αvβ6 CAR comprising a means for specifically binding integrin αvβ6; lxxxviii) an anti-ITGB7 CAR comprising a means for specifically binding ITGB7; lxxxix) an anti-KKLC1 CAR comprising a means for specifically binding KKLC1; xc) an anti-KMA CAR comprising a means for specifically binding KMA; xci) an anti-L1CAM CAR comprising a means for specifically binding L1CAM; xcii) an anti-Lewis-Y antigen CAR comprising a means for specifically binding Lewis-Y antigen; xciii) an anti-LGR5 CAR comprising a means for specifically binding LGR5; xciv) an anti-LILRB4 CAR comprising a means for specifically binding LILRB4; xcv) an anti-LMP1 CAR comprising a means for specifically binding LMP1; xcvi) an anti-MAGEA3 CAR comprising a means for specifically binding MAGEA3; xcvii) an anti-M-CSF CAR comprising a means for specifically binding M-CSF; xcviii) an anti-MG7 CAR comprising a means for specifically binding MG7; xcix) an anti-MICA CAR comprising a means for specifically binding MICA; c) an anti-MMP2 CAR comprising a means for specifically binding MMP2; ci) an anti-MSLN CAR comprising a means for specifically binding MSLN; cii) an anti-MUC1/MUC16 CAR comprising a means for specifically binding MUC1/MUC16; ciii) an anti-NCR3LG1 CAR comprising a means for specifically binding NCR3LG1; civ) an anti-NECTIN2 CAR comprising a means for specifically binding NECTIN2; cv) an anti-nectin-4 CAR comprising a means for specifically binding nectin-4; cvi) an anti-NKG2D CAR comprising a means for specifically binding NKG2D; cvii) an anti-NKG2DL CAR comprising a means for specifically binding NKG2DL; cviii) an anti-NR2F6 CAR comprising a means for specifically binding NR2F6; cix) an anti-NY-ESO-1 CAR comprising a means for specifically binding NY-ESO-1; cx) an anti-OPCML CAR comprising a means for specifically binding OPCML; cxi) an anti-PD-1 CAR comprising a means for specifically binding PD-1; cxii) an anti-PDGFRα CAR comprising a means for specifically binding PDGFRα; cxiii) an anti-PDL1 CAR comprising a means for specifically binding PDL1; cxiv) an anti-PLA2R CAR comprising a means for specifically binding PLA2R; cxv) an anti-PR1 CAR comprising a means for specifically binding PR1; cxvi) an anti-PSCA CAR comprising a means for specifically binding PSCA; cxvii) an anti-PTK7 CAR comprising a means for specifically binding PTK7; cxviii) an anti-PVR CAR comprising a means for specifically binding PVR; cxix) an anti-ROBO1 CAR comprising a means for specifically binding ROBO1; cxx) an anti-ROR1 CAR comprising a means for specifically binding ROR1; cxxi) an anti-ROR2 CAR comprising a means for specifically binding ROR2; cxxii) an anti-SEMA4A CAR comprising a means for specifically binding SEMA4A; cxxiii) an anti-SLAMF7 CAR comprising a means for specifically binding SLAMF7; cxxiv) an anti-TAG-72 CAR comprising a means for specifically binding TAG-72; cxxv) an anti-TGF-β CAR comprising a means for specifically binding TGF-β; cxxvi) an anti-TM4SF1 CAR comprising a means for specifically binding TM4SF1; cxxvii) an anti-TRAIL CAR comprising a means for specifically binding TRAIL; cxxviii) an anti-TRBC1 CAR comprising a means for specifically binding TRBC1; cxxix) an anti-TRBC2 CAR comprising a means for specifically binding TRBC2; cxxx) an anti-Trop-2 CAR comprising a means for specifically binding Trop-2; cxxxi) an anti-TSHR CAR comprising a means for specifically binding TSHR; cxxxii) an anti-TSLPR CAR comprising a means for specifically binding TSLPR; cxxxiii) an anti-ULBP1 CAR comprising a means for specifically binding ULBP1; cxxxiv) an anti-αvβ3 integrin CAR comprising a means for specifically binding αvβ3 integrin; and cxxxv) an anti-K light chain of human immunoglobulin CAR comprising a means for specifically binding K light chain of human immunoglobulin

In some embodiments, the CAR is selected from: a) inaticabtagene autoleucel; b) actalycabtagene autoleucel; c) relmacabtagene autoleucel; d) lisocabtagene maraleucel; e) brexucabtagene autoleucel; f) axicabtagene ciloleucel; g) tisagenlecleucel; h) obecabatagene autoleucel; i) azercabtagene zapreleucel; j) rapcabtagene autoleucel; and k) idecabtagene vicleucel.

In some embodiments, the CAR is an anti-EGFRvIII CAR comprising an ABD that specifically binds EGFRvIII. In some embodiments, the CAR is an anti-EGFRvIII CAR comprising a means for specifically binding EGFRvIII. In some embodiments, the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO: 110, and one or more of heavy chain complementary determining region 1 (HC CDR1), heavy chain complementary determining region 2 (HC CDR2), and heavy chain complementary determining region 3 (HC CDR3) of SEQ ID NO:111. In some embodiments, the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO:117). In some embodiments, the antigen binding domain comprises a light chain variable region comprising sequence SEQ ID NO:110 and a heavy chain variable region comprising sequence SEQ ID NO:111.

In some embodiments, the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.

In some embodiments, the sequence encoding the DR-18 polypeptide is codon optimized for expression in humans. In some embodiments, the sequence encoding the DR-18 polypeptide comprises a DR-18 encoding sequence selected from SEQ ID NOs: 67-84.

In some embodiments, the nucleic acid is configured for in vivo expression of the DR-18 polypeptide and CAR.

In some embodiments, the nucleic acid further comprises a sequence encoding a cytokine selected from: interleukin 2, interleukin 21, interleukin 23, interleukin 27, interleukin 33, tumor necrosis factor (TNF), TNF ligand superfamily member 15, interferon (IFN) alpha, IFN beta, and IFN gamma.

In some embodiments, the nucleic acid comprises a plurality of CAR sequences encoding a plurality of different CARs that bind different antigens. In some embodiments, the plurality of CAR sequences encode a plurality of different CARs that bind to a combination of antigens selected from: CD19 and CD20, CD19 and CD22, BCMA and CD19, CD33 and CLL-1, CD19 and CD276, CD19 and CD20 and CD22, CD19 and IL15R, IL15R and MUC16, IL-15Rα and NKG2D, GPC3 and IL15R, CD16a and CD19 and IL-15Rα, BCMA and CD16a and NKp46, CD123 and CD33, CLDN18.2 and NKG2D, CD20 and CD22, BCMA and SLAMF7, CD19 and CD70, CD19 and IL18R1, GD2 and IL15R, CD70 and IL15R, CD19 and CD8, CD20 and CD79A, CD19 and CD22 and CD8, CD19 and EBV Protein, CD19 and CD7, BCMA and CD38, IL-12 and MUC16, CD33 and FLT3, CD19 and IL-2Rβ, BCMA and CD70, EGFR and IL-13Rα2, HER2 and IL15R, MICA and MICB, BCMA and TACI, GPC3 and IL-2Rβ, CD19 and DR5, CD123 and CD33 and CLL-1, CD3 and CD7, CEACAM5 and CEACAM6, CD20 and CD22 and CD38, CD19 and CLDN18.2, AGRE2 and CLL-1, CD123 and TIM3, CLDN18.2 and PDL1, CTLA4 and MSLN and PD-1, STEAP2 and TGFBR2, CEA and IL-15Rα and IL-21R, CD123 and CD33 and CD38 and CLL-1, CD133 and EGFR, BCMA and HPK1, CD8A and NY-ESO-1, CD38 and DR5, CD19 and EGFR, BCMA and CD19 and HER2 and Trop-2, CD19 and MUC1, PDL1 and VEGFR1, CD123 and CD33 and CD38 and CD56 and CLL-1 and MUC1, BCMA and CD138 and CD19 and CD38, BCMA and CD16a and IL-15Rα, NCR2 and NKG2D, CD38 and CLL-1, and CD276 and FGFR4.

Aspects of the present disclosure include an expression vector comprising such nucleic acids. In some embodiments, the expression vector comprises, in operable linkage to the sequence encoding the DR-18 polypeptide, a sequence encoding a signal peptide selected from: Human OSM signal peptide, VSV-G signal peptide, Mouse Ig Kappa signal peptide, Mouse Ig Heavy signal peptide, BM40 signal peptide, Secrecon signal peptide, Human IgKVIII signal peptide, CD33 signal peptide, tPA signal peptide, Human Chymotrypsinogen signal peptide, Human trypsinogen-2 signal peptide, Human IL-2 signal peptide, Gaussia luc signal peptide, Albumin (HSA) signal peptide, Influenza Haemagglutinin signal peptide, Human insulin signal peptide, Silkworm Fibroin LC signal peptide, Interleukin-18-binding protein signal peptide, Interleukin-12 subunit beta signal peptide, Interleukin-12 subunit alpha signal peptide, Interleukin-18 signal peptide, and CD8 signal peptide. In some embodiments, the expression vector comprises, in operable linkage to the sequence encoding the DR-18 polypeptide, a promoter sequence selected from: cytomegalovirus (CMV) promoter, elongation factor 1 alpha (EF1a) promoter, simian virus 40 (SV40) promoter, phosphoglycerate kinase (PGK) ubiquitin C (UBC) promoter, human beta actin promoter, CAG promoter, LC immunoglobulin promoter, HC immunoglobulin promoter, herpes simplex virus thymidine kinase promoter, and spleen focus-forming virus (SFFV) promoter. In some embodiments, the expression vector comprises, in operable linkage to the sequence encoding the DR-18 polypeptide, a human EF1a promoter. In some embodiments, the sequence encoding the DR-18 polypeptide and the sequence encoding the CAR are operably linked by a multicistronic element. In some embodiments, the multicistronic element is an internal ribosome entry site. In some embodiments, the multicistronic element is a sequence encoding 2A peptide selected from: T2A, P2A, E2A, and F2A.

Aspects of the present disclosure include a cell comprising such nucleic acids or such expression vectors. In some embodiments, the cell is selected from: a T cell, a Naïve T cell (TN), stem cell memory T cell (TSCM), a central memory T cell (TCM), a resident memory T cell (TRM), an effector T cell (TEFF), an effector memory cell (TEM), an alpha-beta (αβ) T cell, a gamma-delta (γδ) T cell, a Natural Killer (NK) cell, a CD4+ T cell, a CD4+Th1 cell, a CD4+Th2 cell, a CD4+Th9 cell, a CD4+Th17 cell, a CD4+Th22 cell, a CD4+T Regulatory Cell (Treg), a CD4+T follicular helper cell (Tfh), a resting Treg cell, an effector Treg cell, an effector memory Treg cell, a CD8+ T cell, a CD8+Tc1 cell, a CD8+Tc2 cell, a CD8+Tc9 cell, a CD8+Th17 cell, a CD8+Treg cell, a macrophage, a B Cell, a dendritic cell, a stem cell, a hematopoietic stem cell (HSC), and an induced pluripotent stem cell (iPS cell).

Aspects of the present disclosure include a method of treating a subject for cancer, the method comprising administering to the subject an effective amount of such nucleic acids, expression vectors, or cells. In some embodiments, the cancer is selected from: Acute Lymphoblastic Leukemia (ALL), Acute Myeloid Leukemia (AML), Adrenocortical Carcinoma, an AIDS-Related Cancer, Anal Cancer, Appendix Cancer, Astrocytomas, Atypical Teratoid/Rhabdoid Tumor, Basal Cell Carcinoma, Bile Duct Cancer, Bladder Cancer, Bone Cancer, Brain Stem Glioma, Brain Tumor, Breast Cancer, Bronchial Tumors, Burkitt Lymphoma, Carcinoid Tumor, Carcinoma of Unknown Primary, Cardiac (Heart) Tumors, Central Nervous System Tumor, Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct Cancer, Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric (Stomach) Cancer, Gastrointestinal Carcinoid Tumor, Gastrointestinal Stromal Tumors (GIST), Germ Cell Tumor, Gestational Trophoblastic Disease, Glioma, Hairy Cell Leukemia, Head and Neck Cancer, Heart Cancer, Hepatocellular (Liver) Cancer, Histiocytosis, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer (Primary), Lobular Carcinoma In Situ (LCIS), Lung Cancer, Lymphoma, Macroglobulinemia, Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma and Malignant Fibrous Histiocytoma of Bone, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer, Uterine Sarcoma, Vaginal Cancer, Vulvar Cancer, Waldenstrom Macroglobulinemia, and Wilms Tumor.

In some embodiments, the subject does not undergo lymphodepletion during or prior to the administering. In some embodiments, the amount of the nucleic acid, the expression vector, or the cell administered to the subject is less than the amount of a corresponding nucleic acid, expression vector, or cell comprising the sequence encoding the CAR administered to the subject to treat the cancer without the sequence encoding the DR-18 polypeptide. In some embodiments, the administering comprises delivering multiple doses of the nucleic acid, the expression vector, or the cell to the subject and the dosing frequency is less than the dosing frequency of a corresponding nucleic acid, expression vector, or cell comprising the sequence encoding the CAR administered to the subject to treat the cancer without the sequence encoding the DR-18 polypeptide.

Aspects of the present disclosure include a method comprising contacting a human cell with the described nucleic acid or expression vector under conditions sufficient for delivery of the nucleic acid or expression vector into the human cell. In some embodiments, the contacting is performed under conditions sufficient for the expression by the human cell of the CAR from the CAR encoding sequence. In some embodiments, the contacting is performed under conditions sufficient for the expression by the human cell of the CAR from the CAR coding sequence, the DR-18 polypeptide from the DR-18 polypeptide coding sequence, or both.

Aspects of the present disclosure include a nucleic acid, or expression vector, comprising or consisting of the nucleic acid, comprising a sequence encoding a decoy-resistant interleukin 18 (DR-18) polypeptide, wherein the sequence encoding the DR-18 polypeptide is codon optimized for expression in human cells. In some embodiments, the nucleic acid is configured for in vivo expression of the DR-18 polypeptide. In some embodiments, the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.

In some embodiments, the sequence encoding the DR-18 polypeptide comprises a DR-18 encoding sequence selected from SEQ ID NOs: 67-84.

In some embodiments, the nucleic acid or expression vector further comprises a sequence encoding a cytokine selected from: interleukin 2, interleukin 21, interleukin 23, interleukin 27, interleukin 33, tumor necrosis factor (TNF), TNF ligand superfamily member 15, interferon (IFN) alpha, IFN beta, and IFN gamma.

In some embodiments, the nucleic acid or expression vector further comprises, in operable linkage to the sequence encoding the DR-18 polypeptide, a sequence encoding a signal peptide selected from: Human OSM signal peptide, VSV-G signal peptide, Mouse Ig Kappa signal peptide, Mouse Ig Heavy signal peptide, BM40 signal peptide, Secrecon signal peptide, Human IgKVIII signal peptide, CD33 signal peptide, tPA signal peptide, Human Chymotrypsinogen signal peptide, Human trypsinogen-2 signal peptide, Human IL-2 signal peptide, Gaussia luc signal peptide, Albumin (HSA) signal peptide, Influenza Haemagglutinin signal peptide, Human insulin signal peptide, Silkworm Fibroin LC signal peptide, Interleukin-18-binding protein signal peptide, Interleukin-12 subunit beta signal peptide, Interleukin-12 subunit alpha signal peptide, Interleukin-18 signal peptide, and CD8 signal peptide.

In some embodiments, the nucleic acid or expression vector further comprises, in operable linkage to the sequence encoding the DR-18 polypeptide, a promoter sequence selected from: cytomegalovirus (CMV) promoter, elongation factor 1 alpha (EF1a) promoter, simian virus 40 (SV40) promoter, phosphoglycerate kinase (PGK) ubiquitin C (UBC) promoter, human beta actin promoter, CAG promoter, LC immunoglobulin promoter, HC immunoglobulin promoter, herpes simplex virus thymidine kinase promoter, and spleen focus-forming virus (SFFV) promoter.

In some embodiments, the nucleic acid or expression vector further comprises a chimeric antigen receptor (CAR) sequence encoding a CAR. In some instances, the CAR is selected from: i) an anti-5T4 CAR comprising an antigen binding domain (ABD) that specifically binds, or a means for specifically binding, 5T4; ii) an anti-AFP CAR comprising an ABD that specifically binds, or a means for specifically binding, AFP; iii) an anti-ALPP CAR comprising an ABD that specifically binds, or a means for specifically binding, ALPP; iv) an anti-ALPPL2 CAR comprising an ABD that specifically binds, or a means for specifically binding, ALPPL2; v) an anti-APN (CD13) CAR comprising an ABD that specifically binds, or a means for specifically binding, APN (CD13); vi) an anti-APRIL CAR comprising an ABD that specifically binds, or a means for specifically binding, APRIL; vii) an anti-AXL CAR comprising an ABD that specifically binds, or a means for specifically binding, AXL; viii) an anti-B7-H3 CAR comprising an ABD that specifically binds, or a means for specifically binding, B7-H3; ix) an anti-B7-H4 CAR comprising an ABD that specifically binds, or a means for specifically binding, B7-H4; x) an anti-BAFF-R CAR comprising an ABD that specifically binds, or a means for specifically binding, BAFF-R; xi) an anti-BAFF-R CAR comprising an ABD that specifically binds, or a means for specifically binding, BAFF-R; xii) an anti-BCMA CAR comprising an ABD that specifically binds, or a means for specifically binding, BCMA; xiii) an anti-BSG CAR comprising an ABD that specifically binds, or a means for specifically binding, BSG; xiv) an anti-CAIX CAR comprising an ABD that specifically binds, or a means for specifically binding, CAIX; xv) an anti-CD123 CAR comprising an ABD that specifically binds, or a means for specifically binding, CD123; xvi) an anti-CD133 CAR comprising an ABD that specifically binds, or a means for specifically binding, CD133; xvii) an anti-CD138 CAR comprising an ABD that specifically binds, or a means for specifically binding, CD138; xviii) an anti-CD19 CAR comprising an ABD that specifically binds, or a means for specifically binding, CD19; xix) an anti-CD1A CAR comprising an ABD that specifically binds, or a means for specifically binding, CD1A; xx) an anti-CD2 CAR comprising an ABD that specifically binds, or a means for specifically binding, CD2; xxi) an anti-CD20 CAR comprising an ABD that specifically binds, or a means for specifically binding, CD20; xxii) an anti-CD22 CAR comprising an ABD that specifically binds, or a means for specifically binding, CD22; xxiii) an anti-CD24 CAR comprising an ABD that specifically binds, or a means for specifically binding, CD24; xxiv) an anti-CD276 CAR comprising an ABD that specifically binds, or a means for specifically binding, CD276; xxv) an anti-CD3 CAR comprising an ABD that specifically binds, or a means for specifically binding, CD3; xxvi) an anti-CD30 CAR comprising an ABD that specifically binds, or a means for specifically binding, CD30; xxvii) an anti-CD33 CAR comprising an ABD that specifically binds, or a means for specifically binding, CD33; xxviii) an anti-CD38 CAR comprising an ABD that specifically binds, or a means for specifically binding, CD38; xxix) an anti-CD4 CAR comprising an ABD that specifically binds, or a means for specifically binding, CD4; xxx) an anti-CD43 CAR comprising an ABD that specifically binds, or a means for specifically binding, CD43; xxxi) an anti-CD44 CAR comprising an ABD that specifically binds, or a means for specifically binding, CD44; xxxii) an anti-CD44v6 CAR comprising an ABD that specifically binds, or a means for specifically binding, CD44v6; xxxiii) an anti-CD45 CAR comprising an ABD that specifically binds, or a means for specifically binding, CD45; xxxiv) an anti-CD5 CAR comprising an ABD that specifically binds, or a means for specifically binding, CD5; xxxv) an anti-CD56 CAR comprising an ABD that specifically binds, or a means for specifically binding, CD56; xxxvi) an anti-CD7 CAR comprising an ABD that specifically binds, or a means for specifically binding, CD7; xxxvii) an anti-CD70 CAR comprising an ABD that specifically binds, or a means for specifically binding, CD70; xxxviii) an anti-CD72 CAR comprising an ABD that specifically binds, or a means for specifically binding, CD72; xxxix) an anti-CD83 CAR comprising an ABD that specifically binds, or a means for specifically binding, CD83; xl) an anti-CD84 CAR comprising an ABD that specifically binds, or a means for specifically binding, CD84; xli) an anti-CD99 CAR comprising an ABD that specifically binds, or a means for specifically binding, CD99; xlii) an anti-CDCP1 CAR comprising an ABD that specifically binds, or a means for specifically binding, CDCP1; xliii) an anti-CDH17 CAR comprising an ABD that specifically binds, or a means for specifically binding, CDH17; xliv) an anti-CEA CAR comprising an ABD that specifically binds, or a means for specifically binding, CEA; xlv) an anti-CEACAM5 CAR comprising an ABD that specifically binds, or a means for specifically binding, CEACAM5; xlvi) an anti-CLDN18.2 CAR comprising an ABD that specifically binds, or a means for specifically binding, CLDN18.2; xlvii) an anti-CLDN6 CAR comprising an ABD that specifically binds, or a means for specifically binding, CLDN6; xlviii) an anti-CLEC4K (CD207) CAR comprising an ABD that specifically binds, or a means for specifically binding, CLEC4K (CD207); xlix) an anti-CLL-1 CAR comprising an ABD that specifically binds, or a means for specifically binding, CLL-1; l) an anti-CSPG4 CAR comprising an ABD that specifically binds, or a means for specifically binding, CSPG4; li) an anti-DLL3 CAR comprising an ABD that specifically binds, or a means for specifically binding, DLL3; lii) an anti-DR5 CAR comprising an ABD that specifically binds, or a means for specifically binding, DR5; liii) an anti-EBV Protein CAR comprising an ABD that specifically binds, or a means for specifically binding, EBV Protein; liv) an anti-EGFR CAR comprising an ABD that specifically binds, or a means for specifically binding, EGFR; lv) an anti-EGFRvIII CAR comprising an ABD that specifically binds, or a means for specifically binding, EGFRvIII; lvi) an anti-EMR1 CAR comprising an ABD that specifically binds, or a means for specifically binding, EMR1; lvii) an anti-enkephalinase (CD10) CAR comprising an ABD that specifically binds, or a means for specifically binding, enkephalinase (CD10); lviii) an anti-EpCAM CAR comprising an ABD that specifically binds, or a means for specifically binding, EpCAM; lix) an anti-EphA2 CAR comprising an ABD that specifically binds, or a means for specifically binding, EphA2; lx) an anti-EphA3 CAR comprising an ABD that specifically binds, or a means for specifically binding, EphA3; lxi) an anti-FAP CAR comprising an ABD that specifically binds, or a means for specifically binding, FAP; lxii) an anti-FGFR4 CAR comprising an ABD that specifically binds, or a means for specifically binding, FGFR4; lxiii) an anti-FLT3 CAR comprising an ABD that specifically binds, or a means for specifically binding, FLT3; lxiv) an anti-FOLR1 CAR comprising an ABD that specifically binds, or a means for specifically binding, FOLR1; lxv) an anti-FSHR CAR comprising an ABD that specifically binds, or a means for specifically binding, FSHR; lxvi) an anti-GC-C CAR comprising an ABD that specifically binds, or a means for specifically binding, GC-C; lxvii) an anti-GD2 CAR comprising an ABD that specifically binds, or a means for specifically binding, GD2; lxviii) an anti-GFRA4 CAR comprising an ABD that specifically binds, or a means for specifically binding, GFRA4; lxix) an anti-Globo H CAR comprising an ABD that specifically binds, or a means for specifically binding, Globo H; lxx) an anti-GM2 (ganglioside M2) CAR comprising an ABD that specifically binds, or a means for specifically binding, GM2 (ganglioside M2); lxxi) an anti-gp100 CAR comprising an ABD that specifically binds, or a means for specifically binding, gp100; lxxii) an anti-GPC1 CAR comprising an ABD that specifically binds, or a means for specifically binding, GPC1; lxxiii) an anti-GPC2 CAR comprising an ABD that specifically binds, or a means for specifically binding, GPC2; lxxiv) an anti-GPC3 CAR comprising an ABD that specifically binds, or a means for specifically binding, GPC3; lxxv) an anti-HAAH (ASPH) CAR comprising an ABD that specifically binds, or a means for specifically binding, HAAH (ASPH); lxxvi) an anti-HER2 CAR comprising an ABD that specifically binds, or a means for specifically binding, HER2; lxxvii) an anti-HGF CAR comprising an ABD that specifically binds, or a means for specifically binding, HGF; lxxviii) an anti-HIV envelope protein gp120 CAR comprising an ABD that specifically binds, or a means for specifically binding, HIV envelope protein gp120; lxxix) an anti-HIV-1 pol CAR comprising an ABD that specifically binds, or a means for specifically binding, HIV-1 pol; lxxx) an anti-HLA-A2 CAR comprising an ABD that specifically binds, or a means for specifically binding, HLA-A2; lxxxi) an anti-HLA-G CAR comprising an ABD that specifically binds, or a means for specifically binding, HLA-G; lxxxii) an anti-HSP70 heat-shock protein CAR comprising an ABD that specifically binds, or a means for specifically binding, HSP70 heat-shock protein; lxxxiii) an anti-ICAM-1 CAR comprising an ABD that specifically binds, or a means for specifically binding, ICAM-1; lxxxiv) an anti-IL-10R CAR comprising an ABD that specifically binds, or a means for specifically binding, IL-10R; lxxxv) an anti-IL13Rα2 CAR comprising an ABD that specifically binds, or a means for specifically binding, IL13Rα2; lxxxvi) an anti-IL-13Rα2 CAR comprising an ABD that specifically binds, or a means for specifically binding, IL-13Rα2; lxxxvii) an anti-integrin αvβ6 CAR comprising an ABD that specifically binds, or a means for specifically binding, integrin αvβ6; lxxxviii) an anti-ITGB7 CAR comprising an ABD that specifically binds, or a means for specifically binding, ITGB7; lxxxix) an anti-KKLC1 CAR comprising an ABD that specifically binds, or a means for specifically binding, KKLC1; xc) an anti-KMA CAR comprising an ABD that specifically binds, or a means for specifically binding, KMA; xci) an anti-L1CAM CAR comprising an ABD that specifically binds, or a means for specifically binding, L1CAM; xcii) an anti-Lewis-Y antigen CAR comprising an ABD that specifically binds, or a means for specifically binding, Lewis-Y antigen; xciii) an anti-LGR5 CAR comprising an ABD that specifically binds, or a means for specifically binding, LGR5; xciv) an anti-LILRB4 CAR comprising an ABD that specifically binds, or a means for specifically binding, LILRB4; xcv) an anti-LMP1 CAR comprising an ABD that specifically binds, or a means for specifically binding, LMP1; xcvi) an anti-MAGEA3 CAR comprising an ABD that specifically binds, or a means for specifically binding, MAGEA3; xcvii) an anti-M-CSF CAR comprising an ABD that specifically binds, or a means for specifically binding, M-CSF; xcviii) an anti-MG7 CAR comprising an ABD that specifically binds, or a means for specifically binding, MG7; xcix) an anti-MICA CAR comprising an ABD that specifically binds, or a means for specifically binding, MICA; c) an anti-MMP2 CAR comprising an ABD that specifically binds, or a means for specifically binding, MMP2; ci) an anti-MSLN CAR comprising an ABD that specifically binds, or a means for specifically binding, MSLN; cii) an anti-MUC1/MUC16 CAR comprising an ABD that specifically binds, or a means for specifically binding, MUC1/MUC16; ciii) an anti-NCR3LG1 CAR comprising an ABD that specifically binds, or a means for specifically binding, NCR3LG1; civ) an anti-NECTIN2 CAR comprising an ABD that specifically binds, or a means for specifically binding, NECTIN2; cv) an anti-nectin-4 CAR comprising an ABD that specifically binds, or a means for specifically binding, nectin-4; cvi) an anti-NKG2D CAR comprising an ABD that specifically binds, or a means for specifically binding, NKG2D; cvii) an anti-NKG2DL CAR comprising an ABD that specifically binds, or a means for specifically binding, NKG2DL; cviii) an anti-NR2F6 CAR comprising an ABD that specifically binds, or a means for specifically binding, NR2F6; cix) an anti-NY-ESO-1 CAR comprising an ABD that specifically binds, or a means for specifically binding, NY-ESO-1; cx) an anti-OPCML CAR comprising an ABD that specifically binds, or a means for specifically binding, OPCML; cxi) an anti-PD-1 CAR comprising an ABD that specifically binds, or a means for specifically binding, PD-1; cxii) an anti-PDGFRα CAR comprising an ABD that specifically binds, or a means for specifically binding, PDGFRα; cxiii) an anti-PDL1 CAR comprising an ABD that specifically binds, or a means for specifically binding, PDL1; cxiv) an anti-PLA2R CAR comprising an ABD that specifically binds, or a means for specifically binding, PLA2R; cxv) an anti-PR1 CAR comprising an ABD that specifically binds, or a means for specifically binding, PR1; cxvi) an anti-PSCA CAR comprising an ABD that specifically binds, or a means for specifically binding, PSCA; cxvii) an anti-PTK7 CAR comprising an ABD that specifically binds, or a means for specifically binding, PTK7; cxviii) an anti-PVR CAR comprising an ABD that specifically binds, or a means for specifically binding, PVR; cxix) an anti-ROBO1 CAR comprising an ABD that specifically binds, or a means for specifically binding, ROBO1; cxx) an anti-ROR1 CAR comprising an ABD that specifically binds, or a means for specifically binding, ROR1; cxxi) an anti-ROR2 CAR comprising an ABD that specifically binds, or a means for specifically binding, ROR2; cxxii) an anti-SEMA4A CAR comprising an ABD that specifically binds, or a means for specifically binding, SEMA4A; cxxiii) an anti-SLAMF7 CAR comprising an ABD that specifically binds, or a means for specifically binding, SLAMF7; cxxiv) an anti-TAG-72 CAR comprising an ABD that specifically binds, or a means for specifically binding, TAG-72; cxxv) an anti-TGF-β CAR comprising an ABD that specifically binds, or a means for specifically binding, TGF-β; cxxvi) an anti-TM4SF1 CAR comprising an ABD that specifically binds, or a means for specifically binding, TM4SF1; cxxvii) an anti-TRAIL CAR comprising an ABD that specifically binds, or a means for specifically binding, TRAIL; cxxviii) an anti-TRBC1 CAR comprising an ABD that specifically binds, or a means for specifically binding, TRBC1; cxxix) an anti-TRBC2 CAR comprising an ABD that specifically binds, or a means for specifically binding, TRBC2; cxxx) an anti-Trop-2 CAR comprising an ABD that specifically binds, or a means for specifically binding, Trop-2; cxxxi) an anti-TSHR CAR comprising an ABD that specifically binds, or a means for specifically binding, TSHR; cxxxii) an anti-TSLPR CAR comprising an ABD that specifically binds, or a means for specifically binding, TSLPR; cxxxiii) an anti-ULBP1 CAR comprising an ABD that specifically binds, or a means for specifically binding, ULBP1; cxxxiv) an anti-αvβ3 integrin CAR comprising an ABD that specifically binds, or a means for specifically binding, αvβ3 integrin; and cxxxv) an anti-K light chain of human immunoglobulin CAR comprising an ABD that specifically binds, or a means for specifically binding, K light chain of human immunoglobulin.

In some embodiments, the CAR is selected from: a) inaticabtagene autoleucel; b) actalycabtagene autoleucel; c) relmacabtagene autoleucel; d) lisocabtagene maraleucel; e) brexucabtagene autoleucel; f) axicabtagene ciloleucel; g) tisagenlecleucel; h) obecabatagene autoleucel; i) azercabtagene zapreleucel; j) rapcabtagene autoleucel; and k) idecabtagene vicleucel.

In some embodiments, the sequence encoding the CAR and the sequence encoding the DR-18 polypeptide are operably linked by a multicistronic element. In some embodiments, the multicistronic element is an internal ribosome entry site. In some embodiments, the multicistronic element is a sequence encoding 2A peptide selected from: T2A, P2A, E2A, and F2A.

In some embodiments, the CAR is an anti-EGFRvIII CAR comprising a means for specifically binding EGFRvIII. In some embodiments, the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO: 110, and one or more of heavy chain complementary determining region 1 (HC CDR1), heavy chain complementary determining region 2 (HC CDR2), and heavy chain complementary determining region 3 (HC CDR3) of SEQ ID NO:111. In some embodiments, the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO:117). In some embodiments, the antigen binding domain comprises a light chain variable region comprising sequence SEQ ID NO:110 and a heavy chain variable region comprising sequence SEQ ID NO:111.

Aspects of the present disclosure include a cell comprising the described nucleic acid or expression vector. In some embodiments, the cell is selected from: a T cell, a Naïve T cell (TN), stem cell memory T cell (TSCM), a central memory T cell (TCM), a resident memory T cell (TRM), an effector T cell (TEFF), an effector memory cell (TEM), an alpha-beta (αβ) T cell, a gamma-delta (γα) T cell, a Natural Killer (NK) cell, a CD4+ T cell, a CD4+Th1 cell, a CD4+Th2 cell, a CD4+Th9 cell, a CD4+Th17 cell, a CD4+Th22 cell, a CD4+T Regulatory Cell (Treg), a CD4+T follicular helper cell (Tfh), a resting Treg cell, an effector Treg cell, an effector memory Treg cell, a CD8+ T cell, a CD8+Tc1 cell, a CD8+Tc2 cell, a CD8+Tc9 cell, a CD8+Th17 cell, a CD8+Treg cell, a macrophage, a B Cell, a dendritic cell, a stem cell, a hematopoietic stem cell (HSC), and an induced pluripotent stem cell (iPS cell).

Aspects of the present disclosure include a method of treating a subject for cancer, the method comprising administering to the subject an effective amount of the described nucleic acid, expression vector, or cells. In some embodiments, the subject does not undergo lymphodepletion during or prior to the administering. In some embodiments, the amount of the nucleic acid, the expression vector, or the cell administered to the subject is less than the amount of a corresponding nucleic acid, expression vector, or cell comprising the sequence encoding the CAR administered to the subject to treat the cancer without the sequence encoding the DR-18 polypeptide. In some embodiments, the administering comprises delivering multiple doses of the nucleic acid, the expression vector, or the cell to the subject and the dosing frequency is less than the dosing frequency of a corresponding nucleic acid, expression vector, or cell comprising the sequence encoding the CAR administered to the subject to treat the cancer without the sequence encoding the DR-18 polypeptide.

Aspects of the present disclosure include a method of making a population of therapeutic cells, the method comprising: a) contacting a human cell with a nucleic acid comprising a chimeric antigen receptor (CAR) sequence encoding a CAR and a sequence encoding a decoy-resistant interleukin 18 (DR-18) polypeptide; wherein the nucleic acid is configured to result in expression and secretion of the DR-18 by the human cell ex vivo; b) culturing the contacted human cell ex vivo in the presence of the secreted DR-18 polypeptide, thereby producing a population of therapeutic cells, wherein the population of therapeutic cells is enhanced as compared to a corresponding population of cells generated without the sequence encoding the DR-18 polypeptide.

In some embodiments, the population of therapeutic cells demonstrates enhanced ex vivo proliferation as compared to the corresponding population of cells. In some embodiments, the population of therapeutic cells demonstrates enhanced in vivo proliferation as compared to the corresponding population of cells. In some embodiments, the population of therapeutic cells demonstrates enhanced in vivo persistence as compared to the corresponding population of cells. In some embodiments, the population of therapeutic cells demonstrates enhanced tumor cell killing as compared to the corresponding population of cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts the architecture of certain anti-epidermal growth factor variant Ill (EGFRvIII) chimeric antigen receptor (CAR) constructs as described herein. “IL-18” indicated in the drawing can refer to either wild-type human IL-18 or DR-18. Elements are not drawn to scale. Arrows indicate read-direction. The presence or absence of linking sequence between elements cannot necessarily be inferred from the existence of space or lack of space between individual elements.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, “codon” refers to a sequence of three nucleotides that together form a unit of genetic code in a DNA or RNA molecule. The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Statistics Database” (see e.g., Subramanian et al. (2022) Molecular Biology and Evolution 39(8)), “Codon Usage Database” at www(dot)kazusa(dot)or(dot)jp/codon/, High-performance Integrated Virtual Environment-Codon Usage Tables (HIVE-CUTs) (see e.g., Athey et al. (2017) BMC Bioinformatics 18(391)), and the like.

“Operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “vector” or “expression vector” is a replicon, such as plasmid, phage, virus, or cosmid, to which another DNA segment, i.e. an “insert”, may be attached so as to bring about the replication of the attached segment in a cell.

Nucleic acids can be delivered into a cell in a variety of ways, including but not limited to e.g., as naked nucleic acid (e.g., naked DNA, naked plasmid, etc.), as a nucleic acid-protein complex (e.g., a DNA-protein complex, an RNA-protein complex such as a ribonucleoprotein (RNP) complex, and the like), as nucleic acid within a viral vector, as nucleic acid within a non-viral vector, and the like. In some instances, proteins may be incorporated into the delivery of a nucleic acid agent, such as but not limited to DNA-binding proteins, RNA-binding proteins, enzymes such as nucleases, and combinations thereof.

Useful viral vectors include non-replicating (i.e., replication deficient) viral vectors. Viral vectors may be integrating or non-integrating. Non-limiting examples of useful viral vectors include retroviral vectors, lentiviral vectors, adenoviral vectors, adeno-associated virus (AAV) vectors, and the like.

Nonviral vectors, which are delivery means that do not employ viral particles, may generally be considered to fall into three categories: naked nucleic acid, particle based (e.g., nanoparticles), or chemical based. Non-limiting examples of nonviral vectors include lipoplexes (e.g., cationic lipid-based lipoplexes), emulsions (such as e.g., lipid nano emulsions), lipid nanoparticles (LNPs), solid lipid nanoparticles, peptide based vectors, polymer based vectors (e.g., polymersomes, polyplexes, polyethylenimine (PEI)-based vectors, chitosan-based vectors, poly (DL-Lactide) (PLA) and poly (DL-Lactide-co-glycoside) (PLGA)-based vectors, dendrimers, vinyl based polymers (e.g., polymethacrylate-based vectors), and the like), inorganic nanoparticles, and the like.

“Heterologous nucleotide” and “heterologous polypeptide” as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “derivative” and “variant” refer without limitation to any compound such as nucleic acid or protein that has a structure or sequence derived from the compounds disclosed herein and whose structure or sequence is sufficiently similar to those disclosed herein such that it has the same or similar activities and utilities or, based upon such similarity, would be expected by one skilled in the art to exhibit the same or similar activities and utilities as the referenced compounds, thereby also interchangeably referred to “functionally equivalent” or as “functional equivalents.” Modifications to obtain “derivatives” or “variants” may include, for example, addition, deletion and/or substitution of one or more of the nucleic acids or amino acid residues.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 and US Patent Application No. 2015/0368342). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013);5(215):215ral72; Glienke et al. Front Pharmacol (2015) 6:21; Kakarla & Gottschalk 52 Cancer J (2014) 20(2): 151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies, and may include post-translational modifications thereof (e.g., C-terminal Lysine clipping in the heavy chain, conversion of glutamine or glutamic acid to pyroglutamate) that may occur when an antibody is recombinantly expressed in host cells (e.g., CHO cells), or during purification/storage. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, fusion proteins comprising an antigen binding fragment thereof that competes with the intact antibody for specific.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “VH.” The variable region of the light chain may be referred to as “VL.”

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. As referred to herein the light chain CDRs are CDRL1, CDRL2 and CDRL3, respectively, and the heavy chain CDRs are CDRH1, CDRH2 and CDRH3, respectively. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved β-sheet framework, and thus are able to adapt to different conformations. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in the following table. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system. Correspondence between the CDR Numbering Systems

| | Kabat + | | | | | |
|---|---|---|---|---|---|---|
| | Chothia | IMGT | Kabat | AbM | Chothia | Contact |
| VH CDR1 | 26-35 | 27-38 | 31-35 | 26-35 | 26-32 | 30-35 |
| VH CDR2 | 50-65 | 56-65 | 50-65 | 50-58 | 52-56 | 47-58 |
| VH CDR3 | 95-102 | 105-117 | 95-102 | 95-102 | 95-102 | 93-101 |
| VL CDR1 | 24-34 | 27-38 | 24-34 | 24-34 | 24-34 | 30-36 |
| VL CDR2 | 50-56 | 56-65 | 50-56 | 50-56 | 50-56 | 46-55 |
| VL CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from nonhuman (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions and the ability to bind specifically to the antigen. An antibody that “specifically binds to” CD20 is an antibody that exhibits preferential binding to CD20 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., CD20). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody (or antigen-binding region of a CAR) for unrelated amino acid sequences. Affinity of an antibody (or antigen-binding region of a CAR) to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, a specific binding member present in the extracellular domain of a chimeric polypeptide of the present disclosure binds specifically to its binding partner, such as an antigen. “Specific binding” refers to binding with an affinity of at least about 10⁷ M or greater, e.g., 5×10⁷ M, 10⁸ M, 5×10⁸ M, and greater. “Non-specific binding” refers to binding with an affinity of less than about 10⁷ M, e.g., binding with an affinity of 10⁶ M, 105 M, 104 M, etc.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The term “subject in need thereof” as used herein refers to a subject diagnosed with or suspected of having a disease, such as, but not necessarily limited to, cancer.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

The terms “stem cell”, “stem cells”, and the like, as used herein refer to cells that are capable of differentiating into two of more different cell types and proliferating. Non limiting examples of stem cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like. Stem cells may be acquired from public or commercial sources or may be newly derived.

The term “synthetic” as used herein generally refers to an artificially derived polypeptide or polypeptide encoding nucleic acid that is not naturally occurring. Such synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “control”, “control reaction”, “control assay”, and the like, refer to a reaction, test, or other portion of an experimental or diagnostic procedure or experimental design for which an expected result is known with high certainty, e.g., in order to indicate whether the results obtained from associated experimental samples are reliable, indicate to what degree of confidence associated experimental results indicate a true result, and/or to allow for the calibration of experimental results. For example, in some instances, a control may be a “negative control” such that an essential component of the assay is excluded from the negative control reaction such that an experimenter may have high certainty that the negative control reaction will not produce a positive result. In some instances, a control may be “positive control” such that all components of a particular assay are characterized and known, when combined, to produce a particular result in the assay being performed such that an experimenter may have high certainty that the positive control reaction will not produce a positive negative result.

A “biological sample” encompasses a variety of sample types obtained from an individual or a population of individuals and can be used in a diagnostic, monitoring or screening assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of includes determining the amount of something present, and/or determining whether it is present or absent. As used herein, the terms “determining,” “measuring,” and “assessing,” and “assaying” are used interchangeably and include both quantitative and qualitative determinations.

### DETAILED DESCRIPTION

Provided are nucleic acids that encode decoy-resistant interleukin 18 (DR-18) polypeptides, as well as vectors and cells comprising such nucleic acids and cells that comprise such vectors. Cells encoding DR-18 polypeptides may be referred to as DR-18 armored cells. The nucleic acids may further encode a chimeric antigen receptor (CAR) or multiple CARs, including CARs that bind to antigens described herein. Methods are also provided, including methods of making the nucleic acids, vectors, and/or cells, as well as methods of use, such as employing the nucleic acids, vectors, and/or cells, in the treatment of a subject having cancer.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Systems & Compositions

The present disclosure provides compositions, including nucleic acid compositions for the expression of polypeptides from a cell into which the nucleic acids have been introduced. For example, a nucleic acid composition of the present disclosure may be contacted with a human cell under conditions sufficient for the nucleic acid composition to enter the cell and one or more encoded polypeptides to be expressed from the cell. The subject nucleic acid compositions may be contacted with cells ex vivo to introduce the nucleic acids into the cells, e.g., outside of a human body. The subject nucleic acid compositions may be contacted with the cells in vivo, e.g., through administration of a nucleic acid composition to a human subject, such that the nucleic acids are introduced in a cell of the subject within the human body.

Nucleic acid compositions of the present disclosure will include a sequence encoding a polypeptide, such as decoy-resistant interleukin 18 (DR-18) polypeptide, which polypeptides are described in more detail below. A cell or cells expressing a DR-18 polypeptide, or transduced or transfected with a DR-18 encoding nucleic acid, may be referred to herein as a DR-18 armored cell or cells. Nucleic acid compositions of the present disclosure may include a sequence encoding a chimeric antigen receptor (CAR) or multiple sequences encoding CARs, including e.g., where the nucleic acid includes multiple CAR-encoding sequences each encoding a different CAR. CARs, and their components, are described in more detail below. Sequences that encode polypeptides in such systems are not limited to DR-18 polypeptides and CAR polypeptides and may include various other polypeptide-encoding sequences such as, but not limited to e.g., various other cytokine-encoding sequences, and the like. A nucleic acid, construct, system, or cell involving the expression of a DR-18 polypeptide and a CAR, such as those configured with a DR-18 encoding nucleic acid and a CAR encoding nucleic acid, may be referred to herein as DR-18 armored, such as a DR-18 armored CAR or a DR-18 armored CAR nucleic acid, construct, vector, system, cell, or any combination thereof.

Systems of multiple nucleic acid sequences are also provided. At least one sequence of such a system will encode a polypeptide. Systems may include multiple polypeptide-encoding sequences each encoding a different polypeptide, such as e.g., one sequence encoding a DR-18 polypeptide and one sequence encoding a CAR. As described in more detail below, multiple polypeptide-encoding sequences in a nucleic acid may be configured in a multicistronic fashion, such as in a multicistronic expression vectors, to allow for co-expression of the multiple polypeptides from the nucleic acid. Accordingly, systems of the present disclosure include wherein multiple different polypeptides are encoded on and expressed from the same polynucleic acid molecule. Systems of the present disclosure include wherein multiple different polypeptides are encoded on and expressed from separate polynucleic acid molecules. Systems of the present disclosure also include combinations wherein one or more polypeptides are encoded on and expressed from a first polynucleic acid molecule and one or more different polypeptides are encoded on and expressed from a second polynucleic acid molecule.

Expressed polypeptides of the herein described systems may or may not directly or indirectly interact. For example, in the case of a DR-18 polypeptide and a CAR expressed from a nucleic acid the polypeptides may not directly bind to one another or may not share a common binding partner and thus will not directly or indirectly interact. In another example, in the case of a split polypeptide, such as a split CAR, the two or more polypeptides of the split polypeptide may be configured to directly bind to one another, and thus directly interact, or to directly bind to a common binding partner, and thus indirectly interact.

Polypeptides encoded in such systems may or may not be modified following translation. For example, an expressed polypeptide may be configured to include a heterologous cleavage site that is cut at some point following translation or an expressed polypeptide may include a portion, endogenous or heterologous, of the polypeptide that is cleaved following translation, such as e.g., an endogenous or heterologous signal peptide. Modification of an expressed polypeptide is not limited to cleavage and may include, e.g., essentially any post-translational modification.

Such systems may also include one or more sequences that do not code for a polypeptide, which may be referred to herein as non-coding sequences. Such non-coding sequences may provide for various functions within the system. For example, useful non-coding sequences include regulatory elements, such as DNA regulatory elements, post-transcriptional regulatory elements, and other functional non-coding elements, such as e.g., promoters, enhancers, introns, kozak sequences, slice acceptor sites, splice donor sites, internal ribosome entry sites, and the like.

### Decoy Resistant IL-18 Polypeptides

As described herein, nucleic acid compositions and/or systems of the present disclosure will include a sequence encoding a DR-18 polypeptide. A DR-18 polypeptide-encoding sequence may be present on the same polynucleic acid molecule (such as e.g., the same vector) as a CAR-encoding sequence or the two encoding sequences may be present on separate polynucleic acid molecules (such as e.g., separate vectors). Useful DR-18 polypeptides that may be encoded by a sequence present in a nucleic acid will vary and are described in more detail below.

Decoy-resistant IL-18 polypeptides bind to, and signal through formation of, the IL-18Rα (IL-18 receptor α) and IL-18Rβ (IL-18 receptor β) receptor complex. Decoy-resistant IL-18 polypeptides do not bind to IL-18 binding protein (IL-18BP), or display substantially reduced binding to IL-18BP, such as substantially reduced binding to IL-18BP relative to wild-type (WT) human IL-18 (SEQ ID NO: 1) (i.e., as compared to the binding of IL-18BP to WT IL-18 (SEQ ID NO: 1)). In some embodiments, the DR IL-18 polypeptide does not bind to IL-18BP. In some embodiments, the DR IL-18 polypeptide has reduced binding to IL-18BP relative to WT IL-18. In some embodiments, the DR IL-18 polypeptide binds to IL-18Rα and does not bind to IL-18BP. In some embodiments, the DR IL-18 polypeptide binds to IL-18Rα and has reduced binding to IL-18BP relative to WT IL-18. In some instances, substantially reduced binding to IL-18BP may be represented by a DR IL-18 polypeptide having a Ko for IL-18BP of at least 10 nM or greater. In some instances, substantially reduced binding to IL-18BP may be represented by a DR IL-18 polypeptide having a Ko for IL-18BP that is at least twice, at least three times, at least four times, at least five times or greater, or at least ten times the Ko of WT human IL-18 for IL-18BP.

DR-18 polypeptides described herein are expressed from DR-18 encoding nucleic acids. Human WT IL-18 is expressed initially in a pre-processed form (SEQ ID NO: 2) that includes a 36 amino acid propeptide (SEQ ID NO: 3). DR-18 encoding nucleic acids may include or exclude sequence encoding the WT human IL-18 propeptide (SEQ ID NO: 3). As such, DR-18 polypeptides may be expressed with or without the WT human IL-18 propeptide. DR-18 polypeptides expressed with a WT human IL-18 propeptide are subsequently processed, similar to human WT IL-18, to produce the active mature form of the polypeptide. Unless clearly intended otherwise, references to WT IL-18 polypeptides and DR-18 polypeptides will generally refer to the active mature forms of the polypeptides (i.e., without a WT human IL-18 propeptide). For example, reference to a DR-18 polypeptide having X mutations relative to WT human IL-18 shall, unless specifically indicated otherwise, refer to X mutations occurring within amino acids 1 to 157 of SEQ ID NO: 1 or amino acids 37 to 193 of SEQ ID NO: 2.

In some embodiments, the DR-18 polypeptide has at least about two mutations, at least about three mutations, at least about four mutations, at least about five mutations, at least about six mutations, at least about seven mutations, at least about eight mutations, at least about nine mutations, or at least about ten mutations, relative to WT IL-18 as set forth in SEQ ID NO: 1. In some embodiments, the DR-18 polypeptide has about two mutations, about three mutations, about four mutations, about five mutations, about six mutations, about seven mutations, about eight mutations, about nine mutations, or about ten mutations, relative to WT IL-18 as set forth in SEQ ID NO: 1. In some instances, a DR-18 polypeptide may comprise an amino acid sequence having at least 85%, 86%, 87%, 88%, 89%, 90%, 91%, 92%, 93%, 94%, 95%, 96%, 97%, 98%, or 99% sequence identity with WT IL-18 as set forth in SEQ ID NO: 1.

Useful DR-18 polypeptides include but are not limited to e.g., a DR-18 polypeptide comprising any of the amino acid sequences of SEQ ID NOs.:4-65. In some instances, a DR-18 polypeptide may comprise an amino acid sequence having at least 85%, 86%, 87%, 88%, 89%, 90%, 91%, 92%, 93%, 94%, 95%, 96%, 97%, 98%, or 99% with at least one of SEQ ID NOs: 4-65. In some instances, a DR-18 polypeptide may comprise an amino acid sequence having 100% or at least 95%, 96%, 97%, 98%, or 99% with SEQ ID NO:04. In some instances, a DR-18 polypeptide may comprise an amino acid sequence having 100% or at least 95%, 96%, 97%, 98%, or 99% with SEQ ID NO:19. In some instances, a DR-18 polypeptide may comprise an amino acid sequence having 100% or at least 95%, 96%, 97%, 98%, or 99% with SEQ ID NO:20. In some instances, a DR-18 polypeptide may comprise an amino acid sequence having 100% or at least 95%, 96%, 97%, 98%, or 99% with SEQ ID NO:21. In some instances, a DR-18 polypeptide may comprise an amino acid sequence having 100% or at least 95%, 96%, 97%, 98%, or 99% with SEQ ID NO:22. In some instances, a DR-18 polypeptide may comprise an amino acid sequence having 100% or at least 95%, 96%, 97%, 98%, or 99% with SEQ ID NO:23. In some instances, a DR-18 polypeptide may comprise an amino acid sequence having 100% or at least 95%, 96%, 97%, 98%, or 99% with SEQ ID NO:24.

In some instances, a useful DR-18 polypeptide comprises at least 1, 2, 3, 4, 5, 6, 7, 8, 9, 10, 12, 13, 14, or 15 mutated residues relative to SEQ ID NO:1. In some instances, a useful DR-18 polypeptide comprises up to 15, 14, 13, 12, 11, or 10 mutated residues relative to SEQ ID NO:1. In some instances, a useful DR-18 polypeptide comprises from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 1 to 18, from 2 to 18, from 3 to 18, from 4 to 18, from 5 to 18, from 6 to 18, from 7 to 18, from 8 to 18, from 9 to 18, from 10 to 18, from 11 to 18, from 12 to 18, from 13 to 18, from 14 to 18, from 15 to 18, from 1 to 16, from 2 to 16, from 3 to 16, from 4 to 16, from 5 to 16, from 6 to 16, from 7 to 16, from 8 to 16, from 9 to 16, from 10 to 16, from 11 to 16, from 12 to 16, from 13 to 16, from 14 to 16, from 1 to 14, from 2 to 14, from 3 to 14, from 4 to 14, from 5 to 14, from 6 to 14, from 7 to 14, from 8 to 14, from 9 to 14, from 10 to 14, from 11 to 14, from 12 to 14, from 1 to 12, from 2 to 12, from 3 to 12, from 4 to 12, from 5 to 12, from 6 to 12, from 7 to 12, from 8 to 12, from 9 to 12, from 10 to 12, from 1 to 10, from 2 to 10, from 3 to 10, from 4 to 10, from 5 to 10, from 6 to 10, from 7 to 10, or from 8 to 10 mutated residues relative to SEQ ID NO:1.

In some instances, a useful DR-18 polypeptide comprises an amino acid sequence of any one of SEQ ID NOs: 4-65 with 0, 1, 2, 3, 4, 5, or 6 additional mutated residues relative to SEQ ID NO:1.

In some instances, a useful DR-18 polypeptide comprises one or more mutations at positions Y1, L5, K8, C38, M51, K53, S55, Q56, P57, G59, M60, C68, E77, Q103, S105, D110, N111, M113, V153, and N155 relative to the WT IL-18 amino acid sequence set forth in SEQ ID NO:1, including e.g., at least 1, at least 2, at least 3, at least 4, at least 5, at least 6, at least 7, at least, 8, at least 9, at least 10, at least 11, or at least 12 of the listed mutated positions. Accordingly, useful DR-18 polypeptides can have one or any combination of mutations at the listed positions, with or without additional mutated residues at other positions. For example, useful polypeptides can have a mutation at position Y1, L5, K8, C38, M51, K53, S55, Q56, P57, G59, M60, C68, E77, Q103, S105, D110, N111, M113, V153, or N155 only; mutations at all positions Y1, L5, K8, C38, M51, K53, S55, Q56, P57, G59, M60, C68, E77, Q103, S105, D110, N111, M113, V153, and N155; or any combinations having more than one but less than all (e.g., 2, 3, 4, 5, 6, 7, 8, 9, 10, 11, 12, 13, 14, 15, 16, 17, 18 or 19) mutations at these positions.

In some instances, the DR-18 polypeptide comprises at least 12 mutations, at least 11 mutations, at least 10 mutations, at least 9 mutations, at least 8 mutations, at least 7 mutations, at least 6 mutations, at least 5 mutations, at least 4 mutations, at least 3 mutations, at least 2 mutations, or at least 1 mutation relative to the wild-type IL-18 amino acid sequence set forth in SEQ ID NO:1, optionally wherein the at least 10, 9, 8, 7, 6, 5, 4, 3, 2, or 1 mutations are at positions selected from Y1, L5, K8, C38, M51, K53, S55, Q56, P57, G59, M60, C68, E77, Q103, S105, D110, N111, M113, V153, and N155.

In some instances, the DR-18 polypeptide has less than 100%, 99%, 98%, 97%, 96%, 95%, 94%, 93%, 92%, 91%, 90%, 89%, 88%, 87%, 86%, or 85% sequence identity to WT human IL-18 (SEQ ID NO: 1) and comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.

In some instances, the DR-18 polypeptide comprises one or more substitutions relative to WT human IL-18 (SEQ ID NO: 1), including 1, 2, 3, 4, 5, 6, 7, 8, 9, 10 or more substitutions selected from: Y1H, Y1R, L5H, L51, L5Y, K8Q, K8R, M51T, M51K, M51D, M51N, M51E, M51R, K53R, K53G, K53S, K53T, S55K, S55R, Q56E, Q56A, Q56R, Q56V, Q56G, Q56K, Q56L, P57L, P57G, P57A, P57K, G59T, G59A, M60K, M60Q, M60R, M60L, E77D, Q103E, Q103K, Q103P, Q103A, Q103R, S105R, S105D, S105K, S105N, S105A, D110H, D110K, D110N, D110Q, D110E, D110S, D110G, N111H, N111Y, N111D, N111R, N111S, N111G, M113V, M113R, M113T, M113K, V1531, V153T, V153A, N155K, and N155H.

In some instances, the DR-18 polypeptide comprises one or more substitutions relative to WT human IL-18 (SEQ ID NO: 1) such that, e.g., the amino acid at pos. 1 is not Y and, e.g., may be R or H; the amino acid at pos. 5 is not L and, e.g., may be H, I or Y; the amino acid at pos. 8 is not K and, e.g., may be Q or R; the amino acid at pos. 38 is not C and, e.g., may be S; the amino acid at pos. 51 is not M and, e.g., may be T, K, D, N, E or R; the amino acid at pos. 53 is not K and, e.g., may be R, G, S or T; the amino acid at pos. 55 is not S and, e.g., may be K or R; the amino acid at pos. 56 is not Q and, e.g., may be E, A, R, V, G, K, L or R; the amino acid at pos. 57 is not P and, e.g., may be L, G, A or K; the amino acid at pos. 59 is not G and, e.g., may be A or T; the amino acid at pos. 60 is not M and, e.g., may be K, Q, R or L; the amino acid at pos. 68 is not C and, e.g., may be S, G, A, V, D, E or N; the amino acid at pos. 76 is not C and, e.g., may be S; the amino acid at pos. 77 is not E and, e.g., may be D; the amino acid at pos. 103 is not Q and, e.g., may be E, K, P, A or R; the amino acid at pos. 105 is not S and, e.g., may be D, N, R, K or A; the amino acid at pos. 110 is not D and, e.g., may be K, H, N, Q, E, S or G; the amino acid at pos. 111 is not N and, e.g., may be H, Y, D, R, S or G; the amino acid at pos. 113 is not M and, e.g., may be V, R, T or K; the amino acid at pos. 127 is not C and, e.g., may be S; the amino acid at pos. 153 is not V and, e.g., may be I, T or A; and/or the amino acid at pos. 155 is not N and, e.g., may be K or H.

In some instances, the DR-18 polypeptide comprises substitution mutations, relative to WT human IL-18 as set forth in SEQ ID NO:1, at positions: (i) M51, M60, S105, D110, and N111; (ii) M51, S55, G59, M60, S105, D110, N111, and V153; (iii) Y1, M51, M60, S105, D110, and N111; (iv) Y1, M51, K53, M60, S105, D110, and N111; (v) K8, M51, S55, G59, M60, S105, D110, and N155; (vi) K8, M51, S55, G59, M60, S105, D110, N111, and V153; (vii) L5, M51, K53, M60, S105, D110, and V153; (viii) L5, M51, S55, G59, M60, S105, D110, N111, and N155; (ix) L5, M51, S55, M60, Q103, S105, D110, N111, and V153; (x) L5, M51, S55, M60, S105, D110, N111, V153, and N155; (xi) L5, M51, S55, G59, M60, S105, D110, N111, V153, and N155; (xii) L5, K8, M51, S55, M60, S105, N111, V153, and N155; (xiii) L5, K8, M51, K53, M60, S105, D110, N111, and N155; (xiv) Y1, L5, M51, K53, M60, S105, D110, and N155; (xv) Y1, M51, K53, G59, M60, S105, D110, N111, V153, and N155; (xvi) Y1, K8, M51, K53, M60, Q103, S105, D110, N111, and N155; (xvii) Y1, K8, M51, M60, S105, D110, and N111; (xviii) Y1, L5, M51, K53, M60, Q103, S105, D110, and N111; (xix) Y1, K8, M51, K53, G59, M60, Q103, S105, D110, N111, V153, and N155; (xx) Y1, K8, M51, K53, G59, M60, S105, D110, N111, and N155; (xxi) Y1, K8, M51, G59, M60, Q103, S105, D110, N111, V153, and N155; (xxii) Y1, L5, M51, G59, M60, E77, S105, D110, and N111; (xxiii) M51, Q56, P57, M60, Q103, S105, D110, N111, and M113; (xxiv) M51, Q56, P57, M60, Q103, S105, D110, and M113; (xxv) M51, K53, Q56, P57, M60, D110, and N111; (xxvi) M51, K53, Q56, P57, M60, Q103, S105, D110, N111, and M113; (xxvii) M51, K53, Q56, M60, Q103, S105, D110, N111, and M113; (xxviii) M51, K53, Q56, P57, Q103, S105, D110, N111, and M113; (xxix) M51, K53, Q56, P57, M60, S105, D110, and N111; (xxx) M51, K53, Q56, P57, M60, Q103, D110, N111, and M113; (xxxi) M51, Q56, P57, M60, Q103, D110, N111, and M113; or (xxxii) M51, K53, Q56, S105, D110, and N111.

In some instances, the DR-18 polypeptide comprises substitution, relative to WT human IL-18 as set forth in SEQ ID NO:1, of: (i) M51T, M60K, S105D, D110K, and N111H; (ii) M51T, S55K, G59A, M60K, S105D, D110K, N111H, and V1531; (iii) Y1R, M51T, M60K, S105D, D110K, and N111H; (iv) Y1R, M51T, K53R, M60K, S105N, D110K, and N111Y; (v) K8Q, M51T, S55K, G59T, M60K, S105R, D110H, and N155K; (vi) K8R, M51K, S55K, G59A, M60Q, S105D, D110K, N111H, and V1531; (vii) K8R, M51D, S55K, G59A, M60X, S105D, D110K, N111H, and V1531; (viii) L5H, M51T, K53R, M60K, S105D, D110N, and V153T; (ix) L51, M51K, S55K, G59A, M60Q, S105K, D110Q, N111H, and N155K; (x) L51, M51T, S55R, M60K, Q103E, S105D, D110H, N111H, and V1531; (xi) L51, M51T, S55K, M60K, S105D, D110K, N111H, V153T, and N155H; (xii) L51, M51T, S55K, G59A, M60K, S105R, D110H, N111H, V1531, and N155K; (xiii) L51, K8R, M51T, S55K, M60K, S105D, N111Y, V1531, and N155K; (xiv) L5Y, K8R, M51T, K53R, M60K, S105D, D110E, N111H, and N155K; (xv) Y1H, L5Y, M51T, K53R, M60K, S105D, D110H, and N155K; (xvi) Y1R, M51T, K53R, G59A, M60K, S105D, D110Q, N111H, V153A, and N155K; (xvii) Y1R, K8R, M51D, K53R, M60R, Q103K, S105N, D110K, N111Y, and N155H; (xviii) Y1R, K8R, M51N, K53R, M60Q, Q103K, S105R, D110N, N111H, and N155K; (xix) Y1R, K8R, M51T, M60K, S105D, D110K, and N111H; (xx) Y1R, L5H, M51T, K53R, M60K, Q103E, S105N, D110K, and N111Y; (xxi) Y1R, K8R, M51T, K53R, G59A, M60K, Q103E, S105D, D110Q, N111H, V1531, and N155X; (xxii) Y1R, K8R, M51T, K53R, G59T, M60K, S105N, D110H, N111D, and N155H; (xxiii) Y1R, K8R, M51T, G59A, M60K, Q103E, S105D, D110Q, N111H, V1531, and N155K; (xxiv) Y1R, L5Y, M51T, G59T, M60K, E77D, S105D, D110K, and N111H; (xxv) Y1R, K8R, M51T, K53R, G59T, M60K, S105K, D110N, N111H, and N155K; (xxvi) M51E, Q56E, P57L, M60R, Q103P, S105A, D110N, N111R, and M113V; (xxvii) M51K, Q56A, P57G, M60L, Q103E, S105D, D110S, and M113V; (xxviii) M51K, K53G, Q56A, P57A, M60L, D110K, and N111R; (xxix) M51K, K53G, Q56R, P57G, M60L, Q103E, S105D, D110N, N111S, and M113R; (xxx) M51K, K53G, Q56V, M60L, Q103A, S105A, D110S, N111R, and M113T; (xxxi) M51K, K53S, Q56G, P57A, M60L, Q103A, S105A, D110G, N111R, and M113T; (xxxii) M51K, K53S, Q56K, P57A, Q103A, S105D, D110S, N111S, and M113R; (xxxiii) M51K, K53S, Q56L, P57A, M60L, S105D, D110S, and N111R; (xxxiv) M51K, K53S, Q56R, P57A, M60L, S105N, D110G, and N111R; (xxxv) M51K, K53S, Q56R, P57A, M60L, Q103A, D110G, N111R, and M113T; (xxxvi) M51K, K53S, Q56R, P57A, M60L, Q103A, S105D, D110S, N111G, and M113R; (xxxvii) M51K, K53T, Q56R, M60L, Q103E, S105D, D110S, N111S, and M113K; (xxxviii) M51K, K53T, Q56R, P57A, Q103E, S105D, D110N, N111D, and M113R; (xxxix) M51R, Q56G, P57K, M60L, Q103R, D110S, N111R, and M113V; (xl) M51K, K53G, Q56G, P57A, M60L, Q103E, S105D, D110S, N111G, and M113V; or (xli) M51K, K53G, Q56R, S105A, D110N, and N111R.

In some embodiments, the DR IL-18 polypeptide comprises: (i) an amino acid sequence having 85% or more (e.g., 90%, 91%, 92%, 93%, 94%, 95%, 96%, 97% or 98%) sequence identity with the any one of the amino sequences set forth in SEQ ID NO: 4-65; and (ii) one or more mutations at a wild-type cysteine present in WT IL-18 as set forth in SEQ ID NO: 1, such as e.g., C38, C68, C76, and/or C127. For example, in some instances, the DR IL-18 polypeptide comprises substitutions at amino acid positions Cysteine-38 and Cysteine-68 relative to WT human IL-18 SEQ ID NO: 1. In some instances, DR-18 polypeptide includes a stabilized DR-18 polypeptide. A stabilized DR-18 polypeptide can include mutations of two cysteine residues (e.g., C38 and C68) relative to human WT IL-18 (SEQ ID NO: 1). In some cases, the mutation is a C to S substitution and as such the stabilized IL-18 polypeptide can in some cases comprise the mutations C38S and C68S. In some cases a stabilized IL-18 polypeptide comprises the mutation pair C38S/C68S, C38S/C68G, C38S/C68A, C38S/C68V, C38S/C68D, C38S/C68E, or C38S/C68N (e.g., in some cases C38S/C68G, C38S/C68A, C38S/C68V, C38S/C68D, C38S/C68E, or C38S/C68N; in some cases C38S/C68S, C38S/C68G, C38S/C68A, C38S/C68D, or C38S/C68N; and in some cases C38S/C68G, C38S/C68A, C38S/C68D, or C38S/C68N).

DR-18 polypeptides can, additionally, be stabilized variants, through the presence of one or more stabilizing mutations. Useful DR-18 polypeptides, including in stabilized and non-stabilized forms, include those described in PCT Pub. Nos. WO2019/051015 and WO2022/094473, the disclosures of which are incorporated herein by reference in their entirety.

Useful IL-18 variants, as well as useful mutations and substitutions thereof, include but are not limited to those variants, mutations and substitutions described in WO2002101049 (such as e.g., variants having mutations at positions E42 and/or K89 and substitutions E42A and K89A (numbering according to SEQ ID NO:2)), US20080206189 (such as e.g., variants having mutations at one or more of C38, C68, C76, N78, E121, C127, L144, and D157 and substitutions, individually or in combination, of C38S, C68D, C68S, N78C, E121C, L144C, and D157C), WO2023118497 (such as e.g., variants having mutations at one or more of Y1, G3, S10, C38, M51, K53, S55, P57, M60, C68, Q103, M113, C127, and N155, and substitutions, individually or in combination, of Y1A, G3Y, S10R, S10K, C38S, M51A, M51Y, M51Q, K53L, K53H, K53A, S55L, S55Y, P57T, M60A, M60E, M60H, C68S, Q103S, M113A, M113E, C127S, and N155Y (inc. those at pos. K53 other than K53R, K53G, K53S, or K53T), WO2023114829 (such as e.g., variants having mutations at one or more of Y37, S43, L45, S46, V47, N50, F57, C74, T81, 185, S86, D90, S91, A97, V98, T99, S101, C104, 1107, T109, C112, N123, P124, Q139, R140, Q150, A162, C163, L174, 1185, V189, Q190, and E192, and substitutions, individually or in combination, of Y37C, S43C, L45C, S46C, V47C, N50C, F57C, C74S, T81C, 185C, S86C, D90C, S91C, A97C, V98C, T99C, S101C, C104S,1107C, T109C, C112S, N123C, P124C, Q139C, R140C, Q150C, A162C, C163S, L174C, 1185C, V189C, Q190C, E192C, (numbering according to SEQ ID NO:2)), WO2023056193 (such as e.g., variants having mutations at one or more of M51, K53, Q56, P57 and M60, and substitutions, individually or in combination, of M51A, M51S, M51G, K53G, K53A, K53S, Q56R, Q56K, Q56H, P57A, P57S, P57G, M60R, M60K, and M60H (and useful sequences encoding such variants, such as e.g., SEQ ID NOs: 7 and 8 of WO2023056193)), US20230146665 (such as e.g., variants having mutations at one or more of Y1, E6, S7, K8, S10, V11, N14, L15, D17, Q18, D23, R27, P28, L29, E31, M33, T34, D35, S36, D37, C38, R39, D40, N41, R44, 146, 149, S50, M51, K53, D54, S55, Q56, P57, M60, A61, V62, T63, S65, K67, C68, E69, 171, C76, E77, 180,181, N87, P88, D90, K93, T95, K96, S97, Q103, H109, D110, N111, M113, S119, A126, C127, D132, L136, L138, K139, E141, L144, D146, R147, 1149, M150, N155, E156, and D157, and substitutions, individually or in combination, of Y1F, Y1H, E6A, E6Q, S7C, S7P, K8E, K8Q, K8Y, S10C, V111, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C381, C38V, C38K, C38D, R39S, R39T, D40N, N41Q, R44Q, 146V, 149C, S50C, S50Y, M511, M51K, M51Q, M51R, M51L, M51H, M51F, M51Y, K53A, K53D, K53E, K53G, K53H, K531, K53L, K53M, K53N, K53Q, K53R, K53S, K53T, K53V, K53Y, K53F, D54C, S55N, S55Q, S55D, S55E, S55T, Q561, Q56L, P57A, P57E, P57T, P57V, P57Q, P57D, P57Y, P57N, M601, M60L, M60K, M60Y, M60F, A61C, V62C, T63C, S65C, K67Q, C68S, C681, C68F, C68Y, C68D, C68N, C68E, C68Q, C68K, E69K, 171M, C76S, C76E, C76K, E77K, 180T, 181L, 181V, N87S, P88C, D90E, K93D, K93N, T95E, K96G, K96Q, S97N, Q103C, Q103E, Q1031, Q103L, H109W, H109Y, D110N, D110Q, D110R, N111D, N111Q, N111S, N111T, N111E, M1131, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, N155C, E156Q, D157A, D157S, and D157N, as well as deletion mutants described therein), US20220056091 (such as e.g., variants having mutations at one or more of Y01, F02, E06, M33, C38, M51, K53, D54, S55, M60, T63, C68, E69, K70, C76, M86, M113, C127, and M150, and substitutions, individually or in combination, of Y01G, F02A, E06K, C38A, C38S, K53A, D54A, S55A, T63A, C68A, C68S, E69C, K70C, C76A, C76S, C127A, and C127S), US20230357342 (such as e.g., variants having mutations at one or more of Y1, F2, E6, K8, S10, V11, D17, E31, T34, D35, S36, D37, C38, D40, N41, 149, M51, K53, D54, S55, T63, C68, E69, K70, C76, Q103, S105, G108, H109, D110, C127, D132, and V153, and substitutions, individually or in combination, of Y1M, Y1G, F2A, E6K, E6R, K8E, K8L, K8R, S10T, V111, D17N, E31A, T34A, D35A, S36A, D37A, C38A, C38Q, C38S, D40A, N41A, 149E, 149M, 149R, M51G, K53A, D54A, S55A, S55H, S55R, S55T, T63A, C68A, C68S, E69C, K70C, C76A, C76S, Q103E, Q103K, Q103R, S1051, S105K, G108A, H109A, D110A, C127A, C127S, D132A, V153E, V153R, and V153Y), and WO2022172944 (such as e.g., variants having mutations at one or more of C38, C68, C76, C127, G3, A6, S7, T34, S50, M51, S72, K112, K119, and G145, and substitutions, individually or in combination, of C38S, C68S, C76S, C127S, G3Y, G3L, A6W, S7C, T34P, C38M, S50C, M51Y, S72Y, S72F, S72M, S72L, S72W, K112W, K119V, and G145N).

A nucleic acid sequence encoding a DR-18 polypeptide may be generated from the DR-18 polypeptide amino acid sequence, including e.g., any of the DR-18 polypeptide amino acid sequences described herein. Such DR-18 polypeptide-encoding sequences may be produced by conversion of each amino acid residue to a corresponding trinucleotide codon that codes for the residue. Such DR-18 polypeptide-encoding sequences may include a stop codon after the codon designating the terminal amino acid. In some instances, a stop codon may be added.

In some instances, DR-18 polypeptide encoding sequences of the present disclosure may be codon optimized, i.e., optimized for expression in a particular host cell or organism. In some instances, a DR-18 polypeptide encoding sequence of the present disclosure is optimized for expression in human cells, i.e., is human codon optimized. Useful methods for codon optimization, including human codon optimization, include but are not limited to e.g., those described in US20140244228, US20210366574, US20190325989, US20230245721, US20160259885, US20140377800, WO2020024917, WO2022221576, WO2024018050, and WO2009009743.

Useful human codon optimized DR-18 polypeptide-encoding sequences include human codon optimized versions encoding DR-18 polypeptides referred to herein as “hCS1” (SEQ ID NO:25), such as e.g., SEQ ID NOs:67 and 68; “hCS2” (SEQ ID NO:26), such as e.g., SEQ ID NOs:69 and 70; “hCS3” (SEQ ID NO:27), such as e.g., SEQ ID NOs:71 and 72; “hCS4” (SEQ ID NO:28), such as e.g., SEQ ID NOs:73 and 74; “6-12” (SEQ ID NO:57), such as e.g., SEQ ID NOs:75 and 76; “sq89v6” (SEQ ID NO:9), such as e.g., SEQ ID NOs:77 and 78; “C68v1” (SEQ ID NO:19), such as e.g., SEQ ID NOs:79 and 80; “C68v2” (SEQ ID NO:20), such as e.g., SEQ ID NOs:81 and 82; and “C68v4” (SEQ ID NO:22), such as e.g., SEQ ID NOs:83 and 84. The forgoing codon optimized sequences are merely examples and alternative codon optimized sequences, encoding these DR-18 polypeptides or any other DR-18 polypeptide, may be readily generated, e.g., by modifying the parameters of codon optimization algorithms that generate codon optimized nucleic acid sequences from input amino acid sequences. Common codon optimization parameters that may be modified include, but are not limited to, the allowance or prohibition of certain restriction enzyme recognition sites, the allowed or prohibited frequency of rare codons, the allowed presence or absence of out-of-frame cryptic translation start sites, the allowed or preferred overall GC concentration, the allowed or preferred GC distribution patterns, guanine and cytosine content at the third codon position (GC3), and the like.

In some embodiments, the sequence encoding the DR-18 polypeptide has at least 80%, 81%, 82%, 83%, 84%, 85%, 86%, 87%, 88%, 89%, 90%, 91%, 92%, 93%, 94%, 95%, 96%, 97%, 98%, or 99%, or has 100%, sequence identity with any one of SEQ ID NOs: 67-84. In some embodiments, the sequence encoding the DR-18 polypeptide has less than 100% but at least 80%, 81%, 82%, 83%, 84%, 85%, 86%, 87%, 88%, 89%, 90%, 91%, 92%, 93%, 94%, 95%, 96%, 97%, 98%, or 99% sequence identity with any one of SEQ ID NOs: 67-84.

### Chimeric Antigen Receptors (CARs)

As described herein, nucleic acid compositions and/or systems of the present disclosure may include a sequence encoding a CAR, or multiple sequences encoding multiple different CARs. A CAR-encoding sequence may be present on the same polynucleic acid molecule (such as e.g., the same vector) as a DR-18-encoding sequence or the two encoding sequences may be present on separate polynucleic acid molecules (such as e.g., separate vectors). Where multiple different CARs are employed, the multiple different CAR-encoding sequence may be present on the same polynucleic acid molecule (such as e.g., the same vector) or on separate polynucleic acid molecules (such as e.g., separate vectors).

A CAR is a synthetic receptor engineered to grant immune cells, such as T cells, the ability to target and destroy specific target cells, including cancer cells, as desired. A CAR is a fusion protein typically including an extracellular recognition domain (extracellular antigen-recognition domain), often but not exclusively derived from a monoclonal antibody, and an intracellular signaling domain that activates the immune cell upon binding of the extracellular recognition domain to its binding partner (e.g., antigen). The extracellular part of a CAR will specifically recognize, or bind to, one or more binding partners (e.g., antigens), often present on the surface of a target cell, such as a tumor cell. While useful binding partners of a CAR will vary widely, for simplicity, the binding partner bound by the extracellular recognition domain of a CAR may be generally referred to herein as an antigen. However, use of the term “antigen”, except where expressly indicated otherwise, should not be interpreted as limiting, e.g., on the nature, type, identity, or function of the CAR or relevant extracellular recognition domain at least because an ordinarily skilled artisan will readily recognize that various types of extracellular recognition domains that have various different types of binding partners may be employed in a CAR.

The intracellular portion of the CAR, once engaged with the antigen, triggers signaling events that lead to cell activation, proliferation, and/or release of signaling molecules, such as endogenous cytokines. Activation enables immune cells modified to express the CAR, such as CAR T cells, e.g., to effectively locate and eliminate cancer cells. CAR-based cell therapy, which often involves the genetic modification (ex vivo or in vivo) of a patient's own (i.e., autologous) cells to express one or more CARs, has shown significant success, resulting in numerous commercial and clinical stage CAR-based therapies. In some instances, the cells employed in a CAR-based cell therapy, i.e., administered to a subject in need thereof, may be derived from a heterologous (e.g., allogeneic) source, i.e., as related to the subject being treated.

CARs have been engineered into T cells; however, their use is not so limited. CARs may be engineered into a variety of cell types, including but not limited to, e.g., immune cells generally, T cells, Naïve T cells (T_{N}), stem cell memory T cells (T_{SCM}), central memory T cells (T_{CM}), resident memory T cells (T_{RM}), effector T cells (T_{EFF}), effector memory cells (T_{EM}), alpha-beta (αβ) T cells, gamma-delta (γα) T cells, Natural Killer (NK) cells, CD4+ T cells, CD4+ T cell subsets (e.g., Th1, Th2, Th9, Th17, Th22, T Regulatory Cells (Tregs), and Tfh), resting Treg cells, effector Treg cells, effector memory Treg cells, CD8+ T cells, CD8+T subsets (e.g., Tc1, Tc2, Tc9, Th17, and Treg), macrophages, B Cells, Dendritic Cells (DCs), stem cells, and the like.

Different populations and subpopulations of cells, including immune cells, stem cells, and the like, can be characterized, separated, and/or subdivided based on expression (e.g., presence/absence, level (inc. relative levels, e.g., high, low, etc.) of cellular (inc. extracellular (e.g., cell surface) and/or intracellular), epigenetic, and genetic markers, as well as expression, activation, or inactivity of transcription factors and metabolic pathways, and combinations thereof. Useful markers include e.g., CD3, CD4, CD8, CCR4, CCR5, CCR6, CCR7, CCR10, CD127, CD27, CD28, CD38, CD40, CD45, CD45RA, CD45RO, CD58, CD69, CD83, CD161, CTLA4, CXCR3, CXCR4, FA5, HLA-DR, IL2RA (CD25), IL2RB, ITGAE, ITGAL, KLRB1 (CD16), NCAM1 (CD56), PECAM1, PTGDR2, CD62L (SELL), IFNG, IL10, IL13, IL17A, IL2, IL21, IL22, IL25, IL26, IL4, IL5, IL9, IL-23R, ITGB1, TNF, AHR, EOMES, FOXO4, FOXP3, GATA3, IRF4, LEF1, PRDM1, RORC, STAT4, STAT5A, TBX21, TCF7, GZMA, alpha-beta (αβ) T cell receptor, gamma-delta (γα) T cell receptor, E-Cadherin, Dectin-1 (CLEC7A), Fas (TNFRSF6/CD95), Fas Ligand (TNFSF6), ICOS, NKG2D (CD314), NKG2E, Occludin, TLR2, TRAIL/TNFSF10, and the like. In some instances, cells may be characterized by their cytokine profiles, such as e.g., Th1: interferon gamma (IFN-γ), tumor necrosis factor (TNF); Th2: IL-1, IL-5, and IL-13; Th9: IL-9; Th17: IL-17, IL-21, IL-22, IL-25, and IL-26; Th22: IL-22; Treg: IL-10 and TGF-β; Tfh: IL-21; and the like. Examples of relevant markers of cytotoxic T cell populations include e.g., Tc1: TNF alpha, INF gamma, IL-2 CXCR3, and TBX21; Tc2: IL-4, IL-5, CCR4, and GATA3; Tc9: IL-9, IL-10, and IRF4; Tc17: CCR6, KLRB1, IL-17, IRF4, and RORC; and the like.

As noted above, a CAR is a fusion protein that generally includes one or more extracellular antigen-recognition domains, a transmembrane domain, and one or more intracellular signaling domains. In some instances, one or more domains of a CAR may be separated onto two or more polypeptide chains that may be joined, reversibly or irreversibly and intracellularly or intracellularly, to form a functional CAR (such as e.g., as described in WO2014127261; WO2016138034; Liu et al. Ther Adv Med Oncol. 2020, Cho et al. Nature Communications. 2021; and the like). Useful CAR domains, and methods of screening for useful combinations of CAR domains, include those described in WO2017040694.

Useful intracellular signaling domains can include one or more immunoreceptor tyrosine-based activation motif (ITAM) containing domains, such as e.g., those derived from the T-cell receptor (TCR) complex. Useful intracellular domains include chains of the CD3 protein complex or portions thereof, such as the CD3 zeta (CD3ζ), CD3 delta (CD3δ), CD3 gamma (CD3γ), and CD3 epsilon (CD3ε) chains and portions thereof, often referred to as CD3ζ, CD3δ, CD3γ, and CD3ε intracellular signaling domains. Examples of ITAM containing intracellular signaling domains that are of particular use include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. Further examples of molecules containing a primary intracellular signaling domain that are of particular use include those of DAP10, DAP12, and CD32. In some embodiments, a CAR of the invention comprises an intracellular signaling domain, e.g., a primary signaling domain of CD3-zeta.The number of ITAMs present in an ITAM-containing intracellular signaling domain will vary and may include e.g., one ITAM or multiple ITAMs, such as e.g., 2, 3, 4, or more ITAMs, and may include the natural number of ITAMs occurring in the endogenous protein or a number that is more or fewer than the natural number of ITAMs occurring in the endogenous protein (e.g., by engineering the protein to introduce, duplicate, or remove ITAMs).

Useful intracellular signaling domains also include downstream molecules of TCR signaling including domains derived from proximal TCR signaling molecules, such as e.g., Lymphocyte-specific PTK (LCK), Tyrosine-protein kinase Fyn (FYN), 70 kDa zeta-chain associated protein (ZAP-70), Linker for activation of T-cells family member 1 (LAT), SH2 domain-containing leukocyte protein of 76 kDa (SLP-76), Phosphoinositide phospholipase C-gamma-1 (PLCy1), and the like. For example, in some instances, a ZAP-70-based intracellular signaling domain and/or a PLCy1-based intracellular signaling domain may be employed as the intracellular signaling domain of a CAR (see e.g., Tousley et al. Nature 2023 615(7952): 507-516).

In some embodiments, a primary signaling domain may be employed. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d.

CARs may include one or more co-stimulatory domains, e.g., in addition to an ITAM-containing intracellular signaling domain (e.g., from a CD3(chain) or other intracellular signaling domain (e.g., from ZAP-70). Useful co-stimulatory domains include but are not limited to those derived from CD28, 4-1BB (CD137), or OX40 (CD134). In some embodiments, the costimulatory domain is a functional signaling domain of a protein selected from: MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand that specifically binds with CD83.

A CAR will further include a transmembrane (TM) region, linking the intracellular and extracellular portions of the CAR, and a hinge region. Common sources for the hinge region include segments from immunoglobulin G (IgG), such as the CH2-CH3 domains, including from IgG4 and IgG1, and segments from other proteins like CD8a or CD28. Common sources for the TM region include, but are not limited to, the TM regions of CD4, CD8, CD28, immunoglobulins (such as IgG1 and IgG4), and the like. In some embodiments, the transmembrane domain is transmembrane domain of a protein selected from: the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. The hinge domain and TM domain of a CAR may share a common source (such as e.g., CD28 hinge+CD28 TM, IgG4 hinge+IgG4 TM, etc.) or may be derived from different sources (such as e.g., IgG4 hinge+CD28 TM, IgG1 hinge+CD4 TM, IgG4 hinge+CD28 TM, etc.).

As previously noted, a CAR may be expressed as a single polypeptide chain (i.e., including extracellular, transmembrane, and intracellular domains in a single polypeptide chain). In some instances, a CAR may be expressed as multiple polypeptides (e.g., with at least one domain of the CAR on a polypeptide that is separate from a different polypeptide that contains one or more other domains of the CAR) that are joined reversibly or irreversibly to form the functional CAR. Useful architectures of a CAR split between two or more polypeptides vary widely.

The extracellular recognition domain of the CAR determines the specificity of the CAR and may be monospecific or multi-specific. Useful extracellular recognition domains, also referred to antigen-binding domains, antigen-recognition domains, or simply recognition domains, are commonly derived from antibodies and various forms can be used including but not limited to e.g., signal-chain variable fragment (scFv) based domains, VHH (nanobody) based domains, Fv fragment based domains, and the like. Useful antigen-recognition domains are not necessarily limited to those derived from antibodies and also include e.g., those derived from natural or engineered ligand-receptor interactions. For example, a receptor, or portion thereof, that binds a ligand may be employed as the recognition domain where the ligand is the target. Alternatively, a ligand, or portion thereof, that binds to a receptor may be employed as the recognition domain where the receptor is the target. Accordingly, essentially any binding partner, or binding portion thereof, that binds to the relevant target, whether natural or synthetic, may be employed including e.g., where the antigen recognition domain is (or is derived from) a ligand, a receptor, a peptide-MHC complex, an aptamer, or the like. A monospecific CAR will have an antigen-recognition domain that binds a single antigen. A multi-specific CAR may have a multi-specific antigen-recognition domain that binds multiple different antigens, multiple different antigen-recognition domains that each bind a single (but different) antigen, or any combination thereof.

Useful antigen-recognition domains include antigen-recognition domains that recognize the following antigens: CD19, BCMA, HER2, CD22, MSLN, CLDN18.2, CD20, CD7, GPC3, CD30, MUC1, CD123, CD276, EGFRvIII, NKG2D, GD2, CD70, CD33, CD5, EGFR, CD38, CEACAM5, ROR1, IL-13Rα2, CEA, FOLR1, PDL1, HLA-A2, PD-1, CLL-1, LGR5, BAFF-R, EpCAM, K light chain of human immunoglobulins, CDH17, DLL3, ALPP, ULBP1, ROR2, CD44v6, SLAMF7, DR5, PSCA, CD4, MMP2, EBV Protein, LILRB4, EphA2, GPC2, FLT3, TAG-72, HLA-G, GC-C, CD133, TRBC1, AFP, BAFF-R, ICAM-1, CD1A, B7-H3, L1CAM, CD44, CD83, CD84, Lewis-Y antigen, CD99, TSHR, TM4SF1, CLDN6, HIV-1 pol, NY-ESO-1, KMA, 5T4, ALPPL2, ROBO1, αvβ3 integrin, CD43, NKG2DL, GM2 (ganglioside M2), CD24, HAAH (ASPH), gp100, CD72, OPCML, FSHR, MAGEA3, NCR3LG1, CLEC4K (CD207), CAIX, BSG, CD45, AXL, ITGB7, FGFR4, PLA2R, MG7, CD138, FAP, Trop-2, APRIL, TRAIL, HSP70 heat-shock proteins, PR1, M-CSF, CSPG4, PVR, B7-H4, nectin-4, KKLC1, Globo H, CD56, LMP1, CD3, TSLPR, TGF-β, SEMA4A, integrin av36, NECTIN2, enkephalinase (CD10), HIV envelope protein gp120, EMR1, EphA3, APN (CD13), TRBC2, GPC1, CDCP1, HGF, IL-10R, GFRA4, CD2, NR2F6, MICA, PTK7, PDGFRα, IL13Rα2, and the like. Useful antigen recognition domain further include antigen-recognition domains that recognize the antigens described in US20220127373, US20230398216, WO2020097395, and WO2022087453; the disclosures of which are incorporated by reference in their entirety. Useful antigen recognition domains, as well as antibodies from which antigen recognition domains can be derived, that bind the aforementioned antigens, and methods of making additional antigen recognition domains to such antigens, are known to the relevant skilled artisan. Examples of useful antigen recognition domains and antibodies from which additional antigen recognition domains can be derived for a representative sampling of antigens are as follows:

CD123 antigen recognition domains are known to the relevant skilled artisan and include, e.g., the heavy and light chain domains of pivekimab, talacotuzumab, vibecotamab, and the like. Useful antigen recognition domains include those derived from the heavy chain, light chain, and/or CDRs of antibodies that bind to CD123, such as but not limited to e.g., pivekimab, talacotuzumab, vibecotamab, and the like.

CD19 antigen recognition domains are known to the relevant skilled artisan and include, e.g., the heavy and light chain domains of budoprutug, coltuximab, denintuzumab, inebilizumab, leronlimab, loncastuximab, mogamulizumab, obexelimab, plozalizumab, tafasitamab, and the like. Useful antigen recognition domains include those derived from the heavy chain, light chain, and/or CDRs of antibodies that bind to CD19, such as but not limited to e.g., budoprutug, coltuximab, denintuzumab, inebilizumab, leronlimab, loncastuximab, mogamulizumab, obexelimab, plozalizumab, tafasitamab, and the like.

CD20 antigen recognition domains are known to the relevant skilled artisan and include, e.g., the heavy and light chain domains of divozilimab, ibritumomab, obinutuzumab, ocaratuzumab, ocrelizumab, ofatumumab, ripertamab, rituximab, ublituximab, veltuzumab, zuberitamab, and the like. Useful antigen recognition domains include those derived from the heavy chain, light chain, and/or CDRs of antibodies that bind to CD20, such as but not limited to e.g., divozilimab, ibritumomab, obinutuzumab, ocaratuzumab, ocrelizumab, ofatumumab, ripertamab, rituximab, ublituximab, veltuzumab, zuberitamab, and the like.

CD22 antigen recognition domains are known to the relevant skilled artisan and include, e.g., the heavy and light chain domains of epratuzumab, inotuzumab, pinatuzumab, suciraslimab, and the like. Useful antigen recognition domains include those derived from the heavy chain, light chain, and/or CDRs of antibodies that bind to CD22, such as but not limited to e.g., epratuzumab, inotuzumab, pinatuzumab, suciraslimab, and the like.

CD30 antigen recognition domains are known to the relevant skilled artisan and include, e.g., the heavy and light chain domains of brentuximab, iratumumab, and the like. Useful antigen recognition domains include those derived from the heavy chain, light chain, and/or CDRs of antibodies that bind to CD30, such as but not limited to e.g., brentuximab, iratumumab, and the like.

CD33 antigen recognition domains are known to the relevant skilled artisan and include, e.g., the heavy and light chain domains of gemtuzumab, lintuzumab, vadastuximab, and the like. Useful antigen recognition domains include those derived from the heavy chain, light chain, and/or CDRs of antibodies that bind to CD33, such as but not limited to e.g., gemtuzumab, lintuzumab, vadastuximab, and the like.

CD38 antigen recognition domains are known to the relevant skilled artisan and include, e.g., the heavy and light chain domains of daratumumab, erzotabart, felzartamab, isatuximab, lumrotatug, mezagitamab, modakafusp, and the like. Useful antigen recognition domains include those derived from the heavy chain, light chain, and/or CDRs of antibodies that bind to CD38, such as but not limited to e.g., daratumumab, erzotabart, felzartamab, isatuximab, lumrotatug, mezagitamab, modakafusp, and the like.

CD70 antigen recognition domains are known to the relevant skilled artisan and include, e.g., the heavy and light chain domains of cusatuzumab, vorsetuzumab, and the like. Useful antigen recognition domains include those derived from the heavy chain, light chain, and/or CDRs of antibodies that bind to CD70, such as but not limited to e.g., cusatuzumab, vorsetuzumab, and the like.

CLDN18.2 antigen recognition domains are known to the relevant skilled artisan and include e.g., the heavy and light chain domains of osemitamab, zolbetuximab, and the like. Useful antigen recognition domains include those derived from the heavy chain, light chain, and/or CDRs of antibodies that bind to CLDN18.2, such as but not limited to e.g., osemitamab, zolbetuximab, and the like.

CLL-1 antigen recognition domains are known to the relevant skilled artisan and include, e.g., the heavy and light chain domains of tepoditamab, and the like. Useful antigen recognition domains include those derived from the heavy chain, light chain, and/or CDRs of antibodies that bind to CLL-1, such as but not limited to e.g., tepoditamab, and the like.

DLL3 antigen recognition domains are known to the relevant skilled artisan and include, e.g., the heavy and light chain domains of rovalpituzumab, tarlatamab, and the like. Useful antigen recognition domains include those derived from the heavy chain, light chain, and/or CDRs of antibodies that bind to DLL3, such as but not limited to e.g., rovalpituzumab, tarlatamab, and the like.

EGFR antigen recognition domains are known to the relevant skilled artisan and include, e.g., the heavy and light chain domains of becotatug, cetuximab, demupitamab, futuximab, imgatuzumab, matuzumab, modotuximab, necitumumab, nimotuzumab, panitumumab, pimurutamab, tomuzotuximab, zalutumumab, and the like. Useful antigen recognition domains include those derived from the heavy chain, light chain, and/or CDRs of antibodies that bind to EGFR, such as but not limited to e.g., becotatug, cetuximab, demupitamab, futuximab, imgatuzumab, matuzumab, modotuximab, necitumumab, nimotuzumab, panitumumab, pimurutamab, tomuzotuximab, zalutumumab, and the like.

EGFRvII antigen recognition domains are known to the relevant skilled artisan and include e.g., the heavy and light chain domains of umizortamig, and the like. Useful antigen recognition domains include those derived from the heavy chain, light chain, and/or CDRs of antibodies that bind to EGFRvIII, such as but not limited to e.g., umizortamig, and the like.

FLT3 antigen recognition domains are known to the relevant skilled artisan and include, e.g., the heavy and light chain domains of emirodatamab, and the like. Useful antigen recognition domains include those derived from the heavy chain, light chain, and/or CDRs of antibodies that bind to FLT3, such as but not limited to e.g., emirodatamab, and the like.

GD2 antigen recognition domains are known to the relevant skilled artisan and include, e.g., the heavy and light chain domains of dinutuximab, lorukafusp, naxitamab, and the like. Useful antigen recognition domains include those derived from the heavy chain, light chain, and/or CDRs of antibodies that bind to GD2, such as but not limited to e.g., dinutuximab, lorukafusp, naxitamab, and the like.

GPC3 antigen recognition domains are known to the relevant skilled artisan and include, e.g., the heavy and light chain domains of codrituzumab, and the like. Useful antigen recognition domains include those derived from the heavy chain, light chain, and/or CDRs of antibodies that bind to GPC3, such as but not limited to e.g., codrituzumab, and the like.

HER2 antigen recognition domains are known to the relevant skilled artisan and include, e.g., the heavy and light chain domains of anvatabart, coprelotamab, disitamab, gancotamab, margetuximab, marstacimab, pertuzumab, timigutuzumab, trastuzumab, and the like. Useful antigen recognition domains include those derived from the heavy chain, light chain, and/or CDRs of antibodies that bind to HER2, such as but not limited to e.g., anvatabart, coprelotamab, disitamab, gancotamab, margetuximab, marstacimab, pertuzumab, timigutuzumab, and the like.

MSLN antigen recognition domains are known to the relevant skilled artisan and include, e.g., the heavy and light chain domains of amatuximab, anetumab, inezetamab, and the like. Useful antigen recognition domains include those derived from the heavy chain, light chain, and/or CDRs of antibodies that bind to MSLN, such as but not limited to e.g., amatuximab, anetumab, inezetamab, and the like.

MUC1 and MUC16 antigen recognition domains are known to the relevant skilled artisan and include, e.g., the heavy and light chain domains of cantuzumab, clivatuzumab, gatipotuzumab, abagovomab, sofituzumab, ubamatamab, and the like. Useful antigen recognition domains include those derived from the heavy chain, light chain, and/or CDRs of antibodies that bind to MUC1 and MUC16, such as but not limited to e.g., cantuzumab, clivatuzumab, gatipotuzumab, abagovomab, sofituzumab, ubamatamab, and the like.

NKG2D antigen recognition domains are known to the relevant skilled artisan and include, e.g., the heavy and light chain domains of tesnatilimab, and the like. Useful antigen recognition domains include those derived from the heavy chain, light chain, and/or CDRs of antibodies that bind to NKG2D, such as but not limited to e.g., tesnatilimab, and the like.

PD-1 antigen recognition domains are known to the relevant skilled artisan and include, e.g., the heavy and light chain domains of acrixolimab, balstilimab, budigalimab, camrelizumab, cemiplimab, cetrelimab, dostarlimab, enlonstobart, ezabenlimab, finotonlimab, geptanolimab, iparomlimab, lipustobart, nivolumab, nofazinlimab, penpulimab, peresolimab, pidilizumab, pimivalimab, pradusinstobart, prolgolimab, pucotenlimab, retifanlimab, rosnilimab, rulonilimab, sasanlimab, serplulimab, sintilimab, spartalizumab, tislelizumab, toripalimab, zeluvalimab, zimberelimab, and the like. Useful antigen recognition domains include those derived from the heavy chain, light chain, and/or CDRs of antibodies that bind to PD-1, such as but not limited to e.g., acrixolimab, balstilimab, budigalimab, camrelizumab, cemiplimab, cetrelimab, dostarlimab, enlonstobart, ezabenlimab, finotonlimab, geptanolimab, iparomlimab, lipustobart, nivolumab, nofazinlimab, penpulimab, peresolimab, pidilizumab, pimivalimab, pradusinstobart, prolgolimab, pucotenlimab, retifanlimab, rosnilimab, rulonilimab, sasanlimab, serplulimab, sintilimab, spartalizumab, tislelizumab, toripalimab, zeluvalimab, zimberelimab, and the like.

ROR1 antigen recognition domains are known to the relevant skilled artisan and include, e.g., the heavy and light chain domains of nebratamig, zilovertamab, and the like. Useful antigen recognition domains include those derived from the heavy chain, light chain, and/or CDRs of antibodies that bind to ROR1, such as but not limited to e.g., nebratamig, zilovertamab, and the like.

SLAMF7 antigen recognition domains are known to the relevant skilled artisan and include, e.g., the heavy and light chain domains of azintuxizumab, elotuzumab, and the like. Useful antigen recognition domains include those derived from the heavy chain, light chain, and/or CDRs of antibodies that bind to SLAMF7, such as but not limited to e.g., azintuxizumab, elotuzumab, and the like.

Useful CLDN6 antigen recognition domains include, e.g., the heavy and light chain domains of those antibodies described in U.S. Pat. Nos. 9,487,584, 9,274,119, 10,745,477, 10,233,253, and 10,053,511. Useful antigen recognition domains include those derived from the heavy chain, light chain, and/or CDRs of antibodies that bind to CLDN6, including e.g., those described in U.S. Pat. Nos. 9,487,584, 9,274,119, 10,745,477, 10,233,253, and 10,053,511.

Useful multi-specific CARs include antigen-recognition domains, or combinations of antigen-recognition domains, targeting the following combinations of antigens: CD19+CD20, CD19+CD22, BCMA+CD19, CD33+CLL-1, CD19+CD276, CD19+CD20+CD22, CD19+IL15R, IL15R+MUC16, IL-15Rα+NKG2D, GPC3+IL15R, CD16a+CD19+IL-15Rα, BCMA+CD16a+NKp46, CD123+CD33, CLDN18.2+NKG2D, CD20+CD22, BCMA+SLAMF7, CD19+CD70, CD19+IL18R1, GD2+IL15R, CD70+IL15R, CD19+CD8, CD20+CD79A, CD19+CD22+CD8, CD19+EBV Protein, CD19+CD7, BCMA+CD38, IL-12+MUC16, CD33+FLT3, CD19+IL-2Rβ, BCMA+CD70, EGFR+IL-13Rα2, HER2+IL15R, MICA+MICB, BCMA+TACI, GPC3+IL-2Rβ, CD19+DR5, CD123+CD33+CLL-1, CD3+CD7, CEACAM5+CEACAM6, CD20+CD22+CD38, CD19+CLDN18.2, AGRE2+CLL-1, CD123+TIM3, CLDN18.2+PDL1, CTLA4+MSLN+PD-1, STEAP2+TGFBR2, CEA+IL-15Rα+IL-21R, CD123+CD33+CD38+CLL-1, CD133+EGFR, BCMA+HPK1, CD8A+NY-ESO-1, CD38+DR5, CD19+EGFR, BCMA+CD19+HER2+Trop-2, CD19+MUC1, PDL1+VEGFR1, CD123+CD33+CD38+CD56+CLL-1+MUC1, BCMA+CD138+CD19+CD38, BCMA+CD16a+IL-15Rα, NCR2+NKG2D, CD38+CLL-1, CD276+FGFR4, and the like.

In some instances, useful CARs (or useful component parts thereof) will include a monospecific CAR targeting CD19, including but not limited to e.g., the CARs of CAR therapies Inaticabtagene Autoleucel, Actalycabtagene autoleucel, Relmacabtagene autoleucel, Lisocabtagene maraleucel, Brexucabtagene Autoleucel, Axicabtagene Ciloleucel, Tisagenlecleucel, Obecabatagene autoleucel, Azercabtagene Zapreleucel, Rapcabtagene autoleucel, and the like.

In some instances, useful CARs (or useful component parts thereof) will include a CAR targeting CD19, including but not limited to e.g., the CARs of CAR therapies ALLO-501A (Cellectis SA|Servier Pharmaceuticals LLC|Allogene Therapeutics, Inc.), CTL-119 (University of Pennsylvania|Novartis Pharma AG), IM19CAR-T (Beijing Imunopharm Technology Co., Ltd.), JCAR-014 (Celgene Corp.|Juno Therapeutics, Inc.), JCAR-015 (Celgene Corp.|Juno Therapeutics, Inc.), MB-CART19.1 (Miltenyi Biotec, Inc.), pCAR-19B (Chongqing Precision Biotechnology Co., Ltd.), TI-1007 (Timmune|Shenzhen Beike Biotechnology Co., Ltd.), UCART-19 (Cellectis SA|Pfizer Inc.|Allogene Therapeutics, Inc.), AT-101 (AbClon, Inc.), CLIC-1901 (Ottawa Hospital Research Institute), CT-032 (CARsgen Therapeutics Co.,Ltd), CTX-112 (CRISPR Therapeutics AG), GC007g (Gracell Biotechnologies, Inc.), GLPG-5101 (Galapagos NV), KYV-101 (Kyverna Therapeutics, Inc.), MB CART19.1 (Miltenyi GmbH), MC150 (Chongqing Precision Biotechnology Co., Ltd.), PL-001 (PELL Bio-Med Technology Co., Ltd.), SCRI-huCAR19v1 (Seattle Children's Hospital), SCRI-huCAR19v2 (Seattle Children's Hospital), 19(T2)28z1xx TRAC T cell (Takeda Pharmaceutical Co., Ltd.), ATA-3219 (Atara Biotherapeutics, Inc.), ATLCAR.CD19 (Bellicum Pharmaceuticals, Inc.), BRL-301 (BRL Medicine Inc.), CC-97540 (Juno Therapeutics, Inc.|Bristol Myers Squibb Co.), CLBR-001/SWI-019 (California Institute for Biomedical Research|AbbVie, Inc.), CNTY-101 (Century Therapeutics, Inc.), CT-RD06 (Nanjing Bioheng Biotech Co., Ltd.), ET019003-T Cells (Eureka Therapeutics, Inc.), ET190L1-ARTEMIS T cells (Eureka Therapeutics, Inc.), FT-522 (Fate Therapeutics, Inc.), FT-819 (Fate Therapeutics, Inc.), PBCAR-19B (Precision BioSciences, Inc.), PCAR-019 (PersonGenAnke Cellular Therapeutics Co., Ltd.), SC-291 (Sana Biotechnology, Inc.), TAK 940 (Takeda Pharmaceutical Co., Ltd.), TBI-2001 (Takara Bio Inc.), and the like.

In some instances, useful CARs (or useful component parts thereof) will include a multi-specific CAR targeting CD19 and at least one other antigen, including but not limited to e.g., the CARs of CAR therapies MB-CART2019.1 (Miltenyi Biotec, Inc.), KITE-363 (Kite Pharma, Inc.), BYC-104 (BRL Medicine Inc.), Anbalcabtagene autoleucel (Curocell, Inc.), Bi-4SCAR CD19/22 T cells (Shenzhen Geno-Immune Medical Institute), Prizloncabtagene autoleucel (AbelZeta Pharma, Inc.), KITE-753 (Kite Pharma, Inc.), CB-010 (Caribou Biosciences, Inc.), CTA-101 (Nanjing Bioheng Biotech Co., Ltd.), Denocabtagene Ciloleucel (Immunotech Applied Science Limited), IMJ-995 (Novartis Pharmaceuticals Corp.), LB1909 (Nanjing Legend Biotechnology Co., Ltd.), LCAR-AIO (Nanjing Legend Biotechnology Co., Ltd.), NKX-019 (Nkarta, Inc.), SYNCAR-001 (Synthekine, Inc.), GC-022 (Gracell Biotechnologies (Shanghai) Co., Ltd.), GC-502 (Gracell Biotechnologies (Shanghai) Co., Ltd.), EPC-001 (Elpis Biopharmaceuticals Corp.), API-192 (Appia Bio, Inc.|Kite Pharma, Inc.), ATA-3431 (Atara Biotherapeutics, Inc.), AVC-203 (AvenCell Therapeutics, Inc.), CNTY-102 (Century Therapeutics, Inc.), CRG-023 (Cargo Therapeutics, Inc.), CTA302 (Nanjing Bioheng Biotech Co., Ltd.), GF-CARTO1 (Genomefrontier Therapeutics, Inc.), MB-CART 2219.1 (Miltenyi Biomedicine GmbH), MTB-002 (Medtherapy Biotechnology, Inc.), ORGCAR19.22 (Orgenesis|Beijing Win Win Technology Co., Ltd.), RG-1970 (Hangzhou Ronggu Biotechnology Co., Ltd.), SC276 (Sana Biotechnology, Inc.), SG-299 (Sana Biotechnology, Inc.), SG233 (Sana Biotechnology, Inc.), MTB-004 (Medtherapy Biotechnology, Inc.), HY-003 (Juventas Cell Therapy Ltd.), ONKT-101 (ONK Therapeutics Ltd.), YT-19/22 (Huaxia Yingtai Beijing Biotechnology Co Ltd.), CABA-201 (Cabaletta Bio, Inc.), GC-012F (Gracell Biotechnologies, Inc.), GLPG-5201 (Galapagos NV), KQ-2003 (Shanghai Keqi Pharmaceutical Technology Co., Ltd.), FT-596 (Fate Therapeutics, Inc.), P-BCMACD19-ALLO1 (Poseida Therapeutics, Inc.), MVR-T7011 (ImmVira, Co., Ltd), MVR-T7012 (ImmVira, Co., Ltd), MVR-T7013 (ImmVira, Co., Ltd), LCAR-AIO (Legend Biotech USA, Inc.), and the like.

In some instances, useful CARs (or useful component parts thereof) will include a multi-specific CAR targeting at least CD19 and CD20, including but not limited to e.g., the CARs of CAR therapies MB-CART2019.1 (Miltenyi Biotec, Inc.), KITE-363 (Kite Pharma, Inc.), HY-004 (Juventas) (Juventas Cell Therapy Ltd.), IMPT-314 (Immpact Bio USA, Inc.), IMPT-514 (Immpact Bio USA, Inc.), Prizloncabtagene autoleucel (AbelZeta Pharma, Inc.), KITE-753 (Kite Pharma, Inc.), CAR20.19.22 T-cells (Miltenyi Biomedicine GmbH), P-CD19CD20-ALLO1 (Poseida Therapeutics, Inc.), HX-s001 (Huaxia Yingtai Beijing Biotechnology Co Ltd.), HXYT-001 (Huaxia Yingtai Beijing Biotechnology Co Ltd.), LCAR-AIO (Nanjing Legend Biotechnology Co., Ltd.), Lcaraio (Legend Biotech USA, Inc.), P-CD19CD20-AL-LO1 (Poseida Therapeutics, Inc.), YT-19/20 (Huaxia Yingtai Beijing Biotechnology Co Ltd.), ICG 132 or ICG 134 (Icar Bio Therapeutics Ltd), API-192 (Appia Bio, Inc.|Kite Pharma, Inc.), ATA-3431 (Atara Biotherapeutics, Inc.), AVC-203 (AvenCell Therapeutics, Inc.), CRG-023 (Cargo Therapeutics, Inc.), and GF-CARTO1 (Genomefrontier Therapeutics, Inc.).

In some instances, useful CARs (or useful component parts thereof) will include a multi-specific CAR targeting at least CD19 and BCMA, including but not limited to e.g., the CARs of CAR therapies GC-012F (Gracell Biotechnologies (Shanghai) Co., Ltd.), KQ-2003 (Shanghai Keqi Pharmaceutical Technology Co., Ltd.), and P-BCMACD19-ALLO1 (Poseida Therapeutics, Inc.).

In some instances, useful CARs (or useful component parts thereof) will include a multi-specific CAR targeting at least CD19 and CD22, including but not limited to e.g., the CARs of CAR therapies CD19/CD22 dual CART Chongqing Precision Biotech (Chongqing Precision Biotechnology Co., Ltd.), Bi-4SCAR CD19/22 T cells (Shenzhen Geno-Immune Medical Institute), CT-120 (Nanjing IASO Biotherapeutics Co., Ltd.), CART2219.1 (KK Women's & Children's Hospital Pte Ltd.), LCAR-L10D (Xi'an Jiaotong University), CD19/CD22 CAR T cells (Orca Biosystems, Inc.), CTA-101 (Nanjing Bioheng Biotech Co., Ltd.), IMJ-995 (Novartis Pharmaceuticals Corp.), LB1909 (Nanjing Legend Biotechnology Co., Ltd.), GC-022 (Gracell Biotechnologies (Shanghai) Co., Ltd.), EPC-001 (Elpis Biopharmaceuticals Corp.), B-019 (Shanghai Pharmaceuticals Holding Co., Ltd.), CTA302 (Nanjing Bioheng Biotech Co., Ltd.), MB-CART 2219.1 (Miltenyi Biomedicine GmbH), MTB-002 (Medtherapy Biotechnology, Inc.), ORGCAR19.22 (Beijing Win Win Technology Co., Ltd.), SC276 (Sana Biotechnology, Inc.), MTB-004 (Medtherapy Biotechnology, Inc.), and YT-19/22 (Huaxia Yingtai Beijing Biotechnology Co Ltd.).

In some instances, useful CARs (or useful component parts thereof) will include a monospecific CAR targeting BCMA, including but not limited to e.g., the CARs of CAR therapies Inaticabtagene Autoleucel, Actalycabtagene autoleucel, Equecabtagene Autoleucel, Ciltacabtagene autoleucel, Relmacabtagene autoleucel, Idecabtagene Vicleucel, Lisocabtagene maraleucel, Brexucabtagene Autoleucel, Axicabtagene Ciloleucel, Tisagenlecleucel, Obecabatagene autoleucel, Zevorcabtagene autoleucel, and the like.

In some instances, useful CARs (or useful component parts thereof) will include a monospecific CAR targeting BCMA, including but not limited to e.g., the CARs of CAR therapies CART-ddBCMA (Arcellx, Inc.), ET-140 or FCARH-143 or ET140-CAR or JCARH-125 or MCARH-171 (Juno Therapeutics, Inc.), Descartes 011 (Cartesian Therapeutics, Inc. (United States)), Descartes-08 (Cartesian Therapeutics, Inc. (United States)), KITE-585 (Kite Pharma, Inc.), PBCAR-269A (Precision BioSciences, Inc.), PHE-885 (Novartis Pharmaceuticals Corp.), PRG-1801 (Shenzhen Puruijin Biological Pharmaceutical Co., Ltd.), ALLO-605 (Allogene Therapeutics, Inc.), Anti-BCMA CAR-NK cell therapy (Asclepius) (Go Better Studio Accessibility Technology (SuZhou) Co., Ltd.), ARI0002H (Instituto De SAlud Carlos lii De Madrid), C-CARO88 (AbelZeta Pharma, Inc.), GLPG-5301 (Galapagos NV), NXC-201 (Nexcella, Inc.), P-BCMA-101 (Poseida Therapeutics, Inc.), ACLX-001 (Arcellx, Inc.), ALLO-715 (Cellectis SA), BB21217 (bluebird bio, Inc.), BCMA-CS1 cCAR-T cell therapy (iCell Gene Therapeutics, Inc.), CB-011 (Caribou Biosciences, Inc.), CBG002 (Zhejiang Kangbaiyu Biotechnology Co., Ltd.), CC-98633 (Celgene Corp.), CT 0590 or KJ-C2111 (CARsgen Therapeutics Co., Ltd), CTX-120 (CRISPR Therapeutics AG), CYAD-211 (Celyad Oncology SA), IBI-346 (Innovent Biologics, Inc.), IM21 CAR-T cells (Beijing Imunopharm Technology Co., Ltd.), JWCAR-129 (Juno Therapeutics, Inc.), LCAR-B4822M (Nanjing Legend Biotechnology Co., Ltd.), LCAR-BCDR (Nanjing Legend Biotechnology Co., Ltd.), LUCAR-B68 Cells (Nanjing Legend Biotechnology Co., Ltd.), MCARH125 (Memorial Sloan Kettering Cancer Center), MCM998 (Novartis AG), MDC-CAR-BCMA001 (−), P-BCMA-ALLO1 (Poseida Therapeutics, Inc.), RG-6538 (Janssen Biotech, Inc.|Transposagen Biopharmaceuticals, Inc.), SENL-103 (Hebei Senlangbio Biotechnology Co., Ltd.), SA-102 (Wuhan Si'an Medical Technology Co., Ltd.), SKB-394 (Sichuan Kelun-Biotech Biopharmaceutical Co., Ltd.), BCMA CAR iNKT (Agenus, Inc.), BCMA GoCAR-NK (Bellicum Pharmaceuticals, Inc.), BG BCMA (Guangzhou Bio-gene Technology Co., Ltd.), CB-020 (Caribou Biosciences, Inc.), GB-5010 (Shanghai Genechem Co., Ltd.), HY-029 (Juventas Cell Therapy Ltd.), MiNK-413 (MiNK Therapeutics, Inc.), Ori-CAR-002 (Yuanqi Biotechnology Shanghai Co Ltd.), RDO9 (Nanjing Bioheng Biotech Co., Ltd.), SA-BCMA-CS1-102 (SA Science, Inc.), SC-255 (Sana Biotechnology, Inc.), SENL302 (Hebei Senlangbio Biotechnology Co., Ltd.), XYF-B08 (Xi'An Yufan Biotechnology), CTX-121 (CRISPR Therapeutics AG), CART-BCMA (University of Pennsylvania), HY-015 (Juventas Cell Therapy Ltd.), and HY-027 (Juventas Cell Therapy Ltd.).

In some instances, useful CARs (or useful component parts thereof) will include a CAR targeting at least CD20, including but not limited to e.g., the CARs of CAR therapies 4SCAR-CD20 (Shenzhen Geno-Immune Medical Institute), bbT-369 (bluebird bio, Inc.), MB-106 CD20 CAR (Fred Hutchinson Cancer Research Center), ELC-301 (Elicera Therapeutics AB), UCART20×22 (Cellectis SA), ADI-001 (Regeneron Pharmaceuticals, Inc.|Adicet Therapeutics, Inc.), LB1905 (Nanjing Legend Biotechnology Co., Ltd.), LY007 (Shanghai Longyao Biotech Co., Ltd.), MB-CART20.1 (Miltenyi Biotec, Inc.), ASP2802 (Astellas Pharma, Inc.), LUCAR-20SD (Nanjing Legend Biotechnology Co., Ltd.), UB-VV300 (Umoja Biopharma, Inc.), UB-VV310 (Umoja Biopharma, Inc.), and PBCAR-20A (Precision BioSciences, Inc.).

In some instances, useful CARs (or useful component parts thereof) will include a CAR targeting at least CD22, including but not limited to e.g., the CARs of CAR therapies CRG-022 (Cargo Therapeutics, Inc.), 4SCAR-CD22 (Shenzhen Geno-Immune Medical Institute), RD102 (Nanjing IASO Biotherapeutics Co., Ltd.), SL22P CART cells (Hebei Senlangbio Biotechnology Co., Ltd.), JCAR-018 (National Cancer Institute), ThisCART 22 (Fundamenta Therapeutics, Ltd.), UCART-22 OR BALLI-01 (Cellectis SA), Anti-CD22-CAR m972 (National Cancer Institute), ICTCAR017 (Innovative Cellular Therapeutics Co., Ltd.), LQ-031 (Shanghai Novamab Biopharmaceuticals Co., Ltd.), OC-2 (Onechain Immunotherapeutics), and SC262 (Sana Biotechnology, Inc.).

In some instances, useful CARs (or useful component parts thereof) will include a CAR targeting at least CD123, including but not limited to e.g., the CARs of CAR therapies MB-102 (Mustang Bio, Inc.), ACLX-002 (Arcellx, Inc.), Allo-RevCAR01-T-CD123 (AvenCell Europe GmbH), CD123CAR-41 BB-CD3zeta-EGFRt (Hrain Biotechnology Co. Ltd.), AVC-201 (AvenCell Therapeutics, Inc.), IM-23 (Beijing Imunopharm Technology Co., Ltd.), UCART-123 (Cellectis SA), UniCAR-T-CD-123 (GEMoaB Monoclonals GmbH), Unicar-T-CD123 OR AVC-101 (AvenCell Therapeutics, Inc.), JD-023 (Beijing Jingda Biotechnology Co., Ltd.), AUTO9 (University College London|Autolus Therapeutics Plc), SENL401 (Hebei Senlangbio Biotechnology Co., Ltd.), EPC-005 (Elpis Biopharmaceuticals Corp.), ARD-102 (Arce Therapeutics, Inc.), HY-007 (Juventas Cell Therapy Ltd.), and 4SCAR-CD123 (Shenzhen Geno-Immune Medical Institute).

In some instances, useful CARs (or useful component parts thereof) will include a CAR targeting at least CD30, including but not limited to e.g., the CARs of CAR therapies ATLCAR.CD30 cells (UNC Lineberger Comprehensive Cancer Center), CD30 CAR-T cell therapy (Tessa Therapeutics Pte Ltd, TT11 CD30 CAR T (The University of North Carolina at Chapel Hill|Baylor College of Medicine), HSP-CAR30 (Josep Carreras Leukaemia Research Institute), ATLCAR.CD30.CCR4 cells (UNC Lineberger Comprehensive Cancer Center) HU30-CD28Z (National Cancer Institute), CD30.CARIH-27721 (Baylor College of Medicine), EBV-specific-CAR.CD30 (Baylor College of Medicine), TT-11X (Tessa Therapeutics Ltd), and RD 111 (Nanjing IASO Biotherapeutics Co., Ltd.).

In some instances, useful CARs (or useful component parts thereof) will include a CAR targeting at least CD33, including but not limited to e.g., the CARs of CAR therapies VCAR-33 (National Cancer Institute), VCAR33ALLO (Vor Biopharma, Inc.), QN-023A (Qihan Biotech), BECAR33 (Great Ormond Street Hospital for Children NHS Foundation Trust), CART33 (University of Pennsylvania), CC-96191 (Dragonfly Therapeutics, Inc.), CLL1-CD33 cCART cell therapy (iCell Gene Therapeutics, Inc.), ICG-136 (iCell Gene Therapeutics, Inc.), LB1910 (Nanjing Legend Biotechnology Co., Ltd.), LCAR-AMDR Cells (Nanjing Legend Biotechnology Co., Ltd.), PRGN-3006 (Precigen, Inc.), SC-DARIC33 (bluebird bio, Inc.), SENTI-202 (Senti Sub|, Inc.), GCK-02 (Shanghai Jingshan Biotechnology Co., Ltd.), HY-002 (Juventas Cell Therapy Ltd.), AntiCD33 CARNK92 cells (PersonGen BioTherapeutics (Suzhou) Co. Ltd), INXN-3004 (Precigen, Inc.|Alaunos Therapeutics, Inc.), AUTO9 (University College London|Autolus Therapeutics Plc), and HY-007 (Juventas Cell Therapy Ltd.).

In some instances, useful CARs (or useful component parts thereof) will include a CAR targeting at least CD38, including but not limited to e.g., the CARs of CAR therapies CART-38 CellIs (University of Pennsylvania), CD38 CAR-T (Sorrento Therapeutics, Inc.), STI-5171 (Sorrento Therapeutics, Inc.), CYT-538 (Cytovia Therapeutics, Inc.), FT-555 (Fate Therapeutics, Inc.), LQ-032 (Shanghai Novamab Biopharmaceuticals Co., Ltd.), ONKT-102 (Universitstsklinikum Hamburg-Eppendorf|ONK Therapeutics Ltd.), GDA-601 (Gamida Cell Ltd.), and 4SCAR-CD38 (Shenzhen Geno-Immune Medical Institute).

In some instances, useful CARs (or useful component parts thereof) will include a CAR targeting at least CD70, including but not limited to e.g., the CARs of CAR therapies 4SCAR-CD70 (Shenzhen Geno-Immune Medical Institute), CAR.70/lL15-transduced CB-NK cells (The University of Texas MD Anderson Cancer Center), CTX-131 (CRISPR Therapeutics AG), ALLO-316 (Allogene Therapeutics, Inc.), HR010 (Hrain Biotechnology Co. Ltd.), ADI-270 (Adicet Bio, Inc.), CAT-248 (Catamaran Bio, Inc.), CD70 CAR-NK cell therapy (Nkarta, Inc.|CRISPR Therapeutics AG), P-CD70-ALLO1 (Poseida Therapeutics, Inc.), CTX-130 (CRISPR Therapeutics AG), C-4-29 (Chongqing Precision Biotechnology Co., Ltd.), and RG-1970 (Hangzhou Ronggu Biotechnology Co., Ltd.).

In some instances, useful CARs (or useful component parts thereof) will include a CAR targeting at least CLDN18.2, including but not limited to e.g., the CARs of CAR therapies CT-041 (CARsgen Therapeutics Co., Ltd), IMC-002 (Suzhou Immunofoco Biotechnology Co. Ltd.), AZD-6422 (AstraZeneca PLC), LY011 (Shanghai Longyao Biotech Co., Ltd.), CLDN18.2 UCAR-T (T-Maximum Pharmaceutical (Suzhou) Co., Ltd.), Dual-targeting CLDN18.2 and PD-L1 CAR-T cells (Sichuan University), IBI-345 (Innovent Biologics, Inc.), IMC-008 (Suzhou Immunofoco Biotechnology Co. Ltd.), KD-022 (Nanjing KAEDI Biotech, Inc.), KD-496 (Nanjing KAEDI Biotech, Inc.), KJ-C1807 (CARsgen Therapeutics Co., Ltd), LB-1904 (Nanjing Legend Biotechnology Co., Ltd.), LB1908 (Nanjing Legend Biotechnology Co., Ltd.), HEC-016 (Sunshine Lake Pharma Co. Ltd.), CTB001 (Nanjing Bioheng Biotech Co., Ltd.), iPD-1-Claudin18.2-CAR-T OR XKDCT086 (The Affiliated Hospital of Qingdao University), CTB001 (Nanjing Bioheng Biotech Co., Ltd.), ALLO-182 (Allogene Therapeutics, Inc.), BNT-212 (BioNTech SE), CLDN18.2-CAR-iNKT (Arovella Therapeutics Ltd.), CT-007 (Guangdong Xiankangda Biotechnology Co., Ltd.), CT-076 (Guangdong Xiankangda Biotechnology Co., Ltd.), CT-086 (Guangdong Xiankangda Biotechnology Co., Ltd.), CT-253 (Guangdong Xiankangda Biotechnology Co., Ltd.), CTD-101 (Nanjing Bioheng Biotech Co., Ltd.), DCTY-1502 (Beijing Dingcheng Peptide Source Biotechnology Co., Ltd.), GB70041 (Shanghai Genechem Co., Ltd.), GC-506 (Gracell Biotechnologies (Shanghai) Co., Ltd.), IM92 (Beijing Imunopharm Technology Co., Ltd.), KD-593 (Nanjing KAEDI Biotech, Inc.), KD-U182 (Nanjing KAEDI Biotech, Inc.), P-2003 (Shandong Boan Biotechnology Co., Ltd.), PM-3023 (Pumis Biotechnology (Zhuhai) Co., Ltd.), PR-401 (Shandong Boan Biotechnology Co., Ltd.), SENL202 (Hebei Senlangbio Biotechnology Co., Ltd.), and YT-claudin18.2 (Huaxia Yingtai Beijing Biotechnology Co Ltd.).

In some instances, useful CARs (or useful component parts thereof) will include a CAR targeting at least CLL-1, including but not limited to e.g., the CARs of CAR therapies CB-012 (Caribou Biosciences), CD371-YSNVZ-IL18 CAR T cells (Memorial Sloan Kettering Cancer Center), ICG-136 (iCell Gene Therapeutics, Inc.), LB1910 (Nanjing Legend Biotechnology Co., Ltd.), ARD103 (Arce Therapeutics, Inc.), HY-032 (Juventas Cell Therapy Ltd.), GCK-02 (Shanghai Jingshan Biotechnology Co., Ltd.), and AUTO9 (University College London|Autolus Therapeutics Plc).

In some instances, useful CARs (or useful component parts thereof) will include a CAR targeting at least DLL3, including but not limited to e.g., the CARs of CAR therapies LB2102 (Nanjing Legend Biotechnology Co., Ltd.), ALLO-213 (Allogene Therapeutics, Inc.), and AMG-119 (Amgen, Inc.).

In some instances, useful CARs (or useful component parts thereof) will include a CAR targeting at least EGFR or EGFRvIII, including but not limited to e.g., the CARs of CAR therapies Anti-EGFR CAR-T cell therapy (AbelZeta Pharma, Inc.), Anti-EGFRvIII-CAR (Kite Pharma, Inc.), Anti-EGFRvII-directed CAR-T cell therapy (Dehe Biotech Energae), LEU-001 (Leucid Bio Ltd.), Anti-EGFR/IL-13R alpha 2 CAR-T cell therapy (University of Pennsylvania| Tmunity Therapeutics, Inc.), EGFR IL12 CART (Shenzhen Puruijin Biological Pharmaceutical Co., Ltd.), EGFR806 CAR-T Cell (Seattle Children's Hospital), KJ-C21120 (CARsgen Therapeutics Co., Ltd), LXF-821 (Novartis AG), DCTY-0801 (Beijing Dingcheng Peptide Source Biotechnology Co., Ltd.), CYT-501 (Cytoimmune Therapeutics, Inc.), EGFRt-IL21 (Asher Biotherapeutics, Inc.), EGFRvII synNotch CAR-T (University of California), GCT-02 (Myrio Therapeutics Pty Ltd.), HY-026 (Juventas Cell Therapy Ltd.), MT-029 (T-Maximum Pharmaceutical (Suzhou) Co., Ltd.), GCK-03 (Shanghai Jingshan Biotechnology Co., Ltd.), LQ-103 (Shanghai Novamab Biopharmaceuticals Co., Ltd.), Anti-EGFRvIII CAR-T cell therapy (Beijing Marion Biotechnology Co., Ltd.|Beijing Sanbo Brain Hospital Co., Ltd.), and EGFRvIII-directed CAR-T-cell tharapy (University of Pennsylvania). Useful EGFRvII targeting CARs further include those described in WO2014130657 and the like.

In some instances, useful CARs (or useful component parts thereof) will include a CAR targeting at least FLT3, including but not limited to e.g., the CARs of CAR therapies Anti-FLT3 CAR-T cell (PersonGen BioTherapeutics (Suzhou)), TAA5 CAR T (PersonGen BioTherapeutics (Suzhou) Co. Ltd), AMG-553 (Amgen, Inc.), CYTO NK-201 (Cytoimmune Therapeutics, Inc.), Anti-FLT3 CAR-T (City of Hope National Medical Center|Cytoimmune Therapeutics, Inc.), HEMO-CAR-T cell therapy (Hemogenyx), and SENTI-202 (Senti Sub|, Inc.).

In some instances, useful CARs (or useful component parts thereof) will include a CAR targeting at least GD2, including but not limited to e.g., the CARs of CAR therapies 4SCAR-GD2 (Shenzhen Geno-Immune Medical Institute), GD2-CART01 (Bambino Gesu Hospital), 1RG-CART (Cancer Research UK), Anti-GD2 CART (Zhujiang Hospital), AUTO6NG (University College London), AUTO 6 (University College London), C7R-GD2.CART cell therapy (Baylor College of Medicine) iC9-GD2-CAR-IL-15 T-cells (UNC Lineberger Comprehensive Cancer Center), VZV-specific-GD2-CAR (Baylor College of Medicine), iC9-GD2.CAR.IL-15 T-cells (UNC Lineberger Comprehensive Cancer Center), AUTO6-NG (University College London|Autolus Ltd.), IKT-703 (AnoVac Inc.), GINAKIT-Cells OR KUR-501 OR CAR.GD2-IL-15 NKTs (Baylor College of Medicine), andGD2.CAR-triVSTs (Baylor College of Medicine).

In some instances, useful CARs (or useful component parts thereof) will include a CAR targeting at least GPC3, including but not limited to e.g., the CARs of CAR therapies GB-5011 (Shanghai Genechem Co., Ltd.), AZD-5851 (AstraZeneca PLC), BOXR-1030 (Cogent Biosciences, Inc.), gpc3-car-ori2 (Yuanqi Biotechnology Shanghai Co Ltd.), C-CAR031 (Zhejiang University), CT-011 (CARsgen Therapeutics Co., Ltd), CT-0181 (CARsgen Therapeutics Co., Ltd), GLYCAR T cell therapy (Baylor College of Medicine), IM 83 (Beijing Imunopharm Technology Co., Ltd.), LCAR-H93T Cells (Nanjing Legend Biotechnology Co., Ltd.), GKL-006 (Beijing Gene Key Life Technology Co., Ltd), ADI-002 (Regenacy Pharmaceuticals LLC|Adicet Therapeutics, Inc.), CYT-150 (Cytovia Therapeutics, Inc.), CYT-503 (National Cancer Institute), KUR-503 (Baylor College of Medicine), SYNCAR002 (Synthekine, Inc.), SA-GPC3-103 (SA Science, Inc.), SENTI-301 (Senti Sub|, Inc.), TT-14 (Tessa Therapeutics Ltd), Anti-GPC3 autologous CART cell therapy (CRISPR Therapeutics AG), CT-017 (CARsgen Therapeutics Co., Ltd), LQ-102 (Shanghai Novamab Biopharmaceuticals Co., Ltd.), andHX-s002 (Huaxia Yingtai Beijing Biotechnology Co Ltd.).

In some instances, useful CARs (or useful component parts thereof) will include a CAR targeting at least HER2, including but not limited to e.g., the CARs of CAR therapies ALETA-002 (Aleta Biotherapeutics, Inc.), AU-101 (Aurora Biopharma, Inc.), AU-105 (Aurora Biopharma, Inc.), AB-201 (Artiva Biotherapeutics, Inc.), BPX-603 (Bellicum Pharmaceuticals, Inc.), CCT3-HER2-0406 (EXUMA Biotech Corp.), BP-2301 (Shinshu University), CIDeCAR (Bellicum Pharmaceuticals, Inc.), CT-0508 (CTx Operations, Inc.), HER2(EQ)BBzeta/CD19 T cells (Mustang Bio, Inc.), HER2.taNK (NantKwest, Inc.), MB-103 (Mustang Bio, Inc.), TT-16 (Tessa Therapeutics Ltd), CT-0525 (Carisma Therapeutics, Inc.), AT-501 (AbClon, Inc.), BM-1901 (Kunshi Biotechnology (Shenzhen) Co., Ltd.), CAT-179 (Catamaran Bio, Inc.), FT825/ONO-8250 (Fate Therapeutics, Inc.|Ono Pharmaceutical Co., Ltd.), MT-028 (T-Maximum Pharmaceutical (Suzhou) Co., Ltd.), OmniCAR HER2 (Prescient Therapeutics Ltd.), UWHer2T (UWELL Biopharma, Inc.), RB-H21 (Refuge Biotechnologies, Inc.), GDA-501 (Gamida Cell Ltd.), GCK-03 (Shanghai Jingshan Biotechnology Co., Ltd.), and MVR-T7 Shenzhen Yinuowei Medical Technology Co., Ltd.).

In some instances, useful CARs (or useful component parts thereof) will include a CAR targeting at least MSLN, including but not limited to e.g., the CARs of CAR therapies A2B-694 (A2 Biotherapeutics, Inc.), GC-008T (Gracell Biotechnologies (Shanghai) Co., Ltd.), iCasp9M28z (Memorial Sloan Kettering Cancer Center), KD-021 (Nanjing KAEDI Biotech, Inc.), LB1902 (Nanjing Legend Biotechnology Co., Ltd.), MCY-M11 (MaxCyte, Inc.), UCLM-802 (UTC Therapeutics, Inc.), KT032 (Nanjing Kati Medical Technology Co., Ltd.), LD-013 (Nanjing Blue Shield Biotechnology Co., Ltd.), TMMSTNO2 (Tmunity Therapeutics, Inc.), ATA-3271 (Atara Biotherapeutics, Inc.), CT 1119 (CTx Operations, Inc.), IM81 (Beijing Imunopharm Technology Co., Ltd.), KBISM Kiromic OR ALEXISISO1 (Kiromic Biopharma, Inc.), KJ-C2113 (CARsgen Therapeutics Co., Ltd), m-28z-T2 (GIBH), MSLN CART(LVV) (UTC Therapeutics, Inc.), PM-3006 (Pumis Biotechnology (Zhuhai) Co., Ltd.), RD11 (Nanjing Bioheng Biotech Co., Ltd.), ATA-2271 (Memorial Sloan Kettering Cancer Center), GC-503 (Gracell Biotechnologies (Shanghai) Co., Ltd.), and UCARTMESO (Cellectis SA).

In some instances, useful CARs (or useful component parts thereof) will include a CAR targeting at least MUC1 or MUC16, including but not limited to e.g., the CARs of CAR therapies TmTNMUC 01 CART (University of Pennsylvania), huMNC2-CAR22 (Minerva Biotechnologies Corp.), huMNC2-CAR44 T cells (Minerva Biotechnologies Corp.), P-MUC1C-ALLO1 (Poseida Therapeutics, Inc.), TmTnMUC1-01 (Tmunity Therapeutics, Inc.), bbT-4015 (2seventy Bio, Inc.), ONKT-103 (ONK Therapeutics Ltd.), TB-201 (Therabest, INC.), LQ-101 (Shanghai Novamab Biopharmaceuticals Co., Ltd.), UCARTMUC1 (Cellectis SA), JCAR-020 (Eureka Therapeutics, Inc.|Memorial Sloan Kettering Cancer Center), and PRGN-3005 (Precigen, Inc.).

In some instances, useful CARs (or useful component parts thereof) will include a CAR targeting at least NKG2D, including but not limited to e.g., the CARs of CAR therapies CYAD-01 (Dartmouth College), NKG2D-ACE2 CAR-NK cell therapy (Sidemu Biotechnology Technology), KD-025 (Nanjing KAEDI Biotech, Inc.), CYAD-203 (Celyad Oncology SA), LEU-011 (Leucid Bio Ltd.), KD-U25 (Nanjing KAEDI Biotech, Inc.), LEU-005 (Leucid Bio Ltd.), LEU-006 (Leucid Bio Ltd.), CYAD-101 (Celyad Oncology SA), KD-496 (Nanjing KAEDI Biotech, Inc.), NKX-101 (Nkarta, Inc.), andCYTO NK 301 (Cytoimmune Therapeutics, Inc.).

In some instances, useful CARs (or useful component parts thereof) will include a CAR targeting at least ROR1, including but not limited to e.g., the CARs of CAR therapies ROR1 CAR-T (Oncternal Therapeutics), JCAR-024 (Fred Hutchinson Cancer Research Center), LYL-797 (Lyell Immunopharma, Inc.), and LYL119 (Lyell Immunopharma, Inc.), ROR1R-CAR (The University of Texas MD Anderson Cancer Center).

In some instances, useful CARs (or useful component parts thereof) will include a CAR targeting at least SLAMF7, including but not limited to e.g., the CARs of CAR therapies IC9-Luc90-CD828Z (National Cancer Institute), UCART-CS1 (Cellectis SA), and CD319 chimeric antigen T cell therapy (Wuhan Bio Raid Biotechnology).

In some instances, useful CARs (or useful component parts thereof) will include a CAR targeting at least CLDN6, including but not limited to e.g., the CAR of CAR therapy BNT-211 (BioNTech SE). In some instances, useful anti-CLDN6 CARs or useful components thereof (including the antigen binding domains and/or CDRs thereof) will include those described in US20220306711, US20220184119, and WO2023105005.

In some instances, useful CARs (or useful component parts thereof, including e.g., the antigen binding domains and/or CDRs thereof) will include, but are not limited to, those described in the following U.S. and PCT patent publications: US20240050568, US20240041921, US20240041929, WO2024023124, US20240018256, U.S. Ser. No. 11/872,249B2, US20240009242, US20240000839, US20240002505, WO2024003786, US20230416390, WO2023248126, WO2023250489, WO2023246578, US20230406953, US20230405122, WO2023241141, US20230399412, US20230390336, WO2023192908, WO2023215746, U.S. Ser. No. 11/834,509B2, WO2023164256, US20230381228, U.S. Ser. No. 11/827,889B2, US20230365699, WO2023220560, US20230357427, US20230355673, US20230357355, WO2023208157, US20230340146, WO2023154890, WO2023205148, U.S. Ser. No. 11/795,238B2, WO2023199069, WO2023201314, WO2023201221, WO2023169555A9, US20230312675, US20230312708, WO2023147293, US20230295319, U.S. Ser. No. 11/753,457B2, WO2023161846, WO2023137069, WO2023158986, US20230256017, US20230250175, WO2023114777, WO2023143497, US20230242666, WO2023137291, US20230226182, WO2023134718, US20230220090, US20230220103, WO2023131285, WO2023131657, WO2023131063, US20230212255, WO2023125975, US20230203160, WO2023122337, US20230192803, US20230190804, US20230192842, WO2023114980, WO2023104099, US20230183371, US20230181634, US20230181640, U.S. Ser. No. 11/673,964B2, US20230174654, US20230174933, US20230167184, WO2023093811, WO2023088482, US20230159644, WO2023086900, WO2023086829, US20230151094, US20230149462, US20230147657, WO2023079135, U.S. Ser. No. 11/643,468B2, US20230136252, WO2023076811, WO2023047098, WO2023044350, WO2023028494, US20230114854, WO2023056429, US20230101046, US20230099646, WO2023047100, US20230087953, US20230084763, US20230074145, US20230072955, WO2023030539, US20230060292, US20230055426, US20230058044, WO2023021477, WO2023021494, U.S. Ser. No. 11/578,126B2, US20230039030, US20230029341, US20230000964, WO2023279095, WO2022240360A9, WO2022216811, WO2022242710, US20220364055, US20220362298, US20220356247, WO2022235662, WO2022236049, US20220348655, US20220348689, WO2022228579, US20220340671, WO2022224241, US20220324964, WO2022216813, US20220315665, WO2022212879, US20220305056, WO2022164886, US20220281994, US20220273710, US20220249563, WO2022166365, US20220242948, US20220233590, WO2022157673, US20220227874, WO2022151960, WO2022147075, US20220204930, US20220204609, US20220193232, US20220193133, US20220184126, US20220184127, US20220184125, US20220184129, US20220177573, WO2022120010, US20220168389, WO2022104424, US20220162301, US20220152106, US20220125840, WO2022078286, WO2022076898, US20220096651, US20220089678, WO2022048523, US20220064254, US20220064316, US20220048978, US20220047633, US20220033509, US20210393689, US20210395329, US20210393690, US20210363272, U.S. Ser. No. 11/180,553B2, US20210347851, U.S. Ser. No. 11/161,908B2, US20210322477, US20210324100, U.S. Ser. No. 11/149,076B2, US20210317209, US20210315985, WO2021207709, US20210309716, US20210284752, WO2021178695, US20210269537, U.S. Ser. No. 11/090,334B2, US20210238309, US20210221880, US20210220404, WO2021127428, US20210177896, US20210171909, U.S. Ser. No. 11/028,177B2, US20210163612, WO2021099944, US20210139595, US20210113618, U.S. Ser. No. 10/981,970B2, WO2021067290, US20210046155, US20210038646, US20210017277, US20210000870, US20200407461, U.S. Ser. No. 10/851,149B2, U.S. Ser. No. 10/815,301 B2, WO2020214937, US20200306304, US20200297760, WO2020191293, U.S. Ser. No. 10/752,684B2, U.S. Ser. No. 10/752,670B2, US20200247867, US20200237821, US20200231686, WO2020123691, US20200147134, US20200079864, US20200055948, US20200023010, U.S. Ser. No. 10/526,406B2, US20190367621, US20190345256, US20190336504, US20190328784, US20190307797, WO2019178078, WO2019152660, WO2019152742, US20190231819, WO2019122875, U.S. Ser. No. 10/316,101B2, WO2019108932, WO2019085102, US20190125840, WO2019067015, US20190085081, US20190048061, US20190030073, WO2018169922, US20180230225, WO2018126369, WO2018068766, WO2017216562, US20170334991, WO2017149515, US20170226183, WO2017091546, US20170066827, US20170066838, WO2016094304, US20160280798, US20160096902, US20160051651, WO2015164739, WO2015063069, and WO2014186469; the disclosures of which are incorporated herein by reference in their entirety.

### Additional Components

Nucleic acid constructs and vectors may include various components in addition to the DR-18 polypeptide and/or CAR encoding sequences such as, but not limited to, e.g., a signal sequence (i.e., signal peptides or signal peptide sequences) to facilitate secretion, polyadenylation signals, transcription terminator sequences (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene, zeocin marker, and/or a detectable reporter). Any of the individual additional components described herein may be combined with any other individual additional components, including e.g., where a construct or vector may include 2, 3, 4, 5, 6, 7, 8, 9, 10, or more individual additional components, in various combinations, as desired.

In some instances, coding sequences, e.g., a DR-18 polypeptide encoding sequence, may be preceded, including fused in-frame to, a sequence encoding a signal peptide. Useful signal peptide sequences include but are not limited to e.g., the signal peptide sequences of Human OSM, VSV-G, Mouse Ig Kappa, Mouse Ig Heavy, BM40, Secrecon, Human IgKVIII, CD33, tPA, Human Chymotrypsinogen, Human trypsinogen-2, Human IL-2, Gaussia luc, Albumin (HSA), Influenza Haemagglutinin, Human insulin, Silkworm Fibroin LC, IL-18BP signal peptide, Interleukin-12 subunit beta, Interleukin-12 subunit alpha, Interleukin-18 propeptide, Human CD8, and the like. In some instances, the coding sequence, e.g., DR-18 polypeptide coding sequence, will include an endogenous signal peptide sequence, e.g., the signal peptide sequence of the wild-type IL-18 propeptide. In some instances, the coding sequence, e.g., DR-18 polypeptide coding sequence, will include a heterologous signal peptide sequence, e.g., an IL-2, IL-12, or IL-18BP signal peptide sequence, or the like. In some instances, a coding sequence may not include a signal peptide sequence.

Coding sequences of the present disclosure, e.g., DR-18 coding sequences, CAR coding sequences, and the like, will generally be operably linked to one or more regulatory elements. Useful regulatory elements to which coding sequences of the present disclosure may be operably linked include, but are not limited to e.g., enhancers, introns, polyadenylation signals, kozak consensus sequences, splice acceptor/donor sequences, response elements, such as woodchuck hepatitis virus (WPRE) elements, NFAT response element(s), and the like, promoters, and the like.

Nonlimiting examples of useful promoters include e.g., endogenous promoters, heterologous promoters, cytomegalovirus (CMV) promoter, elongation factor 1 alpha (EF1a) promoter, simian virus 40 (SV40) promoter, phosphoglycerate kinase (PGK) (e.g., human or mouse) promoter, ubiquitin C (UBC) promoter, human beta actin promoter, CAG promoter, TRE promoter, UAS promoter, LC immunoglobulin promoter, HC immunoglobulin promoter, herpes simplex virus thymidine kinase promoter, spleen focus-forming virus (SFFV) promoter, synthetic inducible promoters, inducible promoters, chemically/biochemically-regulated promoter, physically-regulated promoters, alcohol-regulated promoters, tetracycline-regulated promoters, steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), light-regulated promoters (e.g., light responsive promoters from plant cells), immune cell promoters, CD8 cell-specific promoter, CD4 cell-specific promoter, neutrophil-specific promoter, NK-specific promoter, B29 gene promoter, CD14 gene promoter, CD43 gene promoter, CD45 gene promoter, CD68 gene promoter, IFN-b gene promoter, WASP gene promoter, T-cell receptor b-chain gene promoter, V9 g (TRGV9) gene promoter, V2 d (TRDV2) gene promoter, and the like.

Various types of promoters may be employed in the nucleic acid, compositions, and systems of the present disclosure, including constitutive promoters, inducible promoters, repressible promoters, tissue specific promoters, cell-type specific promoters, and the like.

An example of a promoter that is capable of driving expression of a DR-18 polypeptide and/or a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In some embodiments, an EF1a promoter used in the described nucleic acids and vectors comprises the sequence provided as SEQ ID NO: 87.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Polypeptide encoding nucleic acid sequences of the present disclosure may be expressed from a constitutive promoter. Polypeptide encoding nucleic acid sequences of the present disclosure may be expressed from an inducible promoter. The use of an inducible promoter provides a molecular switch capable of turning on expression of the polynucleotide sequence which it is operatively linked in certain contexts, e.g., such as when expression is desired, or turning off the expression in certain contexts, e.g., such as when expression is not desired. Examples of inducible promoters include, but are not limited to a metallothionine promoter, a glucocorticoid promoter, a progesterone promoter, and a tetracycline promoter. In some instances, promoters responsive to immune cell activation may be employed, such as e.g., a promoter induced upon T cell activation, such as but not limited to e.g., a minimal interleukin 2 promoter (IL-2p) operably linked to one or more NFAT response elements, such as e.g., 2, 3, 4, 5, 6, or more NFAT response elements.

As described herein, nucleic acids of the present disclosure may be configured to include a one coding sequence per nucleic acid molecule or multiple coding sequences per nucleic acid molecule. For example, where at least one nucleic acid molecule includes a single coding sequence, multiple different nucleic acid molecules, including e.g., at least one molecule encoding a DR-18 polypeptide and at least one molecule encoding a CAR, may be employed to deliver all desired coding sequences into a cell. Where a nucleic acid molecule includes multiple coding sequences (e.g., a DR-18 polypeptide coding sequence and a CAR coding sequence) one nucleic acid molecule may be employed to deliver all desired coding sequences into a cell. In some instances, where a nucleic acid molecule includes multiple coding sequences one or more elements for multicistronic expression may be employed (also referred to as multicistronic elements).

Multicistronic expression includes but is not necessarily limited to bicistronic expression and tricistronic expression, which may, in some instances, be achieved through the use of multicistronic vectors, such as bicistronic and tricistronic vectors. Non-limiting examples of useful multicistronic elements include an internal ribosome entry site (IRES), a 2A peptide (e.g., T2A peptide, P2A peptide, E2A peptide, or F2A peptide) coding sequence, and the like. Multicistronic elements may be operably linked to one or more coding sequences.

Nucleic acids of the present disclosure may encode for one or more additional cytokines, where the term “additional cytokine” as used herein generally refers to a cytokine other than IL-18 (including wt and engineered forms of IL-18, such as DR-18). Inclusion of one or more additional cytokines in the nucleic acids, constructs, and/or vectors of the present disclosure provides for multi-armoring of a CAR construct, i.e., armoring the CAR construct with a DR-18 polypeptide and at least one more polypeptide with cytokine activity. Useful additional cytokines include but are not limited to e.g., IL-2, IL-7, IL-10, IL-12, IL-15, IL-21, IL-23, IL-27, IL-33, TNF, TL1A, IFNa, IFNb, and IFNg. Useful additional cytokines include wild-type cytokines and engineered versions thereof.

In order to assess the expression of a polypeptide or portions thereof, the expression vector to be introduced into a cell can also contain either a selectable marker gene or a reporter gene or both to facilitate identification and selection of expressing cells from the population of cells sought to be transduced, transfected or infected through viral or non-viral vectors. In other aspects, the selectable marker may be carried on a separate piece of DNA and used in a co-transfection procedure. Both selectable markers and reporter genes may be flanked with appropriate regulatory sequences to enable expression in the host cells. Useful reporters and selectable markers include, for example, antibiotic-resistance genes, such as neo and the like, fluorescent proteins such as green fluorescent protein or the like, enzymatic reporters such as luciferase, beta-galactosidase, chloramphenicol acetyl transferase, secreted alkaline phosphatase and the like.

### Methods

Methods of the present disclosure include methods of administering nucleic acids encoding a DR-18 polypeptide and at least one CAR to a subject in need thereof. Such subjects in need thereof, include subjects that may benefit from treatment with an expressed DR-18 and/or a CAR, including subjects having cancer, including where the cancer is a cancer to which an antigen binding domain of the CAR is directed. Useful methods include administering a DR-18 armored CAR where the CAR is any CAR described herein or a CAR directed to any antigen described herein. Useful methods include administering a DR-18 armored CAR where the DR-18 is any DR-18 described herein and any DR-18 encoding nucleic acid, including those described herein, may be employed. Nucleic acids of the present disclosure may be administered in such methods by a variety of different means, including e.g., where the nucleic acids are administered in vivo to the subject (e.g., through the use of a vector, such as a viral or non-viral vector), where the nucleic acids are contacted ex vivo with cells and the cells are subsequently administered to the subject. Cells employed for the delivery of DR-18 armored CAR nucleic acids may be derived from the subject receiving treatment (e.g., autologous) or from a donor that is not the subject receiving treatment (i.e., allogeneic). Nucleic acids of the present disclosure are administered to subjects in need thereof in a manner sufficient for the encoded elements of the nucleic acids to be expressed.

Allogeneic cells may include one or more genetic modifications rendering the cells less susceptible to clearance from a hosts immune response, such as but not limited to e.g., modification to disrupt T cell receptor alpha constant (TRAC) gene, modification to disrupt or prevent expression of 32 microglobulin, modification to disrupt endogenous human leukocyte antigen (HLA), modification to express HLA-E, and the like. Allogeneic cells, and methods of modifying cells for allogeneic cell therapy, include but are not limited to e.g., those described in WO2023078287, U.S. Ser. No. 11/389,481 B2, WO2022007784, US20230158070, WO2023093763, WO2019118475, US20230190809, WO2022192895, US20210268028, US20230068949, WO2021202581, US20230011889, WO2023279112, WO2022115864, US20210040449, WO2022018262, US20220041984, WO2023147776, US20230181641, WO2023010018, US20220023344, US20210355230, WO2023023515, US20220251505, US20180360883, WO2023070059, WO2021113759, WO2019089650, WO2020191378, WO2023196980, U.S. Ser. No. 10/538,574B2, WO2021228177, US20220118014, WO2021173630, WO2022187663, WO2019243835, US20220409665, US20220313736, and WO2022132720.

Methods of introducing genes for expression into a cell are known in the art. In the context of an expression vector, the vector can be readily introduced into a host cell, e.g., mammalian, by any method in the art. For example, the expression vector can be transferred into a host cell by physical, chemical, or biological means. Biological methods for introducing a polynucleotide of interest into a host cell include the use of DNA and RNA vectors.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned. Nucleic acid coding sequences can be derived from desired amino acid sequences, and such nucleic acid coding sequences can further be codon optimized for expression in a desired host, such as a human.

Nucleic acids of the present disclosure may be introduced into or inserted into vectors. The nucleic acid can be cloned into a number of types of vectors. For example, the nucleic acid can be cloned into a vector including, but not limited to a plasmid, a phagemid, a phage derivative, an animal virus, and a cosmid. Vectors of interest include expression vectors, replication vectors, transfer vectors, shuttle vectors, donor vectors, and the like. Viruses, which are useful as vectors include, but are not limited to, retroviruses, adenoviruses, adeno-associated viruses, herpes viruses, and lentiviruses.

Vectors derived from retroviruses such as the lentivirus are suitable tools to achieve long-term gene transfer since they allow long-term, stable integration of a transgene and its propagation in daughter cells. They also have the added advantage of low immunogenicity. A retroviral vector may also be, e.g., a gammaretroviral vector. A gammaretroviral vector may include, e.g., a promoter, a packaging signal (tp), a primer binding site (PBS), one or more (e.g., two) long terminal repeats (LTR), and a transgene of interest, e.g., a gene encoding a DR-18 polypeptide and/or a CAR. A gammaretroviral vector may lack viral structural genes such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom.

In another embodiment, the vector comprising a nucleic acid of the present disclose is an adenoviral vector. In another embodiment, the expression of nucleic acids encoding DR-18 polypeptides and/or CARs can be accomplished using transposons such as sleeping beauty, CRISPR nucleases (e.g., CAS9), and zinc finger nucleases. See e.g., June et al. 2009 Nature Reviews Immunology 9.10: 704-716.

Non-viral delivery system may be utilized, where an exemplary delivery vehicle is a nanoparticle, e.g., a liposome, lipid nanoparticle (LNP) or other suitable sub-micron sized delivery system. Lipid formulations are useful for the introduction of the nucleic acids, including DNA or RNA, into a host cell (in vitro, ex vivo or in vivo).

The instant disclosure also includes RNA constructs, encoding elements described herein, that can be directly transfected into a cell. A method for generating mRNA for use in transfection involves in vitro transcription (IVT) of a template with specially designed primers, followed by poly A addition, to produce a construct containing 3′ and 5′ untranslated sequence (“UTR”), a 5 cap and/or Internal Ribosome Entry Site (IRES), 2A sequence (P2A, T2A, etc.), etc., the sequence(s) encoding polypeptide(s) to be expressed, and a poly A tail. RNA so produced can efficiently transfect different kinds of cells. In one embodiment, the template includes sequences for the CAR and/or the DR-18. In an embodiment, an RNA armored CAR vector is transduced into a T cell by electroporation. In an embodiment, an armored CAR vector is associated with a lipid, such as encapsulated in the aqueous interior of a liposome, LNP, or the like, interspersed within the lipid bilayer of a liposome, LNP, or the like, attached to a lipid, liposome, or LNP, via a linking molecule, entrapped in a liposome, LNP, or the like, complexed with an LNP or the like, dispersed in a solution containing a lipid, mixed with a lipid, combined with a lipid, contained as a suspension in a lipid, contained or complexed with a micelle, or otherwise associated with a lipid.

The expression of natural or synthetic nucleic acids encoding armored CARs may be achieved by operably linking a nucleic acid encoding the CAR polypeptide or portions thereof and/or the DR-18 polypeptide to a promoter and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

Methods of making and using CAR encoding cells, which are applicable to making and using DR-18 polypeptide armored cells of the present disclosure, including methods of making vectors, transfection, transduction, cell culture and expansion, lymphodepletion, patient preparation, dose preparation and deliver, dosing, co-administration of other agents, patient monitoring, patient evaluation, and like, are described in e.g., the following U.S. and PCT patents and applications: U.S. Ser. No. 11/872,249B2, US20230416390, US20230381228, WO2023154890, US20230312708, WO2023147293, US20230256017, WO2023114777, US20230220090, US20230220103, U.S. Ser. No. 11/673,964B2, US20230174933, WO2023086900, US20230151094, WO2023044350, US20230084763, WO2023021477, US20230000964, US20220364055, US20220315665, US20220305056, US20220227874, US20220184129, US20220168389, US20220064254, US20220064316, US20220047633, US20210393689, US20210347851, US20210324100, U.S. Ser. No. 11/149,076B2, US20210284752, US20210220404, US20210177896, US20210171909, U.S. Ser. No. 11/028,177B2, US20210163612, WO2021099944, US20210139595, WO2021067290, US20200407461, US20200306304, U.S. Ser. No. 10/752,684B2, U.S. Ser. No. 10/752,670B2, US20200231686, US20200055948, U.S. Ser. No. 10/526,406B2, US20190336504, WO2019152660, U.S. Ser. No. 10/316,101B2, WO2017149515, US20170226183, and US20160051651; the disclosures of which are incorporated herein by reference in their entirety.

Cells into which nucleic acids of the present disclosure are introduced may first be obtained, e.g., prior to expansion and/or genetic modification, from a mammalian subject. Useful mammalian subjects include living mammalian subjects from which an immune response can be elicited, such as e.g., humans, primates, non-human primates, dogs, cats, mice, rats, and transgenic species thereof. Cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, cells are obtained from a subject, prepared, including genetically modified, expanded, etc., and used to treat the subject from which they were obtained. In some embodiments, cells are obtained from a first subject, prepared, including genetically modified, expanded, etc., and used to treat a second subject other than the subject from which they were obtained. In some embodiments, any number of available cell lines, including available immune cell lines, such as available T cell lines, may be employed. Various useful methods of may be employed in cell collection, alone or in combination, such as but not limited to e.g., blood collection kits, density gradients (with or without centrifugation), apheresis, cell washing, isolation of desired cell types, positive selection, depletion of unwanted cell types, negative selection, and the like.

In some instances, cells of the present disclosure may be expanded, e.g., before, after, or during activation; or before, after, or during genetic modification. Any useful and appropriate method for expansion of the relevant cell type may be employed for expansion. Cells may be expanded by contact with one or more agents that stimulates proliferation of the cells, including e.g., where one or more of the agents is bound to a surface. For example, with respect to T cells, the cells may be expanded by contact with one or more agents such as antibodies, such as e.g., an anti-CD3 antibody or binding fragment thereof, an anti-CD2 antibody or binding fragment thereof, an anti-CD28 antibody or binding fragment thereof, and the like, including where one or more such antibodies are in solution, bound to a surface, such as a bead, a plate, a well, or the like, or a combination thereof.

In some embodiments, T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication Nos. 20060121005 and 20230416390.

Agents for the activation and/or expansion of cells of the present disclosure may be cultured together for any useful and appropriate amount of time, including e.g., hours, such as e.g., about 1-2 hours, about 2-4 hours, about 4-6 hours, about 6-12 hours, about 12-24 hours, about 1-24 hours, about 1-12 hours, about 1-6 hours, etc., days, such as e.g., about 1 day, about 2 days, about 1-2 days, about 2-3 days, about 2-4 days, about 3-5 days, about 4-6 days, about 5-7 days, about 7 days, about 6-10 days, about 8 days, about 7-14 days, about 10 days, about 14 days, about 10-20 days, about 15 days, about 18 days, about 20 days, about 14-21 days, about 21 days, etc., weeks, such as e.g., about 1 week, about 2 weeks, about 3 weeks, about 4 weeks, etc. In some instances, multiple cycles of activation and/or stimulation may be employed.

Compositions and methods of the present disclosure may be used to treat a subject who has been characterized as having cells or tissues expressing one or more antigens to which the antigen-binding portion of the relevant CAR binds, or is suspected of having cells or tissues expressing one or more antigens to which the antigen-binding portion of the relevant CAR binds. For example, subjects benefiting from treatment according to the invention include subjects with a cancer, or subjects suspected of having a cancer, for example, as evidenced by the presence of one or more symptoms, clinical manifestations, or diagnostic indicators of the caner, including where the cancer is known or suspected to express one or more antigens to which the antigen-binding portion of the relevant CAR binds.

The present disclosure provides methods for inhibiting the proliferation or reducing a cell population expressing an antigen to which the antigen binding region of the CAR binds (i.e., a target cell), the methods comprising contacting a population of cells comprising a target cell with an armored CAR-expressing cell (or nucleic acids encoding the armored CAR components) described herein, e.g., a T cell, that binds to an antigen expressed by the target cell. In an embodiment, the present disclosure provides methods for inhibiting the proliferation or reducing the population of cancer cells expressing a target cell antigen, the methods comprising contacting the target cell-expressing cancer cell population with armored CAR-expressing cells (or nucleic acids encoding the armored CAR components) described herein, e.g., a T cell, that binds to the target antigen-expressing cell.

Cancers and tumors, the treatment of which may include the use of a DR-18 armored CAR of the instant disclosure, will vary and may include but are not limited to e.g. Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenstrom Macroglobulinemia, Wilms Tumor, and the like.

In some instances, methods of treatment of the present disclosure will include treating a blood cancer. In some instances, methods of treatment of the present disclosure will include treating a solid tumor. In some instances, methods of treatment of the present disclosure will include treating a subject for a naïve cancer. In some instances, methods of treatment of the present disclosure will include treating a subject for a cancer that has been previously treated, including previously treated with one or more of chemotherapy, radiation, immunotherapy, or any combination thereof. In some instances, methods of treatment of the present disclosure will include treating a primary tumor. In some instances, methods of treatment of the present disclosure will include treating a metastatic tumor. In some instances, methods of treatment of the present disclosure will include treating a recurrent cancer or tumor.

Methods of the present disclosure find use in treating treatment naïve cancers, treatment refractory cancers, primary tumors, metastatic tumors, and the like. In some instances, methods of the present disclosure find use in treating and/or managing a cancer that includes cells expressing a target antigen. In some instances, methods of the present disclosure find use in preventing relapse of a cancer that includes cells expressing a target antigen. Methods of the present disclosure include administering an effective amount of armored CAR cells (or nucleic acids encoding the armored CAR components), described herein, to a patient in need thereof, including where the subject in need thereof is a subject, including a human, having cancer, suspected of having cancer, at risk of developing cancer, at risk of relapsing, or the like. In some instances, administration of the armored CAR cells (or nucleic acids encoding the armored CAR components) may be the only therapy administered to the subject during the course of treatment (i.e., monotherapy). In some instances, the armored CAR cells (or nucleic acids encoding the armored CAR components) may be administered in combination with one or more other agents active against the subject's cancer during the course of therapy as part of a combination therapy.

The armored CAR-modified cells (or nucleic acids encoding the armored CAR components) of the present invention may be administered either alone, or as a pharmaceutical composition in combination with diluents and/or with other components such as other cytokines or cell populations or other drug treatments, e.g., described herein. Additional cancer-treating agents, e.g., for use in combination therapies with nucleic acids and/or cells of the present disclosure, are known to the relevant ordinarily skilled artisan. For example, in some instances, armored CAR immune cells (or a vector for armored CAR in vivo therapy) may be administered in combination with one or more cytokines, such as e.g., IL-2, IL-7, IL-10, IL-12, IL-15, IL-21, IL-23, IL-27, IL-33, TNF, TL1A, IFNa, IFNb, and IFNg. In some instances, the additional cytokine administered is not IL-7, IL15, or IL-10.

Methods of the present disclosure may include administering to a subject a nucleic acid that includes a sequence encoding a DR-18 polypeptide and one or more sequences encoding at least one additional cytokine. The term “additional cytokine” as used herein refers to cytokines providing a cytokine function in addition to the IL-18 signaling function provided by the encoded DR-18 polypeptide (i.e., a cytokine other than IL-18). Such additional cytokines include e.g., IL-2, IL-7, IL-10, IL-12, IL-15, IL-21, IL-23, IL-27, IL-33, TNF, TL1A, IFNa, IFNb, and IFNg. In some instances, the additional cytokine is not IL-7, IL15, or IL-10. Accordingly, in some instances, a cell or a treatment of the present disclosure may be referred to a multi- (e.g., dual-, triple-, etc.) armored where such multi-armoring includes armoring with DR-18 polypeptide expression and expression of at least one additional cytokine, such as, e.g., IL-2, IL-7, IL-10, IL-12, IL-15, IL-21, IL-23, IL-27, IL-33, TNF, TL1A, IFNa, IFNb, and IFNg. In some instances, the additional cytokine of the multi-armored DR-18 polypeptide is not (or is a cytokine other than) one or more of IL-2, IL-7, IL-10, IL-12, IL-15, IL-21, IL-23, IL-27, IL-33, TNF, TL1A, IFNa, IFNb, and IFNg, for example, the cytokine is not one or more of IL-7, IL15, or IL-10 or is a cytokine other than one or more of IL-7, IL15, or IL-10.

In one embodiment, a cell or a CAR therapy may be multi-armored with expression of a DR-18 polypeptide and expression of an IL-12, including e.g., an IL-12 with a wild-type IL12p35 with (SEQ ID NO: 88) or without (SEQ ID NO: 89) endogenous signal peptide, an IL-12 with a wild-type IL12p40 with (SEQ ID NO: 90) or without (SEQ ID NO: 91) endogenous signal peptide, an IL-12 partial agonist comprising a variant IL12p35, an IL-12 partial agonist comprising a variant IL12p40, a detuned IL-12, combinations thereof or the like.

As used herein, a “partial agonist” refers to a variant of an agonist that has submaximal signaling through one or more of its receptors (e.g., as compared to the wild-type agonist) and, without being bound by theory, is capable of selectively biasing the activation of cells and/or signaling pathways that possess differential activation thresholds. For example, an IL-12 partial agonist will include a variant IL-12p40 subunit, variant IL-12p35 subunit, or variants of both subunits, that have submaximal signaling (as compared to wild-type IL-12) through one or more of its receptors or receptor subunits. Without being bound by theory, such submaximal signaling may have the effect of selectively activating desired cell populations while avoiding the activation (or minimally activating) undesired cell populations. As used herein, a “detuned” version of a cytokine refers to a variant that has reduced affinity for one or more of its receptors.

In some instances, a subject may be administered one or more nucleic acids including sequences that encode for at least a DR-18 polypeptide, an IL-12 partial agonist or detuned IL-12, and a CAR. Any of the herein described DR-18 polypeptides and CARs, or components thereof, may be employed in an at least dual-armored DR-18 and IL-12 partial agonist or detuned IL-12 CAR therapy. Useful IL-12 partial agonists include but are not limited to those described in PCT Pat. Pub. Nos. WO2023023503 and WO2021212083. Useful IL-12 partial agonists (including variant IL12p40 subunits, variant IL12p35 subunits, or both variant IL12p40 and variant IL12p35 subunits) that may be encoded by one or more sequences included the nucleic acids of the presented disclosure, include e.g., SEQ ID NOs: 92-108. Such IL-12 coding sequences may encode for a wt IL-12p35 subunit, a IL-12p35 variant subunit, a wt IL-12p40 subunit, a IL-12p40 variant subunit, a IL-12p35 subunit (variant or wt) with its endogenous signal peptide, a IL-12p35 subunit (variant or wt) without its endogenous signal peptide (e.g., with a heterologous signal peptide), a IL-12p40 subunit (variant or wt) with its endogenous signal peptide, a IL-12p40 subunit (variant or wt) without its endogenous signal peptide (e.g., with a heterologous signal peptide), or any combination thereof. In some instances, where an endogenous signal peptide is not present the coding sequence may include sequence encoding a heterologous signal peptide.

Methods of the present disclosure that include administering a DR-18 armored CAR immune cell or a vector for in vivo DR-18 armored CAR immune cell therapy will provide for certain advantage as compared to corresponding CAR therapies not employing DR-18 armoring. For example, in some instances, methods of the present disclosure may be performed without lymphodepletion of the subject prior to or during treatment with the DR-18 armored CAR therapy. For example, in the case of autologous CAR T or NK cell therapy, cells may be collected from the subject, modified ex vivo to express the DR-18 polypeptide and CAR, activated and/or expanded as desired, and administered to the subject without the subject having undergone lymphodepletion. In the case of allogeneic CAR T or NK cell therapy, allogeneic DR-18 armored CAR T or NK cells may be administered to the subject without the subject having undergone lymphodepletion. In some instances, the DR-18 armored CAR immune cell therapy without lymphodepletion achieves at least equivalent engraftment, efficacy, and/or persistence of the DR-18 armored CAR immune cells as compared to an equivalent CAR immune cell therapy without DR-18 armoring and with lymphodepletion.

Lymphodepletion is a conditioning step used in autologous and allogeneic CAR immune cell therapies that depletes and modulates endogenous lymphocytes in the recipient to prepare the host to receive the CAR immune cell therapy. Various methods using various agents, combinations of agents, radiation, and combinations thereof are employed in lymphodepletion regimens. Non-limiting examples of agents useful in lymphodepletion include fludarabine, cyclophosphamide, bendamustine, alemtuzumab, oxaliplatin, clofarabine, and combinations thereof. Lymphodepletion and useful lymphodepletion regimens are described e.g., in Lickefett et al. (2023) Front. Immunol. 14:1303935.

In some instances, treatment of a subject with a DR-18 armored CAR immune cell, such as a T cell or NK cell, or a vector for in vivo DR-18 armored CAR immune cell therapy will achieve an equivalent or better effect as compared to the corresponding CAR immune cell therapy performed without DR-18 armoring, including e.g., where the DR-18 armored CAR therapy is administered at a lower dose than the CAR therapy without DR-18 armoring, where the DR-18 armored CAR therapy is administered less frequently as compared to the CAR therapy without DR-18 armoring, or where the DR-18 armored CAR therapy is administered at a lower dose and less frequently as compared to the CAR therapy without DR-18 armoring. In some instances, DR-18 armored CAR immune cells (whether generated ex vivo or in vivo) will persist and/or remain active longer in a subject than corresponding CAR immune cells without DR-18 armoring, and thus require less frequent and/or lower dosing. In some instances, DR-18 armored CAR immune cells (whether generated ex vivo or in vivo) will be less susceptible to exhaustion as compared to corresponding CAR immune cells without DR-18 armoring, and thus require less frequent and/or lower dosing.

Methods of the present disclosure further include methods of making DR-18 armored CAR immune cells. DR-18 armored CAR immune cells, containing at least a sequence encoding a DR-18 polypeptide and a CAR encoding sequence, may be made using any of the components and/or processes described herein. In some instances, methods of the present disclosure may include introducing a DR-18 polypeptide encoding sequence into a cell ex vivo and culturing the cell following introduction of the DR-18 polypeptide. The introduced DR-18 polypeptide encoding sequence may be configured to express the DR-18 polypeptide during culture of the modified cell. In some instances, the presence of DR-18 polypeptide during the cell manufacturing process provides for an enhanced manufacturing process as compared to a corresponding process in the absence of expressed DR-18 polypeptide.

In some instances, culturing a population of cells wherein at least some portion of the cells (e.g., at least 10%, 20%, 30%, 40%, 50%, or more) express the DR-18 polypeptide will provide certain advantages as compared to culturing a corresponding population of cells that do not express the DR-18 polypeptide during culturing. For example, the presence of expressed DR-18 polypeptide during the culturing process may provide for enhanced proliferation or expansion of the cells as compared to corresponding cells cultured in the absence of expressed DR-18 polypeptide. In some instances, the presence of expressed DR-18 polypeptide during the culturing process may provide for enhanced activation of the cells as compared to corresponding cells cultured in the absence of expressed DR-18 polypeptide. In some instances, cells or cell populations produced in the presence of expressed DR-18 polypeptide during the culturing process will have an enhanced phenotype as compared to corresponding cells or cell populations produced in the absence of expressed DR-18 polypeptide. Such enhanced phenotypes may include e.g., enhanced in vivo activity, enhanced persistence in culture, enhanced persistence in vivo, enhanced proliferation in culture, enhanced proliferation in vivo, enhanced tumor cell killing, combinations thereof, and the like.

SEQUENCES

| SEQ | | |
|---|---|---|
| ID | | |
| NO: | Ref. name | Sequence |
| 1 | wt hIL-18 | YFGKLESKLSVIRNLNDQVLFIDQGNRPLFEDMTDSDCRDNAPRTIFIISMYKDSQPRGMAVTI |
| | | SVKCEKISTLSCENKIISFKEMNPPDNIKDTKSDIIFFQRSVPGHDNKMQFESSSYEGYFLACEK |
| | | ERDLFKLILKKEDELGDRSIMFTVQNED |
| 2 | wt hIL-18 | MAAEPVEDNCINFVAMKFIDNTLYFIAEDDENLESDYFGKLESKLSVIRNLNDQVLFIDQGNRP |
| | (unprocessed) | LFEDMTDSDCRDNAPRTIFIISMYKDSQPRGMAVTISVKCEKISTLSCENKIISFKEMNPPDNIK |
| | | DTKSDIIFFQRSVPGHDNKMQFESSSYEGYFLACEKERDLFKLILKKEDELGDRSIMFTVQNED |
| 3 | wt hIL-18 | MAAEPVEDNCINFVAMKFIDNTLYFIAEDDENLESD |
| | propeptide | |
| 4 | sq89v1 | YFGKLESKLSVIRNLNDQVLFIDQGNRPLFEDMTDSDSRDNAPRTIFIISKYSDSLARGLAVTIS |
| | | VKSEKISTLSSENKIISFKEMNPPDNIKDTKSDIIFFQRDVPGHSRKMQFESSSYEGYFLASEKE |
| | | RDLFKLILKKEDELGDRSIMFTVQNED |
| 5 | sq89v2 | YFGKLESKLSVIRNLNDQVLFIDQGNRPLFEDMTDSDCRDNAPRTIFIISKYSDSLARGLAVTIS |
| | | VKSEKISTLSSENKIISFKEMNPPDNIKDTKSDIIFFQRDVPGHSRKMQFESSSYEGYFLASEKE |
| | | RDLFKLILKKEDELGDRSIMFTVQNED |
| 6 | sq89v3 | YFGKLESKLSVIRNLNDQVLFIDQGNRPLFEDMTDSDSRDNAPRTIFIISKYSDSLARGLAVTIS |
| | | VKCEKISTLSSENKIISFKEMNPPDNIKDTKSDIIFFQRDVPGHSRKMQFESSSYEGYFLASEKE |
| | | RDLFKLILKKEDELGDRSIMFTVQNED |
| 7 | sq89v4 | YFGKLESKLSVIRNLNDQVLFIDQGNRPLFEDMTDSDSRDNAPRTIFIISKYSDSLARGLAVTIS |
| | | VKSEKISTLSCENKIISFKEMNPPDNIKDTKSDIIFFQRDVPGHSRKMQFESSSYEGYFLASEKE |
| | | RDLFKLILKKEDELGDRSIMFTVQNED |
| 8 | sq89v5 | YFGKLESKLSVIRNLNDQVLFIDQGNRPLFEDMTDSDSRDNAPRTIFIISKYSDSLARGLAVTIS |
| | | VKSEKISTLSSENKIISFKEMNPPDNIKDTKSDIIFFQRDVPGHSRKMQFESSSYEGYFLACEKE |
| | | RDLFKLILKKEDELGDRSIMFTVQNED |
| 9 | sq89v6 | YFGKLESKLSVIRNLNDQVLFIDQGNRPLFEDMTDSDSRDNAPRTIFIISKYSDSLARGLAVTIS |
| | | VKSEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 10 | sq89v7 | YFGKLESKLSVIRNLNDQVLFIDQGNRPLFEDMTDSDSRDNAPRTIFIISKYSDSLARGLAVTIS |
| | | VKCEKISTLSSENKIISFKEMNPPDNIKDTKSDIIFFQRDVPGHSRKMQFESSSYEGYFLACEKE |
| | | RDLFKLILKKEDELGDRSIMFTVQNED |
| 11 | sq89v8 | YFGKLESKLSVIRNLNDQVLFIDQGNRPLFEDMTDSDSRDNAPRTIFIISKYSDSLARGLAVTIS |
| | | VKCEKISTLSCENKIISFKEMNPPDNIKDTKSDIIFFQRDVPGHSRKMQFESSSYEGYFLASEKE |
| | | RDLFKLILKKEDELGDRSIMFTVQNED |
| 12 | sq89v9 | YFGKLESKLSVIRNLNDQVLFIDQGNRPLFEDMTDSDCRDNAPRTIFIISKYSDSLARGLAVTIS |
| | | VKSEKISTLSSENKIISFKEMNPPDNIKDTKSDIIFFQRDVPGHSRKMQFESSSYEGYFLACEKE |
| | | RDLFKLILKKEDELGDRSIMFTVQNED |
| 13 | q89v10 | YFGKLESKLSVIRNLNDQVLFIDQGNRPLFEDMTDSDCRDNAPRTIFIISKYSDSLARGLAVTIS |
| | | VKSEKISTLSCENKIISFKEMNPPDNIKDTKSDIIFFQRDVPGHSRKMQFESSSYEGYFLASEKE |
| | | RDLFKLILKKEDELGDRSIMFTVQNED |
| 14 | q89v11 | YFGKLESKLSVIRNLNDQVLFIDQGNRPLFEDMTDSDCRDNAPRTIFIISKYSDSLARGLAVTIS |
| | | VKCEKISTLSSENKIISFKEMNPPDNIKDTKSDIIFFQRDVPGHSRKMQFESSSYEGYFLASEKE |
| | | RDLFKLILKKEDELGDRSIMFTVQNED |
| 15 | q89v12 | YFGKLESKLSVIRNLNDQVLFIDQGNRPLFEDMTDSDSRDNAPRTIFIISKYSDSLARGLAVTIS |
| | | VKCEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 16 | q89v13 | YFGKLESKLSVIRNLNDQVLFIDQGNRPLFEDMTDSDCRDNAPRTIFIISKYSDSLARGLAVTIS |
| | | VKSEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 17 | q89v14 | YFGKLESKLSVIRNLNDQVLFIDQGNRPLFEDMTDSDCRDNAPRTIFIISKYSDSLARGLAVTIS |
| | | VKCEKISTLSSENKIISFKEMNPPDNIKDTKSDIIFFQRDVPGHSRKMQFESSSYEGYFLACEKE |
| | | RDLFKLILKKEDELGDRSIMFTVQNED |
| 18 | q89v15 | YFGKLESKLSVIRNLNDQVLFIDQGNRPLFEDMTDSDCRDNAPRTIFIISKYSDSLARGLAVTIS |
| | | VKCEKISTLSCENKIISFKEMNPPDNIKDTKSDIIFFQRDVPGHSRKMQFESSSYEGYFLASEKE |
| | | RDLFKLILKKEDELGDRSIMFTVQNED |
| 19 | C68v1 | YFGKLESKLSVIRNLNDQVLFIDQGNRPLFEDMTDSDSRDNAPRTIFIISKYSDSLARGLAVTIS |
| | | VKGEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 20 | C68v2 | YFGKLESKLSVIRNLNDQVLFIDQGNRPLFEDMTDSDSRDNAPRTIFIISKYSDSLARGLAVTIS |
| | | VKAEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 21 | C68v3 | YFGKLESKLSVIRNLNDQVLFIDQGNRPLFEDMTDSDSRDNAPRTIFIISKYSDSLARGLAVTIS |
| | | VKVEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 22 | C68v4 | YFGKLESKLSVIRNLNDQVLFIDQGNRPLFEDMTDSDSRDNAPRTIFIISKYSDSLARGLAVTIS |
| | | VKDEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 23 | C68v5 | YFGKLESKLSVIRNLNDQVLFIDQGNRPLFEDMTDSDSRDNAPRTIFIISKYSDSLARGLAVTIS |
| | | VKEEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 24 | C68v6 | YFGKLESKLSVIRNLNDQVLFIDQGNRPLFEDMTDSDSRDNAPRTIFIISKYSDSLARGLAVTIS |
| | | VKNEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 25 | hCS1 | YFGKLESKLSVIRNLNDQVLFIDQGNRPLFEDMTDSDCRDNAPRTIFIISTYKDSQPRGKAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTVQNED |
| 26 | hCS2 | YFGKLESKLSVIRNLNDQVLFIDQGNRPLFEDMTDSDCRDNAPRTIFIISTYKDKQPRAKAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTIQNED |
| 27 | hCS3 | RFGKLESKLSVIRNLNDQVLFIDQGNRPLFEDMTDSDCRDNAPRTIFIISTYKDSQPRGKAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTVQNED |
| 28 | hCS4 | RFGKLESKLSVIRNLNDQVLFIDQGNRPLFEDMTDSDCRDNAPRTIFIISTYRDSQPRGKAVTIS |
| | | VKCEKISTLSCENKIISFKEMNPPDNIKDTKSDIIFFQRNVPGHKYKMQFESSSYEGYFLACEKE |
| | | RDLFKLILKKEDELGDRSIMFTVQNED |
| 29 | hC4 | YFGKLESQLSVIRNLNDQVLFIDQGNRPLFEDMTDSDCRDNAPRTIFIISTYKDKQPRTKAVTIS |
| | | VKCEKISTLSCENKIISFKEMNPPDNIKDTKSDIIFFQRRVPGHHNKMQFESSSYEGYFLACEKE |
| | | RDLFKLILKKEDELGDRSIMFTVQKED |
| 30 | hA8 | YFGKLESRLSVIRNLNDQVLFIDQGNRPLFEDMTDSDCRDNAPRTIFIISKYKDKQPRAQAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTIQNED |
| 31 | hD6 | YFGKLESRLSVIRNLNDQVLFIDQGNRPLFEDMTDSDCRDNAPRTIFIISDYKDKQPRAXAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTIQNED |
| 32 | hH12 | YFGKHESKLSVIRNLNDQVLFIDQGNRPLFEDMTDSDCRDNAPRTIFIISTYRDSQPRGKAVTI |
| | | SVKCEKISTLSCENKIISFKEMNPPDNIKDTKSDIIFFQRDVPGHNNKMQFESSSYEGYFLACEK |
| | | ERDLFKLILKKEDELGDRSIMFTTQNED |
| 33 | hB11 | YFGKIESKLSVIRNLNDQVLFIDQGNRPLFEDMTDSDCRDNAPRTIFIISKYKDKQPRAQAVTIS |
| | | VKCEKISTLSCENKIISFKEMNPPDNIKDTKSDIIFFQRKVPGHQHKMQFESSSYEGYFLACEKE |
| | | RDLFKLILKKEDELGDRSIMFTVQKED |
| 34 | hC3 | YFGKIESKLSVIRNLNDQVLFIDQGNRPLFEDMTDSDCRDNAPRTIFIISTYKDRQPRGKAVTIS |
| | | VKCEKISTLSCENKIISFKEMNPPDNIKDTKSDIIFFERDVPGHHHKMQFESSSYEGYFLACEKE |
| | | RDLFKLILKKEDELGDRSIMFTIQNED |
| 35 | hC2 | YFGKIESKLSVIRNLNDQVLFIDQGNRPLFEDMTDSDCRDNAPRTIFIISTYKDKQPRGKAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTTQHED |
| 36 | hG10 | YFGKIESKLSVIRNLNDQVLFIDQGNRPLFEDMTDSDCRDNAPRTIFIISTYKDKQPRAKAVTIS |
| | | VKCEKISTLSCENKIISFKEMNPPDNIKDTKSDIIFFQRRVPGHHHKMQFESSSYEGYFLACEKE |
| | | RDLFKLILKKEDELGDRSIMFTIQKED |
| 37 | hG1 | YFGKIESRLSVIRNLNDQVLFIDQGNRPLFEDMTDSDCRDNAPRTIFIISTYKDKQPRGKAVTIS |
| | | VKCEKISTLSCENKIISFKEMNPPDNIKDTKSDIIFFQRDVPGHDYKMQFESSSYEGYFLACEKE |
| | | RDLFKLILKKEDELGDRSIMFTIQKED |
| 38 | hF1 | YFGKYESRLSVIRNLNDQVLFIDQGNRPLFEDMTDSDCRDNAPRTIFIISTYRDSQPRGKAVTI |
| | | SVKCEKISTLSCENKIISFKEMNPPDNIKDTKSDIIFFQRDVPGHEHKMQFESSSYEGYFLACEK |
| | | ERDLFKLILKKEDELGDRSIMFTVQKED |
| 39 | hD2 | HFGKYESKLSVIRNLNDQVLFIDQGNRPLFEDMTDSDCRDNAPRTIFIISTYRDSQPRGKAVTI |
| | | SVKCEKISTLSCENKIISFKEMNPPDNIKDTKSDIIFFQRDVPGHHNKMQFESSSYEGYFLACEK |
| | | ERDLFKLILKKEDELGDRSIMFTVQKED |
| 40 | hA1 | RFGKLESKLSVIRNLNDQVLFIDQGNRPLFEDMTDSDCRDNAPRTIFIISTYRDSQPRAKAVTIS |
| | | VKCEKISTLSCENKIISFKEMNPPDNIKDTKSDIIFFQRDVPGHQHKMQFESSSYEGYFLACEK |
| | | ERDLFKLILKKEDELGDRSIMFTAQKED |
| 41 | hB3 | RFGKLESRLSVIRNLNDQVLFIDQGNRPLFEDMTDSDCRDNAPRTIFIISDYRDSQPRGRAVTI |
| | | SVKCEKISTLSCENKIISFKEMNPPDNIKDTKSDIIFFKRNVPGHKYKMQFESSSYEGYFLACEK |
| | | ERDLFKLILKKEDELGDRSIMFTVQHED |
| 42 | hB4 | RFGKLESRLSVIRNLNDQVLFIDQGNRPLFEDMTDSDCRDNAPRTIFIISNYRDSQPRGQAVTI |
| | | SVKCEKISTLSCENKIISFKEMNPPDNIKDTKSDIIFFKRRVPGHNHKMQFESSSYEGYFLACEK |
| | | ERDLFKLILKKEDELGDRSIMFTVQKED |
| 43 | hH3 | RFGKLESRLSVIRNLNDQVLFIDQGNRPLFEDMTDSDCRDNAPRTIFIISTYKDSQPRGKAVTIS |
| | | VKCEKISTLSCENKIISFKEMNPPDNIKDTKSDIIFFQRDVPGHKHKMQFESSSYEGYFLACEKE |
| | | RDLFKLILKKEDELGDRSIMFTVQNED |
| 44 | hH5 | RFGKHESKLSVIRNLNDQVLFIDQGNRPLFEDMTDSDCRDNAPRTIFIISTYRDSQPRGKAVTI |
| | | SVKCEKISTLSCENKIISFKEMNPPDNIKDTKSDIIFFERNVPGHKYKMQFESSSYEGYFLACEK |
| | | ERDLFKLILKKEDELGDRSIMFTVQNED |
| 45 | hH4 | RFGKLESRLSVIRNLNDQVLFIDQGNRPLFEDMTDSDCRDNAPRTIFIISTYRDSQPRAKAVTIS |
| | | VKCEKISTLSCENKIISFKEMNPPDNIKDTKSDIIFFERDVPGHQHKMQFESSSYEGYFLACEKE |
| | | RDLFKLILKKEDELGDRSIMFTIQXED |
| 46 | hE1 | RFGKLESRLSVIRNLNDQVLFIDQGNRPLFEDMTDSDCRDNAPRTIFIISTYRDSQPRTKAVTIS |
| | | VKCEKISTLSCENKIISFKEMNPPDNIKDTKSDIIFFQRNVPGHHDKMQFESSSYEGYFLACEKE |
| | | RDLFKLILKKEDELGDRSIMFTVQHED |
| 47 | hG2 | RFGKLESRLSVIRNLNDQVLFIDQGNRPLFEDMTDSDCRDNAPRTIFIISTYKDSQPRAKAVTIS |
| | | VKCEKISTLSCENKIISFKEMNPPDNIKDTKSDIIFFERDVPGHQHKMQFESSSYEGYFLACEKE |
| | | RDLFKLILKKEDELGDRSIMFTIQKED |
| 48 | hE12 | RFGKYESKLSVIRNLNDQVLFIDQGNRPLFEDMTDSDCRDNAPRTIFIISTYKDSQPRTKAVTIS |
| | | VKCEKISTLSCDNKIISFKEMNPPDNIKDTKSDIIFFQRDVPGHKHKMQFESSSYEGYFLACEKE |
| | | RDLFKLILKKEDELGDRSIMFTVQNED |
| 49 | hC5 | RFGKLESRLSVIRNLNDQVLFIDQGNRPLFEDMTDSDCRDNAPRTIFIISTYRDSQPRTKAVTIS |
| | | VKCEKISTLSCENKIISFKEMNPPDNIKDTKSDIIFFQRKVPGHNHKMQFESSSYEGYFLACEKE |
| | | RDLFKLILKKEDELGDRSIMFTVQKED |
| 50 | 5-18 | YFGKLESKLSVIRNLNDQVLFIDQGNRPLFEDMTDSDCRDNAPRTIFIISEYKDSELRGRAVTIS |
| | | VKCEKISTLSCENKIISFKEMNPPDNIKDTKSDIIFFPRAVPGHNRKVQFESSSYEGYFLACEKE |
| | | RDLFKLILKKEDELGDRSIMFTVQNED |
| 51 | 5-29 | YFGKLESKLSVIRNLNDQVLFIDQGNRPLFEDMTDSDCRDNAPRTIFIISKYKDSAGRGLAVTIS |
| | | VKCEKISTLSCENKIISFKEMNPPDNIKDTKSDIIFFERDVPGHSNKVQFESSSYEGYFLACEKE |
| | | RDLFKLILKKEDELGDRSIMFTVQNED |
| 52 | 5-8 | YFGKLESKLSVIRNLNDQVLFIDQGNRPLFEDMTDSDCRDNAPRTIFIISKYGDSAARGLAVTIS |
| | | VKCEKISTLSCENKIISFKEMNPPDNIKDTKSDIIFFQRSVPGHKRKMQFESSSYEGYFLACEKE |
| | | RDLFKLILKKEDELGDRSIMFTVQNED |
| 53 | 5-6 | YFGKLESKLSVIRNLNDQVLFIDQGNRPLFEDMTDSDCRDNAPRTIFIISKYGDSRGRGLAVTIS |
| | | VKCEKISTLSCENKIISFKEMNPPDNIKDTKSDIIFFERDVPGHNSKRQFESSSYEGYFLACEKE |
| | | RDLFKLILKKEDELGDRSIMFTVQNED |
| 54 | 5-27 | YFGKLESKLSVIRNLNDQVLFIDQGNRPLFEDMTDSDCRDNAPRTIFIISKYGDSVPRGLAVTIS |
| | | VKCEKISTLSCENKIISFKEMNPPDNIKDTKSDIIFFARAVPGHSRKTQFESSSYEGYFLACEKE |
| | | RDLFKLILKKEDELGDRSIMFTVQNED |
| 55 | 5-20 | YFGKLESKLSVIRNLNDQVLFIDQGNRPLFEDMTDSDCRDNAPRTIFIISKYSDSGARGLAVTIS |
| | | VKCEKISTLSCENKIISFKEMNPPDNIKDTKSDIIFFARAVPGHGRKTQFESSSYEGYFLACEKE |
| | | RDLFKLILKKEDELGDRSIMFTVQNED |
| 56 | 5-2 | YFGKLESKLSVIRNLNDQVLFIDQGNRPLFEDMTDSDCRDNAPRTIFIISKYSDSKARGMAVTIS |
| | | VKCEKISTLSCENKIISFKEMNPPDNIKDTKSDIIFFARDVPGHSSKRQFESSSYEGYFLACEKE |
| | | RDLFKLILKKEDELGDRSIMFTVQNED |
| 57 | 6-12 | YFGKLESKLSVIRNLNDQVLFIDQGNRPLFEDMTDSDCRDNAPRTIFIISKYSDSLARGLAVTIS |
| | | VKCEKISTLSCENKIISFKEMNPPDNIKDTKSDIIFFQRDVPGHSRKMQFESSSYEGYFLACEKE |
| | | RDLFKLILKKEDELGDRSIMFTVQNED |
| 58 | 5-42 | YFGKLESKLSVIRNLNDQVLFIDQGNRPLFEDMTDSDCRDNAPRTIFIISKYSDSRARGLAVTIS |
| | | VKCEKISTLSCENKIISFKEMNPPDNIKDTKSDIIFFQRNVPGHGRKMQFESSSYEGYFLACEK |
| | | ERDLFKLILKKEDELGDRSIMFTVQNED |
| 59 | 5-13 | YFGKLESKLSVIRNLNDQVLFIDQGNRPLFEDMTDSDCRDNAPRTIFIISKYSDSRARGLAVTIS |
| | | VKCEKISTLSCENKIISFKEMNPPDNIKDTKSDIIFFARSVPGHGRKTQFESSSYEGYFLACEKE |
| | | RDLFKLILKKEDELGDRSIMFTVQNED |
| 60 | 5-12 | YFGKLESKLSVIRNLNDQVLFIDQGNRPLFEDMTDSDCRDNAPRTIFIISKYSDSRARGLAVTIS |
| | | VKCEKISTLSCENKIISFKEMNPPDNIKDTKSDIIFFARDVPGHSGKRQFESSSYEGYFLACEKE |
| | | RDLFKLILKKEDELGDRSIMFTVQNED |
| 61 | 5-1 | YFGKLESKLSVIRNLNDQVLFIDQGNRPLFEDMTDSDCRDNAPRTIFIISKYTDSRPRGLAVTIS |
| | | VKCEKISTLSCENKIISFKEMNPPDNIKDTKSDIIFFERDVPGHSSKKQFESSSYEGYFLACEKE |
| | | RDLFKLILKKEDELGDRSIMFTVQNED |
| 62 | 5-33 | YFGKLESKLSVIRNLNDQVLFIDQGNRPLFEDMTDSDCRDNAPRTIFIISKYTDSRARGMAVTIS |
| | | VKCEKISTLSCENKIISFKEMNPPDNIKDTKSDIIFFERDVPGHNDKRQFESSSYEGYFLACEKE |
| | | RDLFKLILKKEDELGDRSIMFTVQNED |
| 63 | 5-21 | YFGKLESKLSVIRNLNDQVLFIDQGNRPLFEDMTDSDCRDNAPRTIFIISRYKDSGKRGLAVTIS |
| | | VKCEKISTLSCENKIISFKEMNPPDNIKDTKSDIIFFRRSVPGHSRKVQFESSSYEGYFLACEKE |
| | | RDLFKLILKKEDELGDRSIMFTVQNED |
| 64 | 6-31 | YFGKLESKLSVIRNLNDQVLFIDQGNRPLFEDMTDSDCRDNAPRTIFIISKYGDSGARGLAVTIS |
| | | VKCEKISTLSCENKIISFKEMNPPDNIKDTKSDIIFFERDVPGHSGKVQFESSSYEGYFLACEKE |
| | | RDLFKLILKKEDELGDRSIMFTVQNED |
| 65 | 6-20 | YFGKLESKLSVIRNLNDQVLFIDQGNRPLFEDMTDSDCRDNAPRTIFIISKYGDSRPRGMAVTI |
| | | SVKCEKISTLSCENKIISFKEMNPPDNIKDTKSDIIFFQRAVPGHNRKMQFESSSYEGYFLACEK |
| | | ERDLFKLILKKEDELGDRSIMFTVQNED |
| 66 | | XFGKXESXLSVIRNLNDQVLFIDQGNRPLFEDMTDSDXRDNAPRTIFIISXYXDXXXRXXAVTIS |
| | | VKXEKISTLSXXNKIISFKEMNPPDNIKDTKSDIIFFXRXVPGHXXKXQFESSSYEGYFLAXEKE |
| | | RDLFKLILKKEDELGDRSIMFTXQXED |
| 67 | hCS1 | TATTTCGGCAAACTGGAAAGCAAGCTCAGCGTGATCCGGAACCTGAACGACCAAGTGCT |
| | coding1 | GTTCATCGACCAGGGCAACCGGCCTCTGTTTGAGGACATGACAGACAGCGACTGCAGAG |
| | | ATAATGCCCCTCGGACCATCTTCATCATCTCTACCTACAAGGATTCTCAGCCTAGAGGCAA |
| | | GGCCGTGACCATCTCTGTCAAGTGCGAGAAGATCAGCACACTGAGCTGCGAGAACAAGA |
| | | TCATTAGCTTCAAAGAAATGAACCCCCCCGACAATATCAAGGACACCAAGTCCGACATCA |
| | | TCTTTTTCCAGAGAGATGTGCCAGGCCACAAGCACAAAATGCAGTTCGAGAGCAGCTCCT |
| | | ACGAGGGCTACTTCCTGGCTTGTGAAAAGGAAAGAGACCTGTTCAAGCTGATCCTGAAAA |
| | | AGGAAGATGAGCTGGGAGATAGAAGCATCATGTTCACCGTGCAGAACGAGGAC |
| 68 | hCS1 | TACTTCGGCAAACTGGAGTCCAAGCTGAGCGTGATCAGGAATCTGAACGATCAGGTGCTC |
| | coding2 | TTCATCGATCAGGGCAATAGACCTCTGTTTGAGGACATGACTGACTCCGACTGTCGGGAC |
| | | AACGCCCCCCGAACAATCTTTATTATCAGCACCTACAAGGACAGCCAGCCCAGGGGGAA |
| | | GGCCGTGACTATCTCCGTGAAGTGCGAAAAGATCAGCACCCTGTCATGTGAGAACAAGAT |
| | | TATCTCCTTTAAGGAGATGAACCCCCCCGATAACATTAAGGACACCAAGAGCGACATCAT |
| | | CTTCTTCCAGCGCGACGTGCCAGGACACAAGCACAAAATGCAGTTTGAGAGCAGCAGCT |
| | | ACGAAGGGTATTTCCTGGCCTGTGAGAAGGAAAGAGATCTGTTCAAGCTGATCCTGAAGA |
| | | AGGAGGACGAGCTGGGCGACCGGAGCATCATGTTTACCGTGCAGAACGAGGACTGA |
| 69 | hCS2 | TACTTCGGCAAGCTGGAAAGCAAGCTGAGCGTGATCCGGAACCTGAACGACCAAGTGCT |
| | coding1 | GTTCATCGACCAGGGAAATAGACCTCTGTTCGAGGACATGACCGACAGCGACTGCAGAG |
| | | ATAATGCCCCTAGAACAATCTTCATCATCAGCACATACAAAGATAAGCAGCCTCGGGCTAA |
| | | GGCCGTGACCATCAGCGTCAAGTGCGAGAAAATCTCTACCCTGTCTTGTGAAAACAAGAT |
| | | CATCTCCTTCAAGGAAATGAACCCCCCCGACAACATCAAGGACACCAAGTCCGACATCAT |
| | | TTTCTTCCAGCGGGACGTGCCAGGCCACAAGCACAAAATGCAGTTCGAGAGCAGCTCTTA |
| | | TGAGGGCTACTTTCTGGCCTGCGAGAAGGAAAGAGATCTGTTTAAGCTCATCCTGAAGAA |
| | | AGAAGATGAGCTGGGCGACAGAAGCATCATGTTCACCATCCAGAACGAGGAT |
| 70 | hCS2 | TACTTTGGCAAGCTGGAGTCCAAACTGTCCGTGATCCGCAACCTGAATGATCAGGTGCTG |
| | coding2 | TTCATCGACCAGGGCAACCGCCCCCTGTTCGAGGACATGACCGATAGCGACTGCAGGGA |
| | | TAACGCCCCTCGGACTATCTTTATCATCAGCACATACAAGGACAAACAGCCCAGAGCCAA |
| | | GGCCGTGACCATCAGCGTCAAGTGTGAGAAGATTAGCACTCTGTCCTGTGAGAACAAGAT |
| | | CATTAGCTTTAAGGAAATGAACCCACCTGATAATATCAAGGACACCAAGAGCGACATTATC |
| | | TTCTTTCAGAGAGATGTGCCCGGCCACAAGCACAAGATGCAGTTCGAGAGCTCTAGCTAC |
| | | GAGGGCTACTTCCTGGCTTGCGAAAAGGAGAGAGACCTGTTTAAACTCATTCTGAAGAAG |
| | | GAGGACGAACTGGGCGATAGATCCATCATGTTCACCATCCAGAACGAGGATTGA |
| 71 | hCS3 | CGGTTCGGCAAACTGGAATCTAAGCTGTCTGTGATCCGGAACCTGAACGACCAAGTGCT |
| | coding1 | GTTCATCGACCAGGGCAATCGCCCCCTGTTTGAGGATATGACCGACAGCGACTGCAGAG |
| | | ATAACGCCCCTAGAACAATCTTCATCATCAGCACCTACAAGGATAGCCAGCCTCGGGGCA |
| | | AGGCCGTGACAATCAGCGTCAAGTGCGAAAAAATCTCCACCCTGAGCTGTGAAAACAAGA |
| | | TCATCTCTTTTAAGGAAATGAACCCACCTGATAATATCAAGGACACCAAGTCCGACATCAT |
| | | TTTCTTCCAGAGAGATGTGCCCGGACACAAGCACAAAATGCAGTTCGAGAGCAGCAGCTA |
| | | CGAGGGCTACTTCCTGGCTTGCGAGAAGGAAAGAGACCTGTTCAAGCTCATCCTGAAGA |
| | | AAGAGGACGAGCTGGGCGACAGAAGCATCATGTTCACCGTGCAGAACGAGGAC |
| 72 | hCS3 | CGCTTCGGCAAACTGGAGTCCAAGCTGAGCGTGATCAGGAATCTGAACGATCAGGTGCT |
| | coding2 | CTTCATCGATCAGGGCAATAGACCTCTGTTTGAGGACATGACTGACTCCGACTGTCGGGA |
| | | CAACGCCCCCCGAACAATCTTTATTATCAGCACCTACAAGGACAGCCAGCCCAGGGGGA |
| | | AGGCCGTGACTATCTCCGTGAAGTGCGAAAAGATCAGCACCCTGTCATGTGAGAACAAGA |
| | | TTATCTCCTTTAAGGAGATGAACCCCCCCGATAACATTAAGGACACCAAGAGCGACATCA |
| | | TCTTCTTCCAGCGCGACGTGCCAGGACACAAGCACAAAATGCAGTTTGAGAGCAGCAGC |
| | | TACGAAGGGTATTTCCTGGCCTGTGAGAAGGAAAGAGATCTGTTCAAGCTGATCCTGAAG |
| | | AAGGAGGACGAGCTGGGCGACCGGAGCATCATGTTTACCGTGCAGAACGAGGACTGA |
| 73 | hCS4 | AGGTTCGGCAAACTGGAAAGCAAGCTCTCCGTGATCCGGAACCTGAACGACCAGGTGCT |
| | coding1 | GTTTATCGACCAGGGAAATAGACCTCTGTTCGAGGACATGACAGATAGCGACTGCAGAGA |
| | | TAATGCCCCTCGGACCATCTTCATCATCTCTACCTACCGGGACAGCCAGCCTAGAGGCAA |
| | | GGCCGTGACCATCTCTGTCAAGTGCGAGAAGATCAGCACCCTGAGCTGCGAAAACAAGA |
| | | TCATCAGCTTCAAGGAAATGAACCCCCCAGACAACATCAAGGACACAAAGTCCGACATCA |
| | | TTTTCTTCCAGAGAAACGTGCCCGGCCACAAGTACAAGATGCAATTTGAGTCTAGCAGCT |
| | | ACGAGGGCTATTTCCTGGCTTGTGAAAAGGAAAGAGATCTGTTCAAACTGATCCTGAAGA |
| | | AAGAGGACGAGCTGGGCGATAGAAGCATCATGTTCACCGTGCAGAACGAGGAT |
| 74 | hCS4 | CGCTTCGGCAAACTGGAGTCCAAGCTGAGCGTGATCAGGAATCTGAACGATCAGGTGCT |
| | coding2 | CTTCATCGATCAGGGCAATAGACCTCTGTTTGAGGACATGACTGACTCCGACTGTCGGGA |
| | | CAACGCCCCCCGAACAATCTTTATTATCAGCACCTACCGGGACAGCCAGCCCAGGGGGA |
| | | AGGCCGTGACTATCTCCGTGAAGTGCGAAAAGATCAGCACCCTGTCATGTGAGAACAAGA |
| | | TTATCTCCTTTAAGGAGATGAACCCCCCCGATAACATTAAGGACACCAAGAGCGACATCA |
| | | TCTTCTTCCAGCGCAACGTGCCAGGACACAAGTACAAAATGCAGTTTGAGAGCAGCAGCT |
| | | ACGAAGGGTATTTCCTGGCCTGTGAGAAGGAAAGAGATCTGTTCAAGCTGATCCTGAAGA |
| | | AGGAGGACGAGCTGGGCGACCGGAGCATCATGTTTACCGTGCAGAACGAGGACTGA |
| 75 | 6-12 | TATTTCGGCAAGCTGGAAAGCAAGCTCTCTGTGATCCGGAACCTGAACGACCAAGTGCTG |
| | coding1 | TTCATCGACCAGGGCAACCGGCCTCTGTTCGAGGATATGACCGACAGCGACTGCAGAGA |
| | | TAATGCCCCTCGCACAATCTTCATCATTTCTAAATACAGCGATAGCCTGGCCAGAGGCCT |
| | | GGCTGTGACCATCAGCGTCAAGTGCGAGAAAATCTCCACCCTGAGCTGCGAGAACAAGA |
| | | TCATCTCTTTCAAGGAAATGAACCCCCCTGACAATATCAAGGACACAAAGTCCGACATCAT |
| | | CTTTTTCCAGAGAGATGTGCCAGGACACAGCAGAAAGATGCAGTTCGAGAGCAGCTCCTA |
| | | CGAGGGCTACTTCCTGGCCTGTGAAAAGGAACGGGACCTGTTTAAGCTGATCCTGAAGA |
| | | AAGAAGATGAGCTGGGCGACAGAAGCATCATGTTCACCGTGCAGAACGAGGAC |
| 76 | 6-12 | TATTTCGGCAAACTGGAGAGCAAGCTGAGCGTGATTCGGAACCTGAATGACCAGGTGCT |
| | coding2 | GTTCATTGACCAGGGCAATCGCCCTCTGTTCGAAGACATGACCGACTCCGATTGTAGAGA |
| | | CAACGCCCCTCGGACCATCTTCATTATCAGCAAGTACAGCGACAGCCTGGCCCGCGGCC |
| | | TGGCTGTGACAATCAGCGTGAAGTGCGAGAAGATCAGCACACTGTCCTGCGAGAACAAA |
| | | ATCATCAGCTTTAAAGAGATGAATCCCCCTGACAATATCAAAGATACTAAATCTGACATCA |
| | | TCTTCTTCCAGCGGGACGTGCCTGGCCACTCTAGAAAAATGCAGTTCGAGTCCTCCAGTT |
| | | ACGAGGGGTACTTCCTGGCCTGCGAGAAGGAGCGGGACCTGTTCAAACTGATCCTGAAA |
| | | AAAGAAGACGAGCTGGGCGATCGGAGCATCATGTTCACTGTGCAGAACGAGGATTGA |
| 77 | sq89v6 | TACTTCGGCAAGCTGGAAAGCAAGCTCAGCGTCATCCGGAACCTGAACGACCAGGTGCT |
| | coding1 | GTTCATCGACCAAGGCAACCGGCCTCTGTTTGAGGACATGACCGACTCTGATTCTAGAGA |
| | | TAATGCCCCTAGAACCATCTTCATCATCTCCAAGTACAGCGATAGCCTGGCCAGAGGCCT |
| | | GGCTGTGACAATCTCTGTGAAGAGCGAGAAAATCAGCACCCTGTCTTGTGAAAACAAGAT |
| | | CATCAGCTTCAAGGAAATGAACCCTCCAGACAATATCAAGGACACAAAGTCCGACATCAT |
| | | TTTCTTCCAGAGAGATGTGCCCGGCCACAGCAGGAAGATGCAGTTCGAGTCCAGCAGCT |
| | | ACGAAGGATATTTTCTGGCCTGCGAGAAGGAACGGGACCTGTTCAAACTGATCCTGAAGA |
| | | AAGAGGACGAGCTGGGCGACAGAAGCATCATGTTCACCGTGCAGAACGAGGAT |
| 78 | sq89v6 | TATTTTGGGAAGCTGGAGAGCAAACTGAGCGTGATCCGGAATCTGAATGACCAGGTGCT |
| | coding2 | GTTCATTGACCAGGGCAATCGACCACTGTTCGAAGATATGACCGATAGCGACAGCAGGG |
| | | ACAATGCCCCCAGGACCATCTTTATCATCAGCAAGTACAGCGATAGCCTGGCCAGGGGA |
| | | CTGGCCGTGACAATCAGCGTGAAGTCCGAGAAGATCAGCACCCTGTCCTGCGAGAACAA |
| | | GATCATTTCTTTCAAGGAGATGAACCCCCCTGACAACATCAAGGACACAAAGAGCGACAT |
| | | CATCTTCTTTCAGAGAGACGTGCCCGGACACAGCCGCAAGATGCAGTTTGAATCTTCCAG |
| | | CTACGAAGGGTACTTCCTGGCCTGTGAAAAAGAGCGCGATCTGTTCAAACTGATCCTCAA |
| | | GAAGGAGGACGAGCTGGGCGATCGCTCTATCATGTTCACAGTGCAGAACGAGGACTGA |
| 79 | C68v1 | TATTTCGGCAAGCTGGAAAGCAAGCTCTCTGTGATCCGGAACCTGAACGACCAAGTGCTG |
| | coding1 | TTTATCGACCAGGGAAATAGACCTCTGTTCGAGGACATGACCGACAGCGACAGCCGGGA |
| | | TAACGCCCCTCGCACCATCTTCATCATCTCCAAGTACAGCGATAGCCTGGCTAGAGGCCT |
| | | GGCCGTGACCATCAGCGTCAAGGGCGAGAAGATCTCTACACTGAGCTGTGAAAACAAGA |
| | | TCATCAGCTTCAAGGAAATGAACCCCCCCGACAATATCAAGGACACAAAGTCCGACATCA |
| | | TTTTCTTCCAGAGAGATGTGCCTGGCCACAGCAGAAAGATGCAGTTCGAGTCCAGCAGCT |
| | | ACGAGGGCTACTTTCTGGCCTGCGAGAAAGAACGGGACCTGTTCAAACTGATCCTGAAAA |
| | | AGGAAGACGAGCTGGGCGATAGATCTATCATGTTCACCGTGCAGAACGAGGAT |
| 80 | C68v1 | TATTTTGGGAAGCTGGAGAGCAAACTGAGCGTGATCCGGAATCTGAATGACCAGGTGCT |
| | coding2 | GTTCATTGACCAGGGCAATCGACCACTGTTCGAAGATATGACCGATAGCGACAGCAGGG |
| | | ACAATGCCCCCAGGACCATCTTTATCATCAGCAAGTACAGCGATAGCCTGGCCAGGGGA |
| | | CTGGCCGTGACAATCAGCGTGAAGGGCGAGAAGATCAGCACCCTGTCCTGCGAGAACAA |
| | | GATCATTTCTTTCAAGGAGATGAACCCCCCTGACAACATCAAGGACACAAAGAGCGACAT |
| | | CATCTTCTTTCAGAGAGACGTGCCCGGACACAGCCGCAAGATGCAGTTTGAATCTTCCAG |
| | | CTACGAAGGGTACTTCCTGGCCTGTGAAAAAGAGCGCGATCTGTTCAAACTGATCCTCAA |
| | | GAAGGAGGACGAGCTGGGCGATCGCTCTATCATGTTCACAGTGCAGAACGAGGACTGA |
| 81 | C68v2 | TATTTCGGCAAGCTGGAAAGCAAGCTGAGCGTCATCCGGAACCTCAACGACCAAGTGCT |
| | coding1 | GTTTATCGACCAGGGCAACCGGCCTCTGTTCGAGGACATGACAGACAGCGACAGCAGAG |
| | | ATAATGCCCCTAGAACCATCTTCATCATTTCCAAGTACAGCGATTCTCTGGCCAGAGGCCT |
| | | GGCCGTGACCATCTCTGTGAAGGCCGAGAAAATCTCCACCCTGAGCTGCGAAAACAAGA |
| | | TCATCAGCTTCAAGGAGATGAACCCTCCAGACAATATCAAGGACACCAAGAGCGACATCA |
| | | TCTTTTTCCAGCGGGACGTGCCCGGCCACTCTCGCAAGATGCAGTTCGAGTCCAGCAGC |
| | | TACGAGGGCTACTTCCTGGCTTGTGAAAAGGAAAGAGATCTGTTCAAACTGATCCTGAAA |
| | | AAGGAAGATGAGCTGGGAGATAGAAGCATCATGTTCACAGTGCAGAACGAGGAC |
| 82 | C68v2 | TATTTTGGGAAGCTGGAGAGCAAACTGAGCGTGATCCGGAATCTGAATGACCAGGTGCT |
| | coding2 | GTTCATTGACCAGGGCAATCGACCACTGTTCGAAGATATGACCGATAGCGACAGCAGGG |
| | | ACAATGCCCCCAGGACCATCTTTATCATCAGCAAGTACAGCGATAGCCTGGCCAGGGGA |
| | | CTGGCCGTGACAATCAGCGTGAAGGCCGAGAAGATCAGCACCCTGTCCTGCGAGAACAA |
| | | GATCATTTCTTTCAAGGAGATGAACCCCCCTGACAACATCAAGGACACAAAGAGCGACAT |
| | | CATCTTCTTTCAGAGAGACGTGCCCGGACACAGCCGCAAGATGCAGTTTGAATCTTCCAG |
| | | CTACGAAGGGTACTTCCTGGCCTGTGAAAAAGAGCGCGATCTGTTCAAACTGATCCTCAA |
| | | GAAGGAGGACGAGCTGGGCGATCGCTCTATCATGTTCACAGTGCAGAACGAGGACTGA |
| 83 | C68v4 | TATTTCGGCAAGCTGGAATCTAAACTCTCCGTGATCCGGAACCTGAACGACCAAGTGCTG |
| | coding1 | TTCATCGACCAGGGAAATAGACCTCTGTTCGAGGATATGACAGACAGCGATAGCCGCGAT |
| | | AATGCCCCTAGAACCATCTTTATCATTAGCAAGTACAGCGACAGCCTGGCCAGAGGCCTG |
| | | GCCGTGACAATCAGCGTCAAGGACGAGAAAATCTCCACCCTGAGCTGCGAGAACAAGAT |
| | | CATCAGCTTCAAGGAAATGAACCCTCCAGACAACATCAAGGACACCAAGTCCGATATCAT |
| | | CTTCTTCCAGCGGGACGTGCCCGGCCACTCTAGAAAGATGCAGTTCGAGAGCTCTAGCT |
| | | ACGAGGGCTACTTCCTGGCTTGTGAAAAAGAACGGGACCTGTTTAAGCTGATCCTGAAGA |
| | | AGGAAGATGAGCTGGGCGACAGAAGCATCATGTTCACCGTGCAGAACGAGGAC |
| 84 | C68v4 | TATTTTGGGAAGCTGGAGAGCAAACTGAGCGTGATCCGGAATCTGAATGACCAGGTGCT |
| | coding2 | GTTCATTGACCAGGGCAATCGACCACTGTTCGAAGATATGACCGATAGCGACAGCAGGG |
| | | ACAATGCCCCCAGGACCATCTTTATCATCAGCAAGTACAGCGATAGCCTGGCCAGGGGA |
| | | CTGGCCGTGACAATCAGCGTGAAGGACGAGAAGATCAGCACCCTGTCCTGCGAGAACAA |
| | | GATCATTTCTTTCAAGGAGATGAACCCCCCTGACAACATCAAGGACACAAAGAGCGACAT |
| | | CATCTTCTTTCAGAGAGACGTGCCCGGACACAGCCGCAAGATGCAGTTTGAATCTTCCAG |
| | | CTACGAAGGGTACTTCCTGGCCTGTGAAAAAGAGCGCGATCTGTTCAAACTGATCCTCAA |
| | | GAAGGAGGACGAGCTGGGCGATCGCTCTATCATGTTCACAGTGCAGAACGAGGACTGA |
| 85 | wt hIL-18 | TATTTCGGCAAGCTGGAAAGCAAGCTCTCCGTGATCAGAAACCTGAACGACCAAGTGCTG |
| | coding1 | TTCATCGACCAGGGCAACAGACCTCTGTTTGAGGATATGACCGACTCTGATTGCAGAGAT |
| | | AATGCCCCTAGAACCATCTTTATCATCAGCATGTACAAGGACAGCCAGCCTCGGGGAATG |
| | | GCCGTGACCATCAGCGTCAAGTGCGAGAAGATCTCTACCCTGAGCTGCGAAAACAAAATC |
| | | ATCTCCTTCAAGGAAATGAACCCCCCAGACAATATCAAGGATACAAAGAGCGACATCATTT |
| | | TCTTCCAGCGGAGCGTGCCCGGCCACGACAACAAGATGCAGTTCGAGAGCTCTAGCTAC |
| | | GAGGGCTACTTCCTGGCTTGTGAAAAGGAACGGGACCTGTTCAAACTGATCCTGAAGAAA |
| | | GAGGACGAGCTGGGCGATAGAAGCATCATGTTCACAGTGCAGAACGAGGAC |
| 86 | wt hIL-18 | TACTTCGGCAAACTGGAGTCCAAGCTGAGCGTGATCAGGAATCTGAACGATCAGGTGCTC |
| | coding2 | TTCATCGATCAGGGCAATAGACCTCTGTTTGAGGACATGACTGACTCCGACTGTCGGGAC |
| | | AACGCCCCCCGAACAATCTTTATTATCAGCATGTACAAGGACAGCCAGCCCAGGGGGAT |
| | | GGCCGTGACTATCTCCGTGAAGTGCGAAAAGATCAGCACCCTGTCATGTGAGAACAAGAT |
| | | TATCTCCTTTAAGGAGATGAACCCCCCCGATAACATTAAGGACACCAAGAGCGACATCAT |
| | | CTTCTTCCAGCGCTCCGTGCCAGGACACGATAACAAAATGCAGTTTGAGAGCAGCAGCTA |
| | | CGAAGGGTATTTCCTGGCCTGTGAGAAGGAAAGAGATCTGTTCAAGCTGATCCTGAAGAA |
| | | GGAGGACGAGCTGGGCGACCGGAGCATCATGTTTACCGTGCAGAACGAGGACTGA |
| 87 | EF1a | CGTGAGGCTCCGGTGCCCGTCAGTGGGCAGAGCGCACATCGCCCACAGTCCCCGAGAA |
| | promoter | GTTGGGGGGAGGGGTCGGCAATTGAACCGGTGCCTAGAGAAGGTGGCGCGGGGTAAAC |
| | | TGGGAAAGTGATGTCGTGTACTGGCTCCGCCTTTTTCCCGAGGGTGGGGGAGAACCGTA |
| | | TATAAGTGCAGTAGTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTTATGGCCCTTGCG |
| | | TGCCTTGAATTACTTCCACCTGGCTGCAGTACGTGATTCTTGATCCCGAGCTTCGGGTTG |
| | | GAAGTGGGTGGGAGAGTTCGAGGCCTTGCGCTTAAGGAGCCCCTTCGCCTCGTGCTTGA |
| | | GTTGAGGCCTGGCCTGGGCGCTGGGGCCGCCGCGTGCGAATCTGGTGGCACCTTCGCG |
| | | CCTGTCTCGCTGCTTTCGATAAGTCTCTAGCCATTTAAAATTTTTGATGACCTGCTGCGAC |
| | | GCTTTTTTTCTGGCAAGATAGTCTTGTAAATGCGGGCCAAGATCTGCACACTGGTATTTCG |
| | | GTTTTTGGGGCCGCGGGCGGCGACGGGGCCCGTGCGTCCCAGCGCACATGTTCGGCGA |
| | | GGCGGGGCCTGCGAGCGCGGCCACCGAGAATCGGACGGGGGTAGTCTCAAGCTGGCC |
| | | GGCCTGCTCTGGTGCCTGGCCTCGCGCCGCCGTGTATCGCCCCGCCCTGGGCGGCAAG |
| | | GCTGGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCACCC |
| | | ACACAAAGGAAAAGGGCCTTTCCGTCCTCAGCCGTCGCTTCATGTGACTCCACGGAGTAC |
| | | CGGGCGCCGTCCAGGCACCTCGATTAGTTCTCGAGCTTTTGGAGTACGTCGTCTTTAGGT |
| | | TGGGGGGAGGGGTTTTATGCGATGGAGTTTCCCCACACTGAGTGGGTGGAGACTGAAGT |
| | | TAGGCCAGCTTGGCACTTGATGTAATTCTCCTTGGAATTTGCCCTTTTTGAGTTTGGATCT |
| | | TGGTTCATTCTCAAGCCTCAGACAGTGGTTCAAAGTTTTTTTCTTCCATTTCAGGTGTCGT |
| | | GA |
| 88 | wtIL12p35 | MCPARSLLLVATLVLLDHLSLARNLPVATPDPGMFPCLHHSQNLLRAVSNMLQKARQTLEFYP |
| | | CTSEEIDHEDITKDKTSTVEACLPLELTKNESCLNSRETSFITNGSCLASRKTSFMMALCLSSIY |
| | | EDLKMYQVEFKTMNAKLLMDPKRQIFLDQNMLAVIDELMQALNFNSETVPQKSSLEEPDFYKT |
| | | KIKLCILLHAFRIRAVTIDRVMSYLNAS |
| 89 | wtIL12p35_ | RNLPVATPDPGMFPCLHHSQNLLRAVSNMLQKARQTLEFYPCTSEEIDHEDITKDKTSTVEAC |
| | noSP | LPLELTKNESCLNSRETSFITNGSCLASRKTSFMMALCLSSIYEDLKMYQVEFKTMNAKLLMD |
| | | PKRQIFLDQNMLAVIDELMQALNFNSETVPQKSSLEEPDFYKTKIKLCILLHAFRIRAVTIDRVM |
| | | SYLNAS |
| 90 | wtIL12p40 | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 91 | wtIL12p40_ | IWELKKDVYVVELDWYPDAPGEMVVLTCDTPEEDGITWTLDQSSEVLGSGKTLTIQVKEFGD |
| | noSP | AGQYTCHKGGEVLSHSLLLLHKKEDGIWSTDILKDQKEPKNKTFLRCEAKNYSGRFTCWWLT |
| | | TISTDLTFSVKSSRGSSDPQGVTCGAATLSAERVRGDNKEYEYSVECQEDSACPAAEESLPIE |
| | | VMVDAVHKLKYENYTSSFFIRDIIKPDPPKNLQLKPLKNSRQVEVSWEYPDTWSTPHSYFSLT |
| | | FCVQVQGKSKREKKDRVFTDKTSATVICRKNASISVRAQDRYYSSSWSEWASVPCS |
| 92 | H216A | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVAKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 93 | K217A | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHALKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 94 | K219A | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLAYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 95 | HK217 | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVAALKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 96 | HK219 | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVAKLAYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 97 | KK | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHALAYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 98 | HKK | MCHQQLVISWFSLVFLASPLVAIWELKKDVYVVELDWYPDAPGEMVVLTCDTPEEDGITWTL |
| | | DQSSEVLGSGKTLTIQVKEFGDAGQYTCHKGGEVLSHSLLLLHKKEDGIWSTDILKDQKEPKN |
| | | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVAALAYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 99 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKAFGDAGQYTCHKGGEVLSHSLLLLHKKEDGIWSTDILKDQKEPKN |
| | 3 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 100 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKEAGDAGQYTCHKGGEVLSHSLLLLHKKEDGIWSTDILKDQKEPKN |
| | 4 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 101 | WO202121 | MCHQQLVISWFSLVFLASPLVAIWELKKDVYVVELDWYAAAPGEMVVLTCDTPEEDGITWTLD |
| | 2083_SEQ: | QSSEVLGSGKTLTIQVKAAGDAGQYTCHKGGEVLSHSLLLLHKKEDGIWSTDILKDQKEPKNK |
| | 5 | TFLRCEAKNYSGRFTCWWLTTISTDLTFSVKSSRGSSDPQGVTCGAATLSAERVRGDNKEYE |
| | | YSVECQEDSACPAAEESLPIEVMVDAVHKLKYENYTSSFFIRDIIKPDPPKNLQLKPLKNSRQV |
| | | EVSWEYPDTWSTPHSYFSLTFCVQVQGKSKREKKDRVFTDKTSATVICRKNASISVRAQDRY |
| | | YSSSWSEWASVPCS |
| 102 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKEFGDAGQYTCHKGGEVLSHSLLLLHAKEDGIWSTDILKDQKEPKN |
| | 6 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 103 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKEFGDAGQYTCHKGGEVLSHSLLLLHKKEDGIWSTDILKDQKEPKN |
| | 7 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHALKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 104 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKEFGDAGQYTCHKGGEVLSHSLLLLHKKEDGIWSTDILKDQKEPKN |
| | 8 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLAYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 105 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKAAGDAGQYTCHKGGEVLSHSLLLLHKKEDGIWSTDILKDQKEPKN |
| | 13 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 106 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKAAGDAGQYTCHKGGEVLSHSLLLLHAKEDGIWSTDILKDQKEPKN |
| | 14 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 107 | WO202121 | MCHQQLVISWFSLVFLASPLVAIWELKKDVYVVELDAYPDAPGEMVVLTCDTPEEDGITWTLD |
| | 2083_SEQ: | QSSEVLGSGKTLTIQVKAAGDAGQYTCHKGGEVLSHSLLLLHKKEDGIWSTDILKDQKEPKNK |
| | 15 | TFLRCEAKNYSGRFTCWWLTTISTDLTFSVKSSRGSSDPQGVTCGAATLSAERVRGDNKEYE |
| | | YSVECQEDSACPAAEESLPIEVMVDAVHKLKYENYTSSFFIRDIIKPDPPKNLQLKPLKNSRQV |
| | | EVSWEYPDTWSTPHSYFSLTFCVQVQGKSKREKKDRVFTDKTSATVICRKNASISVRAQDRY |
| | | YSSSWSEWASVPCS |
| 108 | WO202121 | MCHQQLVISWFSLVFLASPLVAIWELKKDVYVVELDWYPDAPGEMVVLTCDTPEEDGITWTL |
| | 2083_SEQ: | DQSSEVLGSGKTLTIQVKAAGDAGQYTCHKGGEVLSHSLLLLHAKADGIWSTAILKDQKEPKN |
| | 16 | KTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICRKNASISVRAQDR |
| | | YYSSSWSEWASVPCS |
| 109 | EGFRvIII | MALPVTALLLPLALLLHAARPDVVMTQSPDSLAVSLGERATINCKSSQSLLDSDGKTYLNWLQ |
| | CAR | QKPGQPPKRLISLVSKLDSGVPDRFSGSGSGTDFTLTISSLQAEDVAVYYCWQGTHFPGTFG |
| | | GGTKVEIKGGGGGGGGSGGGGSEIQLVQSGAEVKKPGESLRISCKGSGFNIEDYYIHWVR |
| | | QMPGKGLEWMGRIDPENDETKYGPIFQGHVTISADTSINTVYLQWSSLKASDTAMYYCAFRG |
| | | GVYWGQGTTVTVSSTTTPAPRPPTPAPTIASQPLSLRPEACRPAAGGAVHTRGLDFACDIYIW |
| | | APLAGTCGVLLLSLVITRSKRSRLLHSDYMNMTPRRPGPTRKHYQPYAPPRDFAAYRSRVKF |
| | | SRSADAPAYQQGQNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| 110 | EGFRvIII | DVVMTQSPDSLAVSLGERATINCKSSQSLLDSDGKTYLNWLQQKPGQPPKRLISLVSKLDSG |
| | CAR vL | VPDRFSGSGSGTDFTLTISSLQAEDVAVYYCWQGTHFPGTFGGGTKVEIK |
| 111 | EGFRvIII | EIQLVQSGAEVKKPGESLRISCKGSGFNIEDYYIHWVRQMPGKGLEWMGRIDPENDETKYGPI |
| | CAR vH | FQGHVTISADTSINTVYLQWSSLKASDTAMYYCAFRGGVYWGQGTTVTVSS |
| 112 | EGFRvIII CAR vL CDR1 | KSSQSLLDSDGKTYLN |
| 113 | EGFRvIII CAR vL CDR2 | LVSKLDS |
| 114 | EGFRvIII CAR vL CDR3 | WQGTHFPGT |
| 115 | EGFRVIII CAR vH CDR1 | DYYIH |
| 116 | EGFRvIII CAR vH CDR2 | RIDPENDETKYGPIFQG |
| 117 | EGFRvIII CAR vH CDR3 | RGGVY |
| 118 | Human OSM signal | MGVLLTQRTLLSLVLALLFPSMASM |
| | peptide | |
| 119 | VSV-G signal peptide | MKCLLYLAFLFIGVNC |
| 120 | Mouse Ig Kappa signal | METDTLLLWVLLLWVPGSTGD |
| | peptide | |
| 121 | Mouse Ig Heavy signal | MGWSCIILFLVATATGVHS |
| | peptide | |
| 122 | BM40 signal peptide | MRAWIFFLLCLAGRALA |
| 123 | Secrecon signal | MWWRLWWLLLLLLLLWPMVWA |
| | peptide | |
| 124 | Human IgKVIII signal | MDMRVPAQLLGLLLLWLRGARC |
| | peptide | |
| 125 | CD33 signal peptide | MPLLLLLPLLWAGALA |
| 126 | tPA signal peptide | MDAMKRGLCCVLLLCGAVFVSPS |
| 127 | Human Chymotrypsinogen | MAFLWLLSCWALLGTTFG |
| | signal peptide | |
| 128 | Human trypsinogen-2 | MNLLLILTFVAAAVA |
| | signal peptide | |
| 129 | Human IL-2 signal | MYRMQLLSCIALSLALVTNS |
| | peptide | |
| 130 | Gaussia luc signal | MGVKVLFALICIAVAEA |
| | peptide | |
| 131 | Albumin (HSA) signal | MKWVTFISLLFSSAYS |
| | peptide | |
| 132 | Influenza | MKTIIALSYIFCLVLG |
| | Haemagglutinin signal | |
| | peptide | |
| 133 | Human insulin signal | MALWMRLLPLLALLALWGPDPAAA |
| | peptide | |
| 134 | Silkworm Fibroin LC | MKPIFLVLLVVTSAYA |
| | signal peptide | |
| 135 | Interleukin-18-binding | MRHNWTPDLSPLWVLLLCAHVVTLLVRA |
| | protein signal peptide | |
| 136 | Interleukin-12 subunit | MCHQQLVISWFSLVFLASPLVA |
| | beta signal peptide | |
| 137 | Interleukin-12 subunit | MCPARSLLLVATLVLLDHLSLA |
| | alpha signal peptide | |
| 138 | Interleukin-18 signal | MAAEPVEDNCINFVAMKFIDNTLYFIAEDDENLESD |
| | peptide | |
| 139 | CD8 signal peptide | MALPVTALLLPLALLLHAARP |
| 140 | WO2023023503A1 #3 | MEFGLSWVFLVALFRGVQS |
| | signal peptide | |
| 141 | hIL2 variant signal | MGYRMQLLSCIALSLALVTNSG |
| | peptide | |
| 142 | EMCV | acgttactggccgaagccgcttggaataaggccggtgtgcgtttgtctatatgttattttccacc |
| | IRES | atattgccgtcttttggcaatgtgagggcccggaaacctggccctgtcttcttgacgagcattcc |
| | | taggggtctttcccctctcgccaaaggaatgcaaggtctgttgaatgtcgtgaaggaagcagttc |
| | | ctctggaagcttcttgaagacaaacaacgtctgtagcgaccctttgcaggcagcggaacccccca |
| | | cctggcgacaggtgcctctgcggccaaaagccacgtgtataagatacacctgcaaaggcggcaca |
| | | accccagtgccacgttgtgagttggatagttgtggaaagagtcaaatggctctcctcaagcgtat |
| | | tcaacaaggggctgaaggatgcccagaaggtaccccattgtatgggatctgatctggggcctcgg |
| | | tgcacatgctttacatgtgtttagtcgaggttaaaaaaacgtctaggccccccgaaccacgggga |
| | | cgtggttttcctttgaaaaacacgatgataata |
| 143 | T2A | (GSG)EGRGSLLTCGDVEENPGP |
| 144 | P2A | (GSG)ATNFSLLKQAGDVEENPGP |
| 145 | E2A | (GSG)QCTNYALLKLAGDVESNPGP |
| 146 | F2A | (GSG)VKQTLNFDLLKLAGDVESNPGP |

| | | SEQ ID | | SEQ ID |
|---|---|---|---|---|
| Antibody | Heavy Chain sequence | NO: | Light chain sequence | NO: |
| pivekimab | QVQLVQSGAEVKKPGASVKVSCKASGYIFTS | 147 | EIQMTQSPSSLSASVGDRVTITCRASQ | 148 |
| | SIMHWVRQAPGQGLEWIGYIKPYNDGTKYNE | | DINSYLSWFQQKPGKAPKTLIYRVNRL | |
| | KFKGRATLTSDRSTSTAYMELSSLRSEDTAV | | VDGVPSRFSGSGSGNDYTLTISSLQP | |
| | YYCAREGGNDYYDTMDYWGQGTLVTVSS | | EDFATYYCLQYDAFPYTFGQGTKVEIK | |
| talacotuzumab | EVQLVQSGAEVKKPGESLKISCKGSGYSFTD | 149 | DIVMTQSPDSLAVSLGERATINCESSQ | 150 |
| | YYMKWARQMPGKGLEWMGDIIPSNGATFYN | | SLLNSGNQKNYLTWYQQKPGQPPKPL | |
| | QKFKGQVTISADKSISTTYLQWSSLKASDTAM | | IYWASTRESGVPDRFSGSGSGTDFTL | |
| | YYCARSHLLRASWFAYWGQGTMVTVSS | | TISSLQAEDVAVYYCQNDYSYPYTFG | |
| | | | QGTKLEIK | |
| vibecotamab | QVQLQQSGAEVKKPGASVKVSCKASGYTFT | 151 | DFVMTQSPDSLAVSLGERATINCKSS | 152 |
| | DYYMKWVKQSHGKSLEWMGDIIPSNGATFY | | QSLLNTGNQKNYLTWYQQKPGQPPK | |
| | NQKFKGKATLTVDRSTSTAYMELSSLRSEDT | | LLIYWASTRESGVPDRFTGSGSGTDF | |
| | AVYYCARSHLLRASWFAYWGQGTLVTVSS | | TLTISSLQAEDVAVYYCQNDYSYPYTF | |
| | | | GGGTKLEIK | |
| budoprutug | QVQLEQPGAEVVKPGASVKVSCKTSGYTFTS | 153 | QIVLTQSPATLSASPGEKATMTCSASS | 154 |
| | NWMHWVKQTPGKGLEWIGEIDPSDSYTNYN | | GVNYMHWYQQKPGTSPKRWIYDTDK | |
| | QKFDGKAKLTVDKSSSTAYMEVSDLTAEDSA | | TASGVPARFSGSGSGTSYSLTISSMEA | |
| | TYYCARGSNPYYYAMDYWGQGTSVTVSS | | EDAATYYCHQRGSYTFGGGTKLEIK | |
| coltuximab | QVQLVQPGAEVVKPGASVKLSCKTSGYTFTS | 155 | EIVLTQSPAIMSASPGERVTMTCSASS | 156 |
| | NWMHWVKQAPGQGLEWIGEIDPSDSYTNYN | | GVNYMHWYQQKPGTSPRRWIYDTSK | |
| | QNFQGKAKLTVDKSTSTAYMEVSSLRSDDTA | | LASGVPARFSGSGSGTDYSLTISSMEP | |
| | VYYCARGSNPYYYAMDYWGQGTSVTVSS | | EDAATYYCHQRGSYTFGGGTKLEIK | |
| denintuzumab | QVQLQESGPGLVKPSQTLSLTCTVSGGSIST | 157 | EIVLTQSPATLSLSPGERATLSCSASS | 158 |
| | SGMGVGWIRQHPGKGLEWIGHIWWDDDKRY | | SVSYMHWYQQKPGQAPRLLIYDTSKL | |
| | NPALKSRVTISVDTSKNQFSLKLSSVTAADTA | | ASGIPARFSGSGSGTDFTLTISSLEPE | |
| | VYYCARMELWSYYFDYWGQGTLVTVSS | | DVAVYYCFQGSVYPFTFGQGTKLEIK | |
| inebilizumab | EVQLVESGGGLVQPGGSLRLSCAASGFTFSS | 159 | EIVLTQSPDFQSVTPKEKVTITCRASES | 160 |
| | SWMNWVRQAPGKGLEWVGRIYPGDGDTNY | | VDTFGISFMNWFQQKPDQSPKLLIHEA | |
| | NVKFKGRFTISRDDSKNSLYLQMNSLKTEDTA | | SNQGSGVPSRFSGSGSGTDFTLTINS | |
| | VYYCARSGFITTVRDFDYWGQGTLVTVSS | | LEAEDAATYYCQQSKEVPFTFGGGTK | |
| | | | VEIK | |
| leronlimab | EVQLVESGGGLVKPGGSLRLSCAASGYTFSN | 161 | DIVMTQSPLSLPVTPGEPASISCRSSQ | 162 |
| | YWIGWVRQAPGKGLEWIGDIYPGGNYIRNNE | | RLLSSYGHTYLHWYLQKPGQSPQLLIY | |
| | KFKDKTTLSADTSKNTAYLQMNSLKTEDTAVY | | EVSNRFSGVPDRFSGSGSGTDFTLKIS | |
| | YCGSSFGSNYVFAWFTYWGQGTLVTVSS | | RVEAEDVGVYYCSQSTHVPLTFGQGT | |
| | | | KVEIK | |
| loncastuximab | QVQLVQPGAEVVKPGASVKLSCKTSGYTFTS | 163 | EIVLTQSPAIMSASPGERVTMTCSASS | 164 |
| | NWMHWVKQAPGQGLEWIGEIDPSDSYTNYN | | GVNYMHWYQQKPGTSPRRWIYDTSK | |
| | QNFQGKAKLTVDKSTSTAYMEVSSLRSDDTA | | LASGVPARFSGSGSGTSYSLTISSMEP | |
| | VYYCARGSNPYYYAMDYWGQGTSVTVSS | | EDAATYYCHQRGSYTFGGGTKLEIK | |
| mogamulizumab | EVQLVESGGDLVQPGRSLRLSCAASGFIFSN | 165 | DVLMTQSPLSLPVTPGEPASISCRSSR | 166 |
| | YGMSWVRQAPGKGLEWVATISSASTYSYYP | | NIVHINGDTYLEWYLQKPGQSPQLLIY | |
| | DSVKGRFTISRDNAKNSLYLQMNSLRVEDTA | | KVSNRFSGVPDRFSGSGSGTDFTLKIS | |
| | LYYCGRHSDGNFAFGYWGQGTLVTVSS | | RVEAEDVGVYYCFQGSLLPWTFGQG | |
| | | | TKVEIK | |
| obexelimab | EVQLVESGGGLVKPGGSLKLSCAASGYTFTS | 167 | DIVMTQSPATLSLSPGERATLSCRSSK | 168 |
| | YVMHWVRQAPGKGLEWIGYINPYNDGTKYN | | SLQNVNGNTYLYWFQQKPGQSPQLLI | |
| | EKFQGRVTISSDKSISTAYMELSSLRSEDTAM | | YRMSNLNSGVPDRFSGSGSGTEFTLT | |
| | YYCARGTYYYGTRVFDYWGQGTLVTVSS | | ISSLEPEDFAVYYCMQHLEYPITFGAG | |
| | | | TKLEIK | |
| plozalizumab | EVQLVESGGGLVKPGGSLRLSCAASGFTFSA | 169 | DVVMTQSPLSLPVTLGQPASISCKSSQ | 170 |
| | YAMNWVRQAPGKGLEWVGRIRTKNNNYATY | | SLLDSDGKTFLNWFQQRPGQSPRRLI | |
| | YADSVKDRFTISRDDSKNTLYLQMNSLKTEDT | | YLVSKLDSGVPDRFSGSGSGTDFTLKI | |
| | AVYYCTTFYGNGVWGQGTLVTVSS | | SRVEAEDVGVYYCWQGTHFPYTFGQ | |
| | | | GTRLEIK | |
| tafasitamab | EVQLVESGGGLVKPGGSLKLSCAASGYTFTS | 171 | DIVMTQSPATLSLSPGERATLSCRSSK | 172 |
| | YVMHWVRQAPGKGLEWIGYINPYNDGTKYN | | SLQNVNGNTYLYWFQQKPGQSPQLLI | |
| | EKFQGRVTISSDKSISTAYMELSSLRSEDTAM | | YRMSNLNSGVPDRFSGSGSGTEFTLT | |
| | YYCARGTYYYGTRVFDYWGQGTLVTVSS | | ISSLEPEDFAVYYCMQHLEYPITFGAG | |
| | | | TKLEIK | |
| divozilimab | EVQLVQPGAEVVKPGASVKVSCKASGYTFTS | 173 | QIVLSQSPAILSASPGERVTLTCRASSS | 174 |
| | YNMHWVRQAPGRGLEWMGAIYPGNGDTSY | | VSYIHWFQQKPGKAPKPLIYATSNLAS | |
| | NQKFKGRVTMTRDKSTSTVYMELSSLRSEDT | | GVPSRFSGSGSGTDFSLTISRVEPEDF | |
| | AVYYCARSTYYGGDWYFNVWGQGTLVTVSS | | AVYYCQQWTSNPPTFGGGTKVEIK | |
| ibritumomab | QAYLQQSGAELVRPGASVKMSCKASGYTFT | 175 | QIVLSQSPAILSASPGEKVTMTCRASS | 176 |
| | SYNMHWVKQTPRQGLEWIGAIYPGNGDTSY | | SVSYMHWYQQKPGSSPKPWIYAPSN | |
| | NQKFKGKATLTVDKSSSTAYMQLSSLTSEDS | | LASGVPARFSGSGSGTSYSLTISRVEA | |
| | AVYFCARVVYYSNSYWYFDVWGTGTTVTVS | | EDAATYYCQQWSFNPPTFGAGTKLEL | |
| | A | | K | |
| ocaratuzumab | EVQLVQSGAEVKKPGESLKISCKGSGRTFTS | 177 | EIVLTQSPGTLSLSPGERATLSCRASS | 178 |
| | YNMHWVRQMPGKGLEWMGAIYPLTGDTSYN | | SVPYIHWYQQKPGQAPRLLIYATSALA | |
| | QKSKLQVTISADKSISTAYLQWSSLKASDTAM | | SGIPDRFSGSGSGTDFTLTISRLEPED | |
| | YYCARSTYVGGDWQFDVWGKGTTVTVSS | | FAVYYCQQWLSNPPTFGQGTKLEIK | |
| ocrelizumab | EVQLVESGGGLVQPGGSLRLSCAASGYTFTS | 179 | DIQMTQSPSSLSASVGDRVTITCRASS | 180 |
| | YNMHWVRQAPGKGLEWVGAIYPGNGDTSYN | | SVSYMHWYQQKPGKAPKPLIYAPSNL | |
| | QKFKGRFTISVDKSKNTLYLQMNSLRAEDTAV | | ASGVPSRFSGSGSGTDFTLTISSLQPE | |
| | YYCARVVYYSNSYWYFDVWGQGTLVTVSS | | DFATYYCQQWSFNPPTFGQGTKVEIK | |
| ofatumumab | EVQLVESGGGLVQPGRSLRLSCAASGFTFND | 181 | EIVLTQSPATLSLSPGERATLSCRASQ | 182 |
| | YAMHWVRQAPGKGLEWVSTISWNSGSIGYA | | SVSSYLAWYQQKPGQAPRLLIYDASN | |
| | DSVKGRFTISRDNAKKSLYLQMNSLRAEDTAL | | RATGIPARFSGSGSGTDFTLTISSLEPE | |
| | YYCAKDIQYGNYYYGMDVWGQGTTVTVSS | | DFAVYYCQQRSNWPITFGQGTRLEIK | |
| ripertamab | QVQLQQPGAELVKPGASVKMSCKASGYTFT | 183 | QIVLSQSPAILSASPGEKVTMTCRASS | 184 |
| | SYNMHWVKQTPGRGLEWIGAIYPGNGDTSY | | SVSYIHWFQQKPGSSPKPWIYATSNL | |
| | NQKFKGKATLTADKSSSTAYMQLSSLTSEDS | | ASGVPVRFSGSGSGTSYSLTISRVEAE | |
| | AVYYCARSTYYGGDWYFNVWGAGTTVTVSA | | DAATYYCQQWTSNPPTFGGGTKLEIK | |
| rituximab | QVQLQQPGAELVKPGASVKMSCKASGYTFT | 185 | QIVLSQSPAILSASPGEKVTMTCRASS | 186 |
| | SYNMHWVKQTPGRGLEWIGAIYPGNGDTSY | | SVSYIHWFQQKPGSSPKPWIYATSNL | |
| | NQKFKGKATLTADKSSSTAYMQLSSLTSEDS | | ASGVPVRFSGSGSGTSYSLTISRVEAE | |
| | AVYYCARSTYYGGDWYFNVWGAGTTVTVSA | | DAATYYCQQWTSNPPTFGGGTKLEIK | |
| ublituximab | QAYLQQSGAELVRPGASVKMSCKASGYTFT | 187 | QIVLSQSPAILSASPGEKVTMTCRASS | 188 |
| | SYNMHWVKQTPRQGLEWIGGIYPGNGDTSY | | SVSYMHWYQQKPGSSPKPWIYATSNL | |
| | NQKFKGKATLTVGKSSSTAYMQLSSLTSEDS | | ASGVPARFSGSGSGTSYSFTISRVEAE | |
| | AVYFCARYDYNYAMDYWGQGTSVTVSS | | DAATYYCQQWTFNPPTFGGGTRLEIK | |
| veltuzumab | QVQLQQSGAEVKKPGSSVKVSCKASGYTFT | 189 | DIQLTQSPSSLSASVGDRVTMTCRAS | 190 |
| | SYNMHWVKQAPGQGLEWIGAIYPGMGDTSY | | SSVSYIHWFQQKPGKAPKPWIYATSN | |
| | NQKFKGKATLTADESTNTAYMELSSLRSEDT | | LASGVPVRFSGSGSGTDYTFTISSLQP | |
| | AFYYCARSTYYGGDWYFDVWGQGTTVTVSS | | EDIATYYCQQWTSNPPTFGGGTKLEIK | |
| zuberitamab | EVQLQQSGAELVRPGASVKMSCKASGYTFT | 191 | DIELSQSPAILSASPGEKVTMTCRASS | 192 |
| | SYNMHWVKQTPRQGLEWIGAIYPGNGDTSY | | SVSYMHWYQQKPGSSPKPWIYAPSN | |
| | NQKFKGKATLTVDKSSSTAYMQLSSLTSEDS | | LASGVPARFSGSGSGTSYSLTISRVEA | |
| | AVYFCARVVYYSNSYWYFDVWGTGTTVTVS | | EDAATYYCQQWSFNPPTFGAGTKLEI | |
| | S | | K | |
| epratuzumab | QVQLVQSGAEVKKPGSSVKVSCKASGYTFTS | 193 | DIQLTQSPSSLSASVGDRVTMSCKSS | 194 |
| | YWLHWVRQAPGQGLEWIGYINPRNDYTEYN | | QSVLYSANHKNYLAWYQQKPGKAPKL | |
| | QNFKDKATITADESTNTAYMELSSLRSEDTAF | | LIYWASTRESGVPSRFSGSGSGTDFT | |
| | YFCARRDITTFYWGQGTTVTVSS | | FTISSLQPEDIATYYCHQYLSSWTFGG | |
| | | | GTKLEIK | |
| inotuzumab | EVQLVQSGAEVKKPGASVKVSCKASGYRFTN | 195 | DVQVTQSPSSLSASVGDRVTITCRSS | 196 |
| | YWIHWVRQAPGQGLEWIGGINPGNNYATYR | | QSLANSYGNTFLSWYLHKPGKAPQLLI | |
| | RKFQGRVTMTADTSTSTVYMELSSLRSEDTA | | YGISNRFSGVPDRFSGSGSGTDFTLTI | |
| | VYYCTREGYGNYGAWFAYWGQGTLVTVSS | | SSLQPEDFATYYCLQGTHQPYTFGQG | |
| | | | TKVEIK | |
| pinatuzumab | EVQLVESGGGLVQPGGSLRLSCAASGYEFS | 197 | DIQMTQSPSSLSASVGDRVTITCRSSQ | 198 |
| | RSWMNWVRQAPGKGLEWVGRIYPGDGDTN | | SIVHSVGNTFLEWYQQKPGKAPKLLIY | |
| | YSGKFKGRFTISADTSKNTAYLQMNSLRAED | | KVSNRFSGVPSRFSGSGSGTDFTLTIS | |
| | TAVYYCARDGSSWDWYFDVWGQGTLVTVS | | SLQPEDFATYYCFQGSQFPYTFGQGT | |
| | S | | KVEIK | |
| suciraslimab | QVQLQESGGGLVKPGGSLKLSCAASGFAFSI | 199 | DIQLTQTTSSLSASLGDRVTISCRASQ | 200 |
| | YDMSWVRQTPEKRLEWVAYISSGGGTTYYP | | DISNYLNWYQQKPDGTVKLLIYYTSILH | |
| | DTVKGRFTISRDNAKNTLYLQMSSLKSEDTA | | SGVPSRFSGSGSGTDYSLTISNLEQE | |
| | MYYCARHSGYGSSYGVLFAYWGQGTLVTVS | | DFATYFCQQGNTLPWTFGGGTKLEIK | |
| | S | | | |
| brentuximab | QIQLQQSGPEVVKPGASVKISCKASGYTFTDY | 201 | DIVLTQSPASLAVSLGQRATISCKASQ | 202 |
| | YITWVKQKPGQGLEWIGWIYPGSGNTKYNEK | | SVDFDGDSYMNWYQQKPGQPPKVLI | |
| | FKGKATLTVDTSSSTAFMQLSSLTSEDTAVYF | | YAASNLESGIPARFSGSGSGTDFTLNI | |
| | CANYGNYWFAYWGQGTQVTVSA | | HPVEEEDAATYYCQQSNEDPWTFGG | |
| | | | GTKLEIK | |
| iratumumab | QVQLQQWGAGLLKPSETLSLTCAVYGGSFSA | 203 | DIQMTQSPTSLSASVGDRVTITCRASQ | 204 |
| | YYWSWIRQPPGKGLEWIGDINHGGGTNYNP | | GISSWLTWYQQKPEKAPKSLIYAASSL | |
| | SLKSRVTISVDTSKNQFSLKLNSVTAADTAVY | | QSGVPSRFSGSGSGTDFTLTISSLQPE | |
| | YCASLTAYWGQGSLVTVSS | | DFATYYCQQYDSYPITFGQGTRLEIK | |
| gemtuzumab | EVQLVQSGAEVKKPGSSVKVSCKASGYTITD | 205 | DIQLTQSPSTLSASVGDRVTITCRASE | 206 |
| | SNIHWVRQAPGQSLEWIGYIYPYNGGTDYNQ | | SLDNYGIRFLTWFQQKPGKAPKLLMY | |
| | KFKNRATLTVDNPTNTAYMELSSLRSEDTAFY | | AASNQGSGVPSRFSGSGSGTEFTLTI | |
| | YCVNGNPWLAYWGQGTLVTVSS | | SSLQPDDFATYYCQQTKEVPWSFGQ | |
| | | | GTKVEVK | |
| lintuzumab | QVQLVQSGAEVKKPGSSVKVSCKASGYTFTD | 207 | DIQMTQSPSSLSASVGDRVTITCRASE | 208 |
| | YNMHWVRQAPGQGLEWIGYIYPYNGGTGYN | | SVDNYGISFMNWFQQKPGKAPKLLIYA | |
| | QKFKSKATITADESTNTAYMELSSLRSEDTAV | | ASNQGSGVPSRFSGSGSGTDFTLTIS | |
| | YYCARGRPAMDYWGQGTLVTVSS | | SLQPDDFATYYCQQSKEVPWTFGQG | |
| | | | TKVEIK | |
| vadastuximab | QVQLVQSGAEVKKPGASVKVSCKASGYTFTN | 209 | DIQMTQSPSSLSASVGDRVTINCKASQ | 210 |
| | YDINWVRQAPGQGLEWIGWIYPGDGSTKYN | | DINSYLSWFQQKPGKAPKTLIYRANRL | |
| | EKFKAKATLTADTSTSTAYMELRSLRSDDTAV | | VDGVPSRFSGSGSGQDYTLTISSLQP | |
| | YYCASGYEDAMDYWGQGTTVTVSS | | EDFATYYCLQYDEFPLTFGGGTKVEIK | |
| daratumumab | EVQLLESGGGLVQPGGSLRLSCAVSGFTFNS | 211 | EIVLTQSPATLSLSPGERATLSCRASQ | 212 |
| | FAMSWVRQAPGKGLEWVSAISGSGGGTYYA | | SVSSYLAWYQQKPGQAPRLLIYDASN | |
| | DSVKGRFTISRDNSKNTLYLQMNSLRAEDTA | | RATGIPARFSGSGSGTDFTLTISSLEPE | |
| | VYFCAKDKILWFGEPVFDYWGQGTLVTVSS | | DFAVYYCQQRSNWPPTFGQGTKVEIK | |
| erzotabart | QVQLVQSGAEVKKPGSSVKVSCKAFGGTFS | 213 | DIQMTQSPSSLSASVGDRVTITCRASQ | 214 |
| | SYAISWVRQAPGQGLEWMGRIIRFLGIANYA | | GIRSWLAWYQQKPEKAPKSLIYAASSL | |
| | QKFQGRVTLIADKSTNTAYMELSSLRSEDTAV | | QSGVPSRFSGSGSGTDFTLTISSLQPE | |
| | YYCAGEPGERDPDAVDIWGQGTMVTVSS | | DFATYYCQQYNSYPLTFGGGTKVEIK | |
| felzartamab | QVQLVESGGGLVQPGGSLRLSCAASGFTFSS | 215 | DIELTQPPSVSVAPGQTARISCSGDNL | 216 |
| | YYMNWVRQAPGKGLEWVSGISGDPSNTYYA | | RHYYVYWYQQKPGQAPVLVIYGDSKR | |
| | DSVKGRFTISRDNSKNTLYLQMNSLRAEDTA | | PSGIPERFSGSNSGNTATLTISGTQAE | |
| | VYYCARDLPLVYTGFAYWGQGTLVTVSS | | DEADYYCQTYTGGASLVFGGGTKLTV | |
| | | | L | |
| isatuximab | QVQLVQSGAEVAKPGTSVKLSCKASGYTFTD | 217 | DIVMTQSHLSMSTSLGDPVSITCKASQ | 218 |
| | YWMQWVKQRPGQGLEWIGTIYPGDGDTGYA | | DVSTVVAWYQQKPGQSPRRLIYSASY | |
| | QKFQGKATLTADKSSKTVYMHLSSLASEDSA | | RYIGVPDRFTGSGAGTDFTFTISSVQA | |
| | VYYCARGDYYGSNSLDYWGQGTSVTVSS | | EDLAVYYCQQHYSPPYTFGGGTKLEIK | |
| lumrotatug | QVQLQESGPGLVKPSETLSLTCTVSGFSLTS | 219 | EIVMTQSPPTLSLSPGERVTLSCRASE | 220 |
| | YGIHWLRQPPGKGLEWIGVIWRGGSTDYNPS | | DIYNRLVWYQQKPGQAPRLLISGVTSL | |
| | LKSRVTISKDTSKSQVSLKLSSVTAADTAVYY | | ETSIPARFSGSGSGTDYTLTISSLQPE | |
| | CAKGKVTTGFYFDFWGQGTLVTVSS | | DFAVYYCQQYWSTPYTFGQGTKVEIK | |
| mezagitamab | EVQLLESGGGLVQPGGSLRLSCAASGFTFDD | 221 | QSVLTQPPSASGTPGQRVTISCSGSS | 222 |
| | YGMSWVRQAPGKGLEWVSDISWNGGKTHY | | SNIGDNYVSWYQQLPGTAPKLLIYRDS | |
| | VDSVKGQFTISRDNSKNTLYLQMNSLRAEDT | | QRPSGVPDRFSGSKSGTSASLAISGL | |
| | AVYYCARGSLFHDSSGFYFGHWGQGTLVTV | | RSEDEADYYCQSYDSSLSGSVFGGGT | |
| | SS | | KLTVL | |
| modakafusp | EVQLVQSGAEVKKPGATVKISCKVSGYTFTD | 223 | DIQMTQSPSSLSASVGDRVTITCKASQ | 224 |
| | SVMNWVQQAPGKGLEWMGWIDPEYGRTDV | | NVDSDVDWYQQKPGKAPKLLIYKASN | |
| | AEKFQGRVTITADTSTDTAYMELSSLRSEDTA | | DYTGVPSRFSGSGSGTDFTFTISSLQP | |
| | VYYCARTKYNSGYGFPYWGQGTTVTVSS | | EDIATYYCMQSNTHPRTFGGGTKVEIK | |
| cusatuzumab | EVQLVESGGGLVQPGGSLRLSCAASGFTFSV | 225 | QAVVTQEPSLTVSPGGTVTLTCGLKS | 226 |
| | YYMNWVRQAPGKGLEWVSDINNEGGTTYYA | | GSVTSDNFPTWYQQTPGQAPRLLIYN | |
| | DSVKGRFTISRDNSKNSLYLQMNSLRAEDTA | | TNTRHSGVPDRFSGSILGNKAALTITG | |
| | VYYCARDAGYSNHVPIFDSWGQGTLVTVSS | | AQADDEAEYFCALFISNPSVEFGGGT | |
| | | | QLTVL | |
| vorsetuzumab | QVQLVQSGAEVKKPGASVKVSCKASGYTFTN | 227 | DIVMTQSPDSLAVSLGERATINCRASK | 228 |
| | YGMNWVRQAPGQGLKWMGWINTYTGEPTY | | SVSTSGYSFMHWYQQKPGQPPKLLIY | |
| | ADAFKGRVTMTRDTSISTAYMELSRLRSDDT | | LASNLESGVPDRFSGSGSGTDFTLTIS | |
| | AVYYCARDYGDYGMDYWGQGTTVTVSS | | SLQAEDVAVYYCQHSREVPWTFGQG | |
| | | | TKVEIK | |
| osemitamab | QVQLVQSGAEVKKPGASVKVSCKASGYTFT | 229 | DIVMTQSPDSLAVSLGERATINCKSSQ | 230 |
| | GYNMNWVRQAPGQGLEWMGNIDPYYGGTS | | SLLNSGNLKNYLTWYQQKPGQPPKLLI | |
| | YNQKFKGRVTMTIDKSTSTVYMELSSLRSED | | YWASTRKSGVPDRFSGSGSGTDFTLT | |
| | TAVYYCARMYHGNAFDYWGQGTTVTVSS | | ISSLQAEDVAVYYCQNDYSYPLTFGG | |
| | | | GTKVEIK | |
| zolbetuximab | QVQLQQPGAELVRPGASVKLSCKASGYTFTS | 231 | DIVMTQSPSSLTVTAGEKVTMSCKSS | 232 |
| | YWINWVKQRPGQGLEWIGNIYPSDSYTNYNQ | | QSLLNSGNQKNYLTWYQQKPGQPPK | |
| | KFKDKATLTVDKSSSTAYMQLSSPTSEDSAV | | LLIYWASTRESGVPDRFTGSGSGTDF | |
| | YYCTRSWRGNSFDYWGQGTTLTVSS | | TLTISSVQAEDLAVYYCQNDYSYPFTF | |
| | | | GSGTKLEIK | |
| tepoditamab | QVQLVQSGAEVKKPGASVKVSCKASGYTFTS | 233 | DIQMTQSPSSLSASVGDRVTITCRASQ | 234 |
| | YYMHWVRQAPGQGLEWMGIINPSGGSTSYA | | SISSYLNWYQQKPGKAPKLLIYAASSL | |
| | QKFQGRVTMTRDTSTSTVYMELSSLRSEDTA | | QSGVPSRFSGSGSGTDFTLTISSLQPE | |
| | VYYCAKGTTGDWFDYWGQGTLVTVSS | | DFATYYCQQSYSTPPTFGQGTKVEIK | |
| roval- | QVQLVQSGAEVKKPGASVKVSCKASGYTFTN | 235 | EIVMTQSPATLSVSPGERATLSCKASQ | 236 |
| pituzumab | YGMNWVRQAPGQGLEWMGWINTYTGEPTY | | SVSNDVVWYQQKPGQAPRLLIYYASN | |
| | ADDFKGRVTMTTDTSTSTAYMELRSLRSDDT | | RYTGIPARFSGSGSGTEFTLTISSLQS | |
| | AVYYCARIGDSSPSDYWGQGTLVTVSS | | EDFAVYYCQQDYTSPWTFGQGTKLEI | |
| | | | K | |
| tarlatamab | QVQLQESGPGLVKPSETLSLTCTVSGGSISSY | 237 | EIVLTQSPGTLSLSPGERVTLSCRASQ | 238 |
| | YWSWIRQPPGKCLEWIGYVYYSGTTNYNPSL | | RVNNNYLAWYQQRPGQAPRLLIYGAS | |
| | KSRVTISVDTSKNQFSLKLSSVTAADTAVYYC | | SRATGIPDRFSGSGSGTDFTLTISRLE | |
| | ASIAVTGFYFDYWGQGTLVTVSS | | PEDFAVYYCQQYDRSPLTFGCGTKLEI | |
| | | | K | |
| becotatug | QVQLQESGPGLVKPSETLSLTCTVSGFSLSN | 239 | EIVLTQSPDFQSVTPKEKVTITCRASQ | 240 |
| | YDVHWVRQAPGKGLEWLGVIWSGGNTDYNT | | SIGTNIHWYQQKPDQSPKLLIKYASESI | |
| | PFTSRLTISVDTSKNQFSLKLSSVTAADTAVY | | SGIPSRFSGSGSGTDFTLTINSLEAED | |
| | YCARALDYYDYEFAYWGQGTLVTVSS | | AATYYCQQNNEWPTSFGQGTKLEIK | |
| cetuximab | QVQLKQSGPGLVQPSQSLSITCTVSGFSLTN | 241 | DILLTQSPVILSVSPGERVSFSCRASQ | 242 |
| | YGVHWVRQSPGKGLEWLGVIWSGGNTDYNT | | SIGTNIHWYQQRTNGSPRLLIKYASESI | |
| | PFTSRLSINKDNSKSQVFFKMNSLQSNDTAIY | | SGIPSRFSGSGSGTDFTLSINSVESEDI | |
| | YCARALTYYDYEFAYWGQGTLVTVSA | | ADYYCQQNNNWPTTFGAGTKLELK | |
| demupitamab | QVQLQESGPGLVKPSETLSLTCTVSGGSVSS | 243 | DIQMTQSPSTLSASVGDRVTITCQASQ | 244 |
| | GDYYWTWIRQSPGKGLEWIGHIYYSGNTNYN | | DISNYLNWYQQKPGKAPKLLIYDASNL | |
| | PSLKSRLTISIDTSKTQFSLKLSSVTAADTAIY | | ETGVPSRFSGSGSGTDFTFTISSLQPE | |
| | YCVRDRVTGAFDIWGQGTMVTVSS | | DIATYFCQHFDHLPLAFGGGTKVEIK | |
| futuximab | EVQLQQPGSELVRPGASVKLSCKASGYTFTS | 245 | DIQMTQTTSSLSASLGDRVTISCRTSQ | 246 |
| | YWMHWVKQRPGQGLEWIGNIYPGSRSTNYD | | DIGNYLNWYQQKPDGTVKLLIYYTSRL | |
| | EKFKSKATLTVDTSSSTAYMQLSSLTSEDSAV | | HSGVPSRFSGSGSGTDFSLTINNVEQ | |
| | YYCTRNGDYYVSSGDAMDYWGQGTSVTVSS | | EDVATYFCQHYNTVPPTFGGGTKLEIK | |
| imgatuzumab | QVQLVQSGAEVKKPGSSVKVSCKASGFTFTD | 247 | DIQMTQSPSSLSASVGDRVTITCRASQ | 248 |
| | YKIHWVRQAPGQGLEWMGYFNPNSGYSTYA | | GINNYLNWYQQKPGKAPKRLIYNTNNL | |
| | QKFQGRVTITADKSTSTAYMELSSLRSEDTAV | | QTGVPSRFSGSGSGTEFTLTISSLQPE | |
| | YYCARLSPGGYYVMDAWGQGTTVTVSS | | DFATYYCLQHNSFPTFGQGTKLEIK | |
| matuzumab | QVQLVQSGAEVKKPGASVKVSCKASGYTFTS | 249 | DIQMTQSPSSLSASVGDRVTITCSASS | 250 |
| | HWMHWVRQAPGQGLEWIGEFNPSNGRTNY | | SVTYMYWYQQKPGKAPKLLIYDTSNL | |
| | NEKFKSKATMTVDTSTNTAYMELSSLRSEDT | | ASGVPSRFSGSGSGTDYTFTISSLQPE | |
| | AVYYCASRDYDYDGRYFDYWGQGTLVTVSS | | DIATYYCQQWSSHIFTFGQGTKVEIK | |
| modotuximab | QVQLQQPGAELVEPGGSVKLSCKASGYTFTS | 251 | DIVMTQAAFSNPVTLGTSASISCRSSK | 252 |
| | HWMHWVKQRPGQGLEWIGEINPSSGRNNY | | SLLHSNGITYLYWYLQKPGQSPQLLIY | |
| | NEKFKSKATLTVDKSSSTAYMQFSSLTSEDS | | QMSNLASGVPDRESSSGSGTDFTLRI | |
| | AVYYCVRYYGYDEAMDYWGQGTSVTVSS | | SRVEAEDVGVYYCAQNLELPYTFGGG | |
| | | | TKLEIK | |
| necitumumab | QVQLQESGPGLVKPSQTLSLTCTVSGGSISS | 253 | EIVMTQSPATLSLSPGERATLSCRASQ | 254 |
| | GDYYWSWIRQPPGKGLEWIGYIYYSGSTDYN | | SVSSYLAWYQQKPGQAPRLLIYDASN | |
| | PSLKSRVTMSVDTSKNQFSLKVNSVTAADTA | | RATGIPARFSGSGSGTDFTLTISSLEPE | |
| | VYYCARVSIFGVGTFDYWGQGTLVTVSS | | DFAVYYCHQYGSTPLTFGGGTKAEIK | |
| nimotuzumab | QVQLQQSGAEVKKPGSSVKVSCKASGYTFT | 255 | DIQMTQSPSSLSASVGDRVTITCRSSQ | 256 |
| | NYYIYWVRQAPGQGLEWIGGINPTSGGSNFN | | NIVHSNGNTYLDWYQQTPGKAPKLLIY | |
| | EKFKTRVTITADESSTTAYMELSSLRSEDTAF | | KVSNRFSGVPSRFSGSGSGTDFTFTIS | |
| | YFCTRQGLWFDSDGRGFDFWGQGTTVTVSS | | SLQPEDIATYYCFQYSHVPWTFGQGT | |
| | | | KLQIT | |
| panitumumab | QVQLQESGPGLVKPSETLSLTCTVSGGSVSS | 257 | DIQMTQSPSSLSASVGDRVTITCQASQ | 258 |
| | GDYYWTWIRQSPGKGLEWIGHIYYSGNTNYN | | DISNYLNWYQQKPGKAPKLLIYDASNL | |
| | PSLKSRLTISIDTSKTQFSLKLSSVTAADTAIYY | | ETGVPSRFSGSGSGTDFTFTISSLQPE | |
| | CVRDRVTGAFDIWGQGTMVTVSS | | DIATYFCQHFDHLPLAFGGGTKVEIK | |
| pimurutamab | EVQLVESGGGLVQPGGSLRLSCAASGFSLTN | 259 | EIVLTQSPATLSLSPGERATLSCRASQ | 260 |
| | YGVHWVRQAPGKGLEWLGVIWSGGNTDYG | | SIGTNIHWYQQKPGQAPRLLIKYASESI | |
| | NEFTSRFTISRDNAKNSLYLQMNSLRAEDTAV | | SGIPARFSGSGSGTDFTLTISSLEPEDF | |
| | YYCARALDYYDYEFAYWGQGTMVTVSS | | AVYYCQQNNNWPTSFGGGTKVEIK | |
| tomuzotuximab | QVQLKQSGPGLVQPSQSLSITCTVSGFSLTN | 261 | DILLTQSPVILSVSPGERVSFSCRASQ | 262 |
| | YGVHWVRQSPGKGLEWLGVIWSGGNTDYNT | | SIGTNIHWYQQRTNGSPRLLIKYASESI | |
| | PFTSRLSINKDNSKSQVFFKMNSLQSNDTAIY | | SGIPSRFSGSGSGTDFTLSINSVESEDI | |
| | YCARALTYYDYEFAYWGQGTLVTVST | | ADYYCQQNNNWPTTFGAGTKLELK | |
| zalutumumab | QVQLVESGGGVVQPGRSLRLSCAASGFTFST | 263 | AIQLTQSPSSLSASVGDRVTITCRASQ | 264 |
| | YGMHWVRQAPGKGLEWVAVIWDDGSYKYY | | DISSALVWYQQKPGKAPKLLIYDASSL | |
| | GDSVKGRFTISRDNSKNTLYLQMNSLRAEDT | | ESGVPSRFSGSESGTDFTLTISSLQPE | |
| | AVYYCARDGITMVRGVMKDYFDYWGQGTLV | | DFATYYCQQFNSYPLTFGGGTKVEIK | |
| | TVSS | | | |
| umizortamig | EVQLVESGGGLVQPGGSLRLSCAASGFTIST | 265 | DVVMTQSPSTLSASVGDRVTINCQAS | 266 |
| | NAMSWVRQAPGKGLEWIGVITGRDITYYASW | | ESISSWLAWYQQKPGKAPKLLIYEASK | |
| | AKGRFTISRDNSKNTLYLQMNSLRAEDTAVY | | LASGVPSRFSGSGSGTEFTLTISSLQP | |
| | YCARDGGSSAITSNNIWGQGTLVTVSS | | DDFATYYCQGYFYFISRTYVNSFGGG | |
| | | | TKVEIK | |
| emirodatamab | QVTLKESGPTLVKPTETLTLTCTLSGFSLNNA | 267 | DIQMTQSPSSLSASVGDRVTITCRASQ | 268 |
| | RMGVSWIRQPPGKCLEWLAHIFSNDEKSYST | | GIRNDLGWYQQKPGKAPKRLIYAASTL | |
| | SLKNRLTISKDSSKTQVVLTMTNVDPVDTATY | | QSGVPSRFSGSGSGTEFTLTISSLQPE | |
| | YCARIVGYGSGWYGFFDYWGQGTLVTVSS | | DFATYYCLQHNSYPLTFGCGTKVEIK | |
| dinutuximab | EVQLLQSGPELEKPGASVMISCKASGSSFTG | 269 | EIVMTQSPATLSVSPGERATLSCRSSQ | 270 |
| | YNMNWVRQNIGKSLEWIGAIDPYYGGTSYNQ | | SLVHRNGNTYLHWYLQKPGQSPKLLI | |
| | KFKGRATLTVDKSSSTAYMHLKSLTSEDSAV | | HKVSNRFSGVPDRFSGSGSGTDFTLK | |
| | YYCVSGMEYWGQGTSVTVSS | | ISRVEAEDLGVYFCSQSTHVPPLTFGA | |
| | | | GTKLELK | |
| lorukafusp | EVQLVQSGAEVEKPGASVKISCKASGSSFTG | 271 | DVVMTQTPLSLPVTPGEPASISCRSSQ | 272 |
| | YNMNWVRQNIGKSLEWIGAIDPYYGGTSYNQ | | SLVHRNGNTYLHWYLQKPGQSPKLLI | |
| | KFKGRATLTVDKSTSTAYMHLKSLRSEDTAV | | HKVSNRFSGVPDRFSGSGSGTDFTLK | |
| | YYCVSGMEYWGQGTSVTVSS | | ISRVEAEDLGVYFCSQSTHVPPLTFGA | |
| | | | GTKLELK | |
| naxitamab | QVQLVESGPGVVQPGRSLRISCAVSGFSVTN | 273 | EIVMTQTPATLSVSAGERVTITCKASQ | 274 |
| | YGVHWVRQPPGKGLEWLGVIWAGGITNYNS | | SVSNDVTWYQQKPGQAPRLLIYSASN | |
| | AFMSRLTISKDNSKNTVYLQMNSLRAEDTAM | | RYSGVPARFSGSGYGTEFTFTISSVQS | |
| | YYCASRGGHYGYALDYWGQGTLVTVSS | | EDFAVYFCQQDYSSFGQGTKLEIK | |
| codrituzumab | QVQLVQSGAEVKKPGASVKVSCKASGYTFTD | 275 | DVVMTQSPLSLPVTPGEPASISCRSSQ | 276 |
| | YEMHWVRQAPGQGLEWMGALDPKTGDTAY | | SLVHSNRNTYLHWYLQKPGQSPQLLI | |
| | SQKFKGRVTLTADKSTSTAYMELSSLTSEDTA | | YKVSNRFSGVPDRFSGSGSGTDFTLKI | |
| | VYYCTRFYSYTYWGQGTLVTVSS | | SRVEAEDVGVYYCSQNTHVPPTFGQ | |
| | | | GTKLEIK | |
| anvatabart | EVQLVESGGGLVQPGGSLRLSCAASGFNIKD | 277 | DIQMTQSPSSLSASVGDRVTITCRASQ | 278 |
| | TYIHWVRQAPGKGLEWVARIYPTNGYTRYAD | | DVNTAVAWYQQKPGKAPKLLIYSASFL | |
| | SVKGRFTISADTSKNTAYLQMNSLRAEDTAVY | | YSGVPSRFSGSRSGTDFTLTISSLQPE | |
| | YCSRWGGDGFYAMDYWGQGTLVTVSS | | DFATYYCQQHYTTPPTFGQGTKVEIK | |
| coprelotamab | EVQLVESGGGLVQPGGSLRLSCAASGFNIKD | 279 | DIQMTQSPSSLSASVGDRVTITCRASQ | 280 |
| | TYIHWVRQAPGKGLEWVARIYPTNGYTRYAD | | DVNTAVAWYQQKPGKAPKLLIYSASFL | |
| | SVKGRFTISADTSKNTAYLQMNSLRAEDTAVY | | YSGVPSRFSGSRSGTDFTLTISSLQPE | |
| | YCSRWGGDGFYAMDYWGQGTLVTVSS | | DFATYYCQQHYTTPPTFGQGTKVEIK | |
| disitamab | EVQLVQSGAEVKKPGATVKISCKVSGYTFTD | 281 | DIQMTQSPSSVSASVGDRVTITCKASQ | 282 |
| | YYIHWVQQAPGKGLEWMGRVNPDHGDSYY | | DVGTAVAWYQQKPGKAPKLLIYWASI | |
| | NQKFKDKATITADKSTDTAYMELSSLRSEDTA | | RHTGVPSRFSGSGSGTDFTLTISSLQP | |
| | VYFCARNYLFDHWGQGTLVTVSS | | EDFATYYCHQFATYTFGGGTKVEIK | |
| gancotamab | QVQLVESGGGLVQPGGSLRLSCAASGFTFR | 283 | QSVLTQPPSVSGAPGQRVTISCTGSS | 284 |
| | SYAMSWVRQAPGKGLEWVSAISGRGDNTYY | | SNIGAGYGVHWYQQLPGTAPKLLIYG | |
| | ADSVKGRFTISRDNSKNTLYLQMNSLRAEDT | | NTNRPSGVPDRFSGFKSGTSASLAITG | |
| | AVYYCAKMTSNAFAFDYWGQGTLVTVSS | | LQAEDEADYYCQSYDSSLSGWVFGG | |
| | | | GTKLTVL | |
| margetuximab | QVQLQQSGPELVKPGASLKLSCTASGFNIKD | 285 | DIVMTQSHKFMSTSVGDRVSITCKASQ | 286 |
| | TYIHWVKQRPEQGLEWIGRIYPTNGYTRYDP | | DVNTAVAWYQQKPGHSPKLLIYSASF | |
| | KFQDKATITADTSSNTAYLQVSRLTSEDTAVY | | RYTGVPDRFTGSRSGTDFTFTISSVQA | |
| | YCSRWGGDGFYAMDYWGQGASVTVSS | | EDLAVYYCQQHYTTPPTFGGGTKVEIK | |
| marstacimab | EVQLLESGGGLVQPGGSLRLSCAASGFTFSS | 287 | QSVLTQPPSVSGAPGQRVTISCTGSS | 288 |
| | YAMSWVRQAPGKGLEWVSAISGSGGSTYYA | | SNIGAGYDVHWYQQLPGTAPKLLIYG | |
| | DSVKGRFTISRDNSKNTLYLQMNSLRAEDTA | | NSNRPSGVPDRFSGSKSGTSASLAIT | |
| | VYYCAILGATSLSAFDIWGQGTMVTVSS | | GLQAEDEADYYCQSYDSSLSGSGVFG | |
| | | | GGTKLTVL | |
| pertuzumab | EVQLVESGGGLVQPGGSLRLSCAASGFTFTD | 289 | DIQMTQSPSSLSASVGDRVTITCKASQ | 290 |
| | YTMDWVRQAPGKGLEWVADVNPNSGGSIYN | | DVSIGVAWYQQKPGKAPKLLIYSASYR | |
| | QRFKGRFTLSVDRSKNTLYLQMNSLRAEDTA | | YTGVPSRFSGSGSGTDFTLTISSLQPE | |
| | VYYCARNLGPSFYFDYWGQGTLVTVSS | | DFATYYCQQYYIYPYTFGQGTKVEIK | |
| timigutuzumab | EVQLVESGGGLVQPGGSLRLSCAASGFNIKD | 291 | DIQMTQSPSSLSASVGDRVTITCRASQ | 292 |
| | TYIHWVRQAPGKGLEWVARIYPTNGYTRYAD | | DVNTAVAWYQQKPGKAPKLLIYSASFL | |
| | SVKGRFTISADTSKNTAYLQMNSLRAEDTAVY | | YSGVPSRFSGSRSGTDFTLTISSLQPE | |
| | YCSRWGGDGFYAMDYWGQGTLVTVSS | | DFATYYCQQHYTTPPTFGQGTKVEIK | |
| trastuzumab | EVQLVESGGGLVQPGGSLRLSCAASGFNIKD | 293 | DIQMTQSPSSLSASVGDRVTITCRASQ | 294 |
| | TYIHWVRQAPGKGLEWVARIYPTNGYTRYAD | | DVNTAVAWYQQKPGKAPKLLIYSASFL | |
| | SVKGRFTISADTSKNTAYLQMNSLRAEDTAVY | | YSGVPSRFSGSRSGTDFTLTISSLQPE | |
| | YCSRWGGDGFYAMDYWGQGTLVTVSS | | DFATYYCQQHYTTPPTFGQGTKVEIK | |
| amatuximab | QVQLQQSGPELEKPGASVKISCKASGYSFTG | 295 | DIELTQSPAIMSASPGEKVTMTCSASS | 296 |
| | YTMNWVKQSHGKSLEWIGLITPYNGASSYNQ | | SVSYMHWYQQKSGTSPKRWIYDTSKL | |
| | KFRGKATLTVDKSSSTAYMDLLSLTSEDSAVY | | ASGVPGRFSGSGSGNSYSLTISSVEA | |
| | FCARGGYDGRGFDYWGSGTPVTVSS | | EDDATYYCQQWSKHPLTFGSGTKVEI | |
| | | | K | |
| anetumab | QVELVQSGAEVKKPGESLKISCKGSGYSFTS | 297 | DIALTQPASVSGSPGQSITISCTGTSSD | 298 |
| | YWIGWVRQAPGKGLEWMGIIDPGDSRTRYS | | IGGYNSVSWYQQHPGKAPKLMIYGVN | |
| | PSFQGQVTISADKSISTAYLQWSSLKASDTAM | | NRPSGVSNRFSGSKSGNTASLTISGL | |
| | YYCARGQLYGGTYMDGWGQGTLVTVSS | | QAEDEADYYCSSYDIESATPVFGGGT | |
| | | | KLTVL | |
| inezetamab | QVQLVESGGGLVKPGGSLRLSCAASGFTFSD | 299 | DIQMTQSPSSVSASVGDRVTITCRASQ | 300 |
| | YYMSWIRQAPGKCLEWISYISSSESIIYYVDAV | | DISRWLAWYQQKPGKAPKLLISAASRL | |
| | KGRFTISRDNAKNSLYLQMNSLRAEDTAVYY | | QSGVPSRFSGSGSGTDFTLTISSLQPE | |
| | CARDVGSHFDYWGQGTLVTVSS | | DFAIYYCQQAKSFPRTFGCGTKVEIK | |
| cantuzumab | QVQLVQSGAEVKKPGETVKISCKASDYTFTY | 301 | DIVMTQSPLSVPVTPGEPVSISCRSSK | 302 |
| | YGMNWVKQAPGQGLKWMGWIDTTTGEPTY | | SLLHSNGNTYLYWFLQRPGQSPQLLIY | |
| | AQKFQGRIAFSLETSASTAYLQIKSLKSEDTAT | | RMSNLVSGVPDRFSGSGSGTAFTLRI | |
| | YFCARRGPYNWYFDVWGQGTTVTVSS | | SRVEAEDVGVYYCLQHLEYPFTFGPG | |
| | | | TKLELK | |
| clivatuzumab | QVQLQQSGAEVKKPGASVKVSCEASGYTFP | 303 | DIQLTQSPSSLSASVGDRVTMTCSASS | 304 |
| | SYVLHWVKQAPGQGLEWIGYINPYNDGTQY | | SVSSSYLYWYQQKPGKAPKLWIYSTS | |
| | NEKFKGKATLTRDTSINTAYMELSRLRSDDTA | | NLASGVPARFSGSGSGTDFTLTISSLQ | |
| | VYYCARGFGGSYGFAYWGQGTLVTVSS | | PEDSASYFCHQWNRYPYTFGGGTRL | |
| | | | EIK | |
| gatipotuzumab | EVQLVESGGGLVQPGGSMRLSCVASGFPFS | 305 | DIVMTQSPLSNPVTPGEPASISCRSSK | 306 |
| | NYWMNWVRQAPGKGLEWVGEIRLKSNNYTT | | SLLHSNGITYFFWYLQKPGQSPQLLIY | |
| | HYAESVKGRFTISRDDSKNSLYLQMNSLKTE | | QMSNLASGVPDRFSGSGSGTDFTLRI | |
| | DTAVYYCTRHYYFDYWGQGTLVTVSS | | SRVEAEDVGVYYCAQNLELPPTFGQG | |
| | | | TKVEIK | |
| abagovomab | QVKLQESGAELARPGASVKLSCKASGYTFTN | 307 | DIELTQSPASLSASVGETVTITCQASEN | 308 |
| | YWMQWVKQRPGQGLDWIGAIYPGDGNTRYT | | IYSYLAWHQQKQGKSPQLLVYNAKTL | |
| | HKFKGKATLTADKSSSTAYMQLSSLASEDSG | | AGGVSSRFSGSGSGTHFSLKIKSLQP | |
| | VYYCARGEGNYAWFAYWGQGTTVTVSS | | EDFGIYYCQHHYGILPTFGGGTKLEIK | |
| sofituzumab | EVQLVESGGGLVQPGGSLRLSCAASGYSITN | 309 | DIQMTQSPSSLSASVGDRVTITCKASD | 310 |
| | DYAWNWVRQAPGKGLEWVGYISYSGYTTYN | | LIHNWLAWYQQKPGKAPKLLIYGATSL | |
| | PSLKSRFTISRDTSKNTLYLQMNSLRAEDTAV | | ETGVPSRFSGSGSGTDFTLTISSLQPE | |
| | YYCARWTSGLDYWGQGTLVTVSS | | DFATYYCQQYWTTPFTFGQGTKVEIK | |
| ubamatamab | QVQLVESGGGLVKPGGSLRLSCAASGFTFSN | 311 | DIQMTQSPSSLSASVGDRVTITCRASQ | 312 |
| | YYMSWVRQAPGKGLEWISYISGRGSTIFYAD | | SISTYLNWYQQKPGKAPKLLIYTASSL | |
| | SVKGRITISRDNAKNSLFLQMNSLRAEDTAVY | | QSGVPSRFSGSGSGTDFTLTISSLQPE | |
| | FCVKDRGGYSPYWGQGTLVTVSS | | DFATYYCQQSYSTPPITFGQGTRLEIK | |
| tesnatilimab | QVHLQESGPGLVKPSETLSLTCTVSDDSISSY | 313 | EIVLTQSPGTLSLSPGERATLSCRASQ | 314 |
| | YWSWIRQPPGKGLEWIGHISYSGSANYNPSL | | SVSSSYLAWYQQKPGQAPRLLIYGAS | |
| | KSRVTISVDTSKNQFSLKLSSVTAADTAVYYC | | SRATGIPDRFSGSGSGTDFTLTISRLE | |
| | ANWDDAFNIWGQGTMVTVSS | | PEDFAVYYCQQYGSSPWTFGQGTKV | |
| | | | EIK | |
| acrixolimab | QVQLVESGGGVVQPGRSLRLSCAASGFTFLR | 315 | DIVMTQTPLSLPVTPGEAASISCRSSQ | 316 |
| | YAMHWVRQAPGKGLEWVAVISYDGRYKYYA | | SLLDSEDGNTYLDWYLQKPGQSPQLLI | |
| | DSVKGRFTISRDNSKNTLYLQMNSLRAEDTA | | YTLSHRASGVPDRFSGSGSGTDFTLEI | |
| | VYYCTTTTFDSWGQGTLVTVSS | | SRVEAEDVGVYYCMQRRDFPFTFGQ | |
| | | | GTKVDIK | |
| balstilimab | QVQLVESGGGVVQPGRSLRLSCAASGFTFS | 317 | EIVMTQSPATLSVSPGERATLSCRASQ | 318 |
| | SYGMHWVRQAPGKGLEWVAVIWYDGSNKY | | SVSSNLAWYQQKPGQAPRLLIYGAST | |
| | YADSVKGRFTISRDNSKNTLYLQMNSLRAED | | RATGIPARFSGSGSGTEFTLTISSLQS | |
| | TAVYYCASNGDHWGQGTLVTVSS | | EDFAVYYCQQYNNWPRTFGQGTKVEI | |
| | | | K | |
| budigalimab | EIQLVQSGAEVKKPGSSVKVSCKASGYTFTH | 319 | DVVMTQSPLSLPVTPGEPASISCRSSQ | 320 |
| | YGMNWVRQAPGQGLEWVGWVNTYTGEPTY | | SIVHSHGDTYLEWYLQKPGQSPQLLIY | |
| | ADDFKGRLTFTLDTSTSTAYMELSSLRSEDTA | | KVSNRFSGVPDRFSGSGSGTDFTLKIS | |
| | VYYCTREGEGLGFGDWGQGTTVTVSS | | RVEAEDVGVYYCFQGSHIPVTFGQGT | |
| | | | KLEIK | |
| camrelizumab | EVQLVESGGGLVQPGGSLRLSCAASGFTFSS | 321 | DIQMTQSPSSLSASVGDRVTITCLASQ | 322 |
| | YMMSWVRQAPGKGLEWVATISGGGANTYYP | | TIGTWLTWYQQKPGKAPKLLIYTATSL | |
| | DSVKGRFTISRDNAKNSLYLQMNSLRAEDTA | | ADGVPSRFSGSGSGTDFTLTISSLQPE | |
| | VYYCARQLYYFDYWGQGTTVTVSS | | DFATYYCQQVYSIPWTFGGGTKVEIK | |
| cemiplimab | EVQLLESGGVLVQPGGSLRLSCAASGFTFSN | 323 | DIQMTQSPSSLSASVGDSITITCRASLS | 324 |
| | FGMTWVRQAPGKGLEWVSGISGGGRDTYFA | | INTFLNWYQQKPGKAPNLLIYAASSLH | |
| | DSVKGRFTISRDNSKNTLYLQMNSLKGEDTA | | GGVPSRFSGSGSGTDFTLTIRTLQPED | |
| | VYYCVKWGNIYFDYWGQGTLVTVSS | | FATYYCQQSSNTPFTFGPGTVVDFR | |
| cetrelimab | QVQLVQSGAEVKKPGSSVKVSCKASGGTFS | 325 | EIVLTQSPATLSLSPGERATLSCRASQ | 326 |
| | SYAISWVRQAPGQGLEWMGGIIPIFDTANYA | | SVRSYLAWYQQKPGQAPRLLIYDASN | |
| | QKFQGRVTITADESTSTAYMELSSLRSEDTAV | | RATGIPARFSGSGSGTDFTLTISSLEPE | |
| | YYCARPGLAAAYDTGSLDYWGQGTLVTVSS | | DFAVYYCQQRNYWPLTFGQGTKVEIK | |
| dostarlimab | EVQLLESGGGLVQPGGSLRLSCAASGFTFSS | 327 | DIQLTQSPSFLSAYVGDRVTITCKASQ | 328 |
| | YDMSWVRQAPGKGLEWVSTISGGGSYTYYQ | | DVGTAVAWYQQKPGKAPKLLIYWAST | |
| | DSVKGRFTISRDNSKNTLYLQMNSLRAEDTA | | LHTGVPSRFSGSGSGTEFTLTISSLQP | |
| | VYYCASPYYAMDYWGQGTTVTVSS | | EDFATYYCQHYSSYPWTFGQGTKLEI | |
| | | | K | |
| enlonstobart | QVQLVESGGGVVQPGRSLRLTCKASGLTFSS | 329 | EIVLTQSPATLSLSPGERATLSCRASQ | 330 |
| | SGMHWVRQAPGKGLEWVAVIWYDGSKRYY | | SVSSYLAWYQQKPGQAPRLLIYTASN | |
| | ADSVKGRFTISRDNSKNTLFLQMNSLRAEDT | | RATGIPARFSGSGSGTDFTLTISSLEPE | |
| | AVYYCATNNDYWGQGTLVTVSS | | DFAVYYCQQYSNWPRTFGQGTKVEIK | |
| ezabenlimab | EVMLVESGGGLVQPGGSLRLSCTASGFTFSK | 331 | EIVLTQSPATLSLSPGERATMSCRASE | 332 |
| | SAMSWVRQAPGKGLEWVAYISGGGGDTYYS | | NIDVSGISFMNWYQQKPGQAPKLLIYV | |
| | SSVKGRFTISRDNAKNSLYLQMNSLRAEDTA | | ASNQGSGIPARFSGSGSGTDFTLTISR | |
| | VYYCARHSNVNYYAMDYWGQGTLVTVSS | | LEPEDFAVYYCQQSKEVPWTFGQGTK | |
| | | | LEIK | |
| finotonlimab | EVQLVESGGGLVKPGGSLRLSCAASGFTFSS | 333 | EIVLTQSPATLSLSPGERATLSCRASE | 334 |
| | YGMSWVRQAPGKRLEWVATISGGGRDTYYS | | SVDSYGNSFMHWYQQKPGQPPRLLIY | |
| | DSVKGRFTISRDNAKNNLYLQMNSLRAEDTA | | AASNQGSGVPARFSGSGSGTDFTLTI | |
| | VYYCSRQYGTVWFFNWGQGTLVTVSS | | SSLEPEDFAMYFCQQSKEVPWTFGQ | |
| | | | GTKVEIK | |
| geptanolimab | QIQLVQSGSELKKPGASVKVSCKASGYTFTN | 335 | DIVLTQSPASLAVSPGQRATITCRASE | 336 |
| | FGMNWVRQAPGQGLKWMGWISGYTREPTY | | SVDNYGYSFMNWFQQKPGQPPKLLIY | |
| | AADFKGRFVISLDTSVSTAYLQISSLKAEDTAV | | RASNLESGVPARFSGSGSRTDFTLTIN | |
| | YYCARDVFDYWGQGTLVTVSS | | PVEADDTANYYCQQSNADPTFGQGT | |
| | | | KLEIK | |
| iparomlimab | QVQLVQSGAEVKKPGASVKVSCKASGYTFTN | 337 | DIQMTQSPSSLSASVGDRVTITCKSSQ | 338 |
| | YWIHWVRQAPGQGLEWMGEIDPYDSYTNYN | | SLFNSGNQKNYLAWYQQKPGKVPKLL | |
| | QKFKGRVTMTVDKSTSTVYMELSSLRSEDTA | | IYGASTRDSGVPYRFSGSGSGTDFTLT | |
| | VYYCARPGFTYGGMDFWGQGTLVTVSS | | ISSLQPEDVATYYCQNDHYYPYTFGG | |
| | | | GTKVEIK | |
| lipustobart | QVQLVQSGAEVKKPGASVKVSCKASGYTFTS | 339 | EIVLTQSPATLSLSPGERATISCRASKG | 340 |
| | YYMYWVRQAPGQGLEWMGGVNPSNGGTNF | | VSTSGYSYLHWYQQKPGQAPRLLIYL | |
| | NEKFKSRVTITADKSTSTAYMELSSLRSEDTA | | ASYLESGVPARFSGSGSGTDFTLTISS | |
| | VYYCARRDYRYDMGFDYWGQGTTVTVSS | | LEPEDFATYYCQHSRELPLTFGTGTKV | |
| | | | EIK | |
| nivolumab | QVQLVESGGGVVQPGRSLRLDCKASGITFSN | 341 | EIVLTQSPATLSLSPGERATLSCRASQ | 342 |
| | SGMHWVRQAPGKGLEWVAVIWYDGSKRYY | | SVSSYLAWYQQKPGQAPRLLIYDASN | |
| | ADSVKGRFTISRDNSKNTLFLQMNSLRAEDT | | RATGIPARFSGSGSGTDFTLTISSLEPE | |
| | AVYYCATNDDYWGQGTLVTVSS | | DFAVYYCQQSSNWPRTFGQGTKVEIK | |
| nofazinlimab | QVQLVQSGAEVKKPGSSVKVSCKASGFTFTT | 343 | DVVMTQSPLSLPVTLGQPASISCRSSQ | 344 |
| | YYISWVRQAPGQGLEYLGYINMGSGGTNYNE | | SLLDSDGGTYLYWFQQRPGQSPRRLI | |
| | KFKGRVTITADKSTSTAYMELSSLRSEDTAVY | | YLVSTLGSGVPDRFSGSGSGTDFTLKI | |
| | YCAIIGYFDYWGQGTMVTVSS | | SRVEAEDVGVYYCMQLTHWPYTFGQ | |
| | | | GTKLEIK | |
| penpulimab | EVQLVESGGGLVQPGGSLRLSCAASGFAFSS | 345 | DIQMTQSPSSMSASVGDRVTFTCRAS | 346 |
| | YDMSWVRQAPGKGLDWVATISGGGRYTYYP | | QDINTYLSWFQQKPGKSPKTLIYRANR | |
| | DSVKGRFTISRDNSKNNLYLQMNSLRAEDTA | | LVSGVPSRFSGSGSGQDYTLTISSLQP | |
| | LYYCANRYGEAWFAYWGQGTLVTVSS | | EDMATYYCLQYDEFPLTFGAGTKLELK | |
| peresolimab | QVQLVQSGAEVKKPGASVKVSCKVSGYSLSK | 347 | DIQMTQSPSSLSASVGDRVTITCQASQ | 348 |
| | YDMSWVRQAPGKGLEWMGIIYTSGYTDYAQ | | SPNNLLAWYQQKPGKAPKLLIYGASDL | |
| | KFQGRVTMTEDTSTDTAYMELSSLRSEDTAV | | PSGVPSRFSGSGSGTDFTLTISSLQPE | |
| | YYCATGNPYYTNGFNSWGQGTLVTVSS | | DFATYYCQNNYYVGPVSYAFGGGTKV | |
| | | | EIK | |
| pidilizumab | QVQLVQSGSELKKPGASVKISCKASGYTFTN | 349 | EIVLTQSPSSLSASVGDRVTITCSARSS | 350 |
| | YGMNWVRQAPGQGLQWMGWINTDSGESTY | | VSYMHWFQQKPGKAPKLWIYRTSNLA | |
| | AEEFKGRFVFSLDTSVNTAYLQITSLTAEDTG | | SGVPSRFSGSGSGTSYCLTINSLQPE | |
| | MYFCVRVGYDALDYWGQGTLVTVSS | | DFATYYCQQRSSFPLTFGGGTKLEIK | |
| pimivalimab | QVQLVQSGAEVKKPGASVKVSCKASGYTFPS | 351 | DIQMTQSPSTLSASVGDRVTITCRASQ | 352 |
| | YYMHWVRQAPGQGLEWMGIINPEGGSTAYA | | SISSWLAWYQQKPGKAPKLLIYEASSL | |
| | QKFQGRVTMTRDTSTSTVYMELSSLRSEDTA | | ESGVPSRFSGSGSGTEFTLTISSLQPD | |
| | VYYCARGGTYYDYTYWGQGTLVTVSS | | DFATYYCQQYNSFPPTFGGGTKVEIK | |
| pradusin- | QVTLKESGPALVKPTQTLTLTCTFSGFSLSTS | 353 | NIQMTQSPSSLSASVGDRVTITCKAGQ | 354 |
| stobart | GTCVSWIRQPPGKALEWLATICWEDSKGYNP | | NVNNYLAWYQQKPGKAPKVLIFNANS | |
| | SLKSRLTISKDTSKNQAVLTMTNMDPVDTATY | | LQTGVPSRFSGSGSGTDFTLTISSLQP | |
| | YCARREDSGYFWFPYWGQGTLVTVSS | | EDFATYYCQQYNSWTTFGGGTKVEIK | |
| prolgolimab | QVQLVQSGGGLVQPGGSLRLSCAASGFTFS | 355 | QPVLTQPLSVSVALGQTARITCGGNNI | 356 |
| | SYWMYWVRQVPGKGLEWVSAIDTGGGRTY | | GSKNVHWYQQKPGQAPVLVIYRDSN | |
| | YADSVKGRFAISRVNAKNTMYLQMNSLRAED | | RPSGIPERFSGSNSGNTATLTISRAQA | |
| | TAVYYCARDEGGGTGWGVLKDWPYGLDAW | | GDEADYYCQVWDSSTAVFGTGTKLTV | |
| | GQGTLVTVSS | | L | |
| pucotenlimab | EVQLVQSGGGLVQPGGSLKLSCAASGFTFSS | 357 | DIVLTQSPASLAVSPGQRATITCRASE | 358 |
| | YGMSWVRQAPGKGLDWVATISGGGRDTYYP | | SVDNYGISFMNWFQQKPGQPPKLLIY | |
| | DSVKGRFTISRDNSKNNLYLQMNSLRAEDTA | | AASNKGTGVPARFSGSGSGTDFTLNI | |
| | LYYCARQKGEAWFAYWGQGTLVTVSS | | NPMEENDTAMYFCQQSKEVPWTFGG | |
| | | | GTKLEIK | |
| retifanlimab | QVQLVQSGAEVKKPGASVKVSCKASGYSFTS | 359 | EIVLTQSPATLSLSPGERATLSCRASE | 360 |
| | YWMNWVRQAPGQGLEWIGVIHPSDSETWLD | | SVDNYGMSFMNWFQQKPGQPPKLLI | |
| | QKFKDRVTITVDKSTSTAYMELSSLRSEDTAV | | HAASNQGSGVPSRFSGSGSGTDFTLT | |
| | YYCAREHYGTSPFAYWGQGTLVTVSS | | ISSLEPEDFAVYFCQQSKEVPYTFGGG | |
| | | | TKVEIK | |
| rosnilimab | QVQLVQSGSELKKPGASVKVSCKASNYTFTD | 361 | EIVLTQSPATLSLSPGERATLSCTASSS | 362 |
| | YSMHWVRQAPGQGLEWMGWINIETYYPTYA | | VSSSYFHWYQQKPGQAPRLLIYSTSN | |
| | DQFKGRFAFSLDTSVSTAYLQISSLKAEDTAV | | LASGIPARFSGSGSGTDFTLTISRLEPE | |
| | YYCARDYYGRFYYAMDYWGQGTTVTVSS | | DFAVYYCHQYHRSPLTFGGGTKVEIK | |
| rulonilimab | EVQLVESGGGLVKPGGSLRLSCAASGFTFSS | 363 | EIVLTQSPATLAVSPGERATISCRASES | 364 |
| | YGMSWVRQTPEKRLEWVATISGGGRDTYYP | | VDDYGISFMNWFQQKPGQPPKLLIYV | |
| | DSVKGRFTISRDNAKNNLYLQMSSLRSEDTA | | ASNQGSGVPARFSGSGSGTDFTLNIH | |
| | LYYCARQKDTSWFVHWGQGTLVTVSS | | PMEEDDTAMYFCQQSKEVPWTFGGG | |
| | | | TKLEIK | |
| sasanlimab | QVQLVQSGAEVKKPGASVKVSCKASGYTFTS | 365 | DIVMTQSPDSLAVSLGERATINCKSSQ | 366 |
| | YWINWVRQAPGQGLEWMGNIYPGSSLTNYN | | SLWDSGNQKNFLTWYQQKPGQPPKL | |
| | EKFKNRVTMTRDTSTSTVYMELSSLRSEDTA | | LIYWTSYRESGVPDRFSGSGSGTDFT | |
| | VYYCARLSTGTFAYWGQGTLVTVSS | | LTISSLQAEDVAVYYCQNDYFYPHTFG | |
| | | | GGTKVEIK | |
| serplulimab | QVQLVESGGGLVKPGGSLRLSCAASGFTFSN | 367 | DIQMTQSPSSLSASVGDRVTITCKASQ | 368 |
| | YGMSWIRQAPGKGLEWVSTISGGGSNIYYAD | | DVTTAVAWYQQKPGKAPKLLIYWAST | |
| | SVKGRFTISRDNAKNSLYLQMNSLRAEDTAV | | RHTGVPSRFSGSGSGTDFTLTISSLQP | |
| | YYCVSYYYGIDFWGQGTSVTVSS | | EDFATYYCQQHYTIPWTFGGGTKLEIK | |
| sintilimab | QVQLVQSGAEVKKPGSSVKVSCKASGGTFS | 369 | DIQMTQSPSSVSASVGDRVTITCRASQ | 370 |
| | SYAISWVRQAPGQGLEWMGLIIPMFDTAGYA | | GISSWLAWYQQKPGKAPKLLISAASSL | |
| | QKFQGRVAITVDESTSTAYMELSSLRSEDTAV | | QSGVPSRFSGSGSGTDFTLTISSLQPE | |
| | YYCARAEHSSTGTFDYWGQGTLVTVSS | | DFATYYCQQANHLPFTFGGGTKVEIK | |
| spartalizumab | EVQLVQSGAEVKKPGESLRISCKGSGYTFTT | 371 | EIVLTQSPATLSLSPGERATLSCKSSQ | 372 |
| | YWMHWVRQATGQGLEWMGNIYPGTGGSNF | | SLLDSGNQKNFLTWYQQKPGQAPRLL | |
| | DEKFKNRVTITADKSTSTAYMELSSLRSEDTA | | IYWASTRESGVPSRFSGSGSGTDFTF | |
| | VYYCTRWTTGTGAYWGQGTTVTVSS | | TISSLEAEDAATYYCQNDYSYPYTFGQ | |
| | | | GTKVEIK | |
| tislelizumab | QVQLQESGPGLVKPSETLSLTCTVSGFSLTS | 373 | DIVMTQSPDSLAVSLGERATINCKSSE | 374 |
| | YGVHWIRQPPGKGLEWIGVIYADGSTNYNPS | | SVSNDVAWYQQKPGQPPKLLINYAFH | |
| | LKSRVTISKDTSKNQVSLKLSSVTAADTAVYY | | RFTGVPDRFSGSGYGTDFTLTISSLQA | |
| | CARAYGNYWYIDVWGQGTTVTVSS | | EDVAVYYCHQAYSSPYTFGQGTKLEIK | |
| toripalimab | QGQLVQSGAEVKKPGASVKVSCKASGYTFT | 375 | DVVMTQSPLSLPVTLGQPASISCRSSQ | 376 |
| | DYEMHWVRQAPIHGLEWIGVIESETGGTAYN | | SIVHSNGNTYLEWYLQKPGQSPQLLIY | |
| | QKFKGRVTITADKSTSTAYMELSSLRSEDTAV | | KVSNRFSGVPDRFSGSGSGTDFTLKIS | |
| | YYCAREGITTVATTYYWYFDVWGQGTTVTVS | | RVEAEDVGVYYCFQGSHVPLTFGQGT | |
| | S | | KLEIK | |
| zeluvalimab | EVQLLESGGGLVQPGGSLRLSCAASGFTFSS | 377 | DIQMTQSPSSVSASVGDRVTITCRASQ | 378 |
| | YDMSWVRQAPGKGLEWVSLISGGGSQTYYA | | GISNWLAWYQQKPGKAPKLLIFAASSL | |
| | ESVKGRFTISRDNSKNTLYLQMNSLRAEDTA | | QSGVPSRFSGSGSGTDFTLTISSLQPE | |
| | VYFCASPSGHYFYAMDVWGQGTTVTVSS | | DFATYYCQQAESFPHTFGGGTKVEIK | |
| zimberelimab | QLQLQESGPGLVKPSETLTLTCTVSADSISST | 379 | QSALTQPASVSGSPGQSITISCTGTSS | 380 |
| | TYYWVWIRQPPGKGLEWIGSISYSGSTYYNP | | DVGFYNYVSWYQQHPGKAPELMIYDV | |
| | SLKSRVTVSVDTSKNQFSLKLNSVAATDTALY | | SNRPSGVSDRFSGSKSGNTASLTISGL | |
| | YCARHLGYNGRYLPFDYWGQGTLVTVSS | | QAEDEADYYCSSYTSISTWVFGGGTK | |
| | | | LTVL | |
| nebratamig | EVQLLESGGGLVQPGGSLRLSCAASGFTISR | 381 | DIQMTQSPSSLSASVGDRVTITCQSSQ | 382 |
| | YHMTWVRQAPGKGLEWIGHIYVNNDDTDYA | | SVYNNNDLAWYQQKPGKVPKLLIYYA | |
| | SSAKGRFTISRDNSKNTLYLQMNSLRAEDTAT | | STLASGVPSRFSGSGSGTDFTLTISSL | |
| | YFCARLDVGGGGAYIGDIWGQGTLVTVSS | | QPEDVATYYCAGGYDTDGLDTFAFGG | |
| | | | GTKVEIK | |
| zilovertamab | QVQLQESGPGLVKPSQTLSLTCTVSGYAFTA | 383 | DIVMTQTPLSLPVTPGEPASISCRASK | 384 |
| | YNIHWVRQAPGQGLEWMGSFDPYDGGSSY | | SISKYLAWYQQKPGQAPRLLIYSGSTL | |
| | NQKFKDRLTISKDTSKNQVVLTMTNMDPVDT | | QSGIPPRFSGSGYGTDFTLTINNIESED | |
| | ATYYCARGWYYFDYWGHGTLVTVSS | | AAYYFCQQHDESPYTFGEGTKVEIK | |
| azintuxizumab | EVQLVESGGGLVQPGGSLRLSCAASGFTFSD | 385 | DVVMTQTPLSLSVTPGQPASISCRSS | 386 |
| | YYMAWVRQAPGKGLEWVASINYDGSSTYYV | | QSLVHSNGNTYLHWYLQKPGQSPQLL | |
| | DSVKGRFTISRDNAKNSLYLQMNSLRAEDTA | | IYKVSNRFSGVPDRFSGSGSGTDFTLK | |
| | VYYCARDRGYYFDYWGQGTTVTVSS | | ISRVEAEDVGVYFCSQSTHVPPFTFG | |
| | | | GGTKVEIK | |
| elotuzumab | EVQLVESGGGLVQPGGSLRLSCAASGFDFS | 387 | DIQMTQSPSSLSASVGDRVTITCKASQ | 388 |
| | RYWMSWVRQAPGKGLEWIGEINPDSSTINYA | | DVGIAVAWYQQKPGKVPKLLIYWAST | |
| | PSLKDKFIISRDNAKNSLYLQMNSLRAEDTAV | | RHTGVPDRFSGSGSGTDFTLTISSLQP | |
| | YYCARPDGNYWYFDVWGQGTLVTVSS | | EDVATYYCQQYSSYPYTFGQGTKVEI | |
| | | | K | |

### Nonlimiting Embodiments

Notwithstanding the appended claims, the present disclosure is also defined by the following embodiments:
1. A nucleic acid comprising a sequence encoding a decoy-resistant interleukin 18 (DR-18) polypeptide and a chimeric antigen receptor (CAR) sequence encoding a CAR, wherein the CAR is selected from the group consisting of:
i) an anti-5T4 CAR comprising an antigen binding domain (ABD) that specifically binds 5T4; ii) an anti-AFP CAR comprising an ABD that specifically binds AFP;
iii) an anti-ALPP CAR comprising an ABD that specifically binds ALPP;
iv) an anti-ALPPL2 CAR comprising an ABD that specifically binds ALPPL2;
v) an anti-APN (0013) CAR comprising an ABD that specifically binds APN (0013);
vi) an anti-APRIL CAR comprising an ABD that specifically binds APRIL;
vii) an anti-AXL CAR comprising an ABD that specifically binds AXL;
viii) an anti-B7-1H3 CAR comprising an ABD that specifically binds 1B7-H3;
ix) an anti-B7-H4 CAR comprising an ABD that specifically binds B7-H4;
x) an anti-BAFF-R CAR comprising an ABD that specifically binds BAFF-R;
xi) an anti-BCMA CAR comprising an ABD that specifically binds BCMA;
xii) an anti-BSG CAR comprising an ABD that specifically binds BSG;
xiii) an anti-CAIX CAR comprising an ABD that specifically binds CAIX;
xiv) an anti-CD123 CAR comprising an ABD that specifically binds CD123;
xv) an anti-CD133 CAR comprising an ABD that specifically binds CD133;
xvi) an anti-CD138 CAR comprising an ABD that specifically binds CD138;
xvii) an anti-CD19 CAR comprising an ABD that specifically binds CD19;
xviii) an anti-CD1A CAR comprising an ABD that specifically binds CD1A;
xix) an anti-CD2 CAR comprising an ABD that specifically binds CD2;
xx) an anti-CD20 CAR comprising an ABD that specifically binds CD20;
xxi) an anti-CD22 CAR comprising an ABD that specifically binds CD22;
xxii) an anti-CD24 CAR comprising an ABD that specifically binds CD24;
xxiii) an anti-CD276 CAR comprising an ABD that specifically binds CD276;
xxiv) an anti-CD3 CAR comprising an ABD that specifically binds CD3;
xxv) an anti-CD30 CAR comprising an ABD that specifically binds CD30;
xxvi) an anti-CD33 CAR comprising an ABD that specifically binds CD33;
xxvii) an anti-CD38 CAR comprising an ABD that specifically binds CD38;
xxviii) an anti-CD4 CAR comprising an ABD that specifically binds CD4;
xxix) an anti-CD43 CAR comprising an ABD that specifically binds CD43;
xxx) an anti-CD44 CAR comprising an ABD that specifically binds CD44;
xxxi) an anti-CD44v6 CAR comprising an ABD that specifically binds CD44v6; xxxii) an anti-CD45 CAR comprising an ABD that specifically binds CD45;
xxxiii) an anti-CD5 CAR comprising an ABD that specifically binds CD5;
xxxiv) an anti-CD56 CAR comprising an ABD that specifically binds CD56;
xxxv) an anti-CD7 CAR comprising an ABD that specifically binds CD7;
xxxvi) an anti-CD70 CAR comprising an ABD that specifically binds CD70;
xxxvii) an anti-CD72 CAR comprising an ABD that specifically binds CD72;
xxxviii) an anti-CD83 CAR comprising an ABD that specifically binds CD83;
xxxix) an anti-CD84 CAR comprising an ABD that specifically binds CD84;
xl) an anti-CD99 CAR comprising an ABD that specifically binds CD99;
xli) an anti-CDCP1 CAR comprising an ABD that specifically binds CDCP1;
xlii) an anti-CDH17 CAR comprising an ABD that specifically binds CDH17;
xliii) an anti-CEA CAR comprising an ABD that specifically binds CEA;
xliv) an anti-CEACAM5 CAR comprising an ABD that specifically binds CEACAM5;
xlv) an anti-CLDN18.2 CAR comprising an ABD that specifically binds CLDN18.2;
xlvi) an anti-CLDN6 CAR comprising an ABD that specifically binds CLDN6;
xlvii) an anti-CLEC4K (CD207) CAR comprising an ABD that specifically binds CLEC4K (CD207);
xlviii) an anti-CLL-1 CAR comprising an ABD that specifically binds CLL-1;
xlix) an anti-CSPG4 CAR comprising an ABD that specifically binds CSPG4;
l) an anti-DLL3 CAR comprising an ABD that specifically binds DLL3;
li) an anti-DR5 CAR comprising an ABD that specifically binds DR5;
lii) an anti-EBV Protein CAR comprising an ABD that specifically binds EBV Protein;
liii) an anti-EGFR CAR comprising an ABD that specifically binds EGFR;
liv) an anti-EGFRvIII CAR comprising an ABD that specifically binds EGFRvII;
lv) an anti-EMR1 CAR comprising an ABD that specifically binds EMR1;
lvi) an anti-enkephalinase (CD10) CAR comprising an ABD that specifically bindsenkephalinase (CD10);
lvii) an anti-EpCAM CAR comprising an ABD that specifically binds EpCAM;
lviii) an anti-EphA2 CAR comprising an ABD that specifically binds EphA2;
lix) an anti-EphA3 CAR comprising an ABD that specifically binds EphA3;
lx) an anti-FAP CAR comprising an ABD that specifically binds FAP;
lxi) an anti-FGFR4 CAR comprising an ABD that specifically binds FGFR4;
lxii) an anti-FLT3 CAR comprising an ABD that specifically binds FLT3;
lxiii) an anti-FOLR1 CAR comprising an ABD that specifically binds FOLR1;
lxiv) an anti-FSHR CAR comprising an ABD that specifically binds FSHR;
lxv) an anti-GC-C CAR comprising an ABD that specifically binds GC-C;
lxvi) an anti-GD2 CAR comprising an ABD that specifically binds GD2;
lxvii) an anti-GFRA4 CAR comprising an ABD that specifically binds GFRA4;
lxviii) an anti-Globo H CAR comprising an ABD that specifically binds Globo H;
lxix) an anti-GM2 (ganglioside M2) CAR comprising an ABD that specifically binds GM2 (ganglioside M2);
lxx) an anti-gp100 CAR comprising an ABD that specifically binds gp100;
lxxi) an anti-GPC1 CAR comprising an ABD that specifically binds GPC1;
lxxii) an anti-GPC2 CAR comprising an ABD that specifically binds GPC2;
lxxiii) an anti-GPC3 CAR comprising an ABD that specifically binds GPC3;
lxxiv) an anti-HAAH (ASPH) CAR comprising an ABD that specifically binds HAAH (ASPH);
lxxv) an anti-HER2 CAR comprising an ABD that specifically binds HER2;
lxxvi) an anti-HGF CAR comprising an ABD that specifically binds HGF;
lxxvii) an anti-HIV envelope protein gp120 CAR comprising an ABD that specifically binds HIV envelope protein gp120;
lxxviii) an anti-HIV-1 pol CAR comprising an ABD that specifically binds HIV-1 pol;
lxxix) an anti-HLA-A2 CAR comprising an ABD that specifically binds HLA-A2;
lxxx) an anti-HLA-G CAR comprising an ABD that specifically binds HLA-G;
lxxxi) an anti-HSP70 heat-shock protein CAR comprising an ABD that specifically binds HSP70 heat-shock protein;
lxxxii) an anti-ICAM-1 CAR comprising an ABD that specifically binds ICAM-1;
lxxxiii) an anti-IL-10R CAR comprising an ABD that specifically binds IL-10R;
lxxxiv) an anti-IL-13Rα2 CAR comprising an ABD that specifically binds IL-13Rα2;
lxxxv) an anti-integrin αvβ6 CAR comprising an ABD that specifically binds integrin αvβ6;
lxxxvi) an anti-ITGB7 CAR comprising an ABD that specifically binds ITGB7;
lxxxvii) an anti-KKLC1 CAR comprising an ABD that specifically binds KKLC1;
lxxxviii) an anti-KMA CAR comprising an ABD that specifically binds KMA;
lxxxix) an anti-L1CAM CAR comprising an ABD that specifically binds L1CAM;
xc) an anti-Lewis-Y antigen CAR comprising an ABD that specifically binds Lewis-Y antigen;
xci) an anti-LGR5 CAR comprising an ABD that specifically binds LGR5;
xcii) an anti-LILRB4 CAR comprising an ABD that specifically binds LILRB4;
xciii) an anti-LMP1 CAR comprising an ABD that specifically binds LMP1;
xciv) an anti-MAGEA3 CAR comprising an ABD that specifically binds MAGEA3;
xcv) an anti-M-CSF CAR comprising an ABD that specifically binds M-CSF;
xcvi) an anti-MG7 CAR comprising an ABD that specifically binds MG7;
xcvii) an anti-MICA CAR comprising an ABD that specifically binds MICA;
xcviii) an anti-MMP2 CAR comprising an ABD that specifically binds MMP2;
xcix) an anti-MSLN CAR comprising an ABD that specifically binds MSLN;
c) an anti-MUC1/MUC16 CAR comprising an ABD that specifically binds MUC1/MUC16;
ci) an anti-NCR3LG1 CAR comprising an ABD that specifically binds NCR3LG1;
cii) an anti-NECTIN2 CAR comprising an ABD that specifically binds NECTIN2;
ciii) an anti-nectin-4 CAR comprising an ABD that specifically binds nectin-4;
civ) an anti-NKG2D CAR comprising an ABD that specifically binds NKG2D;
cv) an anti-NKG2DL CAR comprising an ABD that specifically binds NKG2DL;
cvi) an anti-NR2F6 CAR comprising an ABD that specifically binds NR2F6;
cvii) an anti-NY-ESO-1 CAR comprising an ABD that specifically binds NY-ESO-1;
cviii) an anti-OPCML CAR comprising an ABD that specifically binds OPCML;
cix) an anti-PD-1 CAR comprising an ABD that specifically binds PD-1;
cx) an anti-PDGFRα CAR comprising an ABD that specifically binds PDGFRα;
cxi) an anti-PDL1 CAR comprising an ABD that specifically binds PDL1;
cxii) an anti-PLA2R CAR comprising an ABD that specifically binds PLA2R;
cxiii) an anti-PR1 CAR comprising an ABD that specifically binds PR1;
cxiv) an anti-PSCA CAR comprising an ABD that specifically binds PSCA;
cxv) an anti-PTK7 CAR comprising an ABD that specifically binds PTK7;
cxvi) an anti-PVR CAR comprising an ABD that specifically binds PVR;
cxvii) an anti-ROBO1 CAR comprising an ABD that specifically binds ROBO1;
cxviii) an anti-ROR1 CAR comprising an ABD that specifically binds ROR1;
cxix) an anti-ROR2 CAR comprising an ABD that specifically binds ROR2;
cxx) an anti-SEMA4A CAR comprising an ABD that specifically binds SEMA4A;
cxxi) an anti-SLAMF7 CAR comprising an ABD that specifically binds SLAMF7;
cxxii) an anti-TAG-72 CAR comprising an ABD that specifically binds TAG-72;
cxxiii) an anti-TGF-β CAR comprising an ABD that specifically binds TGF-β;
cxxiv) an anti-TM4SF1 CAR comprising an ABD that specifically binds TM4SF1;
cxxv) an anti-TRAIL CAR comprising an ABD that specifically binds TRAIL;
cxxvi) an anti-TRBC1 CAR comprising an ABD that specifically binds TRBC1;
cxxvii) an anti-TRBC2 CAR comprising an ABD that specifically binds TRBC2;
cxxviii) an anti-Trop-2 CAR comprising an ABD that specifically binds Trop-2;
cxxix) an anti-TSHR CAR comprising an ABD that specifically binds TSHR;
cxxx) an anti-TSLPR CAR comprising an ABD that specifically binds TSLPR;
cxxxi) an anti-ULBP1 CAR comprising an ABD that specifically binds ULBP1;
cxxxii) an anti-αvβ3 integrin CAR comprising an ABD that specifically binds αvβ3 integrin; and cxxxiii) an anti-K light chain of human immunoglobulin CAR comprising an ABD that specifically binds K light chain of human immunoglobulin.
2. The nucleic acid of embodiment 1, wherein the CAR is selected from the group consisting of: a) inaticabtagene autoleucel; b) actalycabtagene autoleucel; c) relmacabtagene autoleucel; d) lisocabtagene maraleucel; e) brexucabtagene autoleucel; f) axicabtagene ciloleucel; g) tisagenlecleucel; h) obecabatagene autoleucel; i) azercabtagene zapreleucel; j) rapcabtagene autoleucel; and k) idecabtagene vicleucel.
3. The nucleic acid of embodiment 1, wherein the CAR is an anti-EGFRvIII CAR comprising a means for specifically binding EGFRvIII.
4. The nucleic acid of embodiment 3, wherein the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO:110, and one or more of heavy chain complementary determining region 1 (HC CDR1), heavy chain complementary determining region 2 (HC CDR2), and heavy chain complementary determining region 3 (HC CDR3) of SEQ ID NO:111.
5. The nucleic acid of embodiment 4, wherein the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO:117).
6. The nucleic acid of embodiment 5, wherein the antigen binding domain comprises a light chain variable region comprising sequence SEQ ID NO:110 and a heavy chain variable region comprising sequence SEQ ID NO:111.
7. The nucleic acid of any one of the preceding embodiments, wherein the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.
8. The nucleic acid of any one of the preceding embodiments, wherein the sequence encoding the DR-18 polypeptide is codon optimized for expression in humans.
9. The nucleic acid of embodiment 8, wherein the sequence encoding the DR-18 polypeptide comprises a DR-18 encoding sequence selected from SEQ ID NOs: 67-84.
10. The nucleic acid of any one of the preceding embodiments, wherein the nucleic acid is configured for in vivo expression of the DR-18 polypeptide and CAR.
11. The nucleic acid of any one of the preceding embodiments, further comprising a sequence encoding a cytokine selected from the group consisting of: interleukin 2, interleukin
21, interleukin 23, interleukin 27, interleukin 33, tumor necrosis factor (TNF), TNF ligand superfamily member 15, interferon (IFN) alpha, IFN beta, and IFN gamma.
12. The nucleic acid of any one of the preceding embodiments, comprising a plurality of CAR sequences encoding a plurality of different CARs that bind different antigens.
13. The nucleic acid of embodiment 12, wherein the plurality of CAR sequences encode a plurality of different CARs that bind to a combination of antigens selected from the group consisting of: CD19 and CD20, CD19 and CD22, BCMA and CD19, CD33 and CLL-1, CD19 and CD276, CD19 and CD20 and CD22, CD19 and IL15R, IL15R and MUC16, IL-15Rα and NKG2D, GPC3 and IL15R, CD16a and CD19 and IL-15Rα, BCMA and CD16a and NKp46, CD123 and CD33, CLDN18.2 and NKG2D, CD20 and CD22, BCMA and SLAMF7, CD19 and CD70, CD19 and IL18R1, GD2 and IL15R, CD70 and IL15R, CD19 and CD8, CD20 and CD79A, CD19 and CD22 and CD8, CD19 and EBV Protein, CD19 and CD7, BCMA and CD38, IL-12 and MUC16, CD33 and FLT3, CD19 and IL-2RP, BCMA and CD70, EGFR and IL-13Rα2, HER2 and IL15R, MICA and MICB, BCMA and TACI, GPC3 and IL-2RP, CD19 and DR5, CD123 and CD33 and CLL-1, CD3 and CD7, CEACAM5 and CEACAM6, CD20 and CD22 and CD38, CD19 and CLDN18.2, AGRE2 and CLL-1, CD123 and TIM3, CLDN18.2 and PDL1, CTLA4 and MSLN and PD-1, STEAP2 and TGFBR2, CEA and IL-15Rα and IL-21R, CD123 and CD33 and CD38 and CLL-1, CD133 and EGFR, BCMA and HPK1, CD8A and NY-ESO-1, CD38 and DR5, CD19 and EGFR, BCMA and CD19 and HER2 and Trop-2, CD19 and MUC1, PDL1 and VEGFR1, CD123 and CD33 and CD38 and CD56 and CLL-1 and MUC1, BCMA and CD138 and CD19 and CD38, BCMA and CD16a and IL-15Rα, NCR2 and NKG2D, CD38 and CLL-1, and CD276 and FGFR4.
14. An expression vector comprising the nucleic acid of any one of the preceding embodiments.
15. The expression vector of embodiment 14, wherein the expression vector comprises, in operable linkage to the sequence encoding the DR-18 polypeptide, a sequence encoding a signal peptide selected from the group consisting of: Human OSM signal peptide, VSV-G signal peptide, Mouse Ig Kappa signal peptide, Mouse Ig Heavy signal peptide, BM40 signal peptide, Secrecon signal peptide, Human IgKVIII signal peptide, CD33 signal peptide, tPA signal peptide, Human Chymotrypsinogen signal peptide, Human trypsinogen-2 signal peptide, Human IL-2 signal peptide, Gaussia luc signal peptide, Albumin (HSA) signal peptide, Influenza Haemagglutinin signal peptide, Human insulin signal peptide, Silkworm Fibroin LC signal peptide, Interleukin-18-binding protein signal peptide, Interleukin-12 subunit beta signal peptide, Interleukin-12 subunit alpha signal peptide, Interleukin-18 signal peptide, and CD8 signal peptide.
16. The expression vector of embodiment 14 or 15, wherein the expression vector comprises, in operable linkage to the sequence encoding the DR-18 polypeptide, a promoter sequence selected from the group consisting of: cytomegalovirus (CMV) promoter, elongation factor 1 alpha (EF1a) promoter, simian virus 40 (SV40) promoter, phosphoglycerate kinase (PGK) ubiquitin C (UBC) promoter, human beta actin promoter, CAG promoter, LC immunoglobulin promoter, HC immunoglobulin promoter, herpes simplex virus thymidine kinase promoter, and spleen focus-forming virus (SFFV) promoter.
17. The expression vector of embodiment 16, wherein the expression vector comprises, in operable linkage to the sequence encoding the DR-18 polypeptide, a human EF1a promoter.
18. The expression vector of any one of embodiments 14-17, wherein the sequence encoding the DR-18 polypeptide and the sequence encoding the CAR are operably linked by a multicistronic element.
19. The expression vector of embodiment 18, wherein the multicistronic element is an internal ribosome entry site.
20. The expression vector of embodiment 18, wherein the multicistronic element is a sequence encoding 2A peptide selected from the group consisting of: T2A, P2A, E2A, and F2A.
21. A cell comprising the nucleic acid or expression vector of any one of the preceding embodiments.
22. The cell of embodiment 21, wherein the cell is selected from the group consisting of: a T cell, a Naïve T cell (TN), stem cell memory T cell (T_{SCM}), a central memory T cell (T_{CM}), a resident memory T cell (TRM), an effector T cell (TEFF), an effector memory cell (TEM), an alpha-beta (αβ) T cell, a gamma-delta (γα) T cell, a Natural Killer (NK) cell, a CD4+ T cell, a CD4+Th1 cell, a CD4+Th2 cell, a CD4+Th9 cell, a CD4+Th17 cell, a CD4+Th22 cell, a CD4+T Regulatory Cell (Treg), a CD4+T follicular helper cell (Tfh), a resting Treg cell, an effector Treg cell, an effector memory Treg cell, a CD8+ T cell, a CD8+Tc1 cell, a CD8+Tc2 cell, a CD8+Tc9 cell, a CD8+Th17 cell, a CD8+Treg cell, a macrophage, a B Cell, a dendritic cell, a stem cell, a hematopoietic stem cell (HSC), and an induced pluripotent stem cell (iPS cell).
23. A method of treating a subject for cancer, the method comprising administering to the subject an effective amount of a nucleic acid, an expression vector, or a cell of any one of the preceding embodiments.
24. The method of embodiment 23, wherein the cancer is selected from the group consisting of: Acute Lymphoblastic Leukemia (ALL), Acute Myeloid Leukemia (AML), Adrenocortical Carcinoma, an AIDS-Related Cancer, Anal Cancer, Appendix Cancer, Astrocytomas, Atypical Teratoid/Rhabdoid Tumor, Basal Cell Carcinoma, Bile Duct Cancer, Bladder Cancer, Bone Cancer, Brain Stem Glioma, Brain Tumor, Breast Cancer, Bronchial Tumors, Burkitt Lymphoma, Carcinoid Tumor, Carcinoma of Unknown Primary, Cardiac (Heart) Tumors, Central Nervous System Tumor, Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct Cancer, Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric (Stomach) Cancer, Gastrointestinal Carcinoid Tumor, Gastrointestinal Stromal Tumors (GIST), Germ Cell Tumor, Gestational Trophoblastic Disease, Glioma, Hairy Cell Leukemia, Head and Neck Cancer, Heart Cancer, Hepatocellular (Liver) Cancer, Histiocytosis, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer (Primary), Lobular Carcinoma In Situ (LCIS), Lung Cancer, Lymphoma, Macroglobulinemia, Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma and Malignant Fibrous Histiocytoma of Bone, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer, Uterine Sarcoma, Vaginal Cancer, Vulvar Cancer, Waldenstrom Macroglobulinemia, and Wilms Tumor.
25. The method of embodiment 23 or 24, wherein the subject does not undergo lymphodepletion during or prior to the administering.
26. The method of any one of embodiments 23-25, wherein the amount of the nucleic acid, the expression vector, or the cell administered to the subject is less than the amount of a corresponding nucleic acid, expression vector, or cell comprising the sequence encoding the CAR administered to the subject to treat the cancer without the sequence encoding the DR-18 polypeptide.
27. The method of any one of embodiments 23-26, wherein the administering comprises delivering multiple doses of the nucleic acid, the expression vector, or the cell to the subject and the dosing frequency is less than the dosing frequency of a corresponding nucleic acid, expression vector, or cell comprising the sequence encoding the CAR administered to the subject to treat the cancer without the sequence encoding the DR-18 polypeptide.
28. A method, the method comprising contacting a human cell with the nucleic acid or expression vector of any one of embodiments 1-20 under conditions sufficient for delivery of the nucleic acid or expression vector into the human cell.
29. The method of embodiment 28, wherein the contacting is performed under conditions sufficient for the expression by the human cell of the CAR from the CAR encoding sequence.
30. The method of embodiment 28 or 29, wherein the contacting is performed under conditions sufficient for the expression by the human cell of the CAR from the CAR coding sequence, the DR-18 polypeptide from the DR-18 polypeptide coding sequence, or both.
31. A nucleic acid, or expression vector, comprising or consisting of the nucleic acid, comprising a sequence encoding a decoy-resistant interleukin 18 (DR-18) polypeptide, wherein the sequence encoding the DR-18 polypeptide is codon optimized for expression in human cells.
32. The nucleic acid or expression vector of embodiment 31, wherein the nucleic acid is configured for in vivo expression of the DR-18 polypeptide.
33. The nucleic acid or expression vector of embodiment 31 or 32, wherein the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H. 34. The nucleic acid or expression vector of any one of embodiments 31-33, wherein the sequence encoding the DR-18 polypeptide comprises a DR-18 encoding sequence selected from SEQ ID NOs: 67-84.
35. The nucleic acid or expression vector of any one of embodiments 31-34, further comprising a sequence encoding a cytokine selected from the group consisting of: interleukin 2, interleukin 21, interleukin 23, interleukin 27, interleukin 33, tumor necrosis factor (TNF), TNF ligand superfamily member 15, interferon (IFN) alpha, IFN beta, and IFN gamma.
36. The nucleic acid or expression vector of any one of embodiments 31-35, further comprising, in operable linkage to the sequence encoding the DR-18 polypeptide, a sequence encoding a signal peptide selected from the group consisting of: Human OSM signal peptide, VSV-G signal peptide, Mouse Ig Kappa signal peptide, Mouse Ig Heavy signal peptide, BM40 signal peptide, Secrecon signal peptide, Human IgKVIII signal peptide, CD33 signal peptide, tPA signal peptide, Human Chymotrypsinogen signal peptide, Human trypsinogen-2 signal peptide, Human IL-2 signal peptide, Gaussia luc signal peptide, Albumin (HSA) signal peptide, Influenza Haemagglutinin signal peptide, Human insulin signal peptide, Silkworm Fibroin LC signal peptide, Interleukin-18-binding protein signal peptide, Interleukin-12 subunit beta signal peptide, Interleukin-12 subunit alpha signal peptide, Interleukin-18 signal peptide, and CD8 signal peptide.
37. The nucleic acid or expression vector of any one of embodiments 31-36, further comprising, in operable linkage to the sequence encoding the DR-18 polypeptide, a promoter sequence selected from the group consisting of: cytomegalovirus (CMV) promoter, elongation factor 1 alpha (EF1a) promoter, simian virus 40 (SV40) promoter, phosphoglycerate kinase (PGK) ubiquitin C (UBC) promoter, human beta actin promoter, CAG promoter, LC immunoglobulin promoter, HC immunoglobulin promoter, herpes simplex virus thymidine kinase promoter, and spleen focus-forming virus (SFFV) promoter.
38. The nucleic acid or expression vector of any one of embodiments 31-37, further comprising a chimeric antigen receptor (CAR) sequence encoding a CAR, wherein the CAR is selected from the group consisting of:
i) an anti-5T4 CAR comprising an antigen binding domain (ABD) that specifically binds 5T4; ii) an anti-AFP CAR comprising an ABD that specifically binds AFP;
iii) an anti-ALPP CAR comprising an ABD that specifically binds ALPP;
iv) an anti-ALPPL2 CAR comprising an ABD that specifically binds ALPPL2;
v) an anti-APN (CD13) CAR comprising an ABD that specifically binds APN (CD13);
vi) an anti-APRIL CAR comprising an ABD that specifically binds APRIL;
vii) an anti-AXL CAR comprising an ABD that specifically binds AXL;
viii) an anti-B7-H3 CAR comprising an ABD that specifically binds B7-H3;
ix) an anti-B7-H4 CAR comprising an ABD that specifically binds B7-H4;
x) an anti-BAFF-R CAR comprising an ABD that specifically binds BAFF-R;
xi) an anti-BCMA CAR comprising an ABD that specifically binds BCMA;
xii) an anti-BSG CAR comprising an ABD that specifically binds BSG;
xiii) an anti-CAIX CAR comprising an ABD that specifically binds CAIX;
xiv) an anti-CD123 CAR comprising an ABD that specifically binds CD123;
xv) an anti-CD133 CAR comprising an ABD that specifically binds CD133;
xvi) an anti-CD138 CAR comprising an ABD that specifically binds CD138;
xvii) an anti-CD19 CAR comprising an ABD that specifically binds CD19;
xviii) an anti-CD1A CAR comprising an ABD that specifically binds CD1A;
xix) an anti-CD2 CAR comprising an ABD that specifically binds CD2;
xx) an anti-CD20 CAR comprising an ABD that specifically binds CD20;
xxi) an anti-CD22 CAR comprising an ABD that specifically binds CD22;
xxii) an anti-CD24 CAR comprising an ABD that specifically binds CD24;
xxiii) an anti-CD276 CAR comprising an ABD that specifically binds CD276;
xxiv) an anti-CD3 CAR comprising an ABD that specifically binds CD3;
xxv) an anti-CD30 CAR comprising an ABD that specifically binds CD30;
xxvi) an anti-CD33 CAR comprising an ABD that specifically binds CD33;
xxvii) an anti-CD38 CAR comprising an ABD that specifically binds CD38;
xxviii) an anti-CD4 CAR comprising an ABD that specifically binds CD4;
xxix) an anti-CD43 CAR comprising an ABD that specifically binds CD43;
xxx) an anti-CD44 CAR comprising an ABD that specifically binds CD44;
xxxi) an anti-CD44v6 CAR comprising an ABD that specifically binds CD44v6;
xxxii) an anti-CD45 CAR comprising an ABD that specifically binds CD45;
xxxiii) an anti-CD5 CAR comprising an ABD that specifically binds CD5;
xxxiv) an anti-CD56 CAR comprising an ABD that specifically binds CD56;
xxxv) an anti-CD7 CAR comprising an ABD that specifically binds CD7;
xxxvi) an anti-CD70 CAR comprising an ABD that specifically binds CD70;
xxxvii) an anti-CD72 CAR comprising an ABD that specifically binds CD72;
xxxviii) an anti-CD83 CAR comprising an ABD that specifically binds CD83;
xxxix) an anti-CD84 CAR comprising an ABD that specifically binds CD84;
xl) an anti-CD99 CAR comprising an ABD that specifically binds CD99;
xli) an anti-CDCP1 CAR comprising an ABD that specifically binds CDCP1;
xlii) an anti-CDH17 CAR comprising an ABD that specifically binds CDH17;
xliii) an anti-CEA CAR comprising an ABD that specifically binds CEA;
xliv) an anti-CEACAM5 CAR comprising an ABD that specifically binds CEACAM5;
xlv) an anti-CLDN18.2 CAR comprising an ABD that specifically binds CLDN18.2;
xlvi) an anti-CLDN6 CAR comprising an ABD that specifically binds CLDN6;
xlvii) an anti-CLEC4K (CD207) CAR comprising an ABD that specifically binds CLEC4K (CD207);
xlviii) an anti-CLL-1 CAR comprising an ABD that specifically binds CLL-1;
xlix) an anti-CSPG4 CAR comprising an ABD that specifically binds CSPG4;
l) an anti-DLL3 CAR comprising an ABD that specifically binds DLL3;
li) an anti-DR5 CAR comprising an ABD that specifically binds DR5;
lii) an anti-EBV Protein CAR comprising an ABD that specifically binds EBV Protein;
liii) an anti-EGFR CAR comprising an ABD that specifically binds EGFR;
liv) an anti-EGFRvIII CAR comprising an ABD that specifically binds EGFRvII;
lv) an anti-EMR1 CAR comprising an ABD that specifically binds EMR1;
lvi) an anti-enkephalinase (CD10) CAR comprising an ABD that specifically binds enkephalinase (CD10);
lvii) an anti-EpCAM CAR comprising an ABD that specifically binds EpCAM;
lviii) an anti-EphA2 CAR comprising an ABD that specifically binds EphA2;
lix) an anti-EphA3 CAR comprising an ABD that specifically binds EphA3;
lx) an anti-FAP CAR comprising an ABD that specifically binds FAP;
lxi) an anti-FGFR4 CAR comprising an ABD that specifically binds FGFR4;
lxii) an anti-FLT3 CAR comprising an ABD that specifically binds FLT3;
lxiii) an anti-FOLR1 CAR comprising an ABD that specifically binds FOLR1;
lxiv) an anti-FSHR CAR comprising an ABD that specifically binds FSHR;
lxv) an anti-GC-C CAR comprising an ABD that specifically binds GC-C;
lxvi) an anti-GD2 CAR comprising an ABD that specifically binds GD2;
lxvii) an anti-GFRA4 CAR comprising an ABD that specifically binds GFRA4;
lxviii) an anti-Globo H CAR comprising an ABD that specifically binds Globo H;
lxix) an anti-GM2 (ganglioside M2) CAR comprising an ABD that specifically binds GM2 (ganglioside M2);
lxx) an anti-gp100 CAR comprising an ABD that specifically binds gp100;
lxxi) an anti-GPC1 CAR comprising an ABD that specifically binds GPC1;
lxxii) an anti-GPC2 CAR comprising an ABD that specifically binds GPC2;
lxxiii) an anti-GPC3 CAR comprising an ABD that specifically binds GPC3;
lxxiv) an anti-HAAH (ASPH) CAR comprising an ABD that specifically binds HAAH (ASPH);
lxxv) an anti-HER2 CAR comprising an ABD that specifically binds HER2;
lxxvi) an anti-HGF CAR comprising an ABD that specifically binds HGF;
lxxvii) an anti-HIV envelope protein gpl20 CAR comprising an ABD that specifically binds HIV envelope protein gpl20;
lxxviii) an anti-HIV-1 pol CAR comprising an ABD that specifically binds HIV-1 pol;
lxxix) an anti-HLA-A2 CAR comprising an ABD that specifically binds HLA-A2;
lxxx) an anti-HLA-G CAR comprising an ABD that specifically binds HLA-G;
lxxxi) an anti-HSP70 heat-shock protein CAR comprising an ABD that specifically binds HSP70 heat-shock protein;
lxxxii) an anti-ICAM-1 CAR comprising an ABD that specifically binds ICAM-1;
lxxxiii) an anti-IL-10R CAR comprising an ABD that specifically binds IL-10R;
lxxxiv) an anti-IL-13Rα2 CAR comprising an ABD that specifically binds IL-13Rα2;
lxxxv) an anti-integrin αvβ6 CAR comprising an ABD that specifically binds integrin αvβ6;
lxxxvi) an anti-ITGB7 CAR comprising an ABD that specifically binds ITGB7;
lxxxvii) an anti-KKLC1 CAR comprising an ABD that specifically binds KKLC1;
lxxxviii) an anti-KMA CAR comprising an ABD that specifically binds KMA;
lxxxix) an anti-L1CAM CAR comprising an ABD that specifically binds L1CAM;
xc) an anti-Lewis-Y antigen CAR comprising an ABD that specifically binds Lewis-Y antigen;
xci) an anti-LGR5 CAR comprising an ABD that specifically binds LGR5;
xcii) an anti-LILRB4 CAR comprising an ABD that specifically binds LILRB4;
xciii) an anti-LMP1 CAR comprising an ABD that specifically binds LMP1;
xciv) an anti-MAGEA3 CAR comprising an ABD that specifically binds MAGEA3;
xcv) an anti-M-CSF CAR comprising an ABD that specifically binds M-CSF;
xcvi) an anti-MG7 CAR comprising an ABD that specifically binds MG7;
xcvii) an anti-MICA CAR comprising an ABD that specifically binds MICA;
xcviii) an anti-MMP2 CAR comprising an ABD that specifically binds MMP2;
xcix) an anti-MSLN CAR comprising an ABD that specifically binds MSLN;
c) an anti-MUC1/MUC16 CAR comprising an ABD that specifically binds MUC1/MUC16; ci) an anti-NCR3LG1 CAR comprising an ABD that specifically binds NCR3LG1; cii) an anti-NECTIN2 CAR comprising an ABD that specifically binds NECTIN2; ciii) an anti-nectin-4 CAR comprising an ABD that specifically binds nectin-4; civ) an anti-NKG2D CAR comprising an ABD that specifically binds NKG2D;
cv) an anti-NKG2DL CAR comprising an ABD that specifically binds NKG2DL;
cvi) an anti-NR2F6 CAR comprising an ABD that specifically binds NR2F6;
cvii) an anti-NY-ESO-1 CAR comprising an ABD that specifically binds NY-ESO-1;
cviii) an anti-OPCML CAR comprising an ABD that specifically binds OPCML;
cix) an anti-PD-1 CAR comprising an ABD that specifically binds PD-1;
cx) an anti-PDGFRα CAR comprising an ABD that specifically binds PDGFRα;
cxi) an anti-PDL1 CAR comprising an ABD that specifically binds PDL1;
cxii) an anti-PLA2R CAR comprising an ABD that specifically binds PLA2R;
cxiii) an anti-PR1 CAR comprising an ABD that specifically binds PR1;
cxiv) an anti-PSCA CAR comprising an ABD that specifically binds PSCA;
cxv) an anti-PTK7 CAR comprising an ABD that specifically binds PTK7;
cxvi) an anti-PVR CAR comprising an ABD that specifically binds PVR;
cxvii) an anti-ROBO1 CAR comprising an ABD that specifically binds ROBO1;
cxviii) an anti-ROR1 CAR comprising an ABD that specifically binds ROR1;
cxix) an anti-ROR2 CAR comprising an ABD that specifically binds ROR2;
cxx) an anti-SEMA4A CAR comprising an ABD that specifically binds SEMA4A;
cxxi) an anti-SLAMF7 CAR comprising an ABD that specifically binds SLAMF7;
cxxii) an anti-TAG-72 CAR comprising an ABD that specifically binds TAG-72;
cxxiii) an anti-TGF-β CAR comprising an ABD that specifically binds TGF-β;
cxxiv) an anti-TM4SF1 CAR comprising an ABD that specifically binds TM4SF1;
cxxv) an anti-TRAIL CAR comprising an ABD that specifically binds TRAIL;
cxxvi) an anti-TRBC1 CAR comprising an ABD that specifically binds TRBC1;
cxxvii) an anti-TRBC2 CAR comprising an ABD that specifically binds TRBC2;
cxxviii) an anti-Trop-2 CAR comprising an ABD that specifically binds Trop-2;
cxxix) an anti-TSHR CAR comprising an ABD that specifically binds TSHR;
cxxx) an anti-TSLPR CAR comprising an ABD that specifically binds TSLPR;
cxxxi) an anti-ULBP1 CAR comprising an ABD that specifically binds ULBP1;
cxxxii) an anti-αvβ3 integrin CAR comprising an ABD that specifically binds αvβ3 integrin; and
cxxxiii) an anti-K light chain of human immunoglobulin CAR comprising an ABD that specifically binds K light chain of human immunoglobulin.
39. The nucleic acid or expression vector of embodiment 38, wherein the CAR is selected from the group consisting of: a) inaticabtagene autoleucel; b) actalycabtagene autoleucel; c) relmacabtagene autoleucel; d) lisocabtagene maraleucel; e) brexucabtagene autoleucel; f) axicabtagene ciloleucel; g) tisagenlecleucel; h) obecabatagene autoleucel; i) azercabtagene zapreleucel; j) rapcabtagene autoleucel; and k) idecabtagene vicleucel.
40. The nucleic acid or expression vector of any one of embodiments 31-39, wherein the sequence encoding the CAR and the sequence encoding the DR-18 polypeptide are operably linked by a multicistronic element.
41. The nucleic acid or expression vector of embodiment 40, wherein the multicistronic element is an internal ribosome entry site.
42. The nucleic acid or expression vector of embodiment 40, wherein the multicistronic element is a sequence encoding 2A peptide selected from the group consisting of: T2A, P2A, E2A, and F2A.
43. The nucleic acid or expression vector of any one of embodiments 38-42, wherein the CAR is an anti-EGFRvIII CAR comprising a means for specifically binding EGFRvIII.
44. The nucleic acid or expression vector of embodiment 43, wherein the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO: 110, and one or more of heavy chain complementary determining region 1 (HC CDR1), heavy chain complementary determining region 2 (HC CDR2), and heavy chain complementary determining region 3 (HC CDR3) of SEQ ID NO:111.
45. The nucleic acid or expression vector of embodiment 44, wherein the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO:117).
46. The nucleic acid or expression vector of embodiment 45, wherein the antigen binding domain comprises a light chain variable region comprising sequence SEQ ID NO: 110 and a heavy chain variable region comprising sequence SEQ ID NO:111.
47. The nucleic acid or expression vector of any one of embodiments 31-46, comprising a plurality of CAR sequences encoding a plurality of different CARs that bind different antigens.
48. The nucleic acid or expression vector of embodiment 47, wherein the plurality of CAR sequences encode a plurality of different CARs that bind to a combination of antigens selected from the group consisting of: CD19 and CD20, CD19 and CD22, BCMA and CD19, CD33 and CLL-1, CD19 and CD276, CD19 and CD20 and CD22, CD19 and IL15R, IL15R and MUC16, IL-15Rα and NKG2D, GPC3 and IL15R, CD16a and CD19 and IL-15Rα, BCMA and CD16a and NKp46, CD123 and CD33, CLDN18.2 and NKG2D, CD20 and CD22, BCMA and SLAMF7, CD19 and CD70, CD19 and IL18R1, GD2 and IL15R, CD70 and IL15R, CD19 and CD8, CD20 and CD79A, CD19 and CD22 and CD8, CD19 and EBV Protein, CD19 and CD7, BCMA and CD38, IL-12 and MUC16, CD33 and FLT3, CD19 and IL-2RP, BCMA and CD70, EGFR and IL-13Rα2, HER2 and IL15R, MICA and MICB, BCMA and TACI, GPC3 and IL-2RP, CD19 and DR5, CD123 and CD33 and CLL-1, CD3 and CD7, CEACAM5 and CEACAM6, CD20 and CD22 and CD38, CD19 and CLDN18.2, AGRE2 and CLL-1, CD123 and TIM3, CLDN18.2 and PDL1, CTLA4 and MSLN and PD-1, STEAP2 and TGFBR2, CEA and IL-15Rα and IL-21R, CD123 and CD33 and CD38 and CLL-1, CD133 and EGFR, BCMA and HPK1, CD8A and NY-ESO-1, CD38 and DR5, CD19 and EGFR, BCMA and CD19 and HER2 and Trop-2, CD19 and MUC1, PDL1 and VEGFR1, CD123 and CD33 and CD38 and CD56 and CLL-1 and MUC1, BCMA and CD138 and CD19 and CD38, BCMA and CD16a and IL-15Rα, NCR2 and NKG2D, CD38 and CLL-1, and CD276 and FGFR4.
49. A cell comprising the nucleic acid or expression vector of any one of embodiments 31-48.
50. The cell of embodiment 49, wherein the cell is selected from the group consisting of: a T cell, a Naïve T cell (TN), stem cell memory T cell (T_{SCM}), a central memory T cell (T_{CM}), a resident memory T cell (TRM), an effector T cell (TEFF), an effector memory cell (TEM), an alpha-beta (αβ) T cell, a gamma-delta (γα) T cell, a Natural Killer (NK) cell, a CD4+ T cell, a CD4+Th1 cell, a CD4+Th2 cell, a CD4+Th9 cell, a CD4+Th17 cell, a CD4+Th22 cell, a CD4+T Regulatory Cell (Treg), a CD4+T follicular helper cell (Tfh), a resting Treg cell, an effector Treg cell, an effector memory Treg cell, a CD8+ T cell, a CD8+Tc1 cell, a CD8+Tc2 cell, a CD8+Tc9 cell, a CD8+Th17 cell, a CD8+Treg cell, a macrophage, a B Cell, a dendritic cell, a stem cell, a hematopoietic stem cell (HSC), and an induced pluripotent stem cell (iPS cell).
51. A method of treating a subject for cancer, the method comprising administering to the subject an effective amount of a nucleic acid, an expression vector, or a cell of any one of embodiments 31-50.
52. The method of embodiment 51, wherein the cancer is selected from the group consisting of: Acute Lymphoblastic Leukemia (ALL), Acute Myeloid Leukemia (AML), Adrenocortical Carcinoma, an AIDS-Related Cancer, Anal Cancer, Appendix Cancer, Astrocytomas, Atypical Teratoid/Rhabdoid Tumor, Basal Cell Carcinoma, Bile Duct Cancer, Bladder Cancer, Bone Cancer, Brain Stem Glioma, Brain Tumor, Breast Cancer, Bronchial Tumors, Burkitt Lymphoma, Carcinoid Tumor, Carcinoma of Unknown Primary, Cardiac (Heart) Tumors, Central Nervous System Tumor, Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct Cancer, Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric (Stomach) Cancer, Gastrointestinal Carcinoid Tumor, Gastrointestinal Stromal Tumors (GIST), Germ Cell Tumor, Gestational Trophoblastic Disease, Glioma, Hairy Cell Leukemia, Head and Neck Cancer, Heart Cancer, Hepatocellular (Liver) Cancer, Histiocytosis, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer (Primary), Lobular Carcinoma In Situ (LCIS), Lung Cancer, Lymphoma, Macroglobulinemia, Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma and Malignant Fibrous Histiocytoma of Bone, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer, Uterine Sarcoma, Vaginal Cancer, Vulvar Cancer, Waldenstrom Macroglobulinemia, and Wilms Tumor.
53. The method of embodiment 52, wherein the subject does not undergo lymphodepletion during or prior to the administering.
54. The method of embodiment 52 or 53, wherein the amount of the nucleic acid, the expression vector, or the cell administered to the subject is less than the amount of a corresponding nucleic acid, expression vector, or cell comprising the sequence encoding the CAR administered to the subject to treat the cancer without the sequence encoding the DR-18 polypeptide.
55. The method of any one of embodiments 52-54, wherein the administering comprises delivering multiple doses of the nucleic acid, the expression vector, or the cell to the subject and the dosing frequency is less than the dosing frequency of a corresponding nucleic acid, expression vector, or cell comprising the sequence encoding the CAR administered to the subject to treat the cancer without the sequence encoding the DR-18 polypeptide.
56. A method of making a population of therapeutic cells, the method comprising:
a) contacting a human cell with a nucleic acid comprising a chimeric antigen receptor (CAR) sequence encoding a CAR and a sequence encoding a decoy-resistant interleukin 18 (DR-18) polypeptide; wherein the nucleic acid is configured to result in expression and secretion of the DR-18 by the human cell ex vivo;
b) culturing the contacted human cell ex vivo in the presence of the secreted DR-18 polypeptide, thereby producing a population of therapeutic cells, wherein the population of therapeutic cells is enhanced as compared to a corresponding population of cells generated without the sequence encoding the DR-18 polypeptide.
57. The method of embodiment 56, wherein the population of therapeutic cells demonstrates enhanced ex vivo proliferation as compared to the corresponding population of cells.
58. The method of embodiment 56 or 57, wherein the population of therapeutic cells demonstrates enhanced in vivo proliferation as compared to the corresponding population of cells.
59. The method of any one of embodiments 56-58, wherein the population of therapeutic cells demonstrates enhanced in vivo persistence as compared to the corresponding population of cells.
60. The method of any one of embodiments 56-59, wherein the population of therapeutic cells demonstrates enhanced tumor cell killing as compared to the corresponding population of cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1—DR-18 Armored Anti-EGFRvIII CAR T Cells

The epidermal growth factor receptor (EGFR) is overexpressed in a variety of human epithelial tumors, often as a consequence of gene amplification. A deletion of exons 2-7 of the EGFR gene is the most common extracellular domain mutation of EGFR and is known as epidermal growth factor receptor variant Ill (EGFRvIII). Aberrant EGFRvII signaling has been shown to be important in driving tumor progression and often correlates with poor prognosis. Studies have indicated that chimeric antigen receptor (CAR) T cell therapy directed to EGFRvIII antigen is safe and that CART-EGFRvIII cells engraft and traffic to tumor sites (see e.g., O'Rourke et al. Sci Transl Med. 2017; 9(399): eaaa0984). However, while initial studies observed a specific loss or decreased expression of EGFRvII antigen in tumors that correlated with CART-EGFRvIII treatment, tumor regression from CART-EGFRvIII monotherapy alone was not observed and clinical benefit could not be confirmed in the 10 patients tested (Id.).

Bicistronic DR-18 armored anti-EGFRvIII CAR constructs were designed to encode human DR-18 polypeptide (hDR18) (SEQ ID NO: 22) with a heterologous human IL2 variant signal peptide (SEQ ID NO:141) and anti-EGFRvIII CAR (SEQ ID NO: 109) in various configurations. EGFRvII may be referred to hereafter in this Example as EGFR for brevity. In some instances, coding sequences were separated by a sequence encoding a 2A peptide sequence and expression of both components was driven by a human elongation factor-1 alpha (EF1a) promoter. In other instances, expression of the anti-EGFR CAR was driven by the EF1a promoter and expression of the hDR18 was driven by a nuclear factor of activated T cells (NFAT) responsive promoter that contained a minimal interleukin 2 promoter (mlL2p) operably linked to a six NFAT response elements (6×NFAT-Bs). All constructs further included woodchuck hepatitis virus post-transcriptional regulatory element (WPRE). Corresponding control constructs were also designed, including bicistronic constructs wherein the hDR-18 coding sequence was replaced sequence encoding a luciferase (Luc) reporter and a monocistronic control containing an anti-EGFR CAR coding sequence without a hDR18 coding sequence. Bicistronic constructs where hDR18 was replaced with sequence encoding wild-type (wt) human interleukin 18 (wt-IL18) (SEQ ID NO: 1) were also designed and created for comparative purposes.

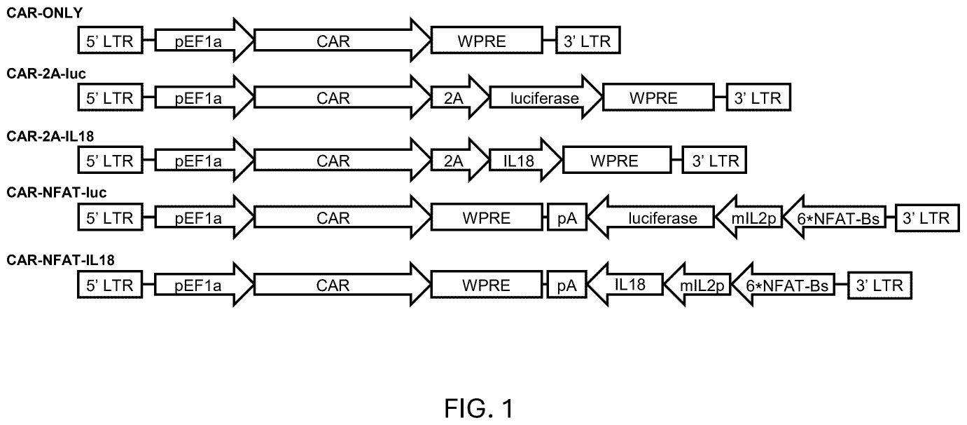

The general architecture of constructs employed in this example, including “EGFR CAR” (i.e., “CAR-ONLY”), “EGFR CAR-2A-hDR18” and “EGFR CAR-2A-wt-IL18” (i.e., “CAR-2A-IL18”), “EGFR CAR-NFAT-hDR18” and “EGFR CAR-NFAT-wt-IL18” (i.e., “CAR-NFAT-IL18”), “EGFR CAR-2A-Luc” (i.e., CAR-2A-lc”), and “EGFR CAR-NFAT-Luc” (i.e., “CAR-NFAT-luc”), are as shown in FIG. 1.

Cells are cultured and CAR lentivirus with the above constructs are packaged, harvested, concentrated, stored, and titred according to standard procedures. To determine expression from titred lentivirus particles, cells are seeded and cultured in the presence of concentrated lentivirus solutions, then CAR expression is measured by flow cytometry following incubation. Once expression from the designed constructs is determined, the produced lentivirus are used to prepare CAR-T cells from activated human pan T cells. CAR T cells transduced with lentivirus carrying each construct are expanded, subcultured, and assessed for transfection efficiency, phenotype (FACS), and IL-18/hDR18 expression (ELISA).

The produced CAR T cells are tested, and activity verified, in an in vitro cytotoxicity assay. In brief, CAR-T cell cytotoxicity is measured by detecting apoptosis of target tumor cells through flow cytometry after co-culture. Cytotoxicity is tested in various effector-to-target cell ratios. In vitro cytotoxicity is tested with and without supplemental cytokines.

In vivo mouse xenograft experiments will be performed. Briefly, EGFR-expressing human tumors are engrafted by subcutaneous injection of five million EGFR-expressing tumor cells into the right flank of immunodeficient (NOG) host mice under conditions that will allow for the tumor to establish and grow within the mouse model. Tumor size, as well as animal health and bodyweight, will be monitored throughout the study. Expression of the EGFR target antigen will be verified in the established tumors and EGFR+tumor bearing mice will be administered CAR-NFAT-luc T cells to validate the inducibility of the transgene by CAR-T cell activation. Appropriate control groups, such as mock T cell treated mice and mice treated with CAR-2A-luc T cells will be used to verify and quantify induced luciferase expression.

After induced expression of luciferase is verified in the EGFR+model, EGFR tumor bearing mice will be intravenously administered mock T cell treatment, CAR-only T cells with cytokine supplementation, CAR-only T cells (without cytokine supplementation), CAR-2A-hDR18 T cells, and CAR-NFAT-hDR18 T cells to assess the relative anti-tumor activity of the various constructs. Corresponding CAR-2A-wt-IL18 and CAR-NFAT-wt-IL18 T cell groups may or may not be further included, e.g., to compare functionality of hDR18 to wt-IL18 in these contexts. In addition to monitoring tumor burden, animal health, and animal bodyweight, blood samples will also be periodically collected for analysis (phenotype by FACS; luciferase, wt-IL18, and hDR18 quantification by ELISA, etc.). Presence, proliferation, and other parameters of the administered cells will also be assessed via samples collected at appropriate timepoints, such as e.g., one day, two days, and one week following administration of the cells. Further analyses may be performed including a cytokine panel analysis, gene expression analyses, cytology, histology, and the like via appropriate collected samples, such as mouse blood, serum, plasma, tumor tissue, and non-tumor tissue.

Constitutive and inducible hDR18 armored anti-EGFR CAR T cell treated mice is expected to show a greater reduction in tumor growth as compared to the mice treated without hDR18 armoring (i.e., the constructs carrying luciferase in place of hDR18 as well as anti-EGFR CAR only T cell control treated animals). Other beneficial characteristics such as increased proliferation and/or enhanced persistence of the administered cells are expected to be observed in samples collected from the hDR18 armored anti-EGFR CAR T cohorts, e.g., as compared to groups without hDR18 armoring. Beneficial in vitro characteristics, such as enhanced in vitro proliferation as compared to non-hDR18-armored CAR T cells, are also expected to be observed. In some instances, certain beneficial in vivo characteristics, such as e.g., enhanced persistence of the administered cells or reduced tumor growth, may be independent of (or observed in the absence of) increased proliferation of the cells in vitro and/or ex vivo.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A nucleic acid comprising a sequence encoding a decoy-resistant interleukin 18 (DR-18) polypeptide and a chimeric antigen receptor (CAR) sequence encoding a CAR, wherein the CAR is selected from:
i) an anti-5T4 CAR comprising an antigen binding domain (ABD) that specifically binds 5T4;
ii) an anti-AFP CAR comprising an ABD that specifically binds AFP;
iii) an anti-ALPP CAR comprising an ABD that specifically binds ALPP;
iv) an anti-ALPPL2 CAR comprising an ABD that specifically binds ALPPL2;
v) an anti-APN (CD13) CAR comprising an ABD that specifically binds APN (CD13);
vi) an anti-APRIL CAR comprising an ABD that specifically binds APRIL;
vii) an anti-AXL CAR comprising an ABD that specifically binds AXL;
viii) an anti-B7-H3 CAR comprising an ABD that specifically binds B7-H3;
ix) an anti-B7-H4 CAR comprising an ABD that specifically binds B7-H4;
x) an anti-BAFF-R CAR comprising an ABD that specifically binds BAFF-R;
xi) an anti-BCMA CAR comprising an ABD that specifically binds BCMA;
xii) an anti-BSG CAR comprising an ABD that specifically binds BSG;
xiii) an anti-CAIX CAR comprising an ABD that specifically binds CAIX;
xiv) an anti-CD123 CAR comprising an ABD that specifically binds CD123;
xv) an anti-CD133 CAR comprising an ABD that specifically binds CD133;
xvi) an anti-CD138 CAR comprising an ABD that specifically binds CD138;
xvii) an anti-CD19 CAR comprising an ABD that specifically binds CD19;
xviii) an anti-CD1A CAR comprising an ABD that specifically binds CD1A;
xix) an anti-CD2 CAR comprising an ABD that specifically binds CD2;
xx) an anti-CD20 CAR comprising an ABD that specifically binds CD20;
xxi) an anti-CD22 CAR comprising an ABD that specifically binds CD22;
xxii) an anti-CD24 CAR comprising an ABD that specifically binds CD24;
xxiii) an anti-CD276 CAR comprising an ABD that specifically binds CD276;
xxiv) an anti-CD3 CAR comprising an ABD that specifically binds CD3;
xxv) an anti-CD30 CAR comprising an ABD that specifically binds CD30;
xxvi) an anti-CD33 CAR comprising an ABD that specifically binds CD33;
xxvii) an anti-CD38 CAR comprising an ABD that specifically binds CD38;
xxviii) an anti-CD4 CAR comprising an ABD that specifically binds CD4;
xxix) an anti-CD43 CAR comprising an ABD that specifically binds CD43;
xxx) an anti-CD44 CAR comprising an ABD that specifically binds CD44;
xxxi) an anti-CD44v6 CAR comprising an ABD that specifically binds CD44v6;
xxxii) an anti-CD45 CAR comprising an ABD that specifically binds CD45;
xxxiii) an anti-CD5 CAR comprising an ABD that specifically binds CD5;
xxxiv) an anti-CD56 CAR comprising an ABD that specifically binds CD56;
xxxv) an anti-CD7 CAR comprising an ABD that specifically binds CD7;
xxxvi) an anti-CD70 CAR comprising an ABD that specifically binds CD70;
xxxvii) an anti-CD72 CAR comprising an ABD that specifically binds CD72;
xxxviii) an anti-CD83 CAR comprising an ABD that specifically binds CD83;
xxxix) an anti-CD84 CAR comprising an ABD that specifically binds CD84;
xl) an anti-CD99 CAR comprising an ABD that specifically binds CD99;
xli) an anti-CDCP1 CAR comprising an ABD that specifically binds CDCP1;
xlii) an anti-CDH17 CAR comprising an ABD that specifically binds CDH17;
xliii) an anti-CEA CAR comprising an ABD that specifically binds CEA;
xliv) an anti-CEACAM5 CAR comprising an ABD that specifically binds CEACAM5;
xlv) an anti-CLDN18.2 CAR comprising an ABD that specifically binds CLDN18.2;
xlvi) an anti-CLDN6 CAR comprising an ABD that specifically binds CLDN6;
xlvii) an anti-CLEC4K (CD207) CAR comprising an ABD that specifically binds CLEC4K (CD207);
xlviii) an anti-CLL-1 CAR comprising an ABD that specifically binds CLL-1;
xlix) an anti-CSPG4 CAR comprising an ABD that specifically binds CSPG4;
l) an anti-DLL3 CAR comprising an ABD that specifically binds DLL3;
li) an anti-DR5 CAR comprising an ABD that specifically binds DR5;
lii) an anti-EBV Protein CAR comprising an ABD that specifically binds EBV Protein;
liii) an anti-EGFR CAR comprising an ABD that specifically binds EGFR;
liv) an anti-EGFRvIII CAR comprising an ABD that specifically binds EGFRvII;
lv) an anti-EMR1 CAR comprising an ABD that specifically binds EMR1;
lvi) an anti-enkephalinase (CD10) CAR comprising an ABD that specifically binds enkephalinase (CD10);
lvii) an anti-EpCAM CAR comprising an ABD that specifically binds EpCAM;
lviii) an anti-EphA2 CAR comprising an ABD that specifically binds EphA2;
lix) an anti-EphA3 CAR comprising an ABD that specifically binds EphA3;
lx) an anti-FAP CAR comprising an ABD that specifically binds FAP;
lxi) an anti-FGFR4 CAR comprising an ABD that specifically binds FGFR4;
lxii) an anti-FLT3 CAR comprising an ABD that specifically binds FLT3;
lxiii) an anti-FOLR1 CAR comprising an ABD that specifically binds FOLR1;
lxiv) an anti-FSHR CAR comprising an ABD that specifically binds FSHR;
lxv) an anti-GC-C CAR comprising an ABD that specifically binds GC-C;
lxvi) an anti-GD2 CAR comprising an ABD that specifically binds GD2;
lxvii) an anti-GFRA4 CAR comprising an ABD that specifically binds GFRA4;
lxviii) an anti-Globo H CAR comprising an ABD that specifically binds Globo H;
lxix) an anti-GM2 (ganglioside M2) CAR comprising an ABD that specifically binds GM2 (ganglioside M2);
lxx) an anti-gp100 CAR comprising an ABD that specifically binds gp100;
lxxi) an anti-GPC1 CAR comprising an ABD that specifically binds GPC1;
lxxii) an anti-GPC2 CAR comprising an ABD that specifically binds GPC2;
lxxiii) an anti-GPC3 CAR comprising an ABD that specifically binds GPC3;
lxxiv) an anti-HAAH (ASPH) CAR comprising an ABD that specifically binds HAAH (ASPH);
lxxv) an anti-HER2 CAR comprising an ABD that specifically binds HER2;
lxxvi) an anti-HGF CAR comprising an ABD that specifically binds HGF;
lxxvii) an anti-HIV envelope protein gp120 CAR comprising an ABD that specifically binds HIV envelope protein gp120;
lxxviii) an anti-HIV-1 pol CAR comprising an ABD that specifically binds HIV-1 pol;
lxxix) an anti-HLA-A2 CAR comprising an ABD that specifically binds HLA-A2;
lxxx) an anti-HLA-G CAR comprising an ABD that specifically binds HLA-G;
lxxxi) an anti-HSP70 heat-shock protein CAR comprising an ABD that specifically binds HSP70 heat-shock protein;
lxxxii) an anti-ICAM-1 CAR comprising an ABD that specifically binds ICAM-1;
lxxxiii) an anti-IL-10R CAR comprising an ABD that specifically binds IL-10R;
lxxxiv) an anti-IL-13Rα2 CAR comprising an ABD that specifically binds IL-13Rα2;
lxxxv) an anti-integrin αvβ6 CAR comprising an ABD that specifically binds integrin αvβ6;
lxxxvi) an anti-ITGB7 CAR comprising an ABD that specifically binds ITGB7;
lxxxvii) an anti-KKLC1 CAR comprising an ABD that specifically binds KKLC1;
lxxxviii) an anti-KMA CAR comprising an ABD that specifically binds KMA;
lxxxix) an anti-L1CAM CAR comprising an ABD that specifically binds L1CAM;
xc) an anti-Lewis-Y antigen CAR comprising an ABD that specifically binds Lewis-Y antigen;
xci) an anti-LGR5 CAR comprising an ABD that specifically binds LGR5;
xcii) an anti-LILRB4 CAR comprising an ABD that specifically binds LILRB4;
xciii) an anti-LMP1 CAR comprising an ABD that specifically binds LMP1;
xciv) an anti-MAGEA3 CAR comprising an ABD that specifically binds MAGEA3;
xcv) an anti-M-CSF CAR comprising an ABD that specifically binds M-CSF;
xcvi) an anti-MG7 CAR comprising an ABD that specifically binds MG7;
xcvii) an anti-MICA CAR comprising an ABD that specifically binds MICA;
xcviii) an anti-MMP2 CAR comprising an ABD that specifically binds MMP2;
xcix) an anti-MSLN CAR comprising an ABD that specifically binds MSLN;
c) an anti-MUC1/MUC16 CAR comprising an ABD that specifically binds MUC1/MUC16;
ci) an anti-NCR3LG1 CAR comprising an ABD that specifically binds NCR3LG1;
cii) an anti-NECTIN2 CAR comprising an ABD that specifically binds NECTIN2;
ciii) an anti-nectin-4 CAR comprising an ABD that specifically binds nectin-4;
civ) an anti-NKG2D CAR comprising an ABD that specifically binds NKG2D;
cv) an anti-NKG2DL CAR comprising an ABD that specifically binds NKG2DL;
cvi) an anti-NR2F6 CAR comprising an ABD that specifically binds NR2F6;
cvii) an anti-NY-ESO-1 CAR comprising an ABD that specifically binds NY-ESO-1;
cviii) an anti-OPCML CAR comprising an ABD that specifically binds OPCML;
cix) an anti-PD-1 CAR comprising an ABD that specifically binds PD-1;
cx) an anti-PDGFRα CAR comprising an ABD that specifically binds PDGFRα;
cxi) an anti-PDL1 CAR comprising an ABD that specifically binds PDL1;
cxii) an anti-PLA2R CAR comprising an ABD that specifically binds PLA2R;
cxiii) an anti-PR1 CAR comprising an ABD that specifically binds PR1;
cxiv) an anti-PSCA CAR comprising an ABD that specifically binds PSCA;
cxv) an anti-PTK7 CAR comprising an ABD that specifically binds PTK7;
cxvi) an anti-PVR CAR comprising an ABD that specifically binds PVR;
cxvii) an anti-ROBO1 CAR comprising an ABD that specifically binds ROBO1;
cxviii) an anti-ROR1 CAR comprising an ABD that specifically binds ROR1;
cxix) an anti-ROR2 CAR comprising an ABD that specifically binds ROR2;
cxx) an anti-SEMA4A CAR comprising an ABD that specifically binds SEMA4A;
cxxi) an anti-SLAMF7 CAR comprising an ABD that specifically binds SLAMF7;
cxxii) an anti-TAG-72 CAR comprising an ABD that specifically binds TAG-72;
cxxiii) an anti-TGF-β CAR comprising an ABD that specifically binds TGF-β;
cxxiv) an anti-TM4SF1 CAR comprising an ABD that specifically binds TM4SF1;
cxxv) an anti-TRAIL CAR comprising an ABD that specifically binds TRAIL;
cxxvi) an anti-TRBC1 CAR comprising an ABD that specifically binds TRBC1;
cxxvii) an anti-TRBC2 CAR comprising an ABD that specifically binds TRBC2;
cxxviii) an anti-Trop-2 CAR comprising an ABD that specifically binds Trop-2;
cxxix) an anti-TSHR CAR comprising an ABD that specifically binds TSHR;
cxxx) an anti-TSLPR CAR comprising an ABD that specifically binds TSLPR;
cxxxi) an anti-ULBP1 CAR comprising an ABD that specifically binds ULBP1;
cxxxii) an anti-αvβ3 integrin CAR comprising an ABD that specifically binds αvβ3 integrin; and
cxxxiii) an anti-K light chain of human immunoglobulin CAR comprising an ABD that specifically binds K light chain of human immunoglobulin.

2. The nucleic acid of claim 1, wherein the CAR is selected from:
a) inaticabtagene autoleucel;
b) actalycabtagene autoleucel;
c) relmacabtagene autoleucel;
d) lisocabtagene maraleucel;
e) brexucabtagene autoleucel;
f) axicabtagene ciloleucel;
g) tisagenlecleucel;
h) obecabatagene autoleucel;
i) azercabtagene zapreleucel;
j) rapcabtagene autoleucel; and
k) idecabtagene vicleucel.

3. The nucleic acid of claim 1, wherein the CAR is an anti-EGFRvIII CAR comprising a means for specifically binding EGFRvIII.

4. The nucleic acid of claim 3, wherein the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO:110, and one or more of heavy chain complementary determining region 1 (HC CDR1), heavy chain complementary determining region 2 (HC CDR2), and heavy chain complementary determining region 3 (HC CDR3) of SEQ ID NO:111.

5. The nucleic acid of claim 4, wherein the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO:117).

6. The nucleic acid of claim 5, wherein the antigen binding domain comprises a light chain variable region comprising sequence SEQ ID NO:110 and a heavy chain variable region comprising sequence SEQ ID NO:111.

7. The nucleic acid of claim 1, wherein the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.

8. The nucleic acid of claim 7, wherein the sequence encoding the DR-18 polypeptide is codon optimized for expression in humans.

9. The nucleic acid of claim 8, wherein the sequence encoding the DR-18 polypeptide comprises a DR-18 encoding sequence selected from SEQ ID NOs: 67-84.

10. A cell comprising the nucleic acid, or an expression vector comprising the nucleic acid, of claim 1.

11. A method of treating a subject for cancer, the method comprising administering to the subject an effective amount of the nucleic acid of claim 1 or an expression vector or a cell comprising the nucleic acid.

12. A method, the method comprising contacting a human cell with the nucleic acid, or an expression vector comprising the nucleic acid, of claim 1 under conditions sufficient for delivery of the nucleic acid or expression vector into the human cell.

13. A nucleic acid, or expression vector comprising or consisting of the nucleic acid, comprising a sequence encoding a decoy-resistant interleukin 18 (DR-18) polypeptide, wherein the sequence encoding the DR-18 polypeptide is codon optimized for expression in human cells, and the DR-18 polypeptide comprises the following amino acid sequence: XFGKXESXLSVIRNLNDQVLFIDQGNRPLFEDMTDSDXRDNAPRTIFlISXYXDXXXRXXAVTISV KXEKISTLSXXNKIISFKEMNPPDNIKDTKSDIIFFXRXVPGHXXKXQFESSSYEGYFLAXEKERD LFKLILKKEDELGDRSIMFTXQXED (SEQ ID NO: 66), wherein the X at pos. 1 is Y, R or H; the X at pos. 5 is L, H, I or Y; the X at pos. 8 is K, Q or R; the X at pos. 38 is C or S; the X at pos. 51 is M, T, K, D, N, E or R; the X at pos. 53 is K, R, G, S or T; the X at pos. 55 is S, K or R; the X at pos. 56 is Q, E, A, R, V, G, K, L or R; the X at pos. 57 is P, L, G, A or K; the X at pos. 59 is G, A or T; the X at pos. 60 is M, K, Q, R or L; the X at pos. 68 is C, S, G, A, V, D, E or N; the X at pos. 76 is C or S; the X at pos. 77 is E or D; the X at pos. 103 is Q, E, K, P, A or R; the X at pos. 105 is S, D, N, R, K or A; the X at pos. 110 is D, K, H, N, Q, E, S or G; the X at pos. 111 is N, H, Y, D, R, S or G; the X at pos. 113 is M, V, R, T or K; the X at pos. 127 is C or S; the X at pos. 153 is V, I, T or A; and the X at pos. 155 is N, K or H.

14. The nucleic acid or expression vector of claim 13, wherein the sequence encoding the DR-18 polypeptide comprises a DR-18 encoding sequence selected from SEQ ID NOs: 67-84.

15. The nucleic acid or expression vector of claim 13 further comprising a chimeric antigen receptor (CAR) sequence encoding a CAR, wherein the CAR is an anti-EGFRvIII CAR comprising a means for specifically binding EGFRvIII.

16. The nucleic acid or expression vector of claim 15, wherein the anti-EGFRvIII CAR comprises an antigen binding domain comprising one or more of light chain complementary determining region 1 (LC CDR1), light chain complementary determining region 2 (LC CDR2), and light chain complementary determining region 3 (LC CDR3) of SEQ ID NO: 110, and one or more of heavy chain complementary determining region 1 (HC CDR1), heavy chain complementary determining region 2 (HC CDR2), and heavy chain complementary determining region 3 (HC CDR3) of SEQ ID NO:111.

17. The nucleic acid or expression vector of claim 16, wherein the antigen binding domain comprises LC CDR1 (SEQ ID NO:112), LC CDR2 (SEQ ID NO:113), and LC CDR3 (SEQ ID NO:114); and HC CDR1 (SEQ ID NO:115), HC CDR2 (SEQ ID NO:116), and HC CDR3 (SEQ ID NO: 117), optionally wherein the antigen binding domain comprises a light chain variable region comprising sequence SEQ ID NO:110 and a heavy chain variable region comprising sequence SEQ ID NO:111.

18. A cell comprising the nucleic acid or expression vector of claim 17.

19. A method of treating a subject for cancer, the method comprising administering to the subject an effective amount of the nucleic acid or expression vector of claim 17, or a cell comprising the nucleic acid or expression vector.

20. A method of making a population of therapeutic cells, the method comprising:
a) contacting a human cell with a nucleic acid comprising a chimeric antigen receptor (CAR) sequence encoding a CAR and a sequence encoding a decoy-resistant interleukin 18 (DR-18) polypeptide; wherein the nucleic acid is configured to result in expression and secretion of the DR-18 by the human cell ex vivo;
b) culturing the contacted human cell ex vivo in the presence of the secreted DR-18 polypeptide, thereby producing a population of therapeutic cells, wherein the population of therapeutic cells is enhanced as compared to a corresponding population of cells generated without the sequence encoding the DR-18 polypeptide.
